(19) United States
(12) Patent Application Publication
(10) Pub. No.: US 2007/0155764 A1
(43) Pub. Date: Jul. 5, 2007

(54) NOVEL SUBSTITUTED PYRIMIDINYLOXY UREAS USEFUL AS INHIBITORS OF PROTEIN KINASES

(75) Inventors: Hengyuan Lang, San Diego, CA (US); Timothy C. Gahman, San Diego, CA (US); Mark R. Herbert, San Diego, CA (US); Paul L. Wash, San Diego, CA (US); Cunxiang Zhao, San Deigo, CA (US); Robert L. Davis, San Diego, CA (US)

Correspondence Address: INTERNATIONAL PATENT GROUP;ATTN: MS LAVERN HALL, P.O. BOX 38129, ST. LOUIS, MO, 63138, US

(73) Assignee: KALYPSYS, INC., San Diego, CA (US)

(21) Appl. No.: 11/615,905

(22) Filed: Dec. 22, 2006

Related U.S. Application Data
(60) Provisional application No. 60/753,999, filed on Dec. 23, 2005.
(60) Provisional application No. 60/851,734, filed on Oct. 13, 2006.

(51) Int. Cl. A61K 31/513; C07D 403/02

(52) U.S. Cl. 514/269; 544/316; 544/319

(57) ABSTRACT

The present invention relates to compounds and methods useful as inhibitors of protein kinases, including B-Raf and several receptor tyrosine and cytoplasmic tyrosine kinases. The present invention is directed to new substituted pyrimidinyloxy urea compounds of Formulas II, III and IV and compositions and their application as pharmaceuticals for the treatment of disease. Methods of modulating of protein kinase activity in a human or animal subject are also provided for the treatment diseases such as cancers.

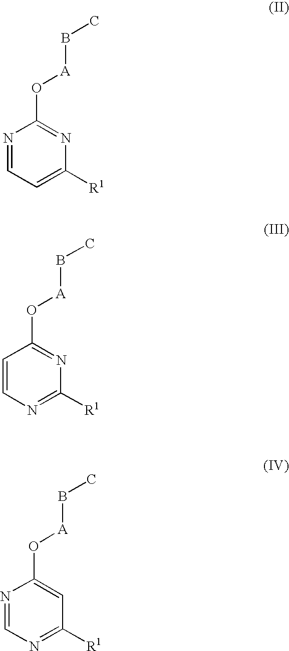

This application claims the benefit of priority of U.S. provisional application No. 60/753,999, filed Dec. 23, 2005 and U.S. provisional application No. 60/851,734, filed Oct. 13, 2006, the disclosures of which are hereby incorporated by reference as if written herein in their entireties.

### FIELD OF THE INVENTION

The present invention is directed to new substituted pyrimidinyloxy urea compounds and compositions and their application as pharmaceuticals for the treatment of disease. Methods of modulating of protein kinase activity in a human or animal subject are also provided for the treatment diseases such as cancers.

### BACKGROUND OF THE INVENTION

Protein kinases catalyze the reversible phosphorylation of serine, threonine, and tyrosine residues of many proteins in mammalian cells. The regulatory control of numerous cell functions depends in part on this post-translational modification to directly or indirectly control enzymatic activities or protein-protein interactions. For instance, growth and increase in mass, cell division, and cell survival (i.e., control of apoptosis) all depend on reversible protein phosphorylation. Dysregulation of phosphorylation is causative of or significantly contributes to a range of human diseases and accompanying pathologies. This dysregulation often takes the form of physiologically excessive protein kinase function that shifts the balance of phosphorylation toward increased serine-, threonine-, and tyrosine-phosphate in cells, resulting in hyperstimulation of key regulatory pathways (Bennasroune, A. et al., Crit Rev Oncol Hematol., 50:23-38, 2004; Fabbro, D. and Garcia-Echeverria, C., Curr Opin Drug Discov Devel., 5:701-712, 2002; Sebolt-Leopold, J. S. and Herrera R., Nat Rev Cancer, 4:937-947, 2004). The successful development of protein kinase inhibitors as therapeutics in the past decade has served to validate kinases in general for future pharmaceutical research (Beeram, M. et al., J Clin Oncol., 23:6771-6790, 2005; Blackhall, F. H. et al., Expert Opin Pharmacother., 6:995-1002, 2005; O'Dwyer, M. E. et al., Cancer Invest., 21:429-438, 2003; Sakamoto, K. M., Curr Opin Investig Drugs, 5:1329-1339, 2004).

Although highly selective allosteric kinase inhibitors are particularly desirable, the most straightforward development of small molecule kinase inhibitors has focused on the ATP binding site in the catalytic domain, with much research on reversible or irreversible ATP-competitive inhibitors (Garcia-Echeverria, C. et al., Med Res Rev., 20:28-57, 2000). Despite the sequence similarities and structural homologies that divide the protein kinase superfamily, or kinome, into various families, the requirements of ATP binding and phosphotransferase activity largely result in ATP-competitive kinase inhibitors that have selectivity profiles across the kinome, rather than exquisite selectivity for only one or a few kinase targets (Fabian, M. A. et al., Nat. Biotechnol., 23:329-336, 2005; Knight, Z. A. and Shokat, K. M., Chem Biol., 12:621-637, 2005). Some additional selectivity can be derived from small molecule interaction in another hydrophobic pocket close to but not overlapping the ATP binding site. This additional pocket is formed in those kinases where the activation loop is in the so-called “out” conformation and the kinase is in an inactive or low specific activity state. Compounds that bind in the ATP pocket and interact with this second pocket can stabilize the inactive conformation of the kinase (Okram, B. et al., Chem Biol., 13:779-786, 2006). Nevertheless, successful pharmaceutical development relies on selectivity profiles compatible with the desired therapeutic index.

With particular respect to disease, especially cancer, it is recognized that dysregulation of members of multiple kinase families can exist concurrently and contribute to pathology. In oncology applications, even the most selective of the clinically useful kinase inhibitors have a multi-kinase profile that has facilitated their successful application in tumor types with different kinase dysregulation patterns (e.g., the use of imatinib in chronic myeloid leukemia [Abl kinase] and in gastrointestinal stromal tumors [C-Kit kinase], O'Dwyer, M. E. and Druker, B. J., Lancet Oncol., 1:207-211, 2000; Steinert, D. M. et al., Expert Opin Pharmacother., 6:105-113, 2005). Our efforts have focused on the discovery of small molecule protein kinase inhibitors with selectivity profiles encompassing key kinases or kinase families described below. Particular attention is focused, but not limited to, the Raf family of serine-threonine kinases (STKs), and particular receptor tyrosine kinases (RTKs) and cytoplasmic tyrosine kinases (CTKs) implicated in both tumor cell biology and tumor blood vessel biology.

The Raf genes code for highly conserved STKs that are essential components of the Ras/Mitogen-Activated Protein Kinase (MAPK) signaling cascade (Beeram, M. et al., J Clin Oncol., 23:6771-6790, 2005). This pathway is best known for its control of a complex response to external cellular stimuli which are commonly mediated by polypeptide growth factors or other small biologically active molecules that bind and activate cell surface receptors. Raf kinases have three distinct isoforms, Raf-1 (C-Raf), A-Raf, and B-Raf, distinguished by their ability to interact with Ras, their ability to activate the MAPK pathway, and their tissue distribution and sub-cellular localization (Kolch, W., Biochem. J., 351: 289-305, 2000; Pritchard, C. A. et. al., Mol. Cell. Biol., 15:6430-6442, 1995; Weber, C. K. et. al., Oncogene, 19:169-176, 2000).

In this pathway, ligand dependent or independent activation of specific RTKs results in activation of Ras family GTPases. Raf kinases are recruited to the inner plasma membrane by active Ras and subsequently activated themselves by phosphorylation. Raf kinases then phosphorylate and activate two isoforms of Mitogen-Activated Protein Kinase Kinase (MAPKK, called Mek1 and Mek2), that are dual specificity threonine/tyrosine kinases. Both Mek isoforms phosphorylate and activate Mitogen Activated Protein Kinases 1 and 2 (MAPK, also called Extracellular Signal-Regulated Kinase 1 and 2 or Erk1 and Erk2). The MAPKs phosphorylate, in particular, various nuclear transcription factors that control gene expression in response to RTK signaling (Cobb, M. H. et al., Semin Cancer Biol., 5:261-268, 1994; Davis, R. J., Mol Reprod Dev., 42:459-467, 1995). Raf kinases are considered to be the primary Ras effectors involved in the proliferation of animal cells, and regulate many other cellular functions such as differentiation, oncogenic transformation and apoptosis (Avruch J. et al., Trends Biochem. Sci., 19:279-283, 1994; Wellbrock, C. et al., Nat Rev Mol Cell Biol., 5:875-885, 2004).

The Ras/Raf/Mek/Erk pathway is hyperactivated in about 30% of all tumors, and much higher percentages in select tumor types such as pancreatic and colon cancer (Bos, J. L., Cancer Res., 49:4682-4689, 1989; Hoshino, R. et. al., Oncogene, 18:813-822, 1999). Recent studies have shown that activating mutations in the kinase domain of B-Raf occur in about 67% of melanomas, 12% of colorectal carcinomas and 14% of ovarian carcinomas, as well as smaller percentages in other tumor types (Brose, M. S. et. al., Cancer Res., 62:6997-7000, 2002; Davies, H. et. al., Nature, 417:949-954, 2002; Yuen, S. T. et. al., Cancer Res., 62:6451-6455, 2002). These activating mutations mostly increase basal B-Raf kinase activity in cells, and uniformly increase basal levels of Erk kinase activity in cells (Wan, P. T. C. et al., Cell, 116:855-867, 2004). Greater than 80% of the B-Raf mutations in melanomas occur at a single residue, valine 600 (previously numbered 599 in some publications because of a sequence discrepancy at the amino terminus), which is substituted with a glutamic acid. Additional studies have shown that B-Raf mutation in skin nevi is a critical step in the initiation of melanocytic neoplasia (Pollock, P. M. et. al., Nature Genetics, 25:1-2, 2002). More recent studies using RNA interference to suppress expression of B-Raf (V600E mutant) in human melanoma cells have demonstrated inhibition of proliferation and induction of apoptosis (Karasarides, M. et al., Oncogene, 23:6292-6298, 2004; Sharma, A. et al., Cancer Res., 65:2412-2421, 2005). These results have underscored the attractiveness of B-Raf as a target in tumor cells that bear B-Raf mutations, especially melanoma.

RTKs are another group of kinases implicated in cancer and other diseases, through excessive expression of cognate ligands, excessive expression of wild-type RTKs (e.g., through gene amplification), or expression of mutant RTKs that are generally ligand-independent and have constitutively activated catalytic domains (Zwick, E. et al., Trends Mol. Med., 8:17-23, 2000). Especially important among the receptor tyrosine kinases implicated in cancer are those that directly mediate signaling that promotes neo-angiogenesis, or new blood vessel formation. Neo-angiogenesis is particularly critical to tumor growth and metastasis as early tumors outgrow their surrounding tissue blood supply (Folkman, J. Curr Mol Med., 3:643-651, 2003). Several other receptor tyrosine kinase activities are directly implicated in controlling lymphatic vessel growth and development, or lymphangiogenesis, which is implicated in tumor metastasis (Cao, Y., Nat Rev Cancer, 5:735-743, 2005).

Specific receptor tyrosine kinases that control and promote neo-angiogenesis are the vascular endothelial growth factor A (VEGF-A) receptors (VEGFR1, or Flt-1 and VEGFR-2 (KDR) or Flk-1), platelet-derived growth factor (PDGF) receptors alpha and beta (PDGFRα and PDGFRβ), and fibroblast growth factor (FGF) receptors (FGFR1-4), while the VEGF-C receptor (VEGFR-3 or Flt-4) controls lymphangiogenesis. Interestingly, these same RTKs can be expressed by tumor cells themselves, providing proliferation and survival signals (Wey, J. S., Clin Adv Hematol Oncol., 3:37-45, 2005). In addition, there are several other RTKs and CTKs directly implicated in cancer (Abl, C-Kit, C-Met, Flt3, Ret) that are desirable targets for the profile of multi-kinase small molecule inhibitors.

Critical signal transduction events for these receptor tyrosine kinases, especially VEGFR-2 and FGFR1, are mediated through Ras activation of Raf kinases. Raf kinase signaling can inhibit apoptosis, thereby promoting cell survival, and this function has been demonstrated particularly in endothelial cells, with implications for targeting tumor neo-angiogenesis (Alavi, A. et al., Science, 301:94-96, 2003). Therefore, small molecule kinase inhibitors whose selectivity profile includes some or all of the RTKs and CTKs referenced above, and Raf kinases, are expected to have improved utility in the direct inhibition of tumor cell proliferation and survival and the inhibition of tumor-promoting neo-angiogenesis.

### SUMMARY OF THE INVENTION

Novel compounds and pharmaceutical compositions that inhibit select disease-relevant serine-threonine kinase (STK), receptor tyrosine kinase (RTK), cytoplasmic tyrosine kinase (CTK) activity have been found, together with methods of synthesizing and using the compounds including methods for the treatment of protein kinase-mediated diseases in a patient by administering the compounds.

The present invention discloses a class of compounds, useful in treating protein kinase-mediated mediated disorders and conditions, defined by structural Formula I:

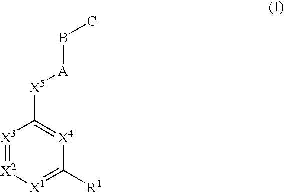

wherein

X¹-X⁴ are each independently selected from the group consisting of C(R²) and N, wherein at least one of X¹-X⁴ are N;

X⁵ is selected from the group consisting of C(R³)(R⁴), N(R³), O and S(O)ₘ;

m is 0, 1 or 2;

A and C are each independently selected from the group consisting of aryl and heteroaryl, either of which may be optionally substituted;

B is selected from the group consisting of —N(H)C(O)N(H)— and —N(H)C(O)N(H)CH₂—;

R¹ is selected from the group consisting of heteroaryl and heterocycloalkyl, either of which may be optionally substituted;

R² is selected from the group consisting of alkenyl, alkoxy, alkoxyalkyl, alkyl, alkynyl, amido, amino, aminoalkyl, cyano, cyanoalkenyl, ester, ether, halo, haloalkyl, haloalkoxy, hydrogen, hydroxy, hydroxyalkyl and nitro, any of which may be optionally substituted; and

R³ and R⁴ are each independently selected from the group consisting of lower alkyl and hydrogen.

Compounds according to the present invention possess useful protein kinase modulating activity, and may be used in the treatment or prophylaxis of a disease or condition in which protein kinase plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for modulating protein kinase. In other embodiments, the present invention provides methods for treating a protein kinase-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the modulation of protein kinase.

### DETAILED DESCRIPTION OF THE INVENTION

In certain embodiments, the compounds of the present invention have structural Formulas II, III or IV:

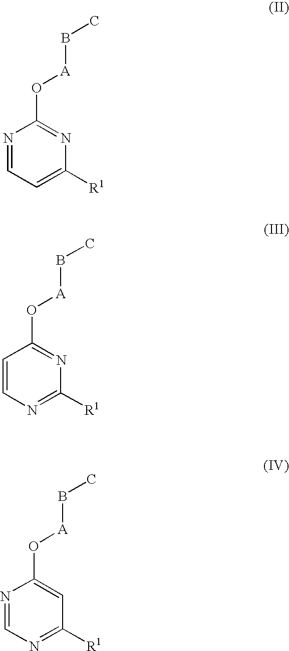

wherein

A is selected from the group consisting of aryl and heteroaryl, either of which may be optionally substituted;

C is selected from the group consisting of aryl and 6-membered heteroaryl, either of which may be optionally substituted;

B is selected from the group consisting of —N(H)C(O)N(H)— and —N(H)C(O)N(H)CH₂—;

R¹ is selected from the group consisting of heteroaryl and heterocycloalkyl, either of which may be optionally substituted.

The invention further provides for compounds of Formulas II, III or IV:

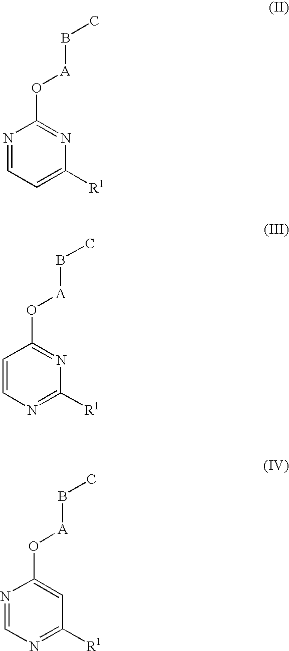

wherein

A is selected from the group consisting of aryl and heteroaryl, either of which may be optionally substituted;

C is selected from the group consisting of aryl and 6-membered heteroaryl, either of which may be optionally substituted;

B is selected from the group consisting of —N(H)C(O)N(H)— and —N(H)C(O)N(H)CH₂—;

R¹ is

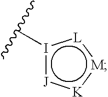

I, J, K, L and M are each independently selected from the group consisting of C(R⁵)(R⁶), S(O)ₙ, O and N(R⁷);

n is 0, 1 or 2;

R⁵ and R⁶ are each independently selected from the group consisting of alkenyl, alkoxy, alkoxyalkyl, alkyl, alkynyl, amido, amidoalkyl, amino, aminoalkyl, aminoalkylamino, cyanoalkyl, cyanoalkenyl, cycloalkyl, ester, esteralkyl, halo, haloalkyl, haloalkoxy, heteroarylalkyl, heterocycloalkenyl, heterocycloalkyl, heterocycloalkylalkyl, heterocycloalkylalkoxy, heterocycloalkylalkylthio, hydrogen, hydroxy, hydroxyalkyl, nitro and null, any of which may be optionally substituted; and

R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted.

The invention further provides for compounds of Formulas II, III or IV:

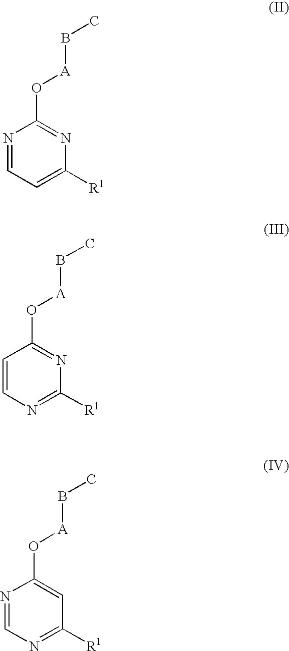

wherein

A is selected from the group consisting of aryl and heteroaryl, either of which may be optionally substituted;

C is selected from the group consisting of aryl and 6-membered heteroaryl, either of which may be optionally substituted;

B is selected from the group consisting of —N(H)C(O)N(H)— and —N(H)C(O)N(H)CH₂—;

R¹ is selected from the group consisting of

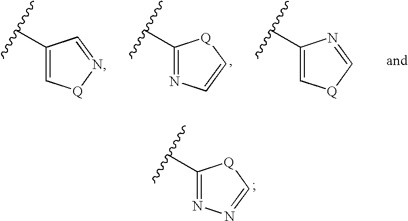

Q is selected from the group consisting of S(O)ₙ, O and N(R⁷); and

n is 0, 1 or 2; and

R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted.

The invention further provides for compounds of Formulas II, III or IV:

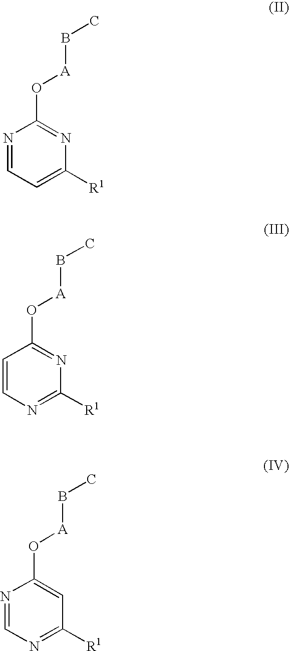

wherein:

A and C are each independently phenyl optionally substituted with halo or haloalkyl;

B is —N(H)C(O)N(H)—;

R¹ is 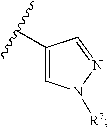
and

R⁷ is selected from the group consisting of alkyl, alkylamino, amidoalkyl, cyanoalkyl, ester, esteralkyl, haloalkyl, heterocycloalkylalkyl, hydrogen, hydroxyalkyl and null, which may be optionally substituted.

The invention further provides for compounds of Formula V:

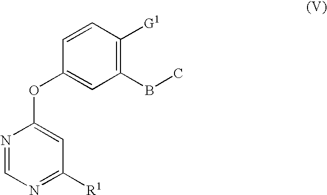

wherein:

C is phenyl optionally substituted with halo or haloalkyl;

B is —N(H)C(O)N(H)—;

R¹ is

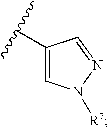

R⁷ is alkyl; and

G¹ is halo.

The invention further provides for compounds of Formula VI:

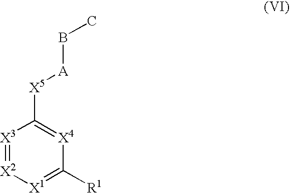

or a therapeutically acceptable salt thereof, wherein

X¹-X⁴ are each independently selected from the group consisting of C(R²) and N;

X⁵ is selected from the group consisting of C(R³)(R⁴), N(R³), O and S(O)ₘ;

m is 0, 1 or 2;

A and C are each independently selected from the group consisting of aryl and heteroaryl, either of which may be optionally substituted;

B is selected from the group consisting of —N(R⁸)C(O)N(R⁹)— and

—N(R¹⁰)C(O)N(R¹⁰)CH₂—;

R¹ is selected from the group consisting of heteroaryl and heterocycloalkyl, either of which may be optionally substituted;

R² is selected from the group consisting of alkenyl, alkoxy, alkoxyalkyl, alkyl, alkynyl, amido, amino, aminoalkyl, cyano, cyanoalkenyl, ester, ether, halo, haloalkyl, haloalkoxy, hydrogen, hydroxy, hydroxyalkyl, nitro and null, any of which may be optionally substituted;

R³ and R⁴ are each independently selected from the group consisting of lower alkyl and hydrogen;

R⁸ is selected from the group consisting of lower alkyl, cycloalkyl and heterocycloalkyl, any of which may be optionally substituted;

R⁹ is selected from the group consisting of lower alkyl, cycloalkyl, heterocycloalkyl and hydrogen, any of which may be optionally substituted; and

R¹⁰ is selected from the group consisting of lower alkyl, cycloalkyl, heterocycloalkyl and hydrogen, any of which may be optionally substituted.

The invention further provides for compounds of Formulas II, III, IV or VII:

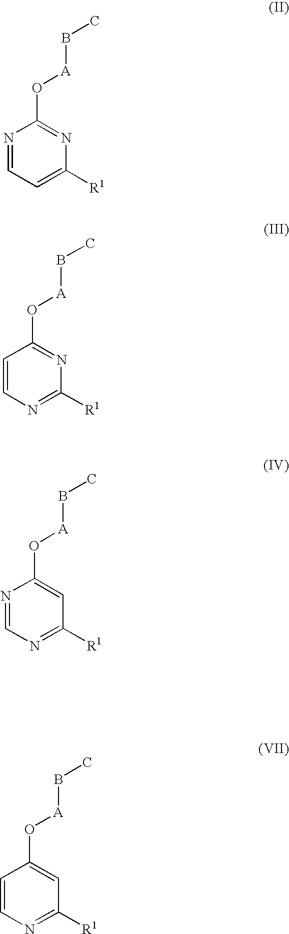

wherein

A and C are each independently selected from the group consisting of aryl and heteroaryl, either of which may be optionally substituted;

B is selected from the group consisting of —N(R⁸)C(O)N(R⁹)— and

—N(R¹⁰)C(O)N(R¹⁰)CH₂—;

R¹ is selected from the group consisting of heteroaryl and heterocycloalkyl, either of which may be optionally substituted;

R⁸ is selected from the group consisting of lower alkyl, cycloalkyl and heterocycloalkyl, any of which may be optionally substituted;

R⁹ is selected from the group consisting of lower alkyl, cycloalkyl, heterocycloalkyl and hydrogen, any of which may be optionally substituted; and

R¹⁰ is selected from the group consisting of lower alkyl, cycloalkyl, heterocycloalkyl and hydrogen, any of which may be optionally substituted.

The invention further provides for compounds of Formulas II, III, IV or VII:

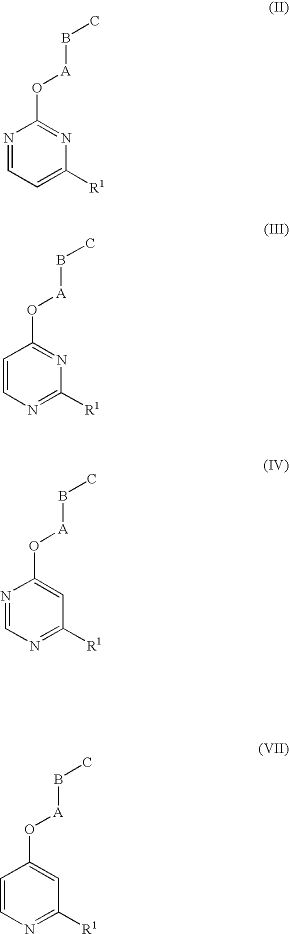

wherein

A and C are each independently selected from the group consisting of aryl and heteroaryl, either of which may be optionally substituted;

B is selected from the group consisting of —N(R⁸)C(O)N(R⁹)— and

—N(R¹⁰)C(O)N(R¹⁰)CH₂—;

R¹ is

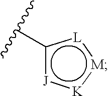

I, J, K, L and M are each independently selected from the group consisting of C(R⁵)(R⁶), S(O)ₙ, O and N(R⁷);

n is 0, 1 or 2;

R⁵ and R⁶ are each independently selected from the group consisting of alkenyl, alkoxy, alkoxyalkyl, alkyl, alkynyl, amido, amidoalkyl, amino, aminoalkyl, aminoalkylamino, cyanoalkyl, cyanoalkenyl, cycloalkyl, ester, esteralkyl, halo, haloalkyl, haloalkoxy, heteroarylalkyl, heterocycloalkenyl, heterocycloalkyl, heterocycloalkylalkyl, heterocycloalkylalkoxy, heterocycloalkylalkylthio, hydrogen, hydroxy, hydroxyalkyl, nitro and null, any of which may be optionally substituted;

R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted;

R⁸ is selected from the group consisting of lower alkyl, cycloalkyl and heterocycloalkyl, any of which may be optionally substituted;

R⁹ is selected from the group consisting of lower alkyl, cycloalkyl, heterocycloalkyl and hydrogen, any of which may be optionally substituted; and

R¹⁰ is selected from the group consisting of lower alkyl, cycloalkyl, heterocycloalkyl and hydrogen, any of which may be optionally substituted.

The invention further provides for compounds of Formulas II, III, IV or VII:

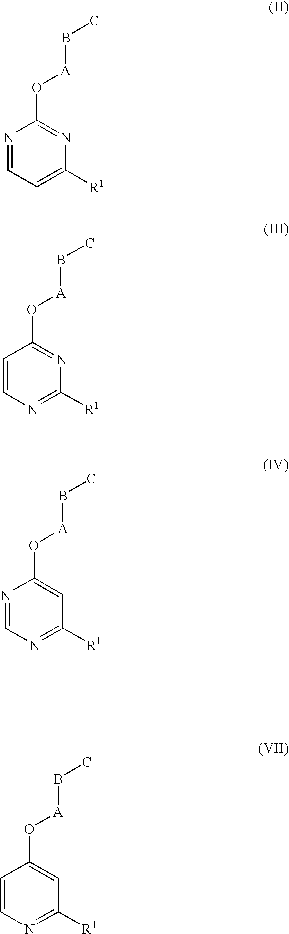

Wherein

A and C are each independently selected from the group consisting of aryl and heteroaryl, either of which may be optionally substituted;

B is selected from the group consisting of —N(R⁸)C(O)N(R⁹)— and

—N(R¹⁰)C(O)N(R¹⁰)CH₂—;

R¹ is 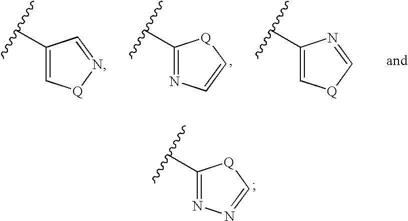
and

Q is selected from the group consisting of S(O)ₙ, O and N(R⁷);

n is 0, 1 or 2;

R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted;

R⁸ is selected from the group consisting of lower alkyl, cycloalkyl and heterocycloalkyl, any of which may be optionally substituted;

R⁹ is selected from the group consisting of lower alkyl, cycloalkyl, heterocycloalkyl and hydrogen, any of which may be optionally substituted; and

R¹⁰ is selected from the group consisting of lower alkyl, cycloalkyl, heterocycloalkyl and hydrogen, any of which may be optionally substituted.

The invention provides for compounds of Formulas I-VII for use in the inhibition of protein kinase for the treatment of disease.

The invention provides for compounds of Formulas I-VII administered in combination with another therapeutic agent.

The invention provides for compounds of Formulas I-VII for use as a medicament.

The invention provides for compounds of Formulas I-VII for use in the manufacture of a medicament for the prevention or treatment of a disease or condition ameliorated by the inhibition of protein kinase.

The invention provides for a pharmaceutical composition comprising a compound of any of Formulas I-VII together with a pharmaceutically acceptable carrier, useful for the treatment or prevention of a protein kinase-mediated disease.

The invention provides for a method of inhibition of protein kinase comprising contacting a protein kinase with a compound of any of Formulas I-VII.

The invention provides for a method of treatment of a protein kinase-mediated disease comprising the administration of a therapeutically effective amount of a compound of any of Formulas I-VII to a patient in need thereof, wherein said disease is selected from the group consisting of cancers, hematological and non-hematologic malignancies, autoimmune diseases, hematopoiesis, malignancies of the skin, psoriasis, dry eye and glaucoma.

As used herein, the terms below have the meanings indicated.

The term “acyl,” as used herein, alone or in combination, refers to a carbonyl attached to an alkenyl, alkyl aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

Protein kinase inhibitor is used herein to refer to a compound that exhibits an IC₅₀ with respect to protein kinase activity of no more than about 100 μM and more typically not more than about 50 μM, as measured in the protein kinase In vitro B-Raf/Mek1 composite kinase assay and In vitro VEGFR2 and PDGFRβ kinase assay described generally hereinbelow. IC₅₀ is that concentration of inhibitor that reduces the activity of an enzyme (e.g., B-Raf) to half-maximal level. Representative compounds of the present invention have been discovered to exhibit inhibition activity against protein kinase. Compounds of the present invention preferably exhibit an IC₅₀ with respect to protein kinase of no more than about 10 μM, more preferably, no more than about 5 μM, even more preferably not more than about 1 μM, and most preferably, not more than about 200 nM, as measured in the protein kinase assay described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Further, the compounds of the invention may be administered on a daily basis or on a schedule containing days where dosing does not take place. In certain embodiments, dosing may take place every other day. In other embodiments, dosing may take place for five consecutive days of a week, then be followed by two non-dosing days. The choice of dosing schedule will depend on many factors, including, for example, the formulation chosen, route of administration, and concurrent pharmacotherapies, and may vary on a patient-to-patient basis. It is considered within the capacity of one skilled in the art to select a schedule that will maximize the therapeutic benefit and minimize any potential side effects in a patient.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with:

For the treatment of oncologic diseases and cancers, compounds according to the present invention may be administered with an agent selected from the group comprising: aromatase inhibitors, antiestrogen, anti-androgen, or a gonadorelin agonists, topoisomerase 1 and 2 inhibitors, microtubule active agents, alkylating agents, antineoplastic, antimetabolite, dacarbazine (DTIC), or platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors.

For the treatment of oncologic diseases and solid tumors, compounds according to the present invention may be administered with an agent selected from the group comprising: dacarbazine (DTIC), alkylating agents (e.g., melphalan) anthracyclines (e.g. doxorubicin), corticosteroids (e.g. dexamethasone), Akt inhibitor (e.g. Perifosine), aromatase inhibitors, antiestrogen, anti-androgen, or a gonadorelin agonists, topoisomerase 1 and 2 inhibitors, microtubule active agents, alkylating agents (e.g. cyclophosphamide, temozolomide), antineoplastic antimetabolite, or platinum containing compounds, MITC, nitrosoureas, taxanes, lipid or protein kinase targeting agents, protein or lipid phosphatase targeting agents, anti-angiogenic agents, IMiDs (e.g. thalidomide, lenalidomide), protease inhibitors (e.g. bortezomib, NPI0052), IGF-1 inhibitors, CD40 antibody, Smac mimetics (e.g. telomestatin), FGF3 modulator (e.g. CHIR258), mTOR inhibitor (Rad 001), HDAC inhibitors (e.g. SAHA, Tubacin), IKK inhibitors, P38MAPK inhibitors, HSP90 inhibitor (e.g. 17-AAG), and other multikinase inhibitors (e.g. sorafenib).

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating protein kinase-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of protein kinase-mediated disorders.

Diseases or disorders in which B-Raf kinase plays a role, include, without limitation: oncologic, hematologic, immunologic, dermatologic and ophthalmologic diseases.

Autoimmune diseases which may be prevented or treated include, without limitation: osteoarthritis, spondyloarthropathies, systemic lupus nephritis, rheumatoid arthritis, inflammatory bowel disease, ulcerative colitis, Crohn's disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs. host disease, or psoriasis. The invention further extends to the particular autoimmune disease rheumatoid arthritis.

Hematopoiesis diseases which may be treated or prevented include, myelodysplastic disorders (MDS), and myeloproliferative disorders (polycythemia vera, myelofibrosis and essential thrombocythemia), sickle cell anemia.

Dermatologic diseases which may be treated or prevented include, without limitation, melanoma, basal cell carcinoma, squamous cell carcinoma, and other non-epithelial skin cancer as well as psoriasis and persistent itch, and other diseases related to skin and skin structure, may be treated or prevented with B-Raf inhibitors of this invention.

Ophthalmologic diseases which may be treated or prevented include, without limitation, dry eye (including Sjögren's syndrome), macular degeneration, closed and wide angle glaucoma, inflammation, and pain of the eye.

Hematological and non-hematological malignancies which may be treated or prevented include but are not limited to multiple myeloma, acute and chronic leukemias including Acute Lymphocytic Leukemia (ALL), Chronic Lymphocytic Leukemia (CLL), and Chronic Myelogenous Leukemia (CLL), lymphomas, including Hodgkin's lymphoma and non-Hodgkin's lymphoma (low, intermediate, and high grade), malignancies of the brain, head and neck, breast, lung, reproductive tract, upper digestive tract, pancreas, liver, renal, bladder, prostate and colorectal.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

The following schemes can be used to practice the present invention.

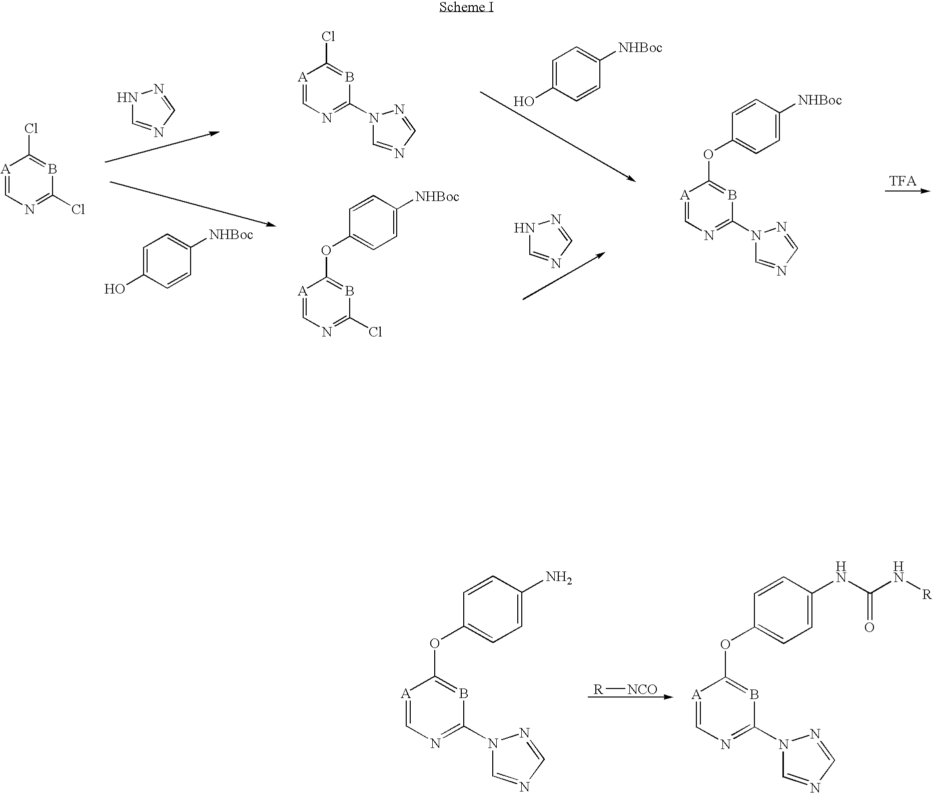

Examples 1 and 2 can be synthesized using the following general synthetic procedure set forth in Scheme I.

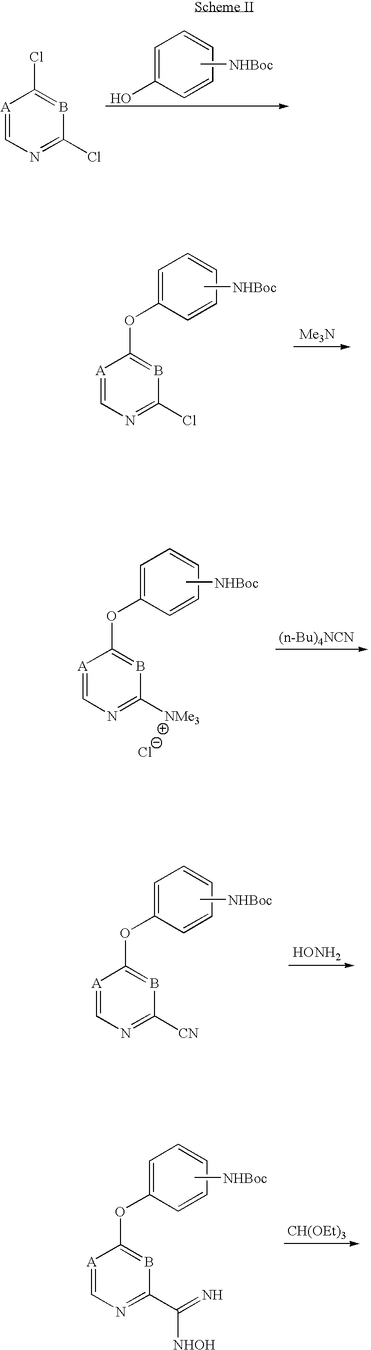

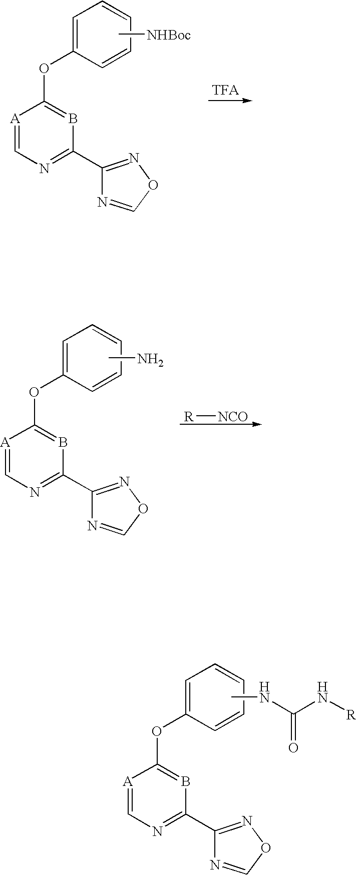

Examples 3, 7 and 10 can be synthesized using the following general synthetic procedure set forth in Scheme II.

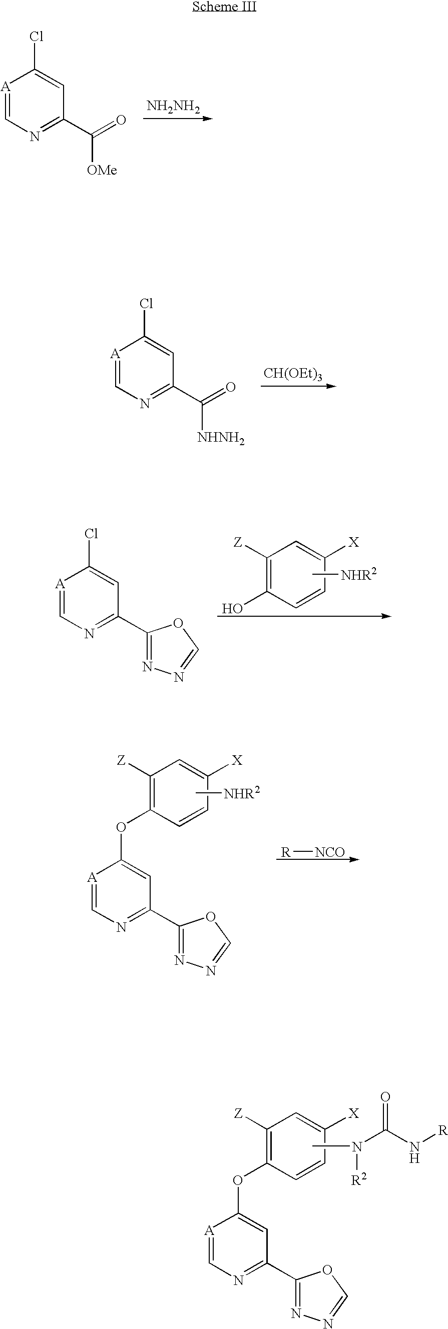

Examples 4-5, 45, 48, 110-111 and 113 can be synthesized using the following general synthetic procedure set forth in Scheme III.

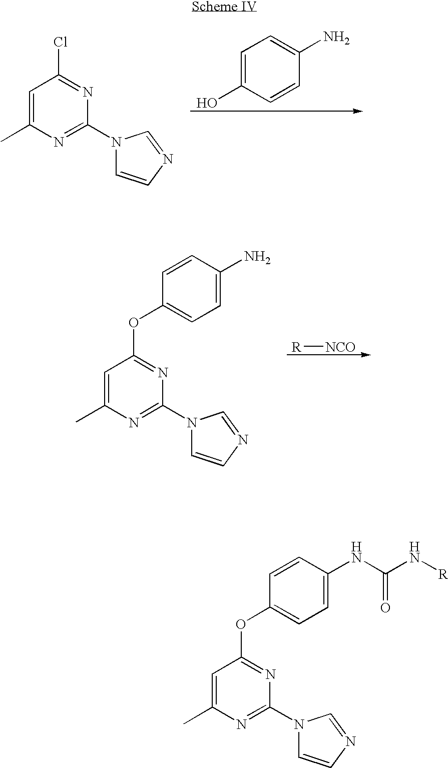

Example 6 can be synthesized using the following general synthetic procedure set forth in Scheme IV.

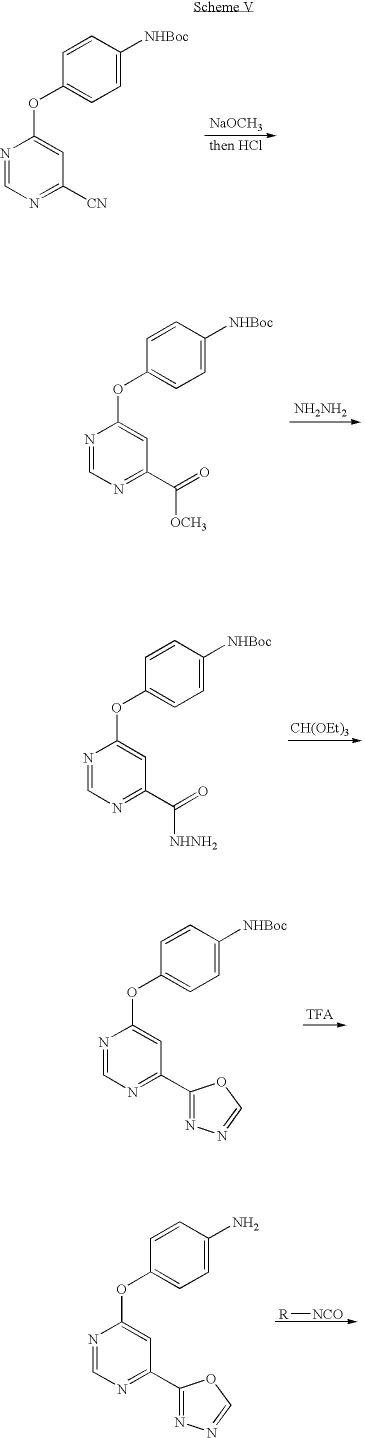

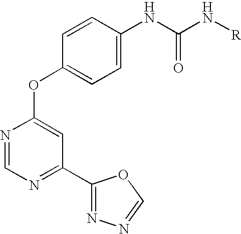

Example 8 can be synthesized using the following general synthetic procedure set forth in Scheme V.

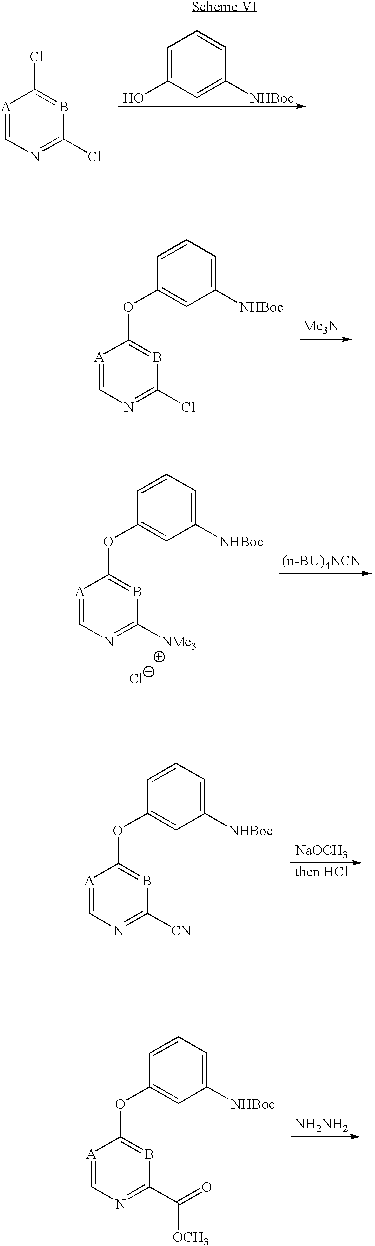

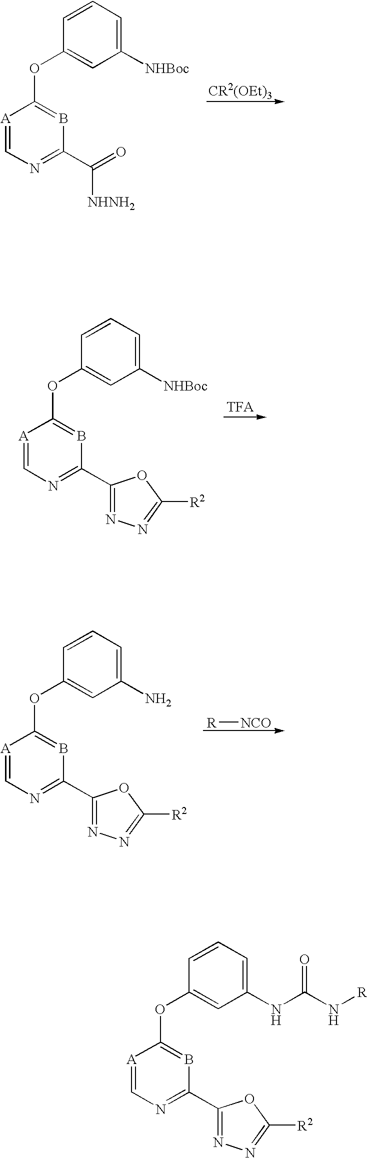

Examples 9, 11-12 and 89 can be synthesized using the following general synthetic procedure set forth in Scheme VI.

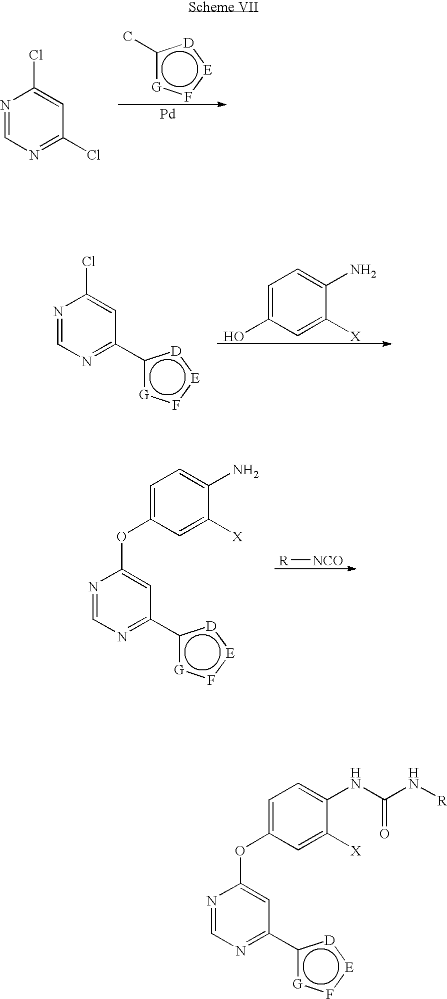

Examples 13, 30, 35, 70, 76, 92 and 101 can be synthesized using the following general synthetic procedure set forth in Scheme VII.

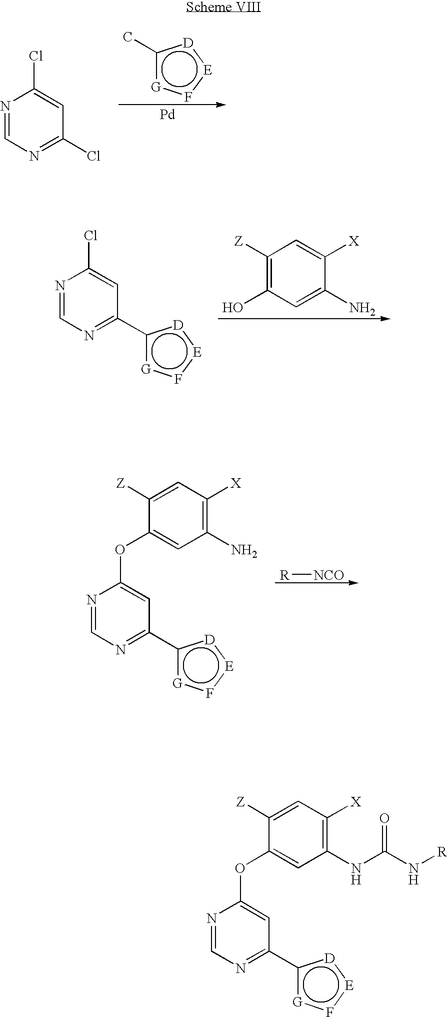

Examples 14, 29, 34, 57, 66, 69, 77, 87, 100 and 103 can be synthesized using the following general synthetic procedure set forth in Scheme VIII.

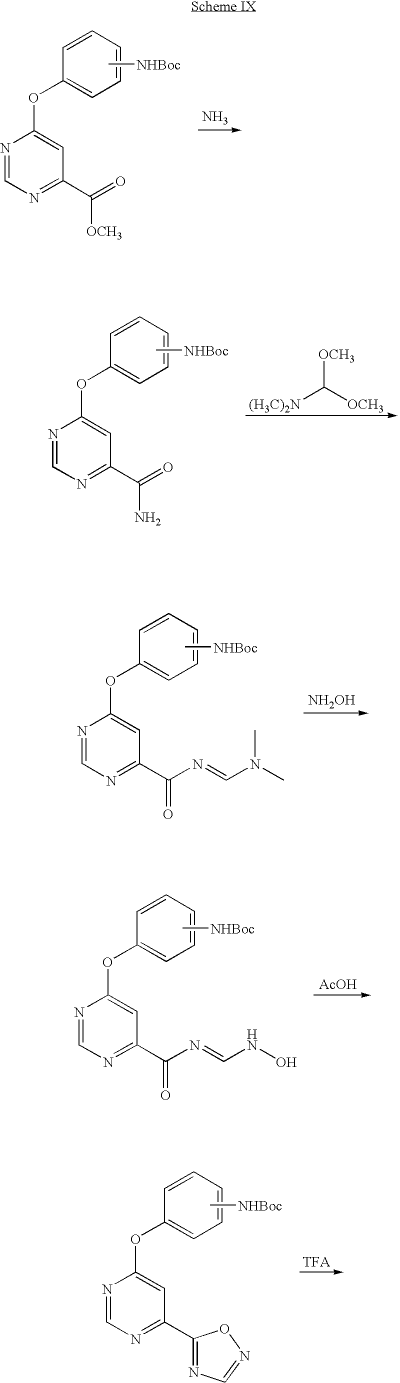

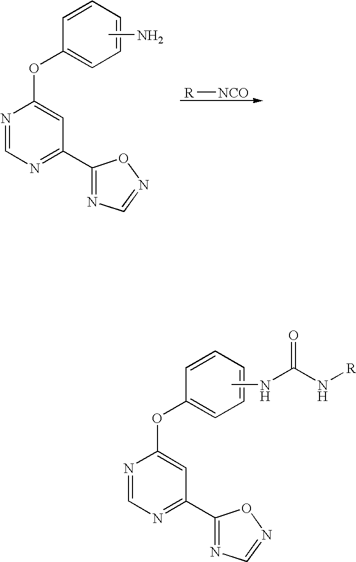

Examples 15 and 18 can be synthesized using the following general synthetic procedure set forth in Scheme IX.

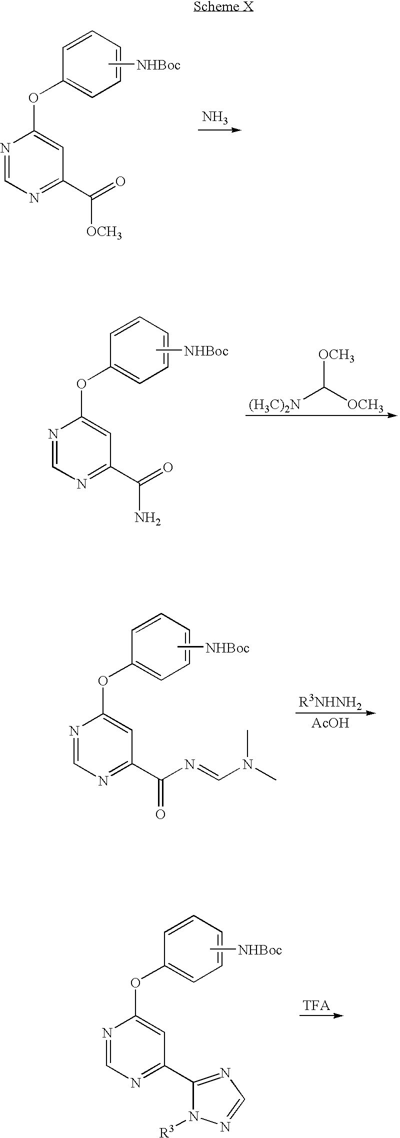

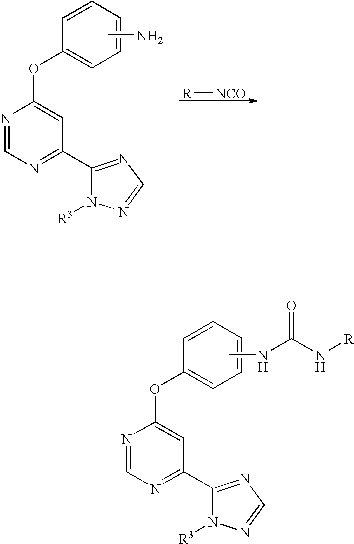

Examples 16-17 and 22-23 can be synthesized using the following general synthetic procedure set forth in Scheme X.

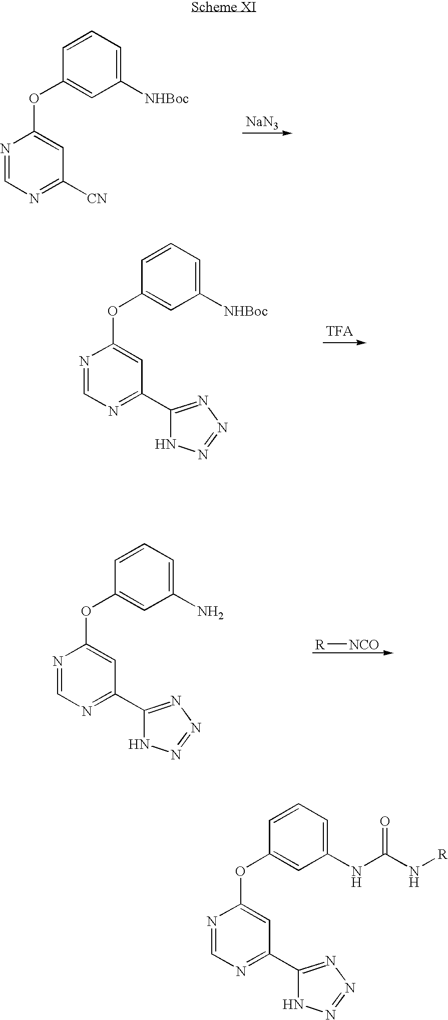

Example 19 can be synthesized using the following general synthetic procedure set forth in Scheme XI.

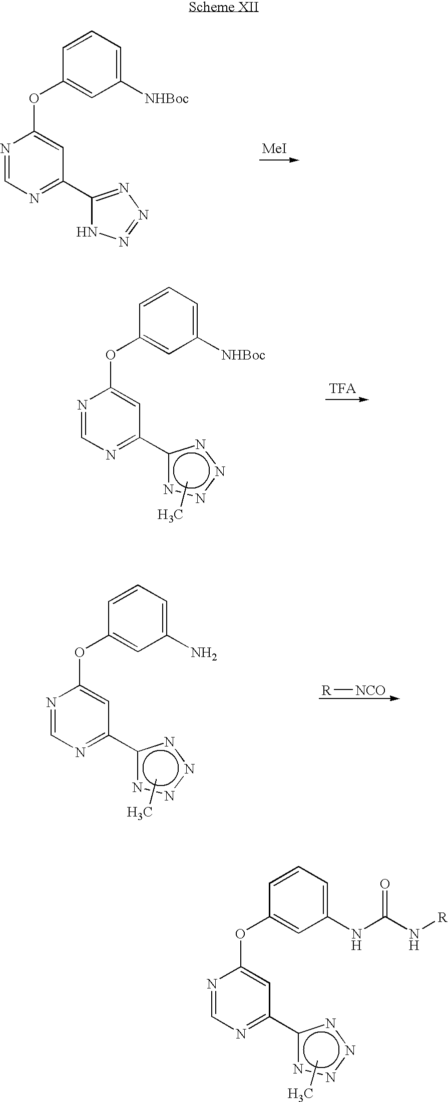

Examples 20 and 21 can be synthesized using the following general synthetic procedure set forth in Scheme XII.

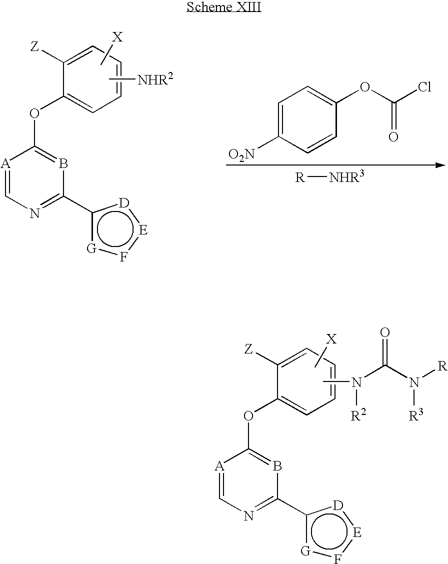

Examples 24-26, 31-33, 36-39, 41, 43-44, 47, 51-53, 75, 86, 90-91, 94-97, 104-105, 108-109 and 112 can be synthesized using the following general synthetic procedure set forth in Scheme XIII.

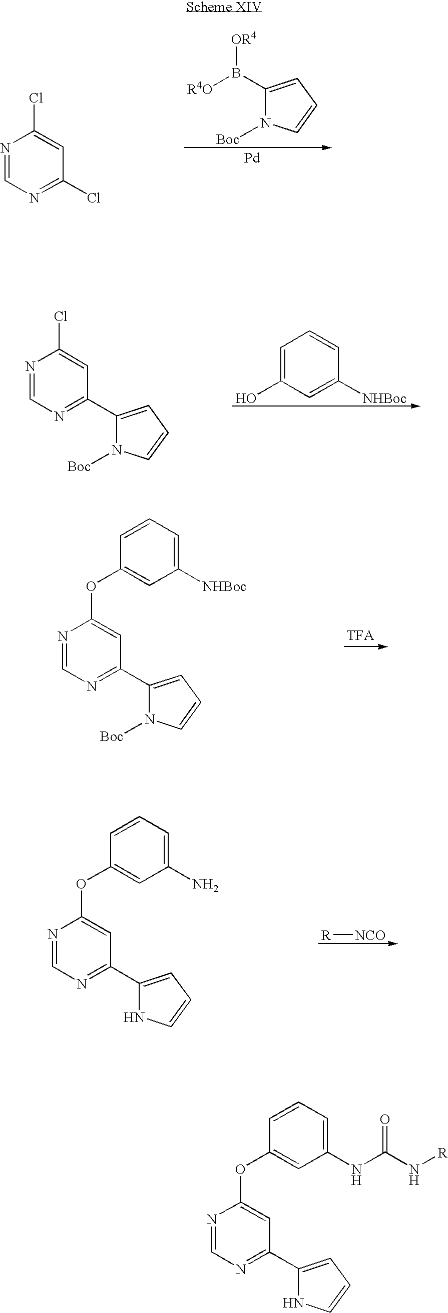

Example 27 can be synthesized using the following general synthetic procedure set forth in Scheme XIV.

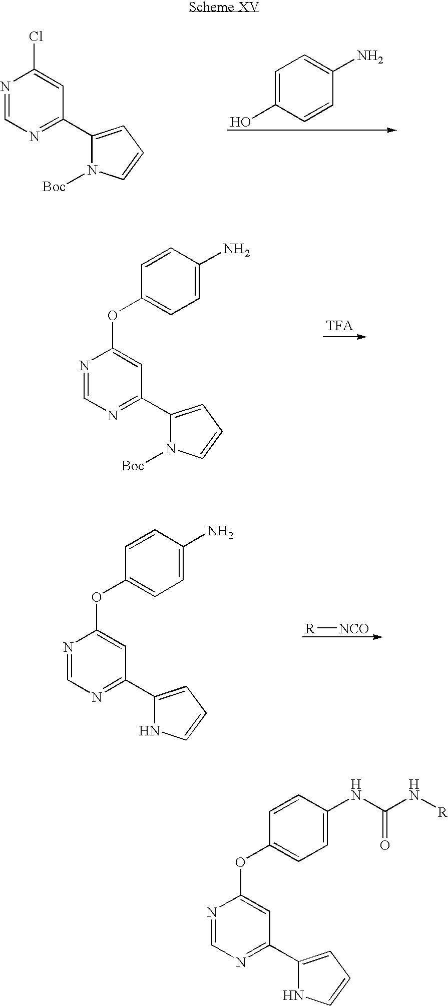

Example 28 can be synthesized using the following general synthetic procedure set forth in Scheme XV.

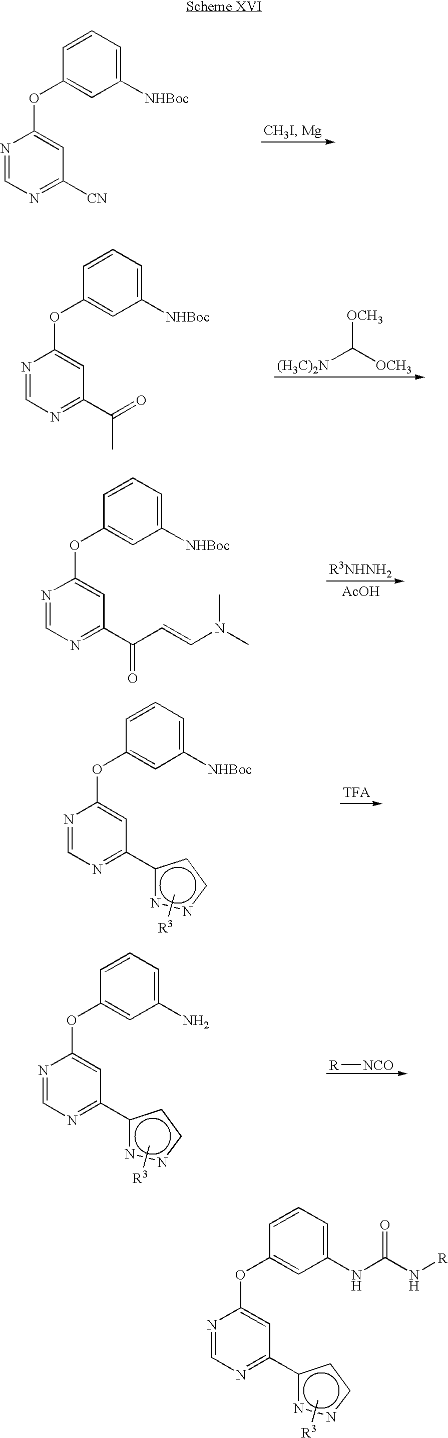

Examples 40 and 61-63 can be synthesized using the following general synthetic procedure set forth in Scheme XVI.

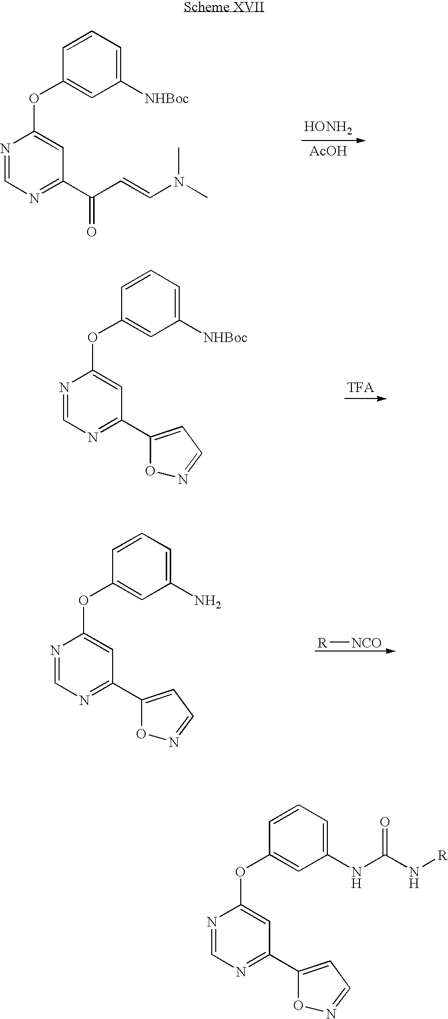

Example 42 can be synthesized using the following general synthetic procedure set forth in Scheme XVII.

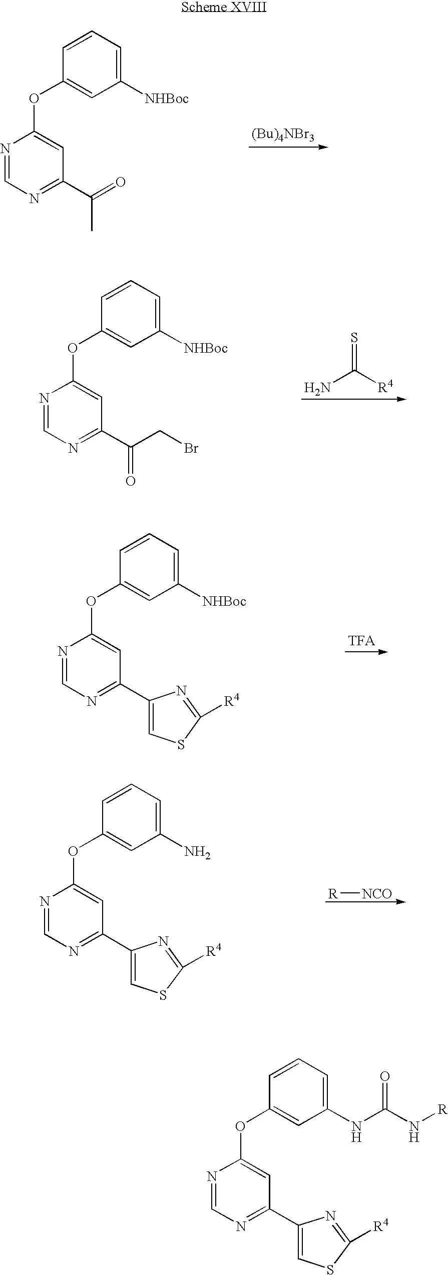

Examples 46 and 50 can be synthesized using the following general synthetic procedure set forth in Scheme XVIII.

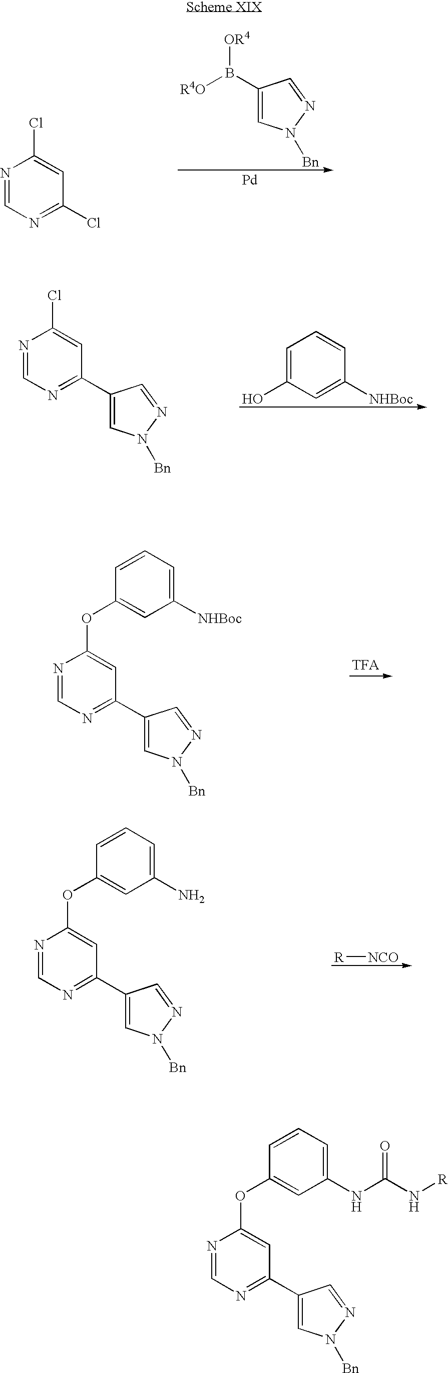

Example 49 can be synthesized using the following general synthetic procedure set forth in Scheme XIX.

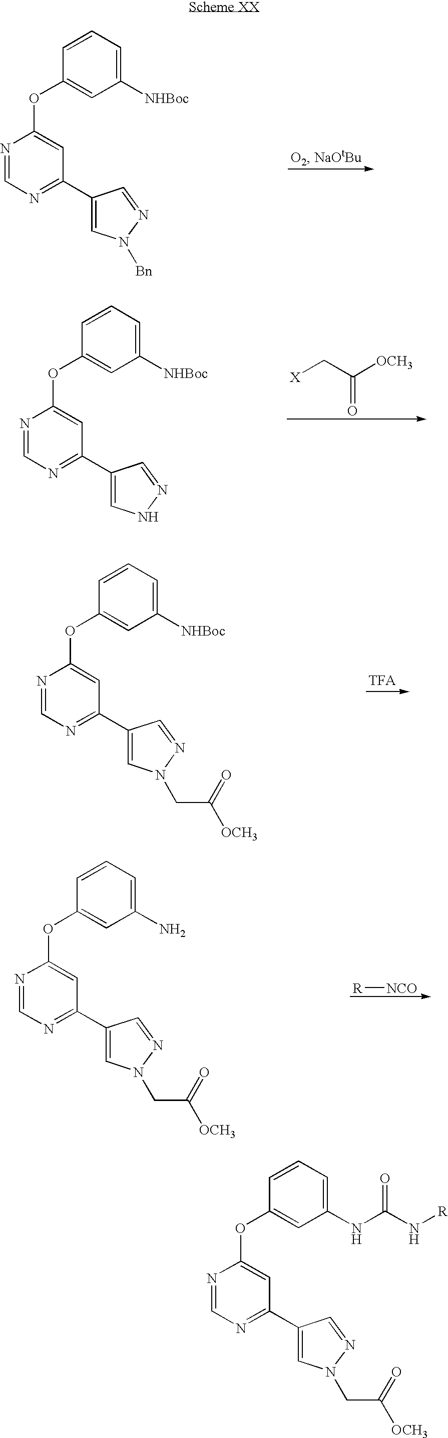

Example 54 can be synthesized using the following general synthetic procedure set forth in Scheme XX.

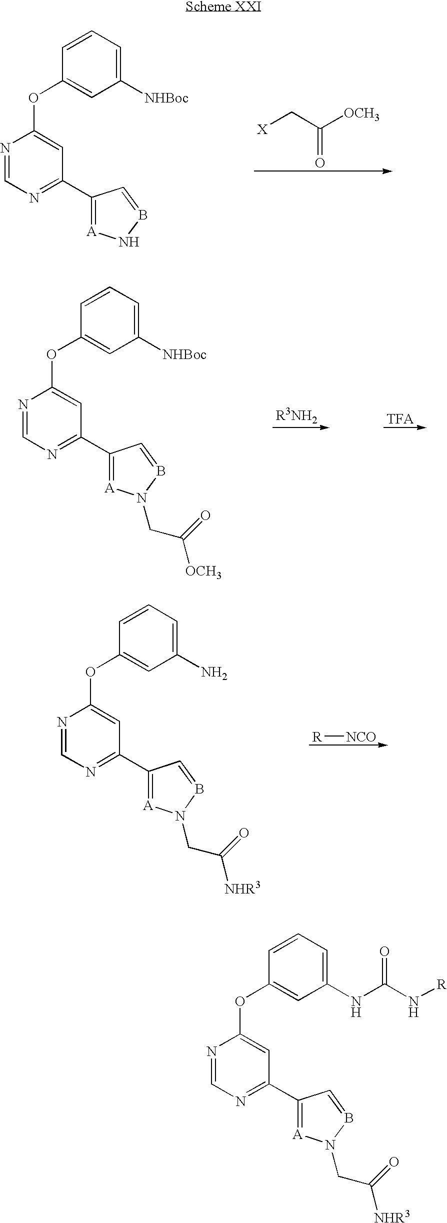

Examples 55-56 and 59-60 can be synthesized using the following general synthetic procedure set forth in Scheme XXI.

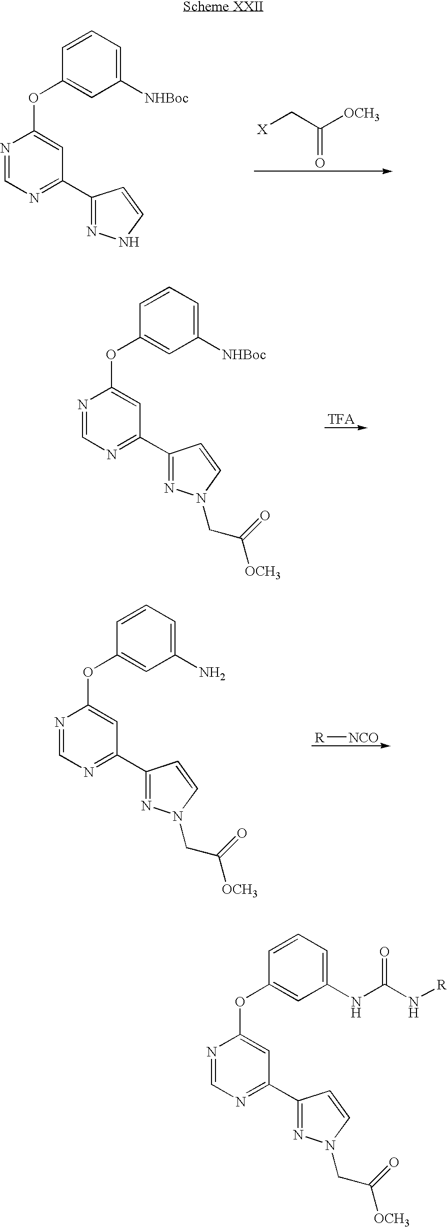

Example 58 can be synthesized using the following general synthetic procedure set forth in Scheme XXII.

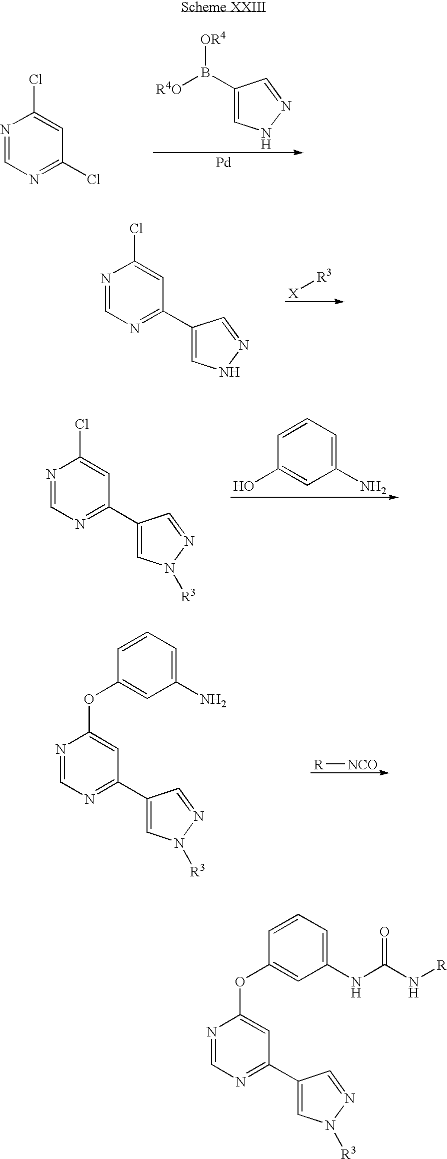

Examples 64 and 68 can be synthesized using the following general synthetic procedure set forth in Scheme XXIII.

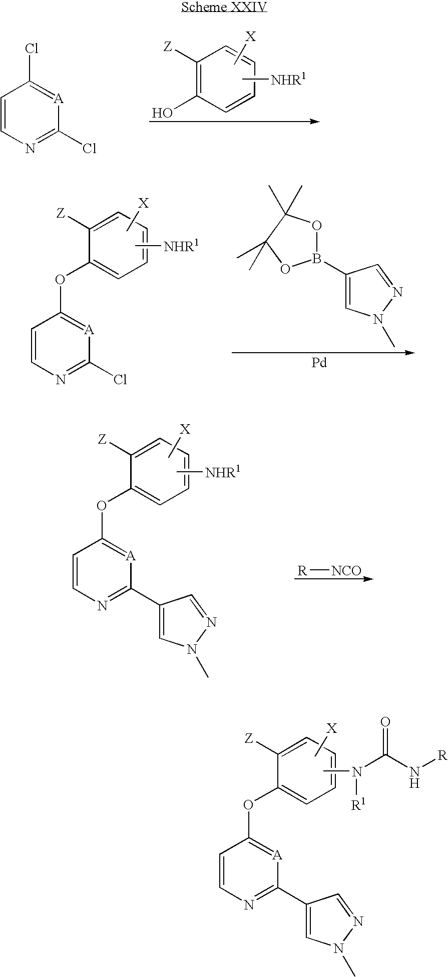

Examples 65, 67, 71, 78-80, 83-84, 88, 93, 98-99, 102 and 119 can be synthesized using the following general synthetic procedure set forth in Scheme XXIV.

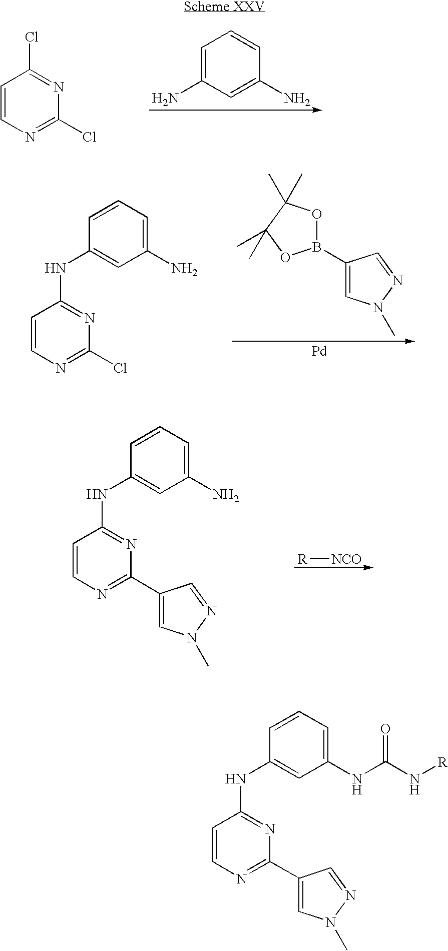

Example 72 can be synthesized using the following general synthetic procedure set forth in Scheme XXV.

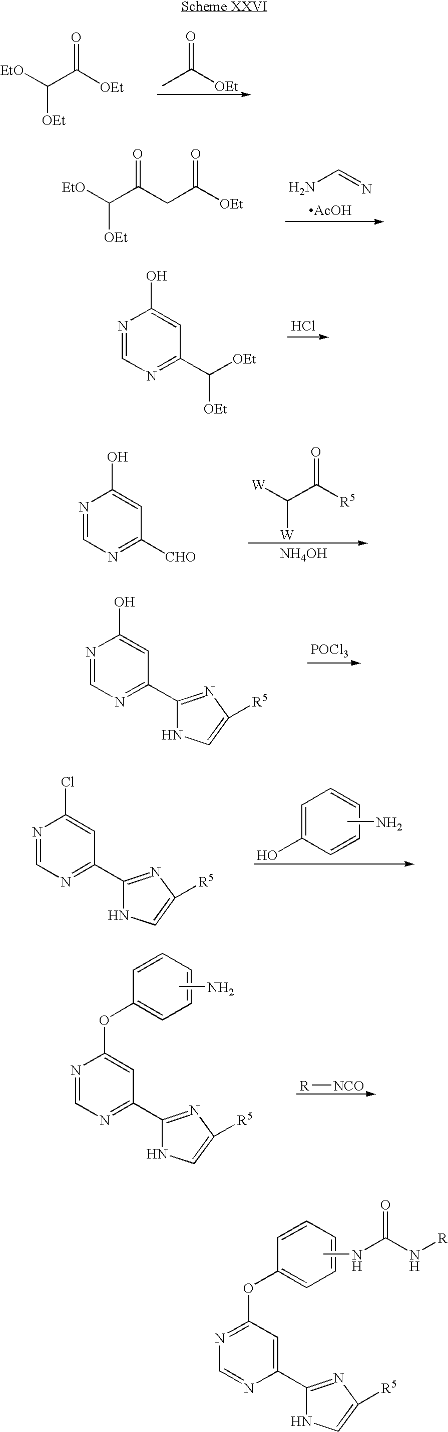

Examples 73-74 can be synthesized using the following general synthetic procedure set forth in Scheme XXVI.

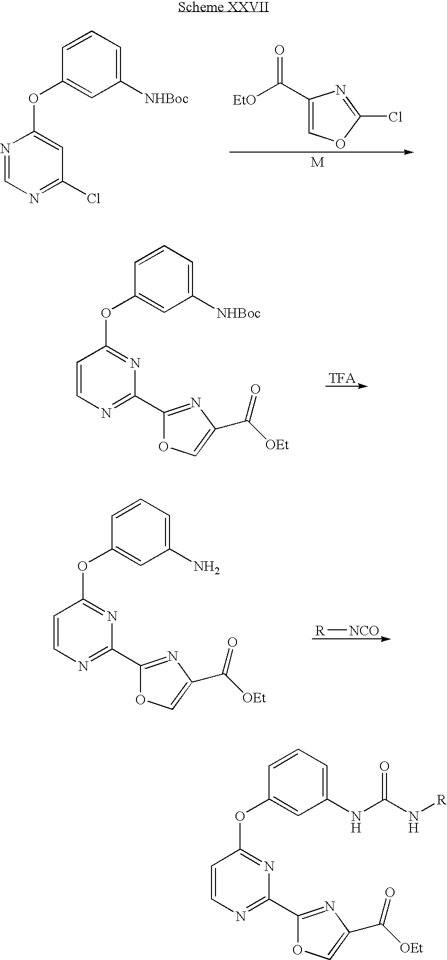

Example 81 can be synthesized using the following general synthetic procedure set forth in Scheme XXVII.

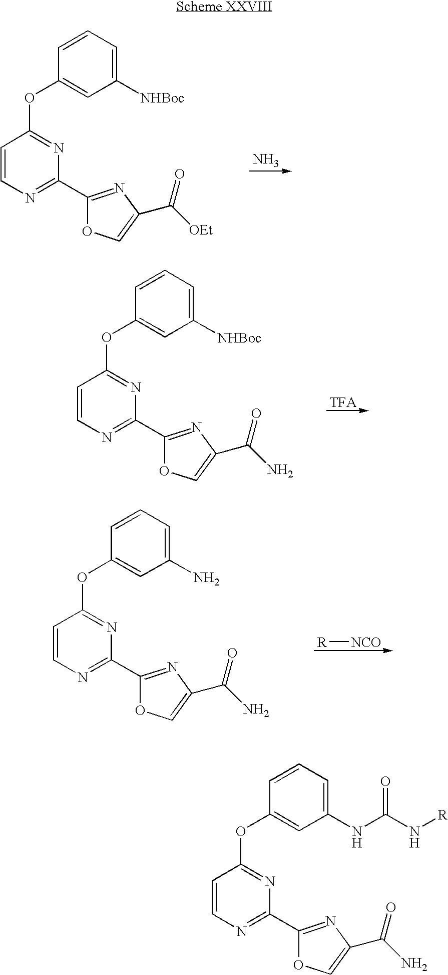

Example 82 can be synthesized using the following general synthetic procedure set forth in Scheme XXVIII.

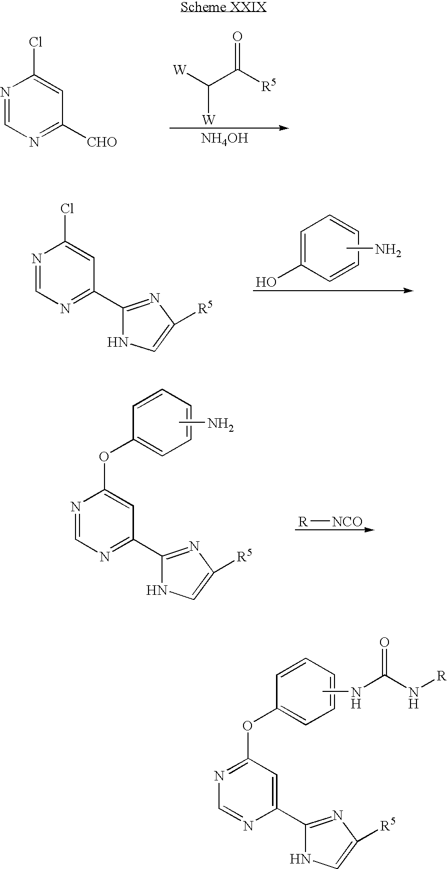

Example 85 can be synthesized using the following general synthetic procedure set forth in Scheme

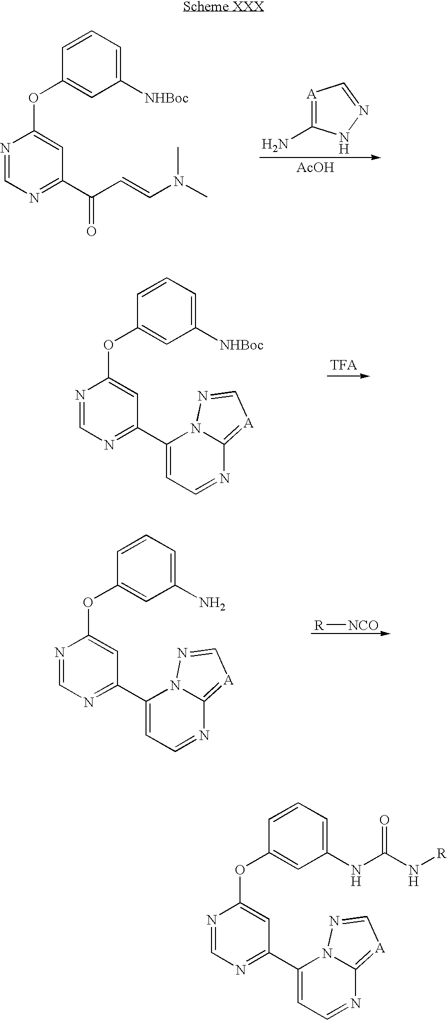

Examples 106-107 can be synthesized using the following general synthetic procedure set forth in Scheme XXX.

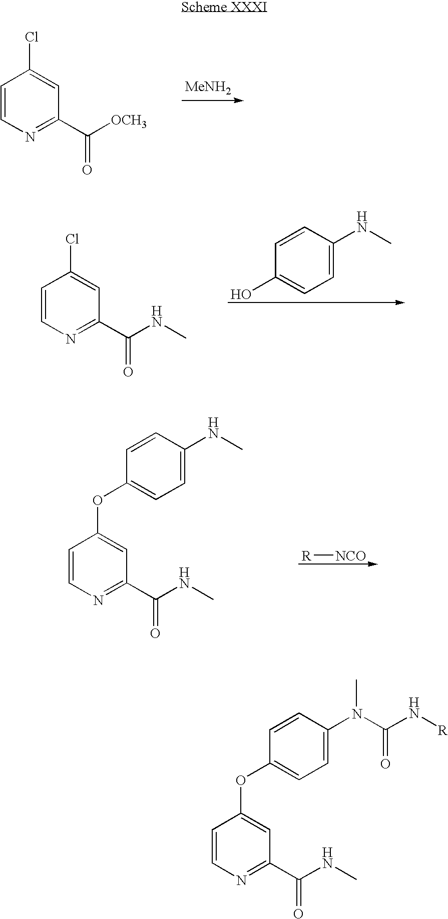

Example 114 can be synthesized using the following general synthetic procedure set forth in Scheme XXXI.

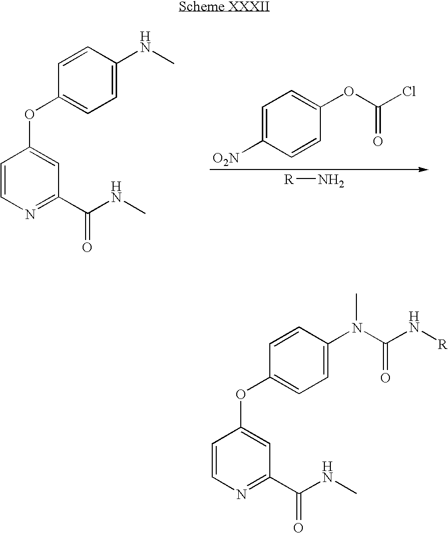

Examples 115-118 can be synthesized using the following general synthetic procedure set forth in Scheme XXXII.

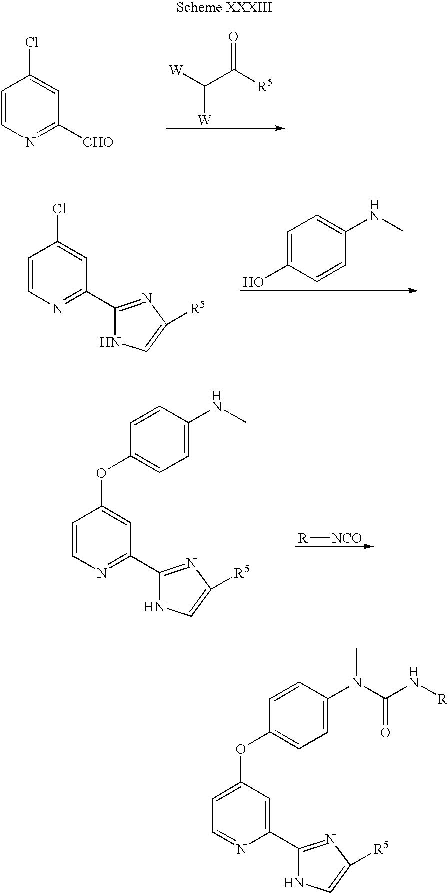

Example 120 can be synthesized using the following general synthetic procedure set forth in Scheme XXXIII.

The invention is further illustrated by the following examples.

### EXAMPLE 1

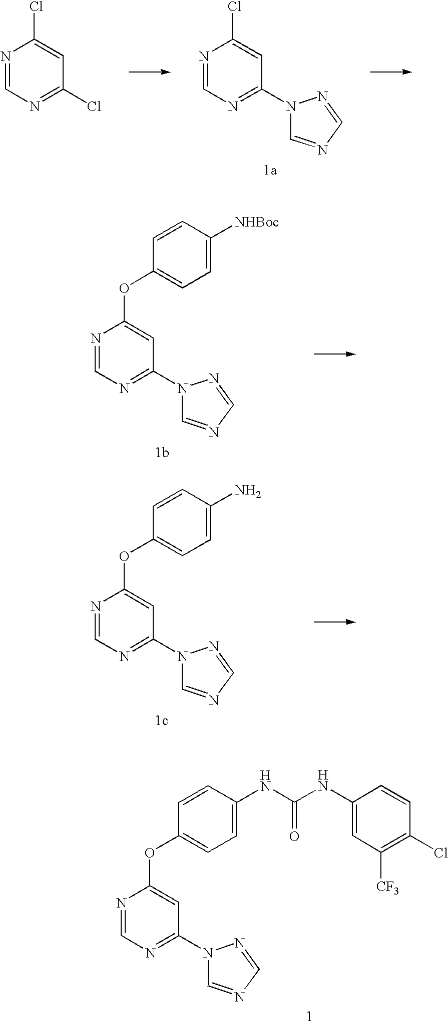 Step 1

### Preparation of compound 1a: 4-Chloro-6-[1,2,4]triazol-1-yl-pyrimidine

A mixture of 4,6-dichloropyrimidine (2.96 g, 19.9 mmol) and THF (10 mL) was added to a −20° C. solution of 1H-1,2,4-triazole (1.38 g, 20.0 mmol), sodium hydroxide (880 mg, 22.0 mmol) and THF (30 mL) under nitrogen. The reaction mixture was warmed to room temperature over 2 h and stirred for an additional 12 h. The mixture was concentrated under vacuum prior to the addition of water (30 mL) and ethyl acetate (30 mL). The phases were separated and the organic layer was concentrated under vacuum. The product was purified using column chromatography (hexanes to 1:1 hexanes/ethyl acetate) to give 700 mg of 4-chloro-6-[1,2,4]triazol-1-yl-pyrimidine as a white solid. ¹H NMR (400 MHz, DMSO) δ 9.52 (s, 1H), 9.02 (s, 1H), 8.42 (s, 1H), 7.99 (s, 1H).

Step 2

### Preparation of compound 1b: [4-(6-[1,2,4]Triazol-1-yl-pyrimidin-4-yloxy)-phenyl]-carbamic acid tert-butyl ester

Potassium t-butoxide (280 mg, 2.50 mmol) was added to a solution of tert-butyl 4-hydroxyphenylcarbamate (630 mg, 3.01 mmol) in DMSO (15 mL) at room temperature under nitrogen. The reaction mixture was stirred for 30 minutes prior to the addition of a solution of 4-chloro-6-(1H-1,2,4-triazol-1-yl)pyrimidine (320 mg, 1.77 mmol) in DMSO (15 mL). The resulting solution was stirred at room temperature for 1 h. Water (20 ml) and ethyl acetate (100 mL) were added and the phases were separated. The aqueous phase was back extracted with ethyl acetate (2×100 mL) and the combined organic layers were concentrated under vacuum. The product was purified by a methylene chloride recrystallization to give 300 mg of [4-(6-[1,2,4]triazol-1-yl-pyrimidin-4-yloxy)-phenyl]-carbamic acid tert-butyl ester as a white solid. [M−H]⁺ 353.00.

Step 3

### Preparation of compound 1c: 4-(6-[1,2,4]Triazol-1-yl-pyrimidin-4-yloxy)phenylamine

A mixture of tert-butyl 4-(6-(1H-1,2,4-triazol-1-yl)pyrimidin-4-yloxy)phenylcarbamate (300 mg, 0.85 mmol), DCM (50 mL) and 2,2,2-trifluoroacetic acid (5 mL) was stirred at room temperature for 3 h under nitrogen. The solution was concentrated under vacuum prior to the addition of H₂O (50 mL). The pH was adjusted to 7-8 with saturated NaHCO₃ solution and the resulting precipitate was collected by vacuum filtration to give 200 mg of 4-(6-(1H-1,2,4-triazol-1-yl)pyrimidin-4-yloxy)phenylamine as a white solid. [M+H]⁺ 255.00.

Step 4

Preparation of compound 1: 1-(4-Chloro-3-trifluoromethyl-phenyl)-3-[4-(6-[1,2,4]triazol-1-yl-pyrimidin-4-yloxy)-phenyl]-urea

A mixture of 4-(6-(1H-1,2,4-triazol-1-yl)pyrimidin-4-yloxy)benzenamine (127 mg, 500 μmol), 4-chloro-3-trifluoromethylphenylisocyanate (111 mg, 500 μmol) and DCM (5 ml) was stirred at room temperature for 1 h under nitrogen. The resulting precipitate was collected by vacuum filtration to give 200 mg of 1-(4-chloro-3-trifluoromethyl-phenyl)-3-[4-(6-[1,2,4]triazol-1-yl-pyrimidin-4-yloxy)-phenyl]-urea as a white solid. [M−H]⁺ 474.00; ¹H NMR (400 MHz, DMSO) δ 9.50 (s, 1H), 9.21 (s, 1H), 8.97 (s, 1H), 8.80 (s, 1H), 8.40 (s, 1H), 8.13-8.12 (d, 1H), 7.68-7.61 (m, 2H), 7.58-7.56 (d, 2H), 7.29 (s, 1H), 7.24-7.22 (d, 2H).

### EXAMPLE 2

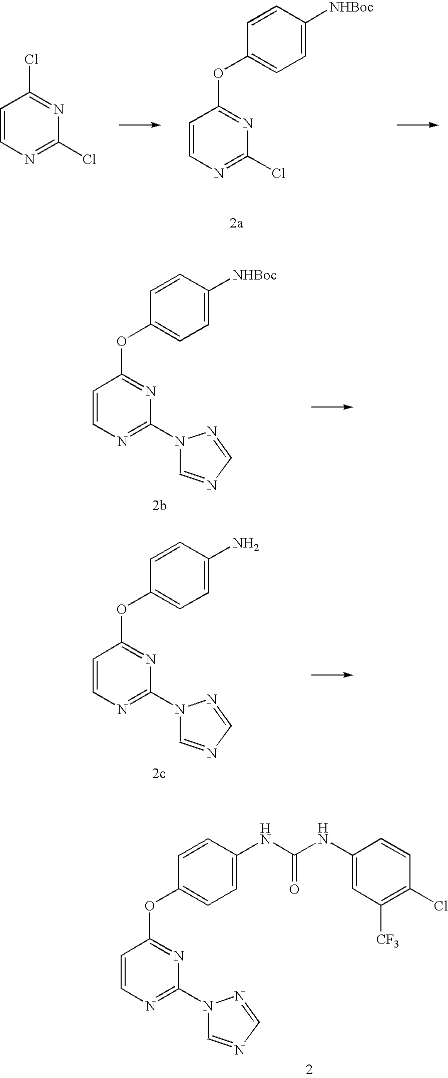 Step 1

Preparation of compound 2a: tert-Butyl 4-(2-chloropyrimidin-4-yloxy)phenylcarbamate was prepared following the procedures described in preparation of Example 1. [M−H]⁺ 320.00.

Step 2

### Preparation of compound 2b: [4-(2-[1,2,4]Triazol-1-yl-pyrimidin-4-yloxy)-phenyl]-carbamic acid tert-butyl ester

Sodium hydride (150 mg, 3.75 mmol) was added to a solution of 1H-1,2,4-triazole (270 mg, 3.91 mmol) in DMSO (20 ml) at room temperature under nitrogen. The reaction mixture was stirred for 30 minutes prior to the addition of a solution of tert-butyl 4-(2-chloropyrimidin-4-yloxy)phenylcarbamate (960 mg, 2.99 mmol) in DMSO (20 mL). The mixture was heated to 80° C. for 2 h, then cooled to room temperature. Water (200 mL) and ethyl acetate (100 mL) were added and the phases were separated. The aqueous layer was back extracted with ethyl acetate (100 mL) and the combined organic layers were concentrated under vacuum to give 1.00 g of tert-butyl 4-(2-(1H-1,2,4-triazol-1-yl)-pyrimidin-4-yloxy)phenylcarbamate as a light brown solid. [M−H]⁺ 353.00.

Step 3

### Preparation of compound 2c: 4-(2-[1,2,4]Triazol-1-yl-pyrimidin-4-yloxy)-phenylamine

2,2,2-Trifluoroacetic acid (5 mL) was added to a solution of tert-butyl 4-(2-(1H-1,2,4-triazol-1-yl)pyrimidin-4-yloxy)phenylcarbamate (354 mg, 1.00 mmol) in DCM (50 ml) at room temperature under nitrogen. The reaction mixture was stirred for 3 h at room temperature prior to concentrating under vacuum. Brine (30 mL) was added and the pH was adjusted to 9-10 using solid Na₂CO₃. The resulting solution was extracted with ethyl acetate (3×100 mL) and the combined organics were concentrated under vacuum. The product was purified using column chromatography (hexanes to ethyl acetate) to give 100 mg of 4-(2-(1H-1,2,4-triazol-1-yl)pyrimidin-4-yloxy)phenylamine as a yellow solid. [M−H]⁺ 253.00; ¹H NMR (300 MHz, DMSO) δ 9.34 (s, 1H), 8.72 (d, 1H), 8.40 (s, 1H), 7.56 (d, 1H), 6.89 (d, 2H), 6.59 (d, 2H), 5.05 (s, 2H).

Step 4

Preparation of compound 2: 1-(4-Chloro-3-trifluoromethyl-phenyl)-3-[4-(2-[1,2,4]triazol-1-yl-pyrimidin-4-yloxy)-phenyl]-urea was prepared following the procedures described in preparation of Example 1. [M−H]⁺ 474.00; ¹H NMR (300 MHz, DMSO) δ 9.34 (s, 1H), 9.17 (s, 1H), 8.91 (s, 1H), 8.78 (s, 1H), 8.42 (s, 1H), 8.12 (s, 1H), 7.66 (m, 3H), 7.57 (m, 2H), 5.76 (s, 2H).

### EXAMPLE 3

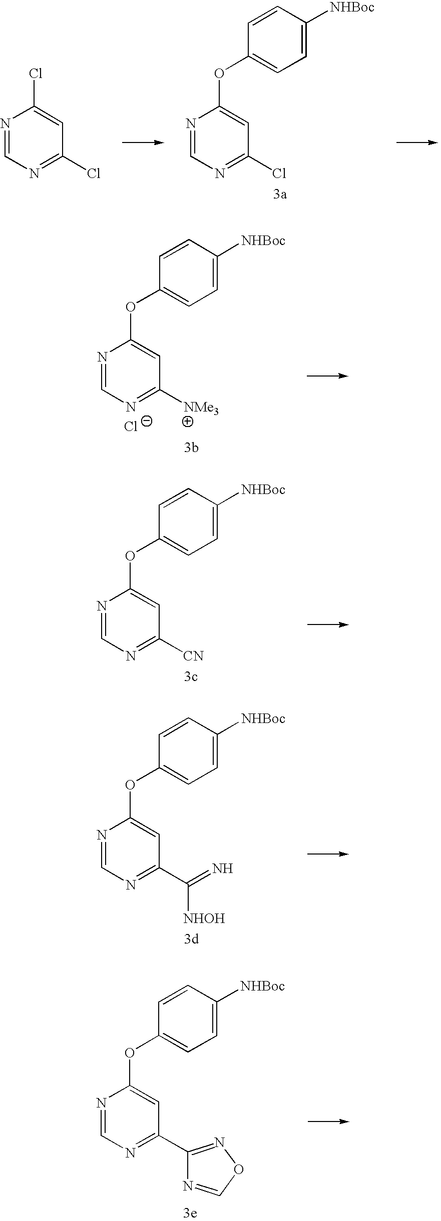 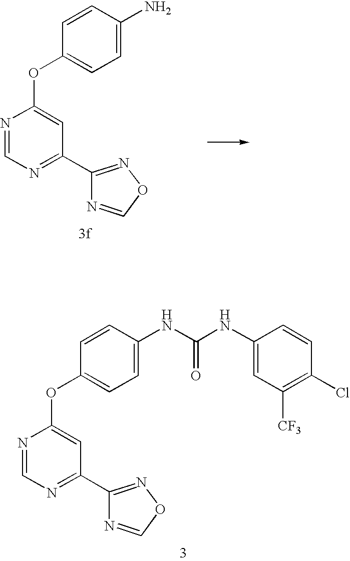 Step 1

Preparation of compound 3a: tert-Butyl 4-(2-chloropyrimidin-4-yloxy)phenylcarbamate was prepared following the procedures described in preparation of Example 2. [M−H]⁺ 320.00.

Step 2

### Preparation of compound 3b: [4-(4-tert-Butoxycarbonylphenoxy)pyrimidin-6-yl]trimethylammonium chloride

Anhydrous trimethylamine (5.90 g, 99.8 mmol) was added dropwise over 20 minutes to a stirred solution of tert-butyl 4-(6-chloropyrimidin-4-yloxy)phenylcarbamate (3.21 g, 10.0 mmol) in benzene (100 mL) at room temperature under nitrogen. The reaction mixture was stirred for 48 h at room temperature and the resulting precipitate was collected by vacuum filtration. The solid was washed with benzene (100 mL) and dried under vacuum to give 3.00 g of [4-(4-tert-butoxycarbonylphenoxy)pyrimidin-6-yl]trimethylammonium chloride as a brown solid. [M+H]⁺ 345.20.

Step 3

### Preparation of compound 3c: tert-Butyl 4-(6-cyanopyrimidin-4-yloxy)phenylcarbamate

A mixture of [4-(4-tert-butoxycarbonylphenoxy)pyrimidin-6-yl]trimethylammonium chloride (2.62 g, 6.88 mmol), tetrabutylammonium cyanide (2.03 g, 7.57 mmol) and DCM (80 ml) was heated to 40° C. for 12 h. The reaction mixture was cooled to room temperature and water (100 mL) was added. The phases were separated and the organic layer was concentrated under vacuum. The product was purified using column chromatography (hexanes to 1:1 hexanes/ethyl acetate) to give 1.00 g of tert-butyl 4-(6-cyanopyrimidin-4-yloxy)phenylcarbamate as a white solid. [M−H]⁺ 311.00; ¹H NMR (400 MHz, DMSO) δ 8.89 (s, 1H), 7.90 (s, 1H), 7.52 (d, 2H), 7.14 (d, 2H).

Step 4

### Preparation of compound 3d: tert-Butyl 4-(6-(N-hydroxycarbamimidoyl)pyrimidin-4-yloxy)phenylcarbamate

Na₂CO₃ (500 mg, 4.72 mmol) was added to a solution of hydroxylamine hydrochloride (3.00 g, 43.2 mmol) in water (10 mL) at room temperature. The reaction mixture was stirred for 30 minutes prior to the addition of a solution of tert-butyl 4-(6-cyanopyrimidin-4-yloxy)phenylcarbamate (500 mg, 1.60 mmol) in EtOH (40 mL). The mixture was stirred for 2 h at room temperature then was concentrated under vacuum. Water (50 mL) and ethyl acetate (50 mL) were added and the phases were separated. The aqueous layer was back extracted with ethyl acetate (50 mL) and the combined organic layers were concentrated under vacuum to give 500 mg of tert-butyl 4-(6-(N-hydroxycarbamimidoyl)pyrimidin-4-yloxy)phenylcarbamate as a white solid. [M−H]⁺ 344.00.

Step 5

### Preparation of compound 3e: [4-(6-[1,2,4]Oxadiazol-3-yl-pyrimidin-4-yloxy)-phenyl]-carbamic acid tert-butyl ester

A mixture of tert-butyl 4-(6-(N-hydroxycarbamimidoyl)pyrimidin-4-yloxy)phenylcarbamate (400 mg, 1.16 mmol) and triethyl orthoformate (10 mL) was stirred at room temperature for 72 h under nitrogen. The solution was concentrated under vacuum and the product was purified using column chromatography (hexanes to ethyl acetate) to give 200 mg of [4-(6-[1,2,4]oxadiazol-3-yl-pyrimidin-4-yloxy)-phenyl]-carbamic acid tert-butyl ester as a white solid. [M−H]⁺ 354.00.

Step 6

Preparation of compound 3f: 4-(6-[1,2,4]Oxadiazol-3-yl)pyrimidin-4-yloxy)-phenylamine was prepared following the procedures described in preparation of Example 2. [M+H]⁺ 256.00; ¹H-NMR (400 MHz, DMSO) δ 9.85 (s, 1H), 8.94 (s, 1H), 7.47 (s, 1H), 6.92 (d, 2H), 6.63 (d, 2H), 5.16 (s, 2H).

Step 7

Preparation of compound 3: 1-(4-Chloro-3-trifluoromethyl-phenyl)-3-[4-(6-[1,2,4]oxadiazol-3-yl-pyrimidin-4-yloxy)-phenyl]-urea was prepared following the procedures described in preparation of Example 1. [M+H]⁺ 475.00; ¹H NMR (400 MHz, DMSO) δ 9.86 (s, 1H), 9.19 (s, 1H), 8.96 (d, 2H), 8.10 (s, 1H), 7.61 (m, 4H), 7.20 (m, 2H).

### EXAMPLE 4

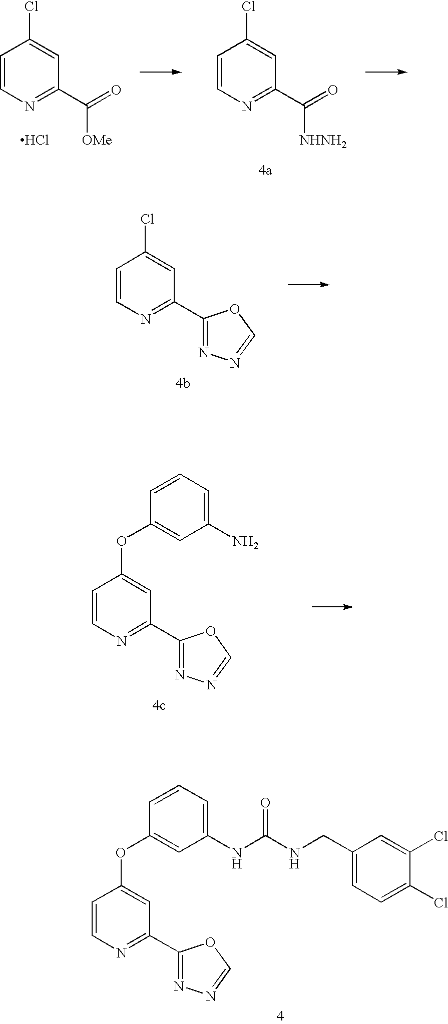 Step 1

### Preparation of compound 4a: 4-Chloropyridinecarboxylic acid hydrazide

Anhydrous hydrazine (10 mL) was added dropwise over 30 minutes to a stirred mixture of methyl 4-chloropyridine-2-carboxylate hydrochloride (10.5 g, 50.0 mmol), THF (100 mL) and methanol (50 mL) at 0° C. under nitrogen. The reaction mixture was stirred at 0° C. for 2 h, then warmed to room temperature. The solution was concentrated under vacuum prior to the addition of water (50 mL) and diethyl ether (50 mL). The resulting precipitate was collected by vacuum filtration to give 8.00 g of 4-chloropyridinecarboxylic acid hydrazide as a white solid. [M+H]⁺ 172.36, 174.37; ¹H-NMR (400 MHz, DMSO) δ 10.01 (s, 1H), 8.55 (d, 1H), 7.96 (s, 1H), 7.70 (d, 1H), 4.62 (s, 2H).

Step 2

### Preparation of compound 4b: 4-Chloro-2-[1,3,4]oxadizol-2-yl-pyridine

A mixture of 4-chloropyridinecarboxylic acid hydrazide (2.20 g, 12.8 mmol), triethyl orthoformate (7 mL) and p-TsOH (200 mg, 1.28 mmol) was heated to 130° C. for 35 min. The reaction mixture was cooled to room temperature prior to the addition of water (50 mL) and diethyl ether (10 mL). The resulting precipitate was collected by vacuum filtration to give 1.60 g of 4-chloro-2-[1,3,4]oxadizol-2-yl-pyridine as a white solid. [M+H]⁺ 182.40, 184.40; ¹H-NMR (400 MHz, DMSO) δ 8.65 (d, 1H), 8.59 (s, 1H), 8.24 (s, 1H), 7.46 (d, 1H), 4.62 (s, 2H).

Step 3

### Preparation of compound 4c: 4-(2-[1,3,4]Oxadiazole-2-yl-pyridin-3-yloxy)phenylamine

Sodium hydride (260 mg of a 60% dispersion in mineral oil, 6.50 mmol) was added to a solution of 3-aminophenol (660 mg, 6.00 mmol) in DMSO (7 mL) at room temperature under nitrogen. The reaction mixture was stirred for 30 minutes prior to the addition of 4-chloro-2-[1,3,4]oxadizol-2-yl-pyridine (900 mg, 5.00 mmol). The solution was heated to 90° C. for 1.5 h, then cooled to room temperature. Water (15 mL) and ethyl acetate (30 mL) were added and the phases were separated. The aqueous layer was back extracted with ethyl acetate (30 mL) and the combined organic layers were concentrated under vacuum. The product was purified using column chromatography (hexanes to ethyl acetate) to give 450 mg of 4-(2-[1,3,4]oxadiazole-2-yl-pyridin-3-yloxy)phenylamine as a white solid. [M+H]⁺ 255.80; ¹H-NMR (400 MHz, CD₃OD) δ 9.08 (s, 1H), 8.53 (d, 1H), 7.67 (d, 1H), 7.16 (m, 1H), 7.08 (m, 1H), 6.62 (d, 1H), 6.48 (d, 1H), 6.40 (d, 1H).

Step 4

Preparation of compound 4: 1-(3,4-Dichlorobenzyl)-3-[3-(2-[1,3,4]oxadiazol-2-yl-pyridin-4-yloxy)-phenyl]-urea was prepared following the procedures described in preparation of Example 1. [M+H]⁺ 456.36, 458.30; ¹H-NMR (400 MHz, CD₃OD) δ 9.75 (s, 1H), 9.45 (d, 1H), 8.38 (m, 2H), 8.32 (d, 1H), 8.28 (m, 1H), 8.17 (t, 1H), 8.11-7.90 (m, 3H), 7.60 (m, 2H), 5.06 (d, 2H), 4.12 (s, 1H).

### EXAMPLE 5

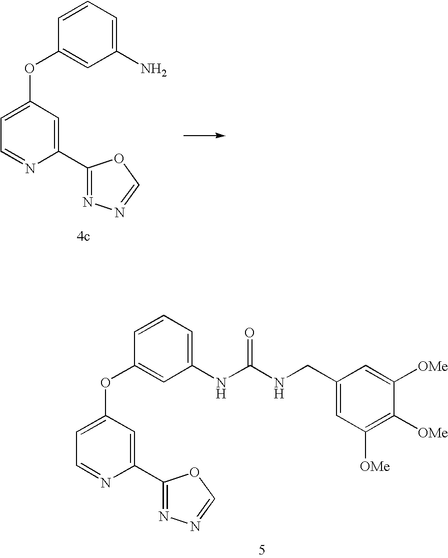

Preparation of compound 5: 1-[3-(2-[1,3,4]Oxadiazol-2-yl-pyridin-4-yloxy)-phenyl]-3-(3,4,5-trimethoxybenzyl)-urea was prepared following the procedures described in preparation of Example 1. [M+H]⁺ 478.10.

### EXAMPLE 6

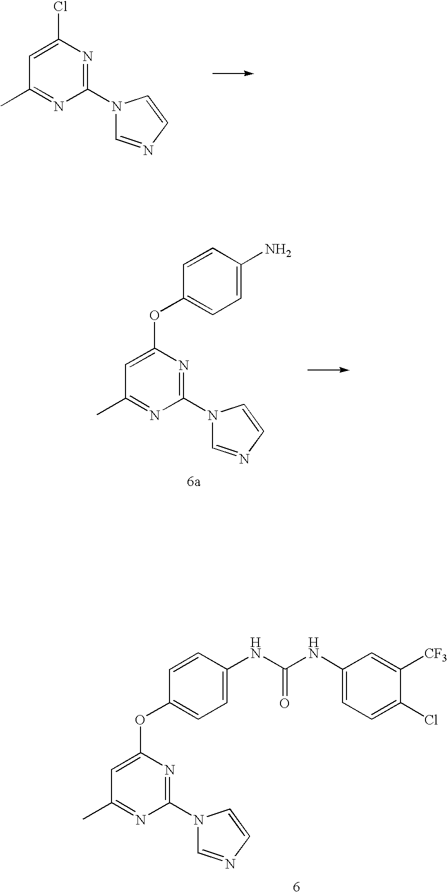 Step 1

Preparation of compound 6a: 4-(2-(1H-Imidazol-1-yl)-6-methylpyrimidin-4-yloxy)aniline was prepared following the procedures described in preparation of Example 1. [M+H]⁺ 268.61.

Step 2

Preparation of compound 6: 1-(4-(2-(1H-imidazol-1-yl)-6-methylpyrimidin-4-yloxy)phenyl)-3-(4-chloro-3-(trifluoromethyl)phenyl)urea was prepared following the procedures described in preparation of Example 1. [M+H]⁺ 489.52, 491.48.

### EXAMPLE 7

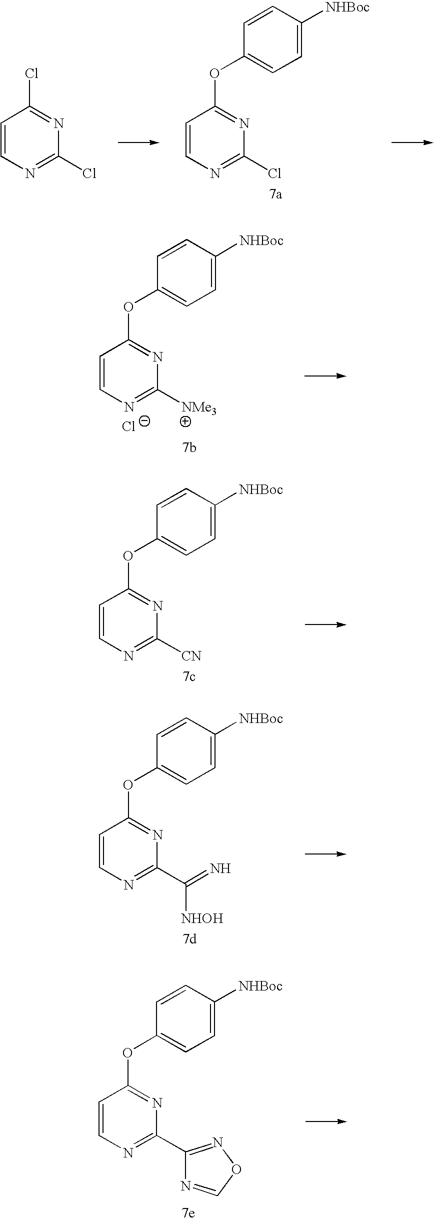 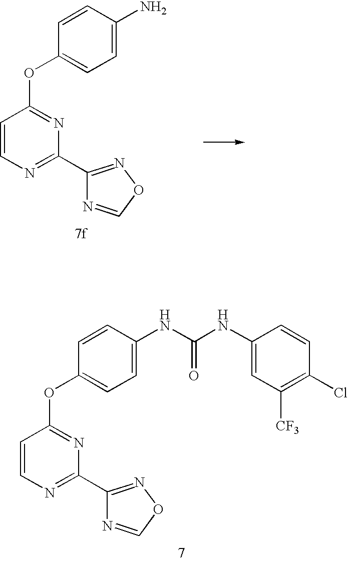 Step 1

Preparation of compound 7a: tert-Butyl 4-(2-chloropyrimidin-4-yloxy)phenylcarbamate was prepared following the procedures described in preparation of Example 3. [M+H]⁺ 322.51.

Step 2

Preparation of compound 7b: 4-(4-(tert-Butoxycarbonylamino)phenoxy)-N,N,N-trimethylpyrimidin-2-aminium chloride was prepared following the procedures described in preparation of Example 3. [M+H]⁺ 345.31.

Step 3

Preparation of compound 7c: tert-Butyl 4-(2-cyanopyrimidin-4-yloxy)phenylcarbamate was prepared following the procedures described in preparation of Example 3. [M−H]⁺ 311.05.

Step 4

Preparation of compound 7d: tert-Butyl 4-(2-(N-hydroxycarbamimidoyl)pyrimidin-4-yloxy)phenylcarbamate was prepared following the procedures described in preparation of Example 3. [M−H]⁺ 344.03.

Step 5

Preparation of compound 7e: tert-Butyl 4-(2-(1,2,4-oxadiazol-3-yl)pyrimidin-4-yloxy)phenylcarbamate was prepared following the procedures described in preparation of Example 3. [M−H]⁺ 354.09.

Step 6

Preparation of compound 7f: 4-(2-(1,2,4-Oxadiazol-3-yl)pyrimidin-4-yloxy)aniline was prepared following the procedures described in preparation of Example 3. [M+H]⁺ 256.04.

Step 7

Preparation of compound 7: 1-(4-(2-(1,2,4-Oxadiazol-3-yl)pyrimidin-4-yloxy)phenyl)-3-(4-chloro-3-(trifluoromethyl)phenyl)urea was prepared following the procedures described in preparation of Example 3. [M+H]⁺ 477.32, 478.87; ¹H-NMR (400 MHz, DMSO) δ 9.87 (s, 1H), 9.22 (s, 1H), 8.98 (s, 1H), 8.95 (s, 1H), 8.10 (s, 1H), 7.61 (m, 5H), 7.23 (d, 2H).

### EXAMPLE 8

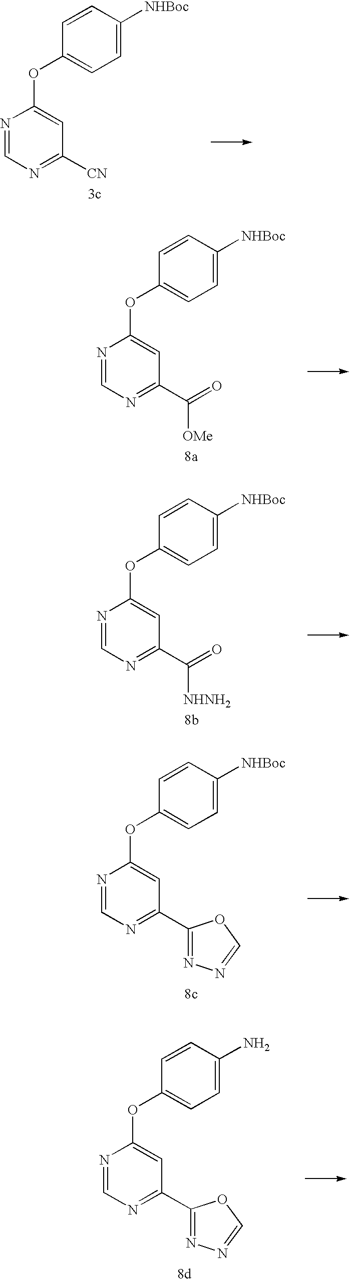 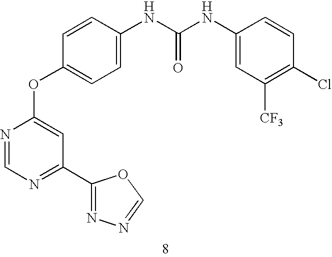 Step 1

### Preparation of compound 8a: Methyl 6-(4-(tert-butoxycarbonylamino)phenoxy)pyrimidine-4-carboxylate

Sodium methoxide (3.50 g, 64.8 mmol) was added to a solution of tert-butyl 4-(6-cyanopyrimidin-4-yloxy)phenylcarbamate (4.00 g, 12.8 mmol) in methanol (250 ml) at 10° C. under nitrogen. The reaction mixture was stirred at 10° C. for 45 minutes, then warmed to room temperature. Water (50 mL) and 1M HCl (6 mL) were added and the mixture was stirred for 1 h. Na₂CO₃ (100 mL of a saturated aqueous solution) and ethyl acetate (300 mL) were added and the phases were separated. The organic layer was concentrated under vacuum to give 4.21 g of methyl 6-(4-(tert-butoxycarbonylamino)phenoxy)pyrimidine-4-carboxylate as a white solid. [M+H]⁺ 345.82.

Step 2

Preparation of compound 8b: tert-Butyl 4-(6-(hydrazinecarbonyl)pyrimidin-4-yloxy)phenylcarbamate was prepared following the procedures described in preparation of Example 4. [M+H]⁺ 346.42.

Step 3

Preparation of compound 8c: tert-Butyl 4-(6-(1,3,4-oxadiazol-2-yl)pyrimidin-4-yloxy)phenylcarbamate was prepared following the procedures described in preparation of Example 4. [M+H]⁺ 356.46; ¹H-NMR (400 MHz, DMSO) δ 8.92 (s, 1H), 8.62 (s, 1H), 7.73 (d, 2H), 7.46 (d, 2H), 7.10 (m, 2H), 6.65 (s, 1H), 1.51 (s, 9H).

Step 4

Preparation of compound 8d: 4-(6-[1,3,4]Oxadiazol-2-yl-pyrimidin-4-yloxy)-phenylamine was prepared following the procedures described in preparation of Example 2. [M+H]⁺ 256.31; ¹H-NMR (400 MHz, DMSO) δ 9.50 (d, 1H), 8.94 (s, 1H), 7.52 (d, 2H), 6.89 (d, 2H), 6.61 (d, 2H), 5.16 (s, 2H).

Step 5

Preparation of compound 8: 1-(4-(6-(1,3,4-Oxadiazol-2-yl)pyrimidin-4-yloxy)phenyl)-3-(4-chloro-3-(trifluoromethyl)phenyl)urea was prepared following the procedures described in preparation of Example 1. [M+H]⁺ 477.32, 478.92; ¹H-NMR (400 MHz, DMSO) δ 9.52 (s, 1H), 9.19 (s, 1H), 8.95 (d, 2H), 8.10 (d, 1H), 7.69 (d, 1H), 7.60 (m, 4H), 7.20 (d, 2H).

### EXAMPLE 9

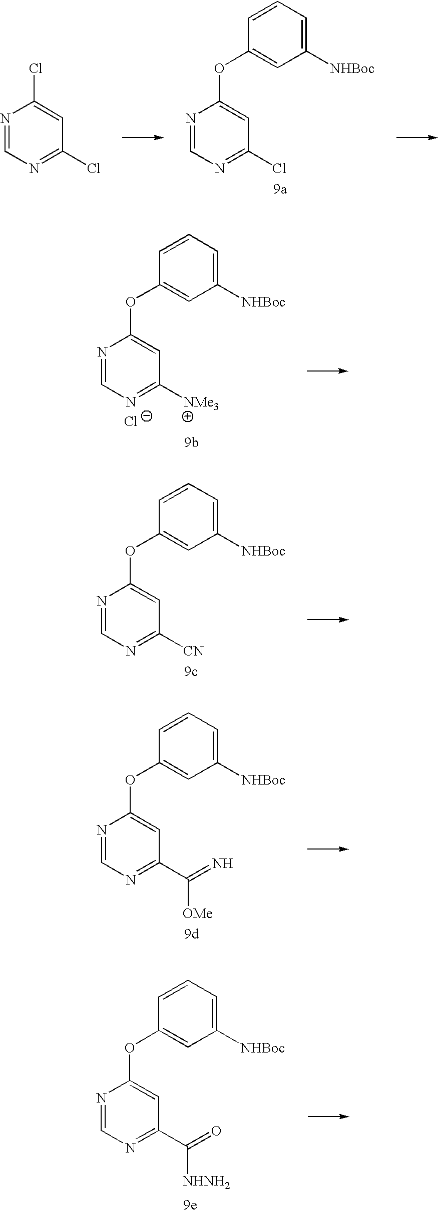 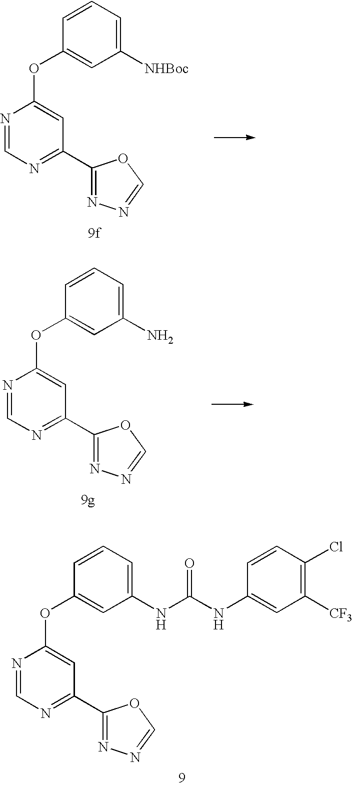 Step 1

Preparation of compound 9a: tert-Butyl 3-(6-chloropyrimidin-4-yloxy)phenylcarbamate was prepared following the procedures described in preparation of Example 1. [M+H]⁺ 322.00.

Step 2

Preparation of compound 9b: 6-(3-(tert-Butoxycarbonylamino)phenoxy)-N,N,N-trimethylpyrimidin-4-aminium chloride was prepared following the procedures described in preparation of Example 3. [M+H]⁺ 346.30.

Step 3

Preparation of compound 9c: tert-Butyl 3-(6-cyanopyrimidin-4-yloxy)phenylcarbamate was prepared following the procedures described in preparation of Example 3. [M−H]⁺ 311.00.

Step 4

Preparation of compound 9d: Methyl 6-(3-(tert-butoxycarbonylamino)phenoxy)pyrimidine-4-carboxylate was prepared following the procedures described in preparation of Example 8. [M+H]⁺ 346.30.

Step 5

Preparation of compound 9e: tert-Butyl 3-(6-(hydrazinecarbonyl)pyrimidin-4-yloxy)phenylcarbamate was prepared following the procedures described in preparation of Example 8. [M+H]⁺ 346.33.

Step 6

Preparation of compound 9f: tert-Butyl 3-(6-(1,3,4-oxadiazol-2-yl)pyrimidin-4-yloxy)phenylcarbamate was prepared following the procedures described in preparation of Example 8. [M+H]⁺ 356.47.

Step 7

Preparation of compound 9g: 3-(6-(1,3,4-Oxadiazol-2-yl)pyrimidin-4-yloxy)aniline was prepared following the procedures described in preparation of Example 8. [M+H]⁺ 256.48.

Step 8

Preparation of compound 9: 1-(3-(6-(1,3,4-Oxadiazol-2-yl)pyrimidin-4-yloxy)phenyl)-3-(4-chloro-3-(trifluoromethyl)phenyl)urea was prepared following the procedures described in preparation of Example 1. [M+H]⁺ 477.32, 478.96; ¹H-NMR (400 MHz, DMSO) δ 9.53 (s, 1H), 9.23 (s, 1H), 9.08 (s, 1H), 8.97 (s, 1H), 8.08 (d, 1H), 7.73 (d, 1H), 7.60 (m, 2H), 7.53 (m, 1H), 7.40 (t, 1H), 7.30 (d, 1H) 6.91 (dd, 1H).

### EXAMPLE 10

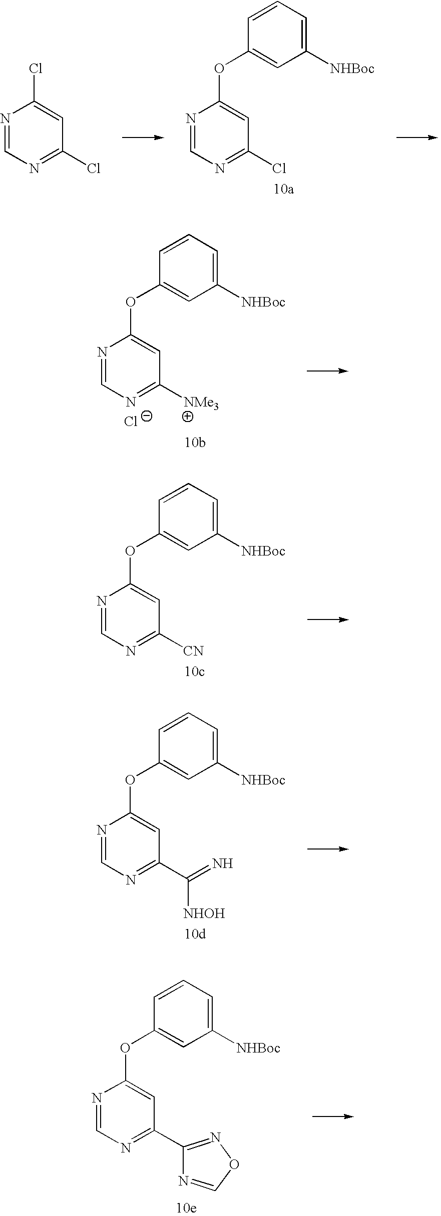 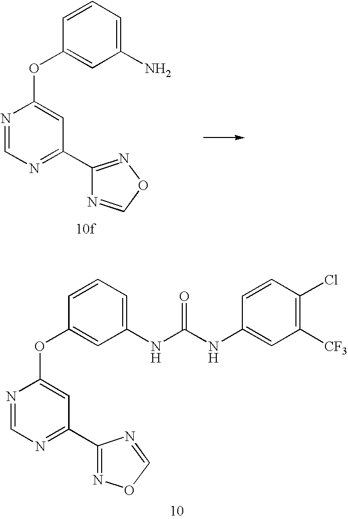 Step 1

Preparation of compound 10a: tert-Butyl 3-(2-chloropyridin-4-yloxy)phenylcarbamate was prepared following the procedures described in preparation of Example 3. [M+H]⁺ 321.09.

Step 2

Preparation of compound 10b: 6-(3-(tert-Butoxycarbonylamino)phenoxy)-N,N,N-trimethylpyrimidin-4-aminium chloride was prepared following the procedures described in preparation of Example 3. [M+H]⁺ 345.40.

Step 3

Preparation of compound 10c: tert-Butyl 3-(6-cyanopyrimidin-4-yloxy)phenylcarbamate was prepared following the procedures described in preparation of Example 3. [M−H]⁺ 311.00.

Step 4

Preparation of compound 10d: tert-Butyl 3-(6-(N-hydroxycarbamimidoyl)pyrimidin-4-yloxy)phenylcarbamate was prepared following the procedures described in preparation of Example 3. [M+H]⁺ 346.47.

Step 5

Preparation of compound 10e: tert-Butyl 3-(6-(1,2,4-oxadiazol-3-yl)pyrimidin-4-yloxy)phenylcarbamate was prepared following the procedures described in preparation of Example 3. [M+H]⁺ 356.76.

Step 6

Preparation of compound 10f: 3-(6-(1,2,4-Oxadiazol-3-yl)pyrimidin-4-yloxy)aniline was prepared following the procedures described in preparation of Example 3. [M+H]⁺ 256.50.

Step 7

Preparation of compound 10: 1-(3-(6-(1,2,4-Oxadiazol-3-yl)pyrimidin-4-yloxy)phenyl)-3-(4-chloro-3-(trifluoromethyl)phenyl)urea was prepared following the procedures described in preparation of Example 3. [M+H]⁺ 477.25, 478.82; ¹H-NMR (400 MHz, DMSO) δ 9.87 (s, 1H), 9.22 (s, 1H), 9.07(s, 1H), 8.95 (s, 1H), 8.08 (s, 1H), 7.64 (m, 2H), 7.52 (s, 1H), 7.53 (m, 1H), 7.39 (t, 1H), 7.30 (d, 1H), 6.91 (d, 1H).

### EXAMPLE 11

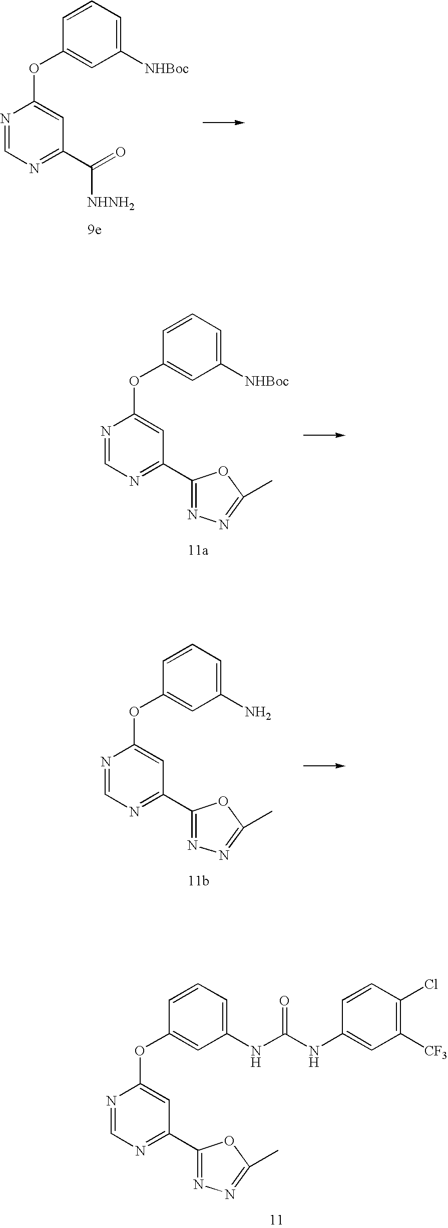 Step 1

Preparation of compound 11a: tert-Butyl 3-(6-(5-methyl-1,3,4-oxadiazol-2-yl)pyrimidin-4-yloxy)phenylcarbamate was prepared following the procedures described in preparation of Example 1. [M+H]⁺ 370.48.

Step 2

Preparation of compound 11b: 3-(6-(5-Methyl-1,3,4-oxadiazol-2-yl)pyrimidin-4-yloxy)aniline was prepared following the procedures described in preparation of Example 1. [M+H]⁺ 270.42.

Step 3

Preparation of compound 11: 1-(4-Chloro-3-(trifluoromethyl)phenyl)-3-(3-(6-(5-methyl-1,3,4-oxadiazol-2-yl)pyrimidin-4-yloxy)phenyl)urea was prepared following the procedures described in preparation of Example 1. [M+H]⁺ 491.36, 492.89; ¹H-NMR (400 MHz, DMSO) δ 9.20 (s, 1H), 9.06 (s, 1H), 8.94 (s, 1H), 8.07 (d, 1H), 7.65 (d, 1H), 7.59 (m, 2H), 7.53 (m, 1H), 7.39 (t, 1H), 7.30 (d, 1H), 6.90 (dd, 1H), 2.62 (s, 3H).

### EXAMPLE 12

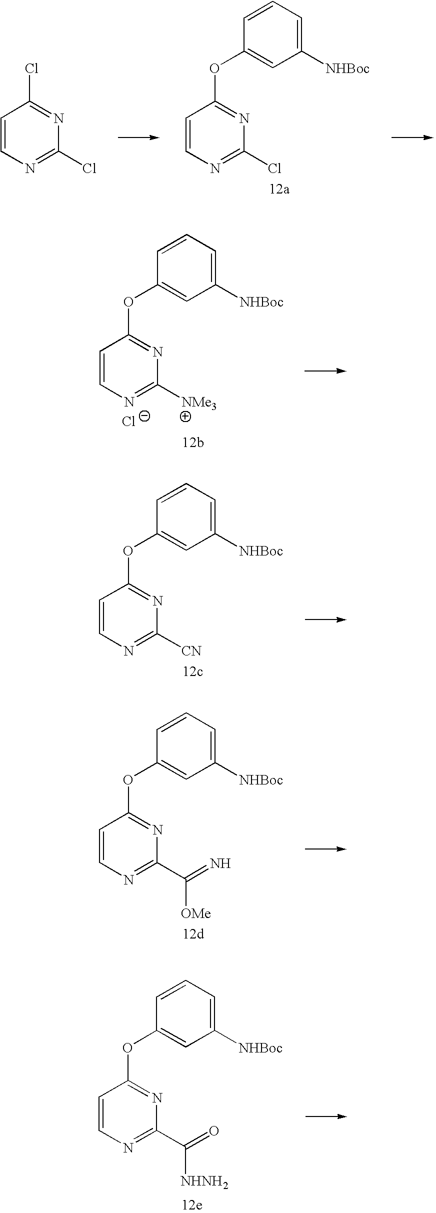 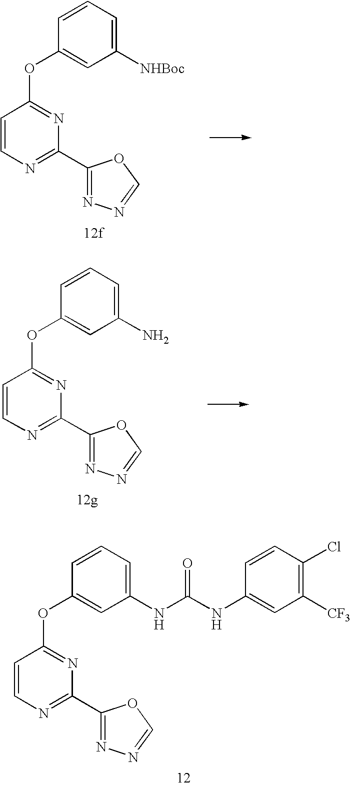 Step 1

Preparation of compound 12a: tert-Butyl 3-(2-chloropyrimidin-4-yloxy)phenylcarbamate was prepared following the procedures described in preparation of Example 3. [M+H]⁺ 321.99.

Step 2

Preparation of compound 12b: 4-(3-(tert-Butoxycarbonylamino)phenoxy)-N,N,N-trimethylpyrimidin-2-aminium chloride was prepared following the procedures described in preparation of Example 3. [M+H]⁺ 345.31.

Step 3

Preparation of compound 12c: tert-Butyl 3-(2-cyanopyrimidin-4-yloxy)phenylcarbamate was prepared following the procedures described in preparation of Example 3. [M−H]⁺ 311.03.

Step 4

Preparation of compound 12d: Methyl 4-(3-(tert-butoxycarbonylamino)phenoxy)pyrimidine-2-carboxylate was prepared following the procedures described in preparation of Example 8. [M+H]⁺ 346.79.

Step 5

Preparation of compound 12e: tert-Butyl 3-(2-(hydrazinecarbonyl)pyrimidin-4-yloxy)phenylcarbamate was prepared following the procedures described in preparation of Example 8. [M+H]⁺ 346.33.

Step 6

Preparation of compound 12f: tert-Butyl 3-(2-(1,3,4-oxadiazol-2-yl)pyrimidin-4-yloxy)phenylcarbamate was prepared following the procedures described in preparation of Example 8. [M+H]⁺ 356.47.

Step 7

Preparation of compound 12g: 3-(2-(1,3,4-Oxadiazol-2-yl)pyrimidin-4-yloxy)aniline was prepared following the procedures described in preparation of Example 8. [M+H]⁺ 256.43.

Step 8

Preparation of compound 12: 1-(3-(2-(1,3,4-oxadiazol-2-yl)pyrimidin-4-yloxy)phenyl)-3-(4-chloro-3-(trifluoromethyl)phenyl)urea was prepared following the procedures described in preparation of Example 1. [M+H]⁺ 477.32, 478.90; ¹H-NMR (400 MHz, DMSO) δ 9.40 (s, 1H), 9.21 (s, 1H), 9.06 (s, 1H), 8.89 (d, 1H), 8.07 (d, 1H), 7.60 (m, 2H), 7.51 (m, 1H), 7.39 (t, 1H), 7.33 (d, 1H), 7.27 (d, 1H), 6.92 (dd, 1H).

### EXAMPLE 13

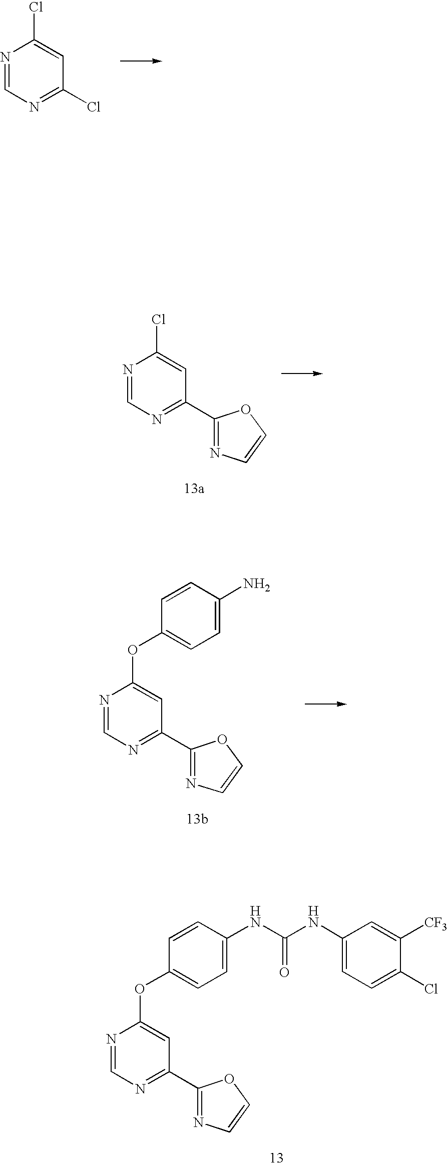 Step 1

### Preparation of compound 13a: 2-(6-Chloropyrimidin-4-yl)oxazole

Bis(triphenylphosphine)palladium(II) dichloride (94.0 mg, 134 μmol) was added to a nitrogen purged mixture of 4,6-dichloropyrimidine (200 mg, 1.34 mmol), 2-(tri-n-butylstannyl)oxazole (281 μL, 1.34 mmol) and DMF (5 mL) at room temperature under nitrogen. The solution was heated to 90° C. for 16 h, then cooled to room temperature. KF (50 mL of a saturated aqueous solution) was added and the mixture stirred for 1 h. Ethyl acetate (30 mL) was added and the layers were separated. The organic layer was washed with brine and concentrated under vacuum. The product was purified using column chromatography (hexanes to 1:1 hexanes/ethyl acetate) to give 141 mg of 2-(6-chloropyrimidin-4-yl)oxazole as an off-white solid. [M+H]⁺ 182.33.

Step 2

Preparation of compound 13b: 4-(6-(Oxazol-2-yl)pyrimidin-4-yloxy)aniline was prepared following the procedures described in preparation of Example 1. [M+H]⁺ 254.88.

Step 3

Preparation of compound 13: 1-(4-Chloro-3-(trifluoromethyl)phenyl)-3-(4-(6-(oxazol-2-yl)pyrimidin-4-yloxy)phenyl)urea was prepared following the procedures described in preparation of Example 1. [M+H]⁺ 476.27; ¹H-NMR (400 MHz, DMSO) δ 9.21 (s, 1H), 8.97 (s, 1H), 8.88 (d, 1H), 8.41 (s, 1H), 8.10 (d, 1H), 7.65-7.52 (m, 6H), 7.20 (d, 2H).

### EXAMPLE 14

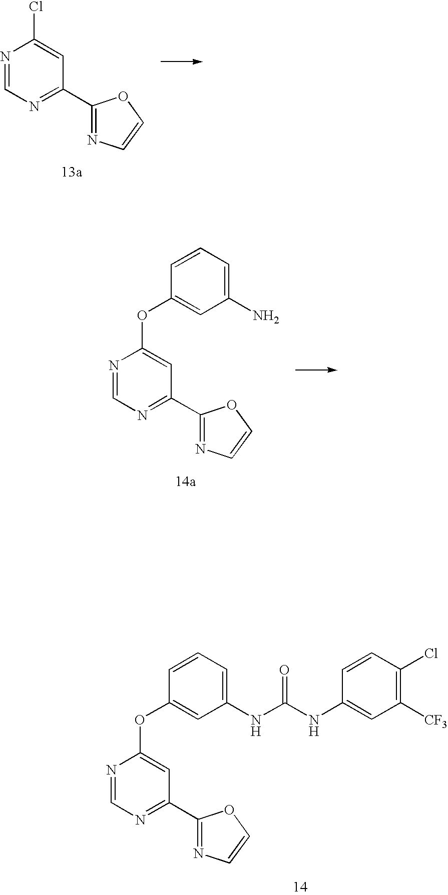 Step 1

Preparation of compound 14a: 3-(6-Oxazol-2-yl-pyrimidin-4-yloxy)-phenylamine was prepared following the procedures described in preparation of Example 1. [M+H]⁺ 255.08.

Step 2

Preparation of compound 14: 1-(4-chloro-3-(trifluoromethyl)phenyl)-3-(3-(6-(oxazol-2-yl)pyrimidin-4-yloxy)phenyl)urea was prepared following the procedures described in preparation of Example 1. [M+H]⁺ 276.27.

### EXAMPLE 15

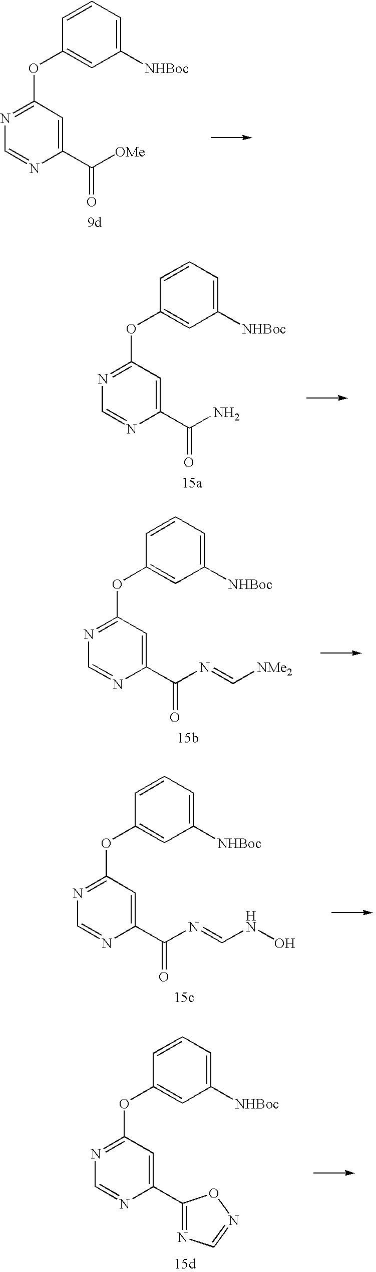 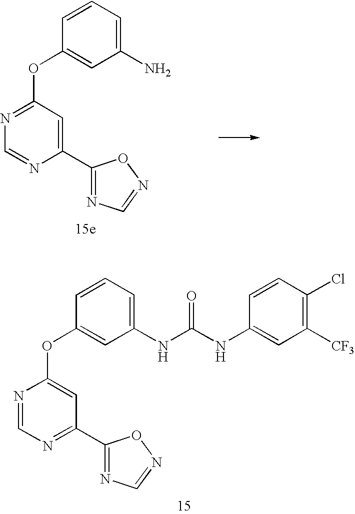 Step 1

### Preparation of compound 15a: tert-Butyl 3-(6-carbamoylpyrimidin-4-yloxy)phenylcarbamate

Ammonia (50 mL of a 2M solution in methanol, 100 mmol) was added to a stirred solution of 6-(4-(tert-butoxycarbonylamino)phenoxy)pyrimidine-4-carboxylate (4.00 g, 11.6 mmol) in methanol (20 mL) at 0° C. under nitrogen. The reaction mixture was warmed to room temperature over 30 minutes and the resulting precipitate was collected by vacuum filtration to give 3.20 g of tert-butyl 3-(6-carbamoylpyrimidin-4-yloxy)phenylcarbamate as a white solid. [M+H]⁺ 331.47.

Step 2

### Preparation of compound 15b: (E)-tert-Butyl 3-(6-((dimethylamino)methylenecarbamoyl)pyrimidin-4-yloxy)phenylcarbamate

A mixture of tert-butyl 3-(6-carbamoylpyrimidin-4-yloxy)phenylcarbamate (1.30 g, 3.93 mmol), N,N-dimethylformamide dimethyl acetal (2 mL) and THF (10 mL) was stirred at room temperature for 14 h under nitrogen. The solution was concentrated under vacuum and the product was purified using column chromatography (hexanes to ethyl acetate) to give 800 mg of (E)-tert-butyl 3-(6-((dimethylamino)methylenecarbamoyl)pyrimidin-4-yloxy)phenylcarbamate as a white solid. [M+H]⁺ 385.89.

Step 3

### Preparation of compound 15c: (E)-tert-Butyl 3-(6-((hydroxyamino)methylenecarbamoyl)pyrimidin-4-yloxy)phenylcarbamate

A mixture of hydroxlyamine hydrochloride (195 mg, 2.81 mmol) and acetic acid (3 mL) was added to a solution of KOH (153 mg, 2.73 mmol) in water (0.8 mL) at room temperature. The reaction mixture was stirred for 30 minutes prior to the addition of a solution of (E)-tert-butyl 3-(6-((dimethylamino)methylenecarbamoyl)pyrimidin-4-yloxy)phenylcarbamate (800 mg, 2.08 mmol) in dioxane (3 mL). The reaction mixture was stirred for 10 minutes, then water (5 mL) was added. The mixture was cooled to 0° C. for 30 minutes and the resulting precipitate was collected by vacuum filtration to give 640 mg of (E)-tert-butyl 3-(6-((hydroxyamino)methylenecarbamoyl)pyrimidin-4-yloxy)phenylcarbamate as a white solid. [M+H]⁺ 374.47.

Step 4

### Preparation of compound 15d: tert-Butyl 3-(6-(1,2,4-oxadiazol-5-yl)pyrimidin-4-yloxy)phenylcarbamate

A mixture of (E)-tert-butyl 3-(6-((hydroxyamino)methylenecarbamoyl)pyrimidin-4-yloxy)phenylcarbamate (640 mg, 1.72 mmol), dioxane (5 mL) and acetic acid (5 mL) was heated to 90° C. for 2.5 h. The reaction mixture was cooled to room temperature prior to dropwise addition of K₂CO₃ (50 mL of a saturated aqueous solution). Water (50 mL) and ethyl acetate (100 mL) were added and the phases were separated. The aqueous layer was back extracted with ethyl acetate (2×100 mL) and the combined organic layers were concentrated under vacuum. The product was purified using column chromatography (hexanes to 1:1 hexanes/ethyl acetate) to give 450 mg of tert-butyl 3-(6-(1,2,4-oxadiazol-5-yl)pyrimidin-4-yloxy)phenylcarbamate as a white solid. [M+H]⁺ 356.40.

Step 5

Preparation of compound 15e: 3-(6-[1,2,4]Oxadiazol-5-yl-pyrimidin-4-yloxy)-phenylamine was prepared following the procedures described in preparation of Example 2. [M+H]⁺ 256.05, 257.46; ¹H-NMR (400 MHz, CD₃OD) δ 8.89 (s, 1H), 8.88 (s, 1H), 6.68 (d, 1H), 7.15 (t, 1H), 6.64 (m, 1H), 6.51 (m, 1H), 6.46 (m, 1H).

Step 6

Preparation of compound 15: 1-(3-(6-(1,2,4-oxadiazol-5-yl)pyrimidin-4-yloxy)phenyl)-3-(4-chloro-3-(trifluoromethyl)phenyl)urea was prepared following the procedures described in preparation of Example 1. [M+H]⁺ 477.32, 478.78; ¹H-NMR (400 MHz, DMSO) δ 9.30 (s, 1H), 9.21 (s, 1H), 9.07 (s, 1H), 9.90 (d, 1H), 8.08 (d, 1H), 7.80 (s, 1H), 7.60 (m, 2H), 7.53 (m, 1H), 7.40 (t, 1H), 7.29 (d, 1H), 6.91 (dd, 1H).

### EXAMPLE 16

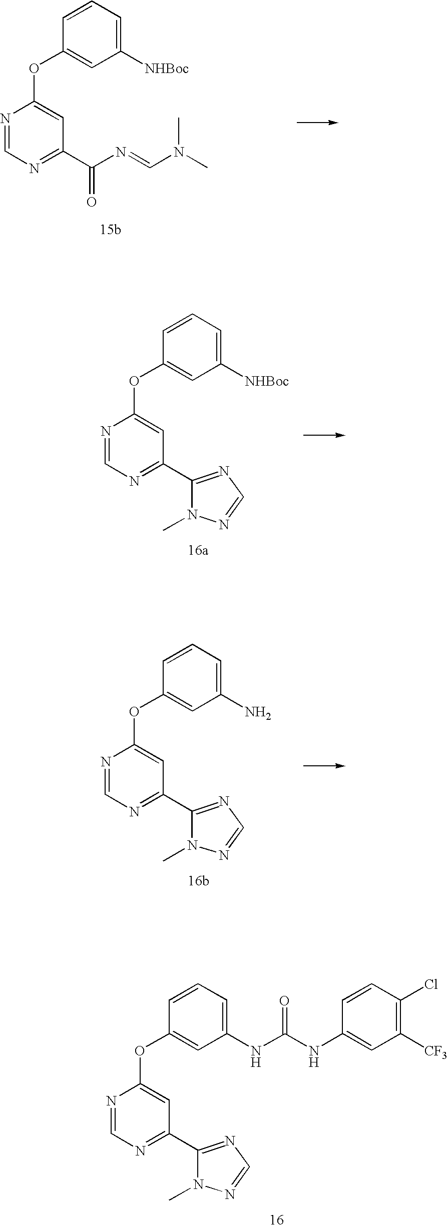 Step 1

### Preparation of compound 16a: tert-Butyl 3-(6-(1-methyl-1H-1,2,4-triazol-5-yl)pyrimidin-4-yloxy)phenylcarbamate

Methylhydrazine (45 μL, 860 μmol) was added to a solution of 15b (330 mg, 860 μmol) and acetic acid (3.0 mL) at room temperature under nitrogen. The reaction mixture was heated to 90° C. for 45 minutes, then cooled to room temperature. K₂CO₃ (40 mL of a saturated aqueous solution) was added and the mixture was stirred for 15 minutes. The solution was extracted with ethyl acetate (2×15 mL) and the combined organic layers were concentrated under vacuum. The product was purified using column chromatography (DCM to 4:1 DCM/MeOH) to give 20 mg of tert-butyl 3-(6-(1-methyl-1H-1,2,4-triazol-5-yl)pyrimidin-4-yloxy)phenylcarbamate as a white solid. [M+H]⁺ 369.54.

Step 2

Preparation of compound 16b: 3-(6-(1-Methyl-1H-1,2,4-triazol-5-yl)pyrimidin-4-yloxy)aniline was prepared following the procedures described in preparation of Example 1. [M+H]⁺ 269.46.

Step 3

Preparation of compound 16: 1-(4-Chloro-3-(trifluoromethyl)phenyl)-3-(3-(6-(1-methyl-1H-1,2,4-triazol-5-yl)pyrimidin-4-yloxy)phenyl)urea was prepared following the procedures described in preparation of Example 1. [M+H]⁺ 490.37; ¹H-NMR (400 MHz, DMSO) δ 9.21 (s, 1H), 9.06 (s, 1H), 8.36 (d, 1H), 8.64 (s, 1H), 8.07 (s, 1H), 7.64-7.58 (m, 2H), 7.46 (d, 1H), 7.39 (t, 1H), 7.31-7.29 (m, 2H), 6.90-6.88 (m, 1H), 3.96 (s, 3H).

### EXAMPLE 17

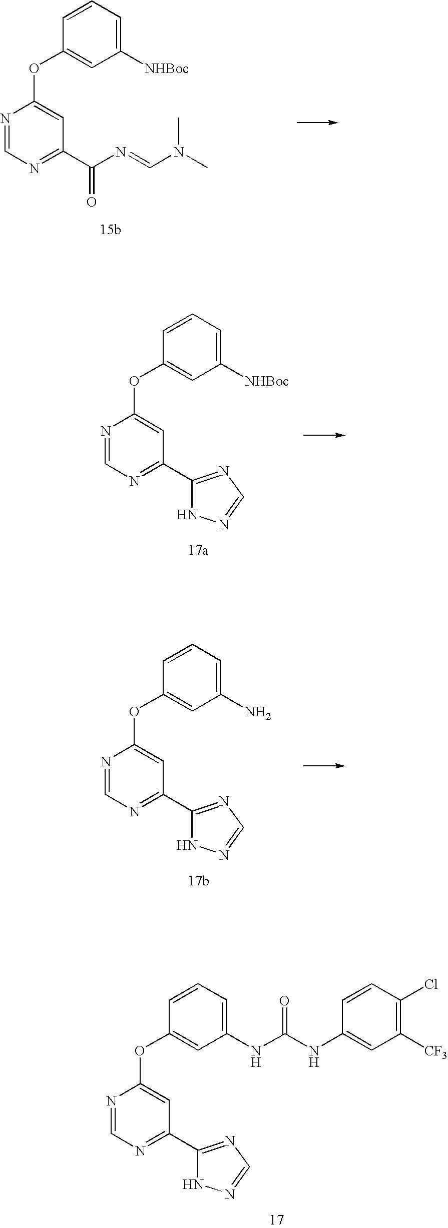 Step 1

Preparation of compound 17a: tert-Butyl 3-(6-(1H-1,2,4-triazol-5-yl)pyrimidin-4-yloxy)phenylcarbamate was prepared following the procedures described in preparation of Example 16. [M+H]⁺ 355.46.

Step 2

Preparation of compound 17b: 3-(6-(1H-1,2,4-Triazol-5-yl)pyrimidin-4-yloxy)aniline was prepared following the procedures described in preparation of Example 1. [M+H]⁺ 255.44.

Step 3

Preparation of compound 17: 1-(3-(6-(1H-1,2,4-Triazol-5-yl)pyrimidin-4-yloxy)phenyl)-3-(4-chloro-3-(trifluoromethyl)phenyl)urea was prepared following the procedures described in preparation of Example 1. [M+H]⁺ 476.31; ¹H-NMR (400 MHz, DMSO) δ 9.36 (s, 1H), 9.18 (s, 1H), 8.86 (s, 1H), 8.48 (br s, 1H), 8.08 (s, 1H), 7.65-7.58 (m, 3H), 7.50 (s, 1H), 7.39 (t, 1H), 7.33-7.32 (m, 2H), 6.91-6.89 (m, 1H).

### EXAMPLE 18

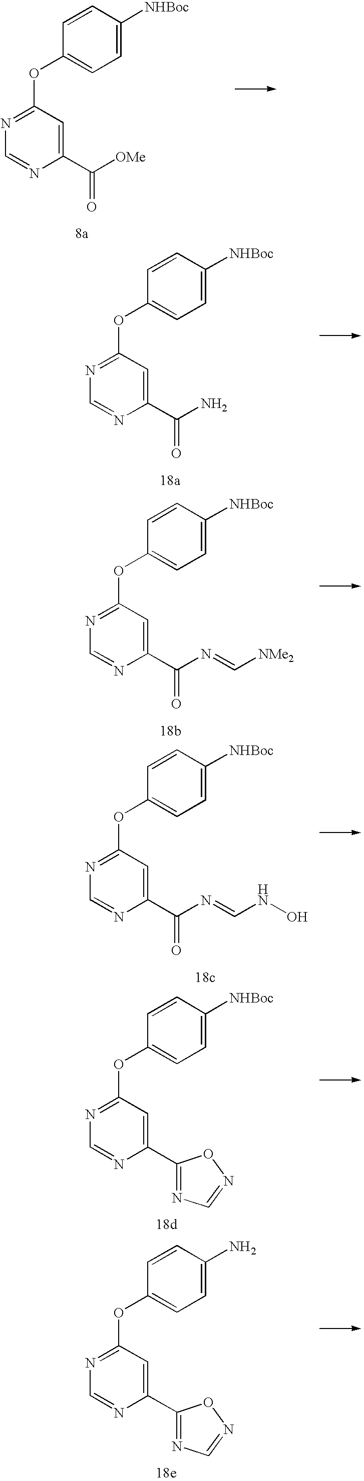 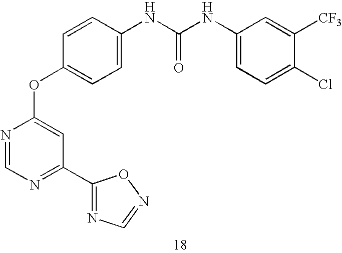 Step 1

Preparation of compound 18a: [4-(6-Carbamoyl-pyrimidin-4-yloxy)-phenyl]-carbamic acid tert-butyl ester was prepared following the procedures described in preparation of Example 15. [M+H]⁺ 331.45.

Step 2

Preparation of compound 18b: (E)-{4-[6-(Dimethylaminomethylene-carbamoyl)-pyrimidin-4-yloxy]-phenyl}-carbamic acid tert-butyl ester was prepared following the procedures described in preparation of Example 15. [M+H]⁺ 386.02.

Step 3

Preparation of compound 18c: (E)-tert-Butyl 4-(6-((hydroxyamino)methylenecarbamoyl)pyrimidin-4-yloxy)phenylcarbamate was prepared following the procedures described in preparation of Example 15. [M+H]⁺ 373.40.

Step 4

Preparation of compound 18d: [4-(6-[1,2,4]Oxadiazol-5-yl-pyrimidin-4-yloxy)-phenyl]-carbamic acid tert-butyl ester was prepared following the procedures described in preparation of Example 15. [M+H]⁺ 356.44.

Step 5

Preparation of compound 18e: 4-(6-[1,2,4]Oxadiazol-5-yl-pyrimidin-4-yloxy)-phenylamine was prepared following the procedures described in preparation of Example 15. [M+H]⁺ 257.18.

Step 6

Preparation of compound 18: 1-(4-(6-(1,2,4-Oxadiazol-5-yl)pyrimidin-4-yloxy)phenyl)-3-(4-chloro-3-(trifluoromethyl)phenyl)urea was prepared following the procedures described in preparation of Example 15. [M+H]⁺ 477.32, 478.85.

### EXAMPLE 19

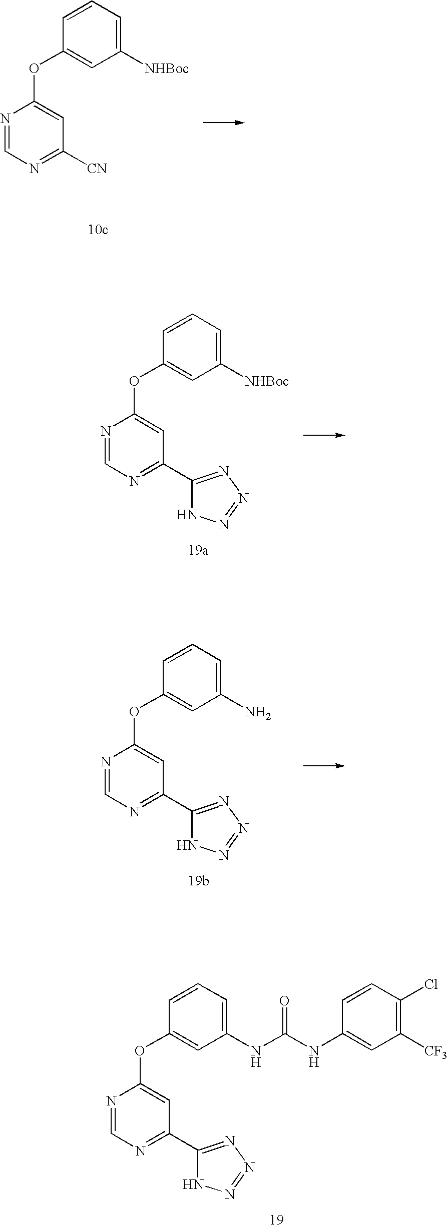 Step 1

### Preparation of compound 19a: tert-Butyl 3-(6-(1H-tetrazol-5-yl)pyrimidin-4-yloxy)phenylcarbamate

A suspension of [3-(6-cyano-pyrimidin-4-yloxy)-phenyl]-carbamic acid tert-butyl ester (500 mg, 1.60 mmol), sodium azide (124 mg, 1.92 mmol), ammonium chloride (103 mg, 1.92 mmol) and DME (8 mL) was heated to 85° C. under nitrogen for 39 h. The reaction mixture was cooled to room temperature and directly purified using column chromatography (DCM to 4:1 DCM/MeOH) to give 280 mg of tert-butyl 3-(6-(1H-tetrazol-5-yl)pyrimidin-4-yloxy)phenylcarbamate. [M+H]⁺ 356.47.

Step 2

Preparation of compound 19b: 3-(6-(1H-Tetrazol-5-yl)pyrimidin-4-yloxy)aniline was prepared following the procedures described in preparation of Example 1. [M+H]⁺ 256.45.

Step 3

Preparation of compound 19: 1-(3-(6-(1H-Tetrazol-5-yl)pyrimidin-4-yloxy)phenyl)-3-(4-chloro-3-(trifluoromethyl)phenyl)urea was prepared following the procedures described in preparation of Example 1. [M+H]⁺ 477.32; ¹H-NMR (400 MHz, DMSO) δ 9.23 (s, 1H), 9.09 (s, 1H), 9.00 (s, 1H), 8.08 (s, 1H), 7.72 (s, 1H), 7.62-7.58 (m, 2H), 7.54 (s, 1H), 7.40 (t, 1H), 7.31-7.29 (m, 2H), 6.91-6.89 (m, 1H).

### EXAMPLES 20 AND 21

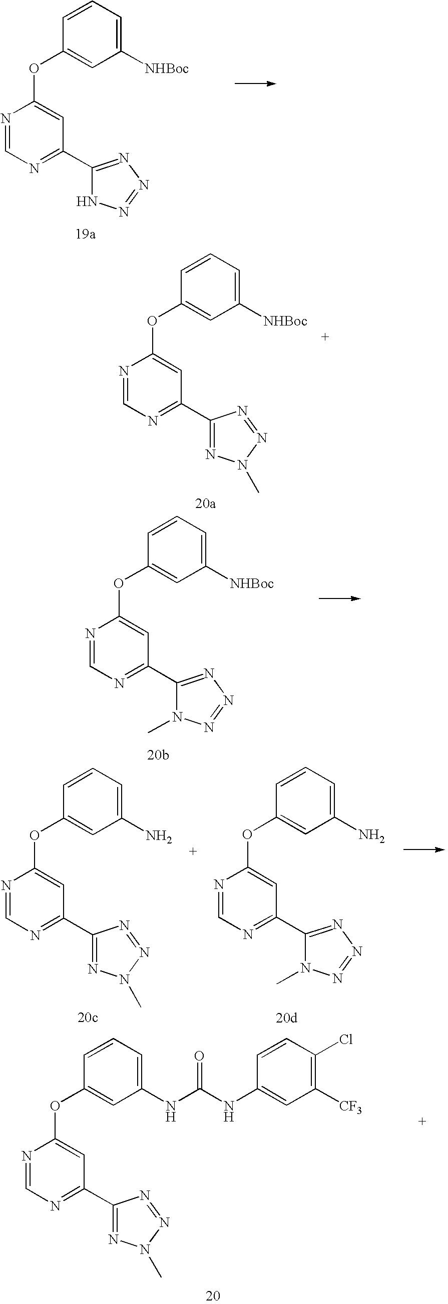 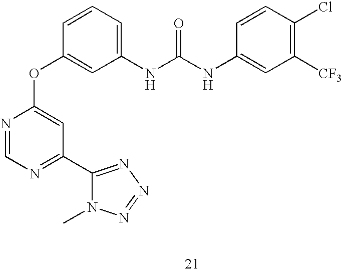 Step 1

### Preparation of compounds 20a and 20b: tert-Butyl 3-(6-(2-methyl-2H-tetrazol-5-yl)pyrimidin-4-yloxy)phenylcarbamate and tert-butyl 3-(6-(1-methyl-1H-tetrazol-5-yl)pyrimidin-4-yloxy)phenylcarbamate

Methyl iodide (26 μL, 409 μmol) was added to a mixture of 19a (97 mg, 272 Amos), K₂CO₃ (75 mg, 550 Amos) and acetone (3 mL) at room temperature under nitrogen. The reaction mixture was stirred for 24 h then concentrated under vacuum. The products were purified using column chromatography (DCM to 9:1 DCM/MeOH) to give a mixture of tert-butyl 3-(6-(2-methyl-2H-tetrazol-5-yl)pyrimidin-4-yloxy)phenylcarbamate and tert-butyl 3-(6-(1-methyl-1H-tetrazol-5-yl)pyrimidin-4-yloxy)phenylcarbamate. [M+H]⁺ 370.24.

Step 2

Preparation of compounds 20c and 20d: 3-(6-(2-Methyl-2H-tetrazol-5-yl)pyrimidin-4-yloxy)aniline and 3-(6-(1-methyl-1H-tetrazol-5-yl)pyrimidin-4-yloxy)aniline were prepared following the procedures described in preparation of Example 1. [M+H]⁺ 270.45.

Step 3

Preparation of compound 20 and 21: 1-(4-Chloro-3-(trifluoromethyl)phenyl)-3-(3-(6-(2-methyl-2H-tetrazol-5-yl)pyrimidin-4-yloxy)phenyl)urea and 1-(4-chloro-3-(trifluoromethyl)phenyl)-3-(3-(6-(1-methyl-1H-tetrazol-5-yl)pyrimidin-4-yloxy)phenyl)urea were prepared following the procedures described in preparation of Example 1. Data for 20: [M+H]⁺ 491.36; ¹H-NMR (400 MHz, DMSO) δ 9.22 (s, 1H), 9.07 (s, 1H), 8.94 (s, 1H), 8.07 (s, 1H), 7.63-7.52 (m, 3H), 7.40 (t, 1H), 7.31-7.29 (m, 2H), 6.91-6.89 (m, 1H), 4.47 (s, 3H). Data for 21: [M+H]⁺ 491.36; ¹H-NMR (400 MHz, DMSO) δ 9.22 (s, 1H), 9.08 (s, 1H), 9.03 (s, 1H), 8.07 (s, 1H), 7.75 (s, 1H), 7.60-7.55 (m, 3H), 7.41 (t, 1H), 7.31-7.29 (m, 1H), 6.91-6.89 (m, 1H), 4.42 (s, 3H).

### EXAMPLE 22

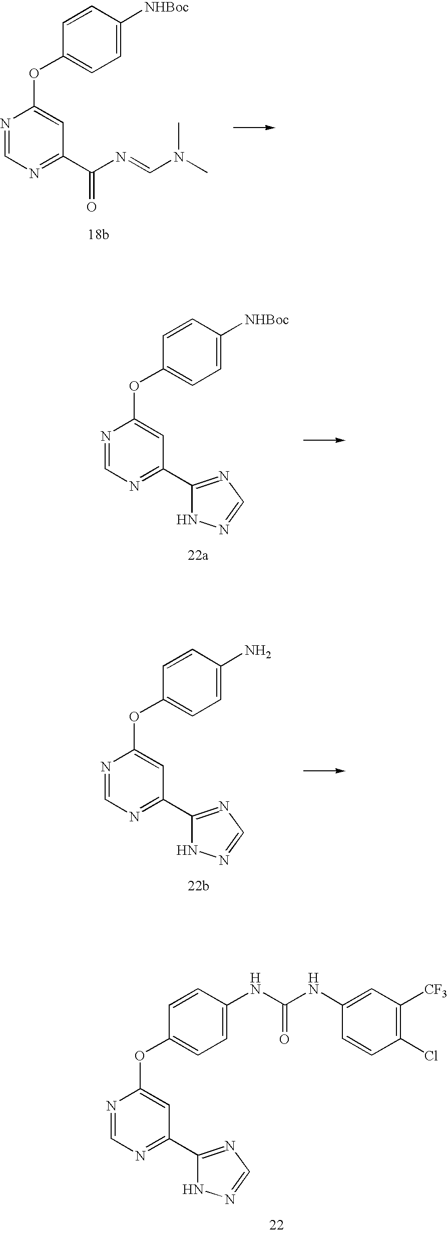 Step 1

Preparation of compound 22a: tert-Butyl 4-(6-(1H-1,2,4-triazol-5-yl)pyrimidin-4-yloxy)phenylcarbamate was prepared following the procedures described in preparation of Example 16. [M+H]⁺ 355.04.

Step 2

Preparation of compound 22b: 4-(6-(1H-1,2,4-Triazol-5-yl)pyrimidin-4-yloxy)aniline was prepared following the procedures described in preparation of Example 1. [M+H]⁺ 255.41.

Step 3

Preparation of compound 22: 1-(4-(6-(1H-1,2,4-Triazol-5-yl)pyrimidin-4-yloxy)phenyl)-3-(4-chloro-3-(trifluoromethyl)phenyl)urea was prepared following the procedures described in preparation of Example 1. [M+H]⁺ 476.36; ¹H-NMR (400 MHz, DMSO) δ 9.24 (s, 1H), 9.00 (s, 1H), 8.86 (s, 1H), 8.11 (s, 1H), 7.66-7.49 (m, 6H), 7.20 (m, 3H).

### EXAMPLE 23

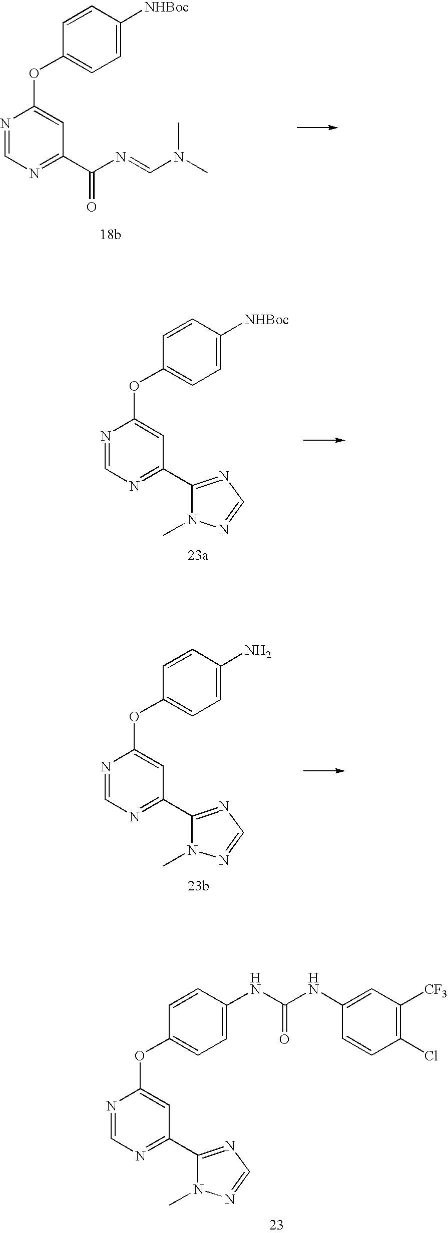 Step 1

Preparation of compound 23a: tert-Butyl 4-(6-(1-methyl-1H-1,2,4-triazol-5-yl)pyrimidin-4-yloxy)phenylcarbamate was prepared following the procedures described in preparation of Example 16. [M+H]⁺ 369.37.

Step 2

Preparation of compound 23b: 4-(6-(1-Methyl-1H-1,2,4-triazol-5-yl)pyrimidin-4-yloxy)aniline was prepared following the procedures described in preparation of Example 1. [M+H]⁺ 269.38.

Step 3

Preparation of compound 23: 1-(4-Chloro-3-(trifluoromethyl)phenyl)-3-(4-(6-(1-methyl-1H-1,2,4-triazol-5-yl)pyrimidin-4-yloxy)phenyl)urea was prepared following the procedures described in preparation of Example 1. [M+H]⁺ 490.01; ¹H-NMR (400 MHz, DMSO) δ 9.21 (s, 1H), 8.96 (s, 1H), 8.82 (s, 1H), 8.64 (s, 1H), 8.10 (d, 1H), 7.66-7.49 (m, 4H), 7.42 (s, 1H), 7.20 (m, 2H), 3.96 (s, 3H).

### EXAMPLE 24

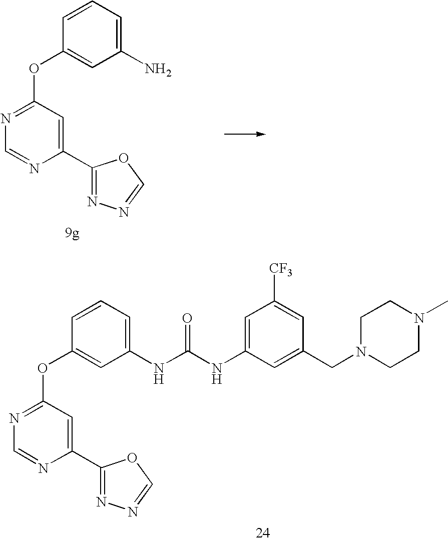

### Preparation of compound 24: 1-(3-(6-(1,3,4-Oxadiazol-2-yl)pyrimidin-4-yloxy)phenyl)-3-(3-((4-methylpiperazin-1-yl)methyl)-5-(trifluoromethyl)phenyl)urea

Pyridine (50 μL) was added to a stirred solution of 3-((4-methylpiperazin-1-yl)methyl)-5-(trifluoromethyl)aniline (109 mg, 400 μmol) and 4-nitrophenyl chloroformate (81.0 mg, 400 μmol) in anhydrous dichloromethane (5 mL) at room temperature under nitrogen. The reaction mixture was stirred for 5 minutes prior to sequential addition of 3-(6-(1,3,4-oxadiazol-2-yl)pyrimidin-4-yloxy)aniline (103 mg, 400 μmol) and diisopropylethylamine (250 μL). The resulting solution was stirred for 1 h, then water (5 mL) and ethyl acetate (10 mL) were added. The phases were separated and the organic layer was concentrated under vacuum. The product was purified using column chromatography (DCM to 4:1 DCM/methanol) to give 155 mg of 1-(3-(6-(1,3,4-oxadiazol-2-yl)pyrimidin-4-yloxy)phenyl)-3-(3-((4-methylpiperazin-1-yl)methyl)-5-(trifluoromethyl)phenyl)urea as a white solid. [M+H]⁺ 555.02, 556.29; ¹H-NMR (400 MHz, CD₃OD) δ 9.19 (s, 1H), 8.88 (d, 1H), 7.77 (s, 1H), 7.74 (d, 1H), 7.63 (s, 1H), 7.56 (t, 1H), 7.38 (t, 1H), 7.30 (m, 2H), 6.90 (m, 1H), 3.58 (s, 2H), 2.70-2.50 (m, 8H), 2.44 (s, 3H).

### EXAMPLE 25

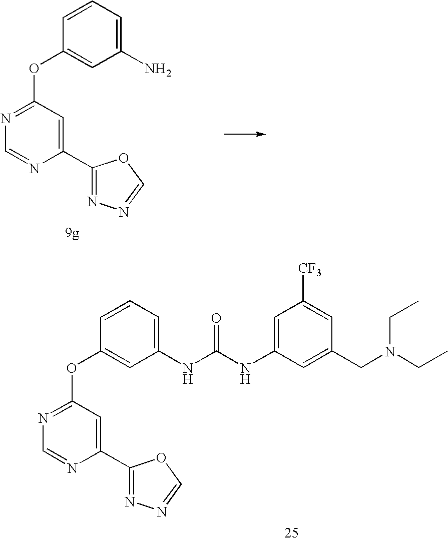

Preparation of compound 25: 1-(3-(6-(1,3,4-Oxadiazol-2-yl)pyrimidin-4-yloxy)phenyl)-3-(3-((diethylamino)methyl)-5-(trifluoromethyl)phenyl)urea was prepared following the procedures described in preparation of Example 24. [M+H]⁺ 529.47; ¹H-NMR (400 MHz, CD₃OD) δ 9.18 (s, 1H), 8.87 (d, 1H), 7.80 (s, 1H), 7.72 (d, 1H), 7.59 (s, 1H), 7.55 (t, 1H), 7.38 (t, 1H), 7.28 (m, 2H), 6.89 (m, 1H), 3.66 (s, 2H), 2.60 (q, 4H), 1.09 (t, 6H).

### EXAMPLE 26

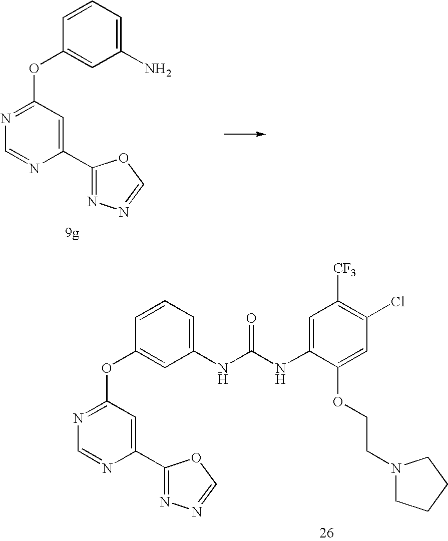

Preparation of compound 26: 1-(3-(6-(1,3,4-Oxadiazol-2-yl)pyrimidin-4-yloxy)phenyl)-3-(4-chloro-2-(2-(pyrrolidin-1-yl)ethoxy)-5-(trifluoromethyl)phenyl)urea was prepared following the procedures described in preparation of Example 24. [M+H]⁺ 590.20, 592.40; ¹H-NMR (400 MHz, CD₃OD) δ 9.18 (s, 1H), 8.87 (d, 1H), 8.62 (s, 1H), 7.74 (d, 1H), 7.57 (t, 1H), 7.38 (t, 1H), 7.34 (m, 1H), 7.22 (s, 1H), 6.88 (m, 1H), 4.38 (t, 2H), 3.50-3.10 (m, 6H), 2.03 (m, 4H).

### EXAMPLE 27

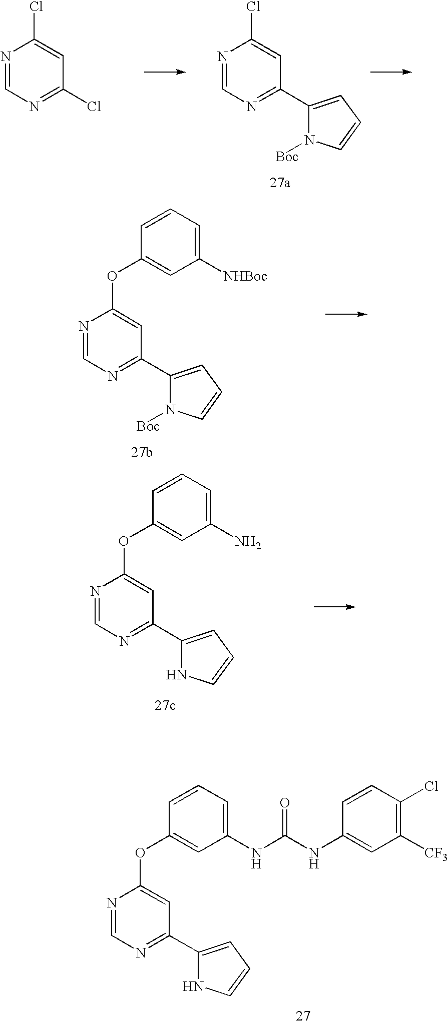 Step 1

### Preparation of compound 27a: tert-Butyl 2-(6-chloropyrimidin-4-yl)-1H-pyrrole-1-carboxylate

Bis(triphenylphosphine)palladium(II) dichloride (83.0 mg, 119 μmol) was added to a nitrogen purged mixture of 4,6-dichloropyrimidine (353 mg, 2.37 mmol), 1-tert-butoxycarbonyl-2-pyrrolylboronic acid (500 mg, 2.37 mmol), Na₂CO₃ (3.50 mL of a 2.0 M aqueous solution) and THF (12 mL) at room temperature. The solution was heated to 70° C. for 2 h then cooled to room temperature. The mixture was concentrated under vacuum prior to addition of brine (15 mL) and ethyl acetate (15 mL). The phases were separated and the organic layer was concentrated under vacuum. The product was purified using column chromatography (hexanes to 4:1 hexanes/ethyl acetate) to give 528 mg of tert-butyl 2-(6-chloropyrimidin-4-yl)-1H-pyrrole-1-carboxylate as a white solid. ¹H-NMR (400 MHz, CDCl₃) δ 9.92 (d, 1H), 7.44 (dd, 1H), 4.20 (d, 1H), 6.71 (dd, 1H), 6.29 (t, 1H), 1.44 (s, 9H).

Step 2

Preparation of compound 27b: tert-Butyl 2-(6-(3-(tert-butoxycarbonylamino)phenoxy)pyrimidin-4-yl)-1H-pyrrole-1-carboxylate was prepared following the procedures described in preparation of Example 1. [M+H]⁺ 452.98.

Step 3

Preparation of compound 27c: 3-(6-(1H-Pyrrol-2-yl)pyrimidin-4-yloxy)aniline was prepared following the procedures described in preparation of Example 1. [M+H]⁺ 253.84.

Step 4

Preparation of compound 27: 1-(3-(6-(1H-Pyrrol-2-yl)pyrimidin-4-yloxy)phenyl)-3-(4-chloro-3-(trifluoromethyl)phenyl)urea was prepared following the procedures described in preparation of Example 1. [M+H]⁺ 474.23; ¹H-NMR (400 MHz, DMSO) δ 11.78 (s, 1H), 9.19 (s, 1H), 9.00 (s, 1H), 8.60 (s, 1H), 8.07 (d, 1H), 7.60 (m, 2H), 7.35 (m, 1H), 7.40-7.25 (m, 4H), 7.02 (m, 1H), 6.83 (m, 1H), 6.21 (m, 1H).

### EXAMPLE 28

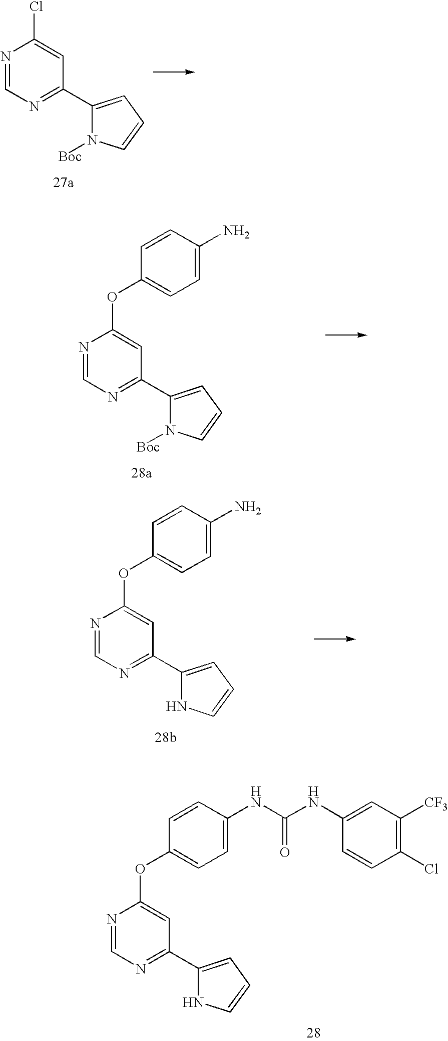 Step 1

Preparation of compound 28a: tert-Butyl 2-(6-(4-aminophenoxy)pyrimidin-4-yl)-1H-pyrrole-1-carboxylate was prepared following the procedures described in preparation of Example 1. [M+H]⁺ 352.94.

Step 2

Preparation of compound 28b: 4-(6-(1H-Pyrrol-2-yl)pyrimidin-4-yloxy)aniline was prepared following the procedures described in preparation of Example 1. [M+H]⁺ 252.96.

Step 3

Preparation of compound 28: 1-(4-(6-(1H-Pyrrol-2-yl)pyrimidin-4-yloxy)phenyl)-3-(4-chloro-3-(trifluoromethyl)phenyl)urea was prepared following the procedures described in preparation of Example 1. [M+H]⁺ 474.32; ¹H-NMR (400 MHz, DMSO) δ 11.78 (s, 1H), 9.17 (s, 1H), 8.92 (s, 1H), 8.58 (s, 1H), 8.10 (d, 1H), 7.67-7.58 (m, 2H), 7.52-7.49 (m, 2H), 7.26 (d, 1H), 7.16-7.11 (m, 2H), 7.00 (m, 2H), 6.21 (m, 1H).

### EXAMPLE 29

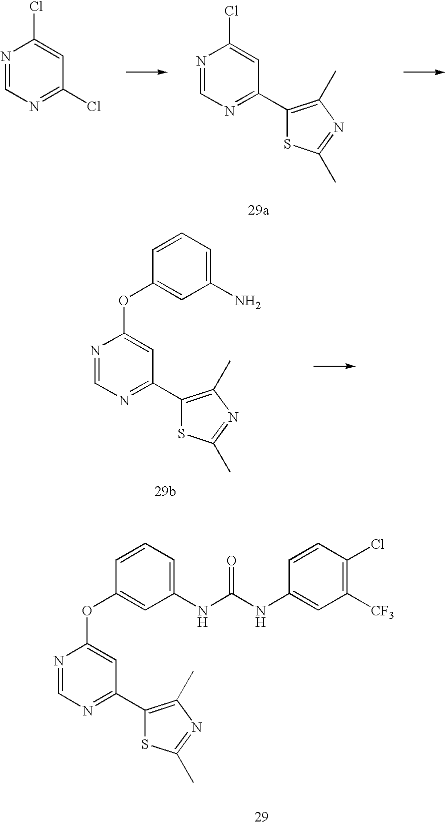 Step 1

Preparation of compound 29a: 5-(6-Chloropyrimidin-4-yl)-2,4-dimethylthiazole was prepared following the procedures described in preparation of Example 27. ¹H-NMR (400 MHz, CDCl₃) δ 8.92 (d, 1H), 7.49 (d, 1H), 2.74 (s, 3H), 2.72 (s, 3H).

Step 2

Preparation of compound 29b: 3-(6-(2,4-Dimethylthiazol-5-yl)pyrimidin-4-yloxy)aniline was prepared following the procedures described in preparation of Example 1. [M+H]⁺ 299.65.

Step 3

Preparation of compound 29: 1-(4-Chloro-3-(trifluoromethyl)phenyl)-3-(3-(6-(2,4-dimethylthiazol-5-yl)pyrimidin-4-yloxy)phenyl)urea was prepared following the procedures described in preparation of Example 1. [M+H]⁺ 519.78; ¹H-NMR (400 MHz, DMSO) δ 9.20 (s, 1H), 9.03 (s, 1H), 8.71 (s, 1H), 8.07 (s, 1H), 7.61-7.59 (m, 2H), 7.48 (t, 1H), 7.35 (t, 1H), 7.27-7.25 (m, 2H), 6.89-6.86 (m, 1H), 2.63 (s, 3H), 2.62 (s, 3H).

### EXAMPLE 30

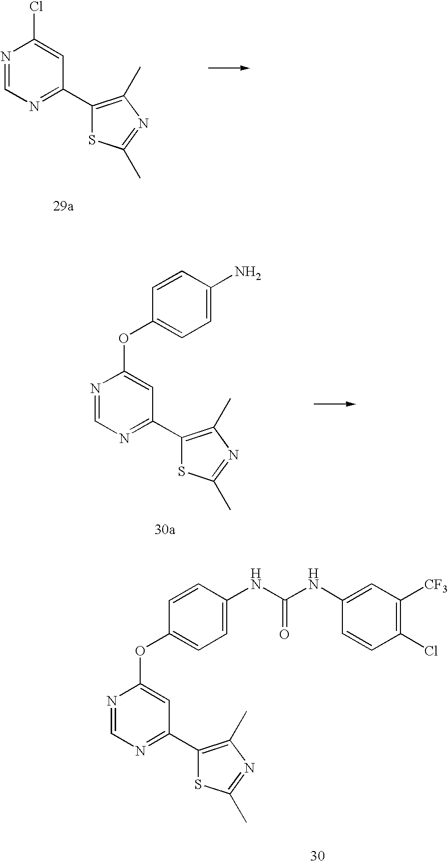 Step 1

Preparation of compound 30a: 4-(6-(2,4-Dimethylthiazol-5-yl)pyrimidin-4-yloxy)aniline was prepared following the procedures described in preparation of Example 1. [M+H]⁺ 298.96.

Step 2

Preparation of compound 30: 1-(4-Chloro-3-(trifluoromethyl)phenyl)-3-(4-(6-(2,4-dimethylthiazol-5-yl)pyrimidin-4-yloxy)phenyl)urea was prepared following the procedures described in preparation of Example 1. [M+H]⁺ 519.79; ¹H-NMR (400 MHz, DMSO) δ 9.18 (s, 1H), 8.94 (s, 1H), 8.70 (s, 1H), 8.10 (d, 1H), 7.66-7.50 (m, 4H), 7.22 (s, 1H), 7.16 (d, 2H), 2.63 (s, 3H), 2.62 (s, 3H).

### EXAMPLE 31

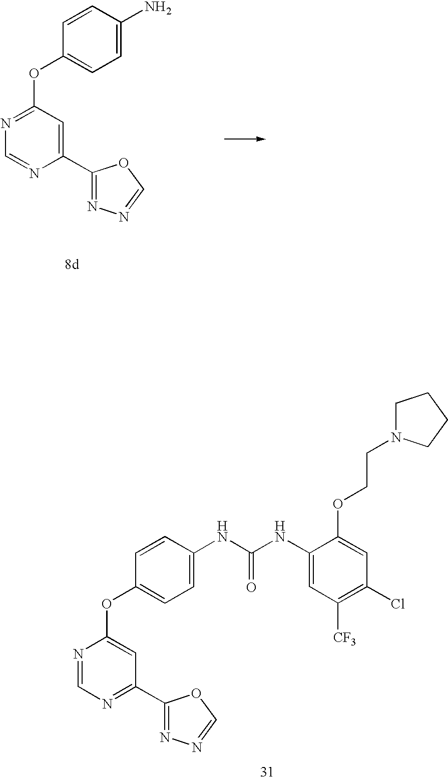

Preparation of compound 31: 1-(4-(6-(1,3,4-Oxadiazol-2-yl)pyrimidin-4-yloxy)phenyl)-3-(4-chloro-2-(2-(pyrrolidin-1-yl)ethoxy)-5-(trifluoromethyl)phenyl)urea was prepared following the procedures described in preparation of Example 24. [M+H]⁺ 590.20, 592.39; ¹H-NMR (400 MHz, CD₃OD) δ 9.52 (s, 1H), 8.96 (d, 1H), 8.75 (s, 1H), 7.70 (d, 1H), 7.57 (d, 1H), 7.39 (s, 1H), 7.34 (m, 1H), 7.22 (d, 1H), 4.41 (s, 2H), 3.70-3.00 (m, 4H), 2.48 (m, 2H), 1.90 (m, 4H).

### EXAMPLE 32

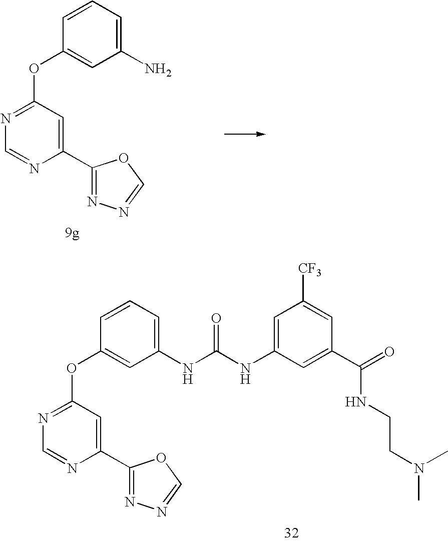

Preparation of compound 32: 3-(3-(3-(6-(1,3,4-Oxadiazol-2-yl)pyrimidin-4-yloxy)phenyl)ureido)-N-(2-(diethylamino)ethyl)-5-(trifluoromethyl)benzamide was prepared following the procedures described in preparation of Example 24. [M+H]⁺ 584.97, 586.28.

### EXAMPLE 33

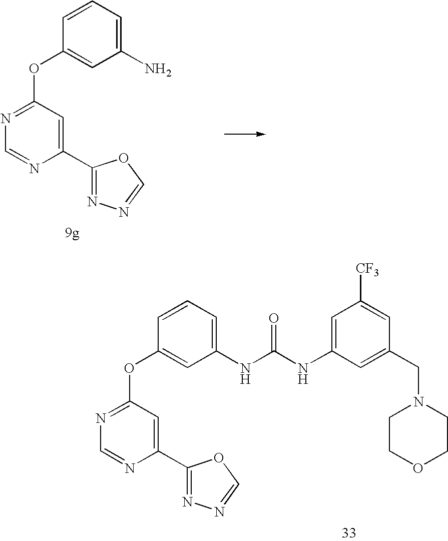

Preparation of compound 33: 1-(3-(6-(1,3,4-Oxadiazol-2-yl)pyrimidin-4-yloxy)phenyl)-3-(3-(morpholinomethyl)-5-(trifluoromethyl)phenyl)urea was prepared following the procedures described in preparation of Example 24. [M+H]⁺ 542.94.

### EXAMPLE 34

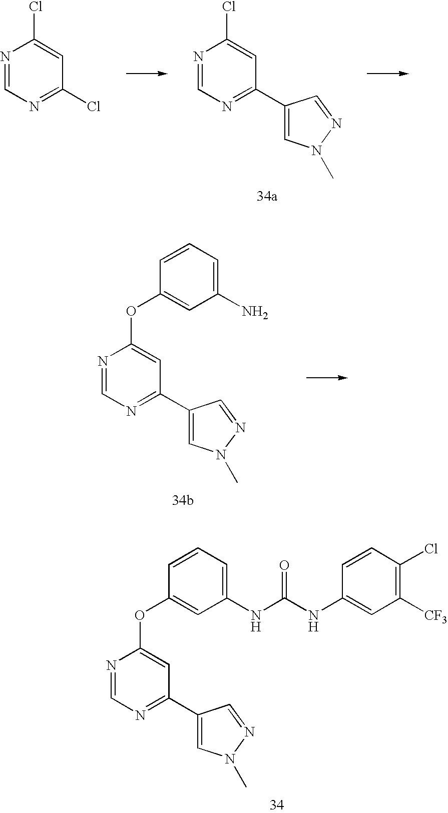 Step 1

Preparation of compound 34a: 4-Chloro-6-(1-methyl-1H-pyrazol-4-yl)pyrimidine was prepared following the procedures described in preparation of Example 27. [M+H]⁺ 194.95; ¹H-NMR (400 MHz, CDCl₃) δ 8.85 (d, 1H), 8.05 (s, 1H), 8.01 (s, 1H), 7.41 (s, 1H), 3.98 (s, 3H).

Step 2

Preparation of compound 34b: 3-(6-(1-Methyl-1H-pyrazol-4-yl)pyrimidin-4-yloxy)aniline was prepared following the procedures described in preparation of Example 1. [M+H]⁺ 268.92.

Step 3

Preparation of compound 34: 1-(4-Chloro-3-(trifluoromethyl)phenyl)-3-(3-(6-(1-methyl-1H-pyrazol-4-yl)pyrimidin-4-yloxy)phenyl)urea was prepared following the procedures described in preparation of Example 1. [M+H]⁺ 489.17; ¹H-NMR (400 MHz, DMSO) δ 9.19 (s, 1H), 9.01 (s, 1H), 8.63 (d, 1H), 8.45 (s, 1H), 8.14 (s, 1H), 8.07 (d, 1H), 7.64-7.56 (m, 2H), 7.43 (t, 1H), 7.36 (dd, 1H), 7.33 (s, 1H), 7.28-7.24 (m, 1H), 6.86-6.82 (m, 1H), 3.89 (s, 3H).

### EXAMPLE 35

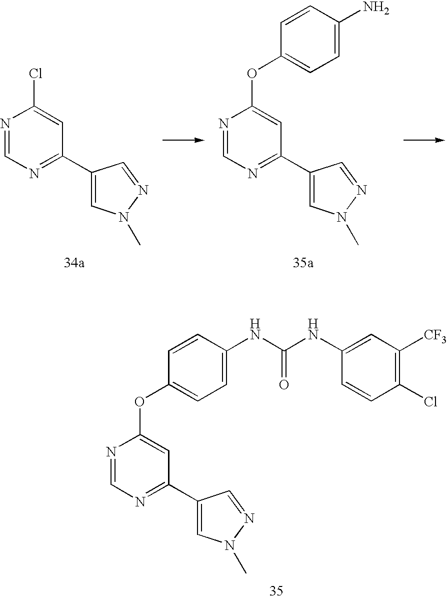 Step 1

Preparation of compound 35a: 4-(6-(1-methyl-1H-pyrazol-4-yl)pyrimidin-4-yloxy)aniline was prepared following the procedures described in preparation of Example 1. [M+H]⁺ 267.93.

Step 2

Preparation of compound 35: 1-(4-Chloro-3-(trifluoromethyl)phenyl)-3-(4-(6-(1-methyl-1H-pyrazol-4-yl)pyrimidin-4-yloxy)phenyl)urea was prepared following the procedures described in preparation of Example 1. [M+H]⁺ 489.14; ¹H-NMR (400 MHz, DMSO) δ 9.17 (s, 1H), 8.92 (s, 1H), 8.61 (d, 1H), 8.44 (s, 1H), 8.13 (s, 1H), 8.10 (d, 1H), 7.66-7.58 (m, 2H), 7.52-7.49 (d, 2H), 7.31 (s, 1H), 7.16-7.12 (d, 2H), 3.88 (s, 3H).

### EXAMPLE 36

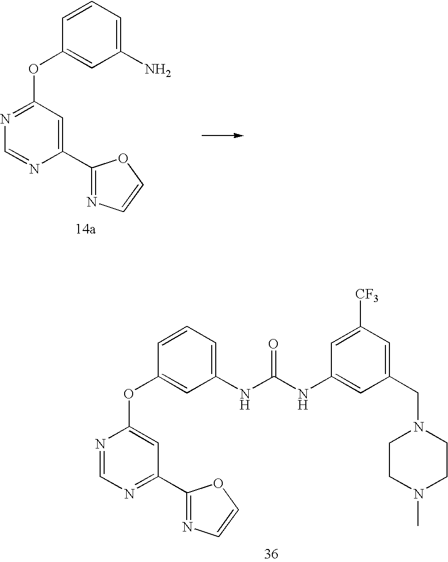

Preparation of compound 36: 1-(3-((4-Methylpiperazin-1-yl)methyl)-5-(trifluoromethyl)phenyl)-3-(3-(6-(oxazol-2-yl)pyrimidin-4-yloxy)phenyl)urea was prepared following the procedures described in preparation of Example 24. [M+H]⁺ 553.97; ¹H-NMR (400 MHz, CD₃OD) δ 8.72 (d, 1H), 8.14 (s, 1H), 7.82 (d, 1H), 7.78 (s, 1H), 7.62 (s, 1H), 7.51 (t, 1H), 7.45 (s, 1H), 7.33 (t, 1H), 7.30 (m, 2H), 6.88 (m, 1H), 3.57 (s, 2H), 2.80-2.50 (m, 8H), 2.44 (s, 3H).

### EXAMPLE 37

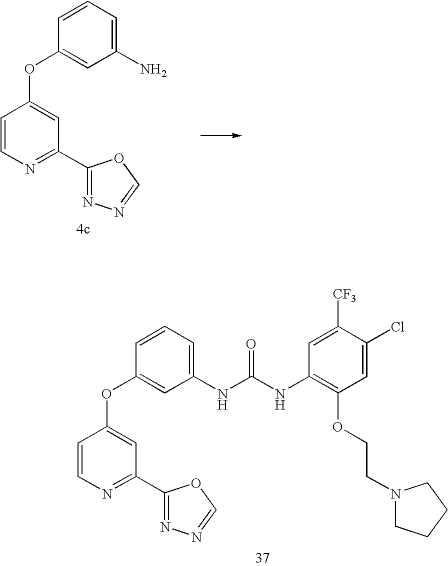

Preparation of compound 37: 1-(3-(2-(1,3,4-Oxadiazol-2-yl)pyridin-4-yloxy)phenyl)-3-(4-chloro-2-(2-(pyrrolidin-1-yl)ethoxy)-5-(trifluoromethyl)phenyl)urea was prepared following the procedures described in preparation of Example 24. [M+H]⁺ 589.40; ¹H-NMR (400 MHz, CD₃OD) δ 9.07 (s, 1H), 8.57 (m, 2H), 7.71 (d, 1H), 7.53 (t, 1H), 7.37 (t, 1H), 7.24 (m, 1H), 7.14 (m, 2H), 6.84 (m, 1H), 4.26 (t, 2H), 3.04 (t, 2H), 2.72 (m, 4H), 1.89 (m, 4H).

### EXAMPLE 38

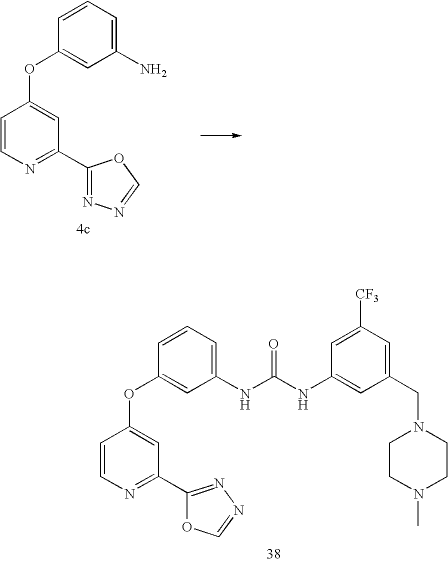

Preparation of compound 38: 1-(3-(2-(1,3,4-Oxadiazol-2-yl)pyridin-4-yloxy)phenyl)-3-(3-((4-methylpiperazin-1-yl)methyl)-5-(trifluoromethyl)phenyl)urea was prepared following the procedures described in preparation of Example 24. [M+H]⁺ 554.40; ¹H-NMR (400 MHz, CD₃OD) δ 9.09 (s, 1H), 8.59 (d, 2H), 7.77 (s, 1H), 7.74 (d, 1H), 7.59 (s, 1H), 7.53 (t, 1H), 7.42 (t, 1H), 7.28 (m, 2H), 7.16 (dd, 1H), 6.86 (m, 2H), 3.55 (s, 2H), 2.60-2.40 (br s, 8H), 2.85 (s, 3H).

### EXAMPLE 39

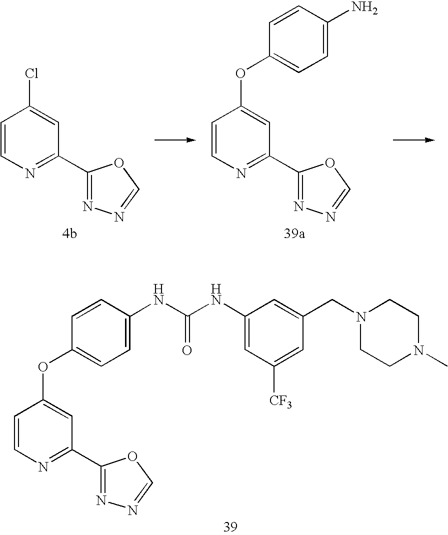 Step 1

Preparation of compound 39a: 4-(2-(1,3,4-Oxadiazol-2-yl)pyridin-4-yloxy)aniline was prepared following the procedures described in preparation of Example 1. [M+H]⁺ 255.80.

Step 2

Preparation of compound 39: 1-(4-(2-(1,3,4-Oxadiazol-2-yl)pyridin-4-yloxy)phenyl)-3-(3-((4-methylpiperazin-1-yl)methyl)-5-(trifluoromethyl)phenyl)urea was prepared following the procedures described in preparation of Example 24. [M+H]⁺ 554.04; ¹H-NMR (400 MHz, CD₃OD) δ 9.09 (s, 1H), 8.56 (d, 2H), 7.82 (s, 1H), 7.70 (d, 1H), 7.59 (m, 3H), 7.28 (m, 1H), 7.16 (m, 3H), 3.45(s, 2H), 2.60-2.40 (br s, 8H), 2.28 (s, 3H).

### EXAMPLE 40

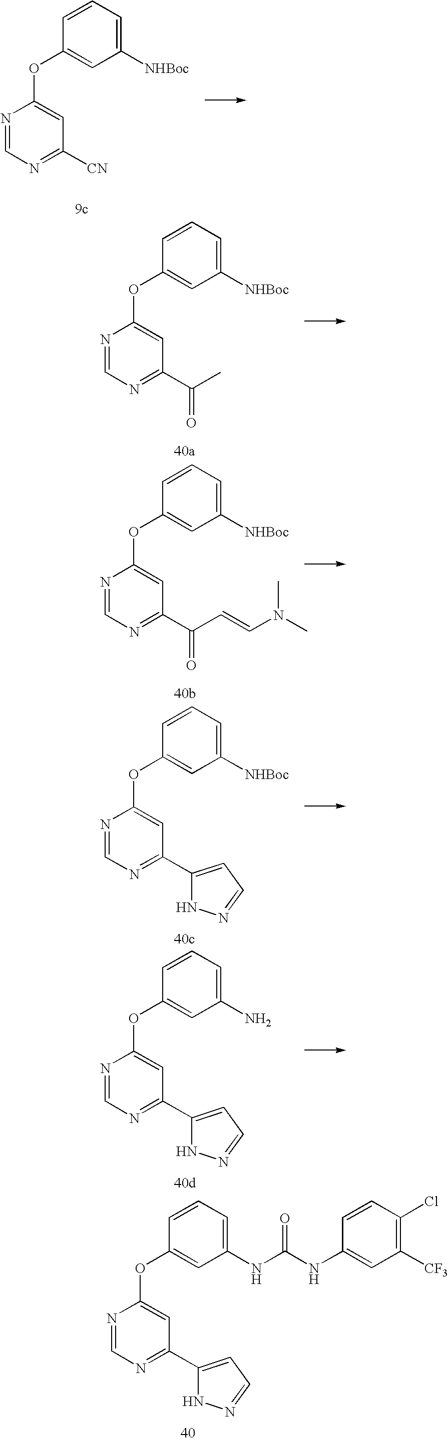 Step 1

### Preparation of compound 40a: tert-Butyl 3-(6-acetylpyrimidin-4-yloxy)phenylcarbamate

Methyl iodide (90.9 g, 641 mmol) was added dropwise over 3 h to a solution of magnesium (15.6 g, 641 mmol) in diethyl ether (200 mL) at room temperature under nitrogen. The resulting solution was transferred dropwise over 1 h via a cannula to a 0° C. mixture of 9c (50.0 g, 160 mmol) and THF (200 mL). Additional THF (500 mL) was added and the reaction mixture was warmed to room temperature. The mixture was stirred for 3 h prior to addition of NH₄Cl (300 mL of a saturated aqueous solution). The phases were separated and the aqueous layer was extracted with ethyl acetate (3×200 mL). The combined organic layers were concentrated under vacuum and the product was purified using column chromatography (hexanes to 20:1 hexanes/ethyl acetate) to give 16.5 g of tert-butyl 3-(6-acetylpyrimidin-4-yloxy)phenylcarbamate as a white solid. [M+H]⁺ 329.95.

Step 2

### Preparation of compound 40b: (E)-tert-Butyl 3-(6-(3-(dimethylamino)acryloyl)pyrimidin-4-yloxy)phenylcarbamate

A mixture of 40a (7.0 g, 21 mmol) and dimethylformamide dimethyl acetal (90 mL) was heated to 80° C. for 1.5 h prior to cooling to room temperature. The solution was concentrated under vacuum and the product was recrystallized using methanol to give 5.6 g of (E)-tert-butyl 3-(6-(3-(dimethylamino)acryloyl)pyrimidin-4-yloxy)phenylcarbamate as a white solid. [M+H]⁺ 385.05.

Step 3

### Preparation of compound 40c: tert-Butyl 3-(6-(1H-pyrazol-5-yl)pyrimidin-4-yloxy)phenylcarbamate

Hydrazine monohydrate (3.0 g, 60 mmol) was added to a mixture of 40b (900 mg, 2.3 mmol) and ethanol (200 mL) at room temperature. The solution was heated to 40-50° C. for 3 h prior to cooling to room temperature. NH₄Cl (300 mL of a saturated aqueous solution) was added and the mixture was extracted with DCM (2×200 mL). The combined organic layers were concentrated under vacuum to give 780 mg of tert-butyl 3-(6-(1H-pyrazol-5-yl)pyrimidin-4-yloxy)phenylcarbamate as a white solid. [M+H]⁺ 354.00.

Step 4

Preparation of compound 40d: 3-(6-(1H-Pyrazol-5-yl)pyrimidin-4-yloxy)aniline was prepared following the procedures described in preparation of Example 1. [M+H]⁺ 253.82.

Step 5

Preparation of compound 40: 1-(3-(6-(1H-Pyrazol-5-yl)pyrimidin-4-yloxy)phenyl)-3-(4-chloro-3-(trifluoromethyl)phenyl)urea was prepared following the procedures described in preparation of Example 1. [M+H]⁺ 475.14; ¹H-NMR (400 MHz, DMSO) δ 13.33 (s, 1H), 9.20 (s, 1H), 9.04 (s, 1H), 8.76 (s, 1H), 8.07 (d, 1H), 7.89 (s, 1H), 7.63-7.57 (m, 2H), 7.47 (t, 1H), 7.38-7.27 (m, 4H), 6.91-6.86 (m, 1H).

### EXAMPLE 41

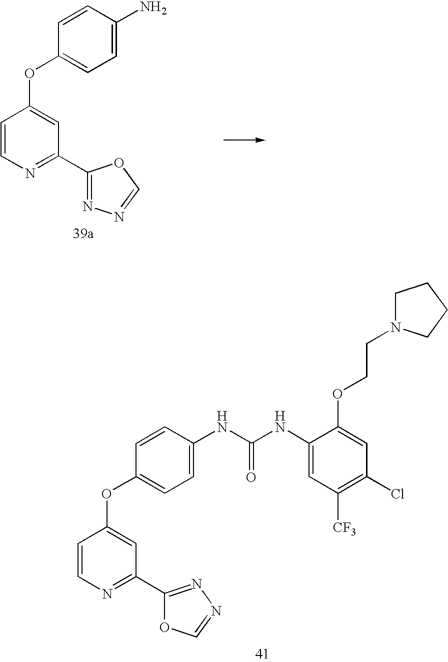

Preparation of compound 41: 1-(4-(2-(1,3,4-Oxadiazol-2-yl)pyridin-4-yloxy)phenyl)-3-(4-chloro-2-(2-(pyrrolidin-1-yl)ethoxy)-5-(trifluoromethyl)phenyl)urea was prepared following the procedures described in preparation of Example 24. [M+H]⁺ 589.23; ¹H-NMR (400 MHz, DMSO) δ 10.48 (s, 1H), 9.39 (s, 1H), 9.25 (s, 1H), 8.76 (s, 1H), 8.62 (d, 1H), 7.61 (m, 2H), 7.55 (m, 1H), 7.38 (s, 1H), 7.16 (m, 3H), 4.49 (t, 2H), 3.39-3.31 (m, 4H), 2.48 (m, 2H), 2.00 (m, 4H).

### EXAMPLE 42

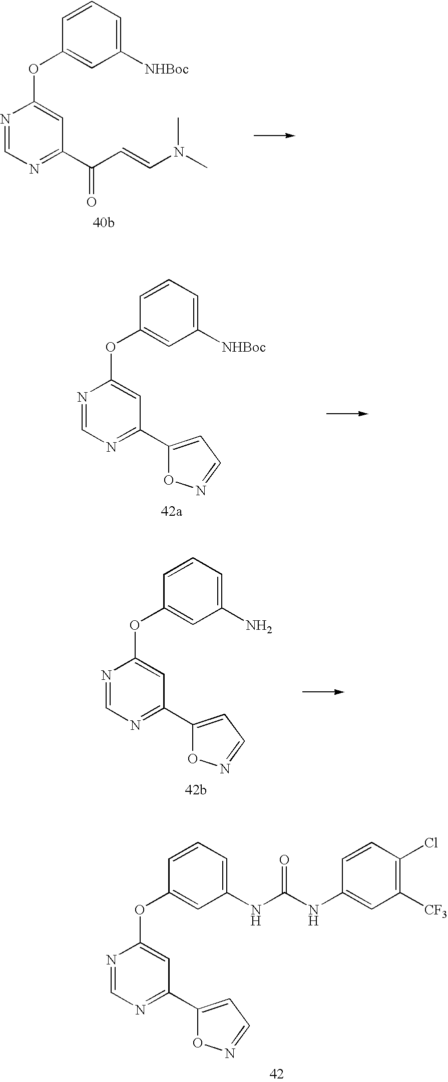 Step 1

Preparation of compound 42a: tert-Butyl 3-(6-(isoxazol-5-yl)pyrimidin-4-yloxy)phenylcarbamate was prepared following the procedures described in preparation of Example 40. [M+H]⁺ 345.93.

Step 2

Preparation of compound 42b: 3-(6-(Isoxazol-5-yl)pyrimidin-4-yloxy)aniline was prepared following the procedures described in preparation of Example 1. [M+H]⁺ 255.86.

Step 3

Preparation of compound 42: 1-(4-chloro-3-(trifluoromethyl)phenyl)-3-(3-(6-(isoxazol-5-yl)pyrimidin-4-yloxy)phenyl)urea was prepared following the procedures described in preparation of Example 1. [M+H]⁺ 475.80; ¹H-NMR (400 MHz, DMSO) δ 9.20 (s, 1H), 9.05 (s, 1H), 8.87 (d, 1H), 8.83 (d, 1H), 8.08 (d, 1H), 7.64-7.57 (m, 3H), 7.50 (t, 1H), 7.40-7.35 (m, 2H), 7.30-7.27 (m, 1H), 6.91-6.87 (m, 1H).

### EXAMPLE 43

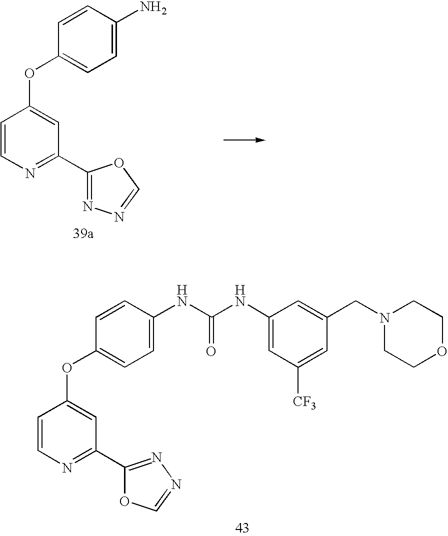

Preparation of compound 43: 1-(4-(2-(1,3,4-Oxadiazol-2-yl)pyridin-4-yloxy)phenyl)-3-(3-(morpholinomethyl)-5-(trifluoromethyl)phenyl)urea was prepared following the procedures described in preparation of Example 24. [M+H]⁺ 542.21; ¹H-NMR (400 MHz, CD₃OD) δ 9.08 (s, 1H), 8.53 (d, 1H), 7.80 (s, 1H), 7.66 (d, 1H), 7.58 (m, 3H), 7.28 (s, 1H), 7.13 (m, 2H), 7.08 (dd, 1H), 3.70 (m, 4H), 3.55 (s, 2H), 2.42 (m, 4H).

### EXAMPLE 44

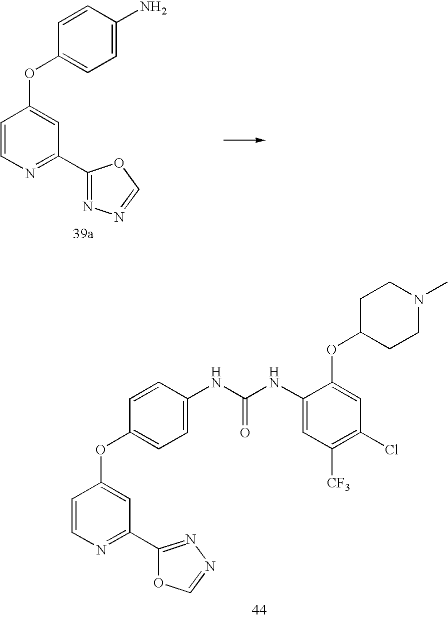

Preparation of compound 44: 1-[4-Chloro-2-(1-methyl-piperidin-4-yloxymethyl)-5-trifluoromethyl-phenyl]-3-[4-(2-[1,3,4]oxadiazol-2-yl-pyridin-4-yloxy)-phenyl]-urea was prepared following the procedures described in preparation of Example 24. [M+H]⁺ 589.16; ¹H-NMR (400 MHz, CD₃OD) δ 9.09 (s, 1H), 8.63 (s, 1H), 8.55 (d, 1H), 7.69 (d, 1H), 7.62 (m, 2H), 7.26 (s, 1H), 7.16 (m, 2H), 7.11 (dd, 1H), 7.06 (s, 1H), 7.02 (s, 1H), 4.65 (m, 1H), 3.10-1-90 (m, 11H).

### EXAMPLE 45

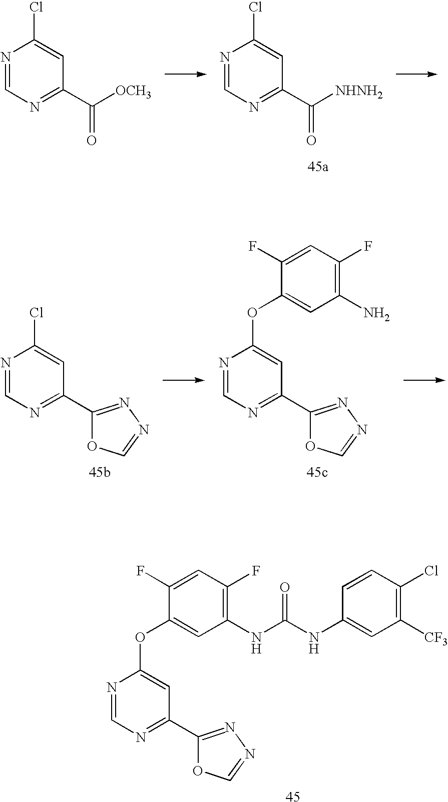 Step 1

Preparation of compound 45a: 6-Chloro-pyrimidine-4-carboxylic acid hydrazide was prepared following the procedures described in preparation of Example 8. [M+H]⁺ 173.

Step 2

Preparation of compound 45b: 4-Chloro-6-[1,3,4]oxadiazol-2-yl-pyrimidine was prepared following the procedures described in preparation of Example 8. [M+H]⁺ 183.

Step 3

Preparation of compound 45c: 2,4-Difluoro-5-(6-[1,3,4]oxadiazol-2-yl-pyrimidin-4-yloxy)-phenylamine was prepared following the procedures described in preparation of Example 8. [M+H]⁺ 292.

Step 4

Preparation of compound 45: 1-(5-(6-(1,3,4-Oxadiazol-2-yl)pyrimidin-4-yloxy)-2,4-difluorophenyl)-3-(4-chloro-3-(trifluoromethyl)phenyl)urea was prepared following the procedures described in preparation of Example 1. [M+H]⁺ 513; ¹H-NMR (400 MHz, DMSO) δ 9.57 (s, 1H), 9.53 (br s, 1H), 9.00 (d, 1H), 8.53 (d, 1H), 8.15 (t, 1H), 8.11 (d, 1H), 7.97 (d, 1H), 7.61 (m, 3H).

### EXAMPLE 46

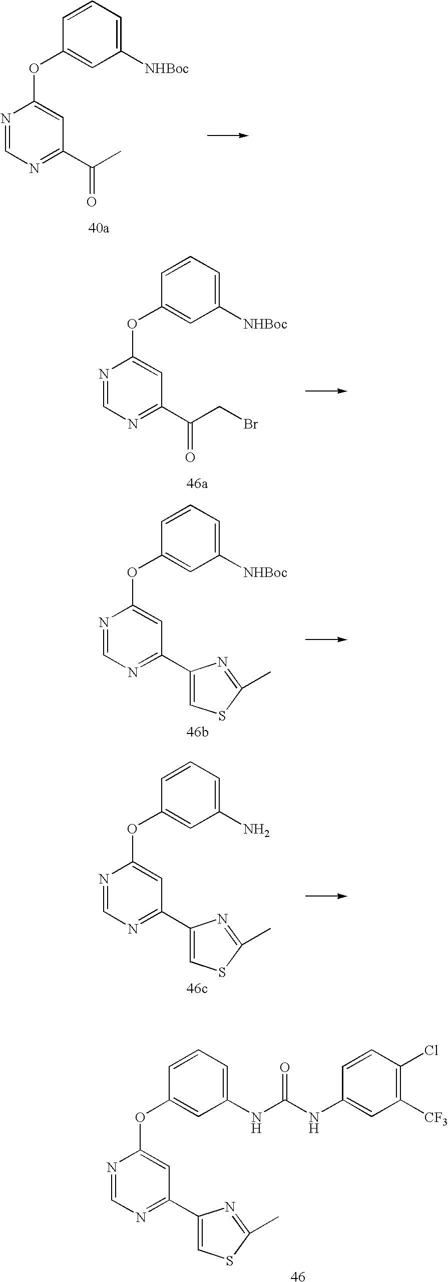 Step 1

### Preparation of compound 46a: tert-Butyl 3-(6-(2-bromoacetyl)pyrimidin-4-yloxy)phenylcarbamate

A solution of tetrabutylammonium tribromide (7.5 g, 16 mmol) in DCM (30 mL) was added dropwise to a mixture of 40a (5.0 g, 15 mmol) and DCM (120 mL) at room temperature under nitrogen. The reaction mixture was stirred for 2 h prior to the addition of water (100 mL). The phases were separated and the aqueous layer was extracted with ethyl acetate (2×100 mL). The combined organic layers were concentrated under vacuum to give 5.8 g of tert-butyl 3-(6-(2-bromoacetyl)pyrimidin-4-yloxy)phenylcarbamate as a dark oil. [M+H]⁺ 408.89, 410.78.

Step 2

### Preparation of compound 46b: tert-Butyl 3-(6-(2-methylthiazol-4-yl)pyrimidin-4-yloxy)phenylcarbamate

Thioacetamide (900 mg, 12.0 mmol) was added to a solution of 46a (5.80 g, 14.2 mmol) in ethanol (100 mL) at room temperature under nitrogen. The reaction mixture was heated to 40° C. for 1.5 h prior to cooling to room temperature. NaHCO₃ (200 mL of a saturated aqueous solution) and ethyl acetate (150 mL) were added. The phases were separated and the aqueous back extracted with ethyl acetate (150 mL). The combined organic layers were concentrated under vacuum and the product was purified using column chromatography (hexanes to 20:1 hexanes/ethyl acetate) to give 300 mg of tert-butyl 3-(6-(2-methylthiazol-4-yl)pyrimidin-4-yloxy)phenylcarbamate as a white solid. [M+H]⁺ 384.95.

Step 3

Preparation of compound 46c: 3-(6-(2-Methylthiazol-4-yl)pyrimidin-4-yloxy)aniline was prepared following the procedures described in preparation of Example 1. [M+H]⁺ 285.54.

Step 4

Preparation of compound 46: 1-(4-Chloro-3-(trifluoromethyl)phenyl)-3-(3-(6-(2-methylthiazol-4-yl)pyrimidin-4-yloxy)phenyl)urea was prepared following the procedures described in preparation of Example 1. [M+H]⁺ 505.81; ¹H-NMR (400 MHz, DMSO) δ 9.21 (s, 1H), 9.05 (s, 1H), 8.78 (s, 1H), 8.37 (s, 1H), 8.07 (s, 1H), 7.64-7.57 (m, 2H), 7.41 (t, 1H), 7.42-7.36 (m, 2H), 7.31-7.28 (m, 1H), 6.90-6.87 (m, 1H), 2.71 (s, 3H).

### EXAMPLE 47

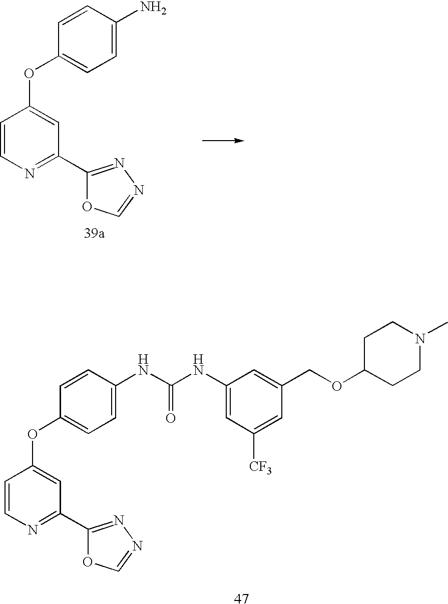

Preparation of compound 47: 1-(4-(2-(1,3,4-Oxadiazol-2-yl)pyridin-4-yloxy)phenyl)-3-(3-((1-methylpiperidin-4-yloxy)methyl)-5-(trifluoromethyl)phenyl)urea was prepared following the procedures described in preparation of Example 24. [M+H]⁺ 570.33.

### EXAMPLE 48

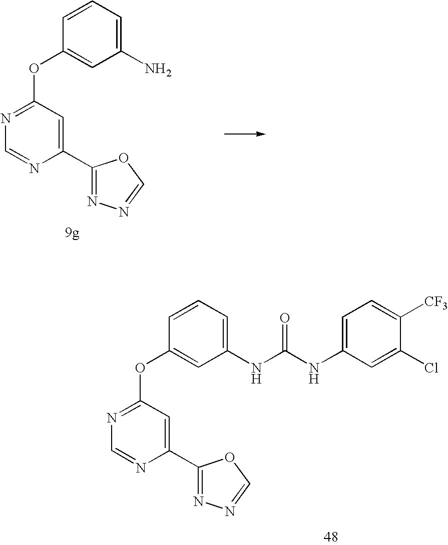

Preparation of compound 48: 1-(3-(6-(1,3,4-Oxadiazol-2-yl)pyrimidin-4-yloxy)phenyl)-3-(3-chloro-4-(trifluoromethyl)phenyl)urea was prepared following the procedures described in preparation of Example 1. [M+H]⁺ 476.78.

### EXAMPLE 49

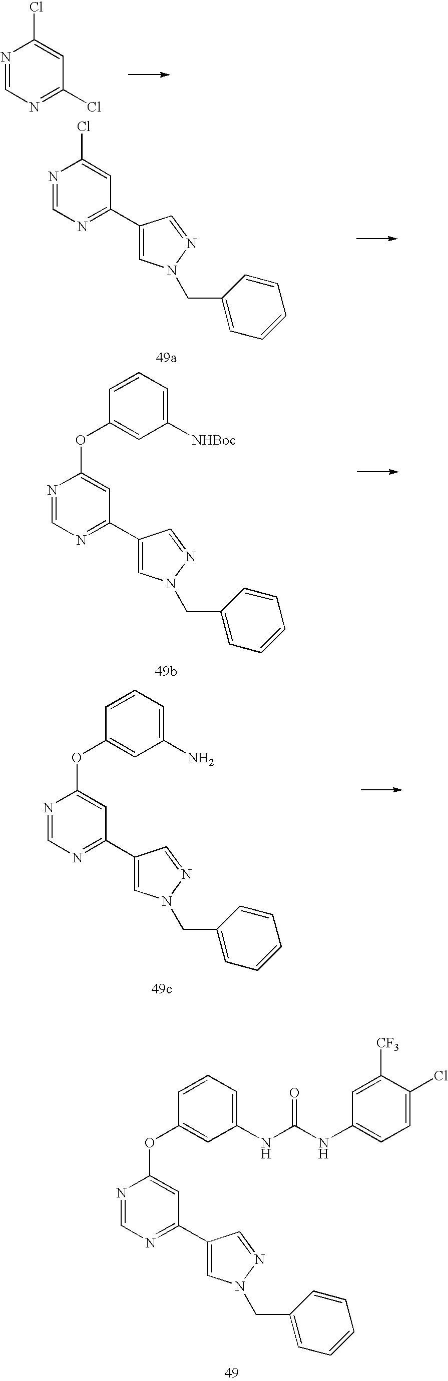 Step 1

Preparation of compound 49a: 4-(1-Benzyl-1H-pyrazol-4-yl)-6-chloropyrimidine was prepared following the procedures described in preparation of Example 27. [M+H]⁺ 270.90.

Step 2

Preparation of compound 49b: {3-[6-(1-Benzyl-1H-pyrazol-4-yl)-pyrimidin-4-yloxy]-phenyl}-carbamic acid tert-butyl ester was prepared following the procedures described in preparation of Example 1. [M+H]⁺ 444.85.

Step 3

Preparation of compound 49c: 3-(6-(1-Benzyl-1H-pyrazol-4-yl)pyrimidin-4-yloxy)aniline was prepared following the procedures described in preparation of Example 1. [M+H]⁺ 344.18.

Step 4

Preparation of compound 49: 1-(3-(6-(1-Benzyl-1H-pyrazol-4-yl)pyrimidin-4-yloxy)phenyl)-3-(4-chloro-3-(trifluoromethyl)phenyl)urea was prepared following the procedures described in preparation of Example 1. [M+H]⁺ 564.91; ¹H-NMR (400 MHz, DMSO) δ 9.18 (s, 1H), 9.01 (s, 1H), 8.63 (s, 1H), 8.60 (s, 1H), 8.20 (s, 1H), 8.07 (d, 1H), 7.64-7.57 (m, 2H), 7.43 (t, 1H), 7.38-7.24 (m, 8H), 6.85-6.81 (m, 1H), 5.39 (s, 2H).

### EXAMPLE 50

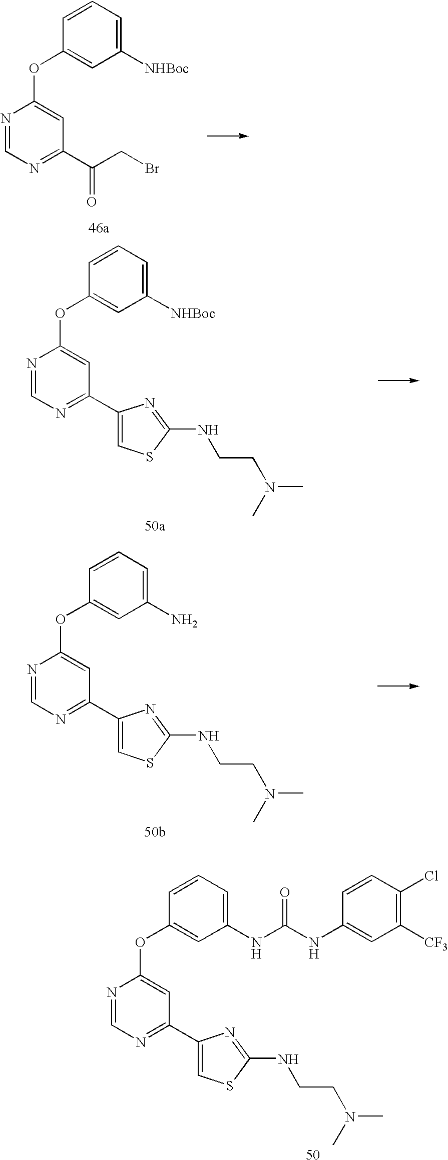 Step 1

Preparation of compound 50a: tert-Butyl 3-(6-(2-(2-(dimethylamino)ethylamino)thiazol-4-yl)pyrimidin-4-yloxy)phenylcarbamate was prepared following the procedures described in preparation of Example 46. [M+H]⁺ 455.25

Step 2

Preparation of compound 50b: N1-(4-(6-(3-Aminophenoxy)pyrimidin-4-yl)thiazol-2-yl)-N2,N2-dimethylethane-1,2-diamine was prepared following the procedures described in preparation of Example 1. [M+H]⁺ 357.02.

Step 3

Preparation of compound 50: 1-(4-Chloro-3-(trifluoromethyl)phenyl)-3-(3-(6-(2-(2-(dimethylamino)ethylamino)thiazol-4-yl)pyrimidin-4-yloxy)phenyl)urea was prepared following the procedures described in preparation of Example 1. [M+H]⁺ 578.30; ¹H-NMR (400 MHz, DMSO) δ 9.41 (s, 1H), 9.22 (s, 1H), 8.70 (d, 1H), 8.08 (d, 1H), 7.90 (br s, 1H), 7.64-7.58 (m, 2H), 7.47 (s, 1H), 7.39-7.26 (m, 3H), 6.87-6.85 (m, 1H), 3.48 (br s, 2H), 3.31 (s, 6H), 2.83 (br s, 1H), 2.43 (br s, 2H).

### EXAMPLE 51

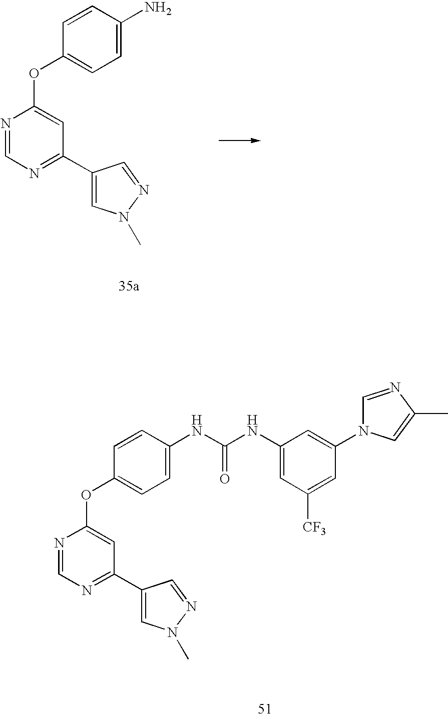

Preparation of compound 51: 1-(3-(4-Methyl-1H-imidazol-1-yl)-5-(trifluoromethyl)phenyl)-3-(4-(6-(1-methyl-1H-pyrazol-4-yl)pyrimidin-4-yloxy)phenyl)urea was prepared following the procedures described in preparation of Example 24. [M+H]⁺ 534.68; ¹H-NMR (400 MHz, DMSO) δ 9.32 (s, 1H), 9.14 (s, 1H), 8.61 (d, 1H), 8.44 (s, 1H), 8.24 (d, 1H), 8.13 (d, 1H), 7.85 (m, 2H), 7.52 (m, 4H), 7.32 (d, 1H), 7.14 (d, 2H), 3.89 (s, 3H), 2.17 (s, 3H).

### EXAMPLE 52

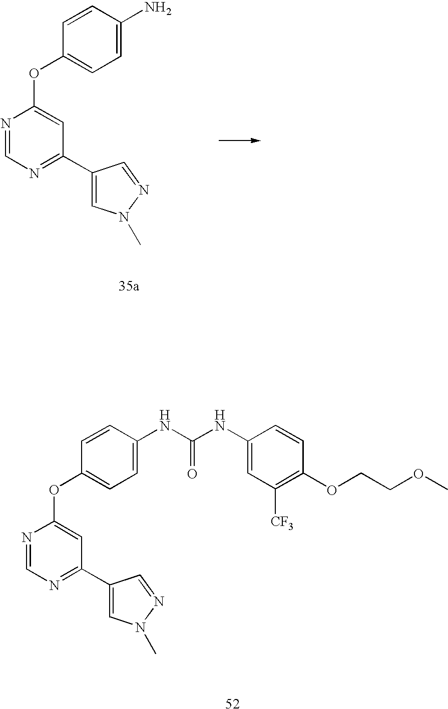

Preparation of compound 52: 1-(4-(2-Methoxyethoxy)-3-(trifluoromethyl)phenyl)-3-(4-(6-(1-methyl-1H-pyrazol-4-yl)pyrimidin-4-yloxy)phenyl)urea was prepared following the procedures described in preparation of Example 24. [M+H]⁺ 529.84; ¹H-NMR (400 MHz, DMSO) δ 8.80 (s, 1H), 8.79 (s, 1H), 8.61 (d, 1H), 8.43 (s, 1H), 8.12 (s, 1H), 7.83 (d, 1H), 7.58 (m, 1H), 7.50 (d, 2H), 7.30 (d, 1H), 7.21 (d, 1H), 7.12 (d, 2H), 4.16 (t, 2H), 3.88 (s, 3H), 3.65 (t, 2H).

### EXAMPLE 53

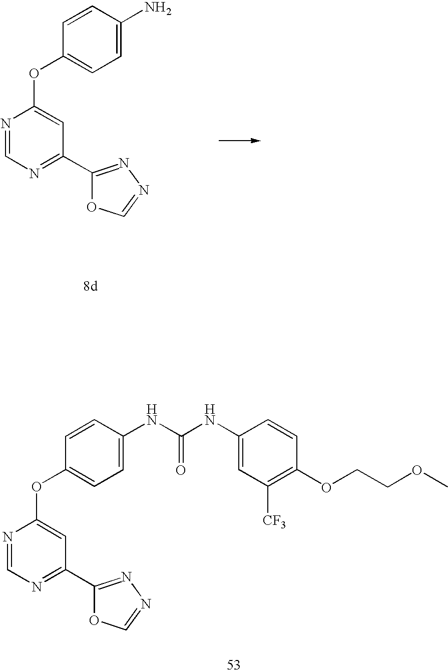

Preparation of compound 53: 1-(4-(6-(1,3,4-Oxadiazol-2-yl)pyrimidin-4-yloxy)phenyl)-3-(4-(2-methoxyethoxy)-3-(trifluoromethyl)phenyl)urea was prepared following the procedures described in preparation of Example 24. [M+H]⁺ 516.88.

### EXAMPLE 54

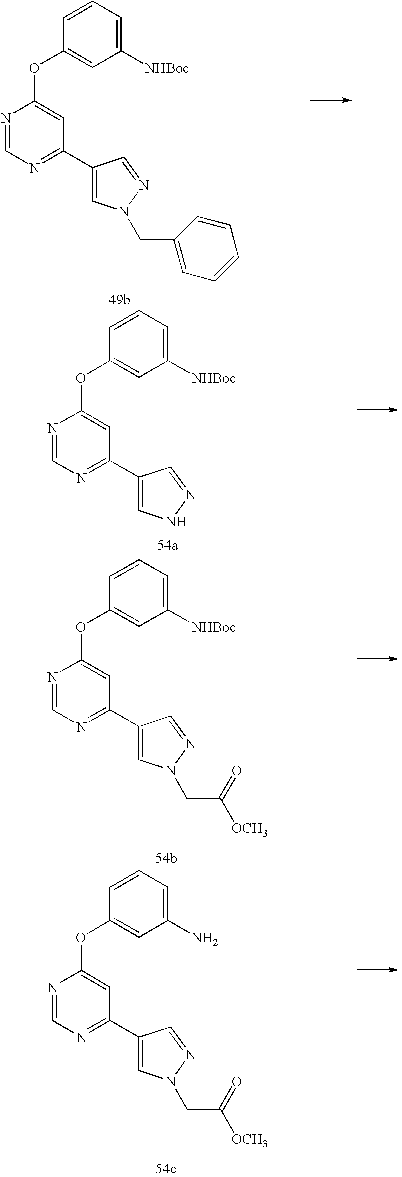 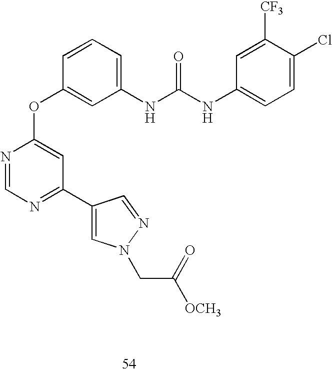 Step 1

### Preparation of compound 54a: tert-Butyl 3-(6-(1H-pyrazol-4-yl)pyrimidin-4-yloxy)phenylcarbamate

O₂ was bubbled through a solution of 49b (384 mg, 866 μmol), sodium tert-butoxide (583 mg, 6.06 mmol) and DMSO (5 mL) at room temperature for 20 minutes. Brine (100 mL) was added and the mixture extracted with ethyl acetate (15 mL). The organic layer was concentrated under vacuum and the product was purified using column chromatography (hexanes to ethyl acetate) to give 215 mg of tert-butyl 3-(6-(1H-pyrazol-4-yl)pyrimidin-4-yloxy)phenylcarbamate. [M+H]⁺ 354.81.

Step 2

### Preparation of compound 54b: Methyl 2-(4-(6-(3-(tert-butoxycarbonylamino)phenoxy)pyrimidin-4-yl)-1H-pyrazol-1-yl)acetate

Methyl bromoacetate (70 μL, 730 μmol) was added to a solution of 54a (220 mg, 610 μmol), sodium tert-butoxide (88 mg, 910 μmol) and DMSO (3 mL) at room temperature under nitrogen. The reaction mixture was stirred for 3 h prior to addition of brine (50 mL). The mixture was extracted with ethyl acetate (10 mL) and the organic layer was concentrated under vacuum. The product was purified using column chromatography (hexanes to 1:9 hexanes/ethyl acetate) to give 256 mg of methyl 2-(4-(6-(3-(tert-butoxycarbonylamino)phenoxy)pyrimidin-4-yl)-1H-pyrazol-1-yl)acetate. [M+H]⁺ 425.98.

Step 3

Preparation of compound 54c: Methyl 2-(4-(6-(3-aminophenoxy)pyrimidin-4-yl)-1H-pyrazol-1-yl)acetate was prepared following the procedures described in preparation of Example 1. [M+H]⁺ 325.99.

Step 4

Preparation of compound 54: Methyl 2-(4-(6-(3-(3-(4-chloro-3-(trifluoromethyl)phenyl)ureido)phenoxy)pyrimidin-4-yl)-1H-pyrazol-1-yl)acetate was prepared following the procedures described in preparation of Example 1. [M+H]⁺ 546.82; ¹H-NMR (400 MHz, DMSO) δ 9.20 (s, 1H), 9.02 (s, 1H), 8.65 (s, 1H), 8.50 (s, 1H), 8.22 (s, 1H), 8.07 (d, 1H), 7.64-7.58 (m, 2H), 7.43 (t, 1H), 7.40 (s, 1H), 7.35 (t, 1H), 7.29-7.25 (m, 1H), 6.87-6.83 (m, 1H), 5.17 (s, 2H), 3.69 (s, 3H).

### EXAMPLE 55

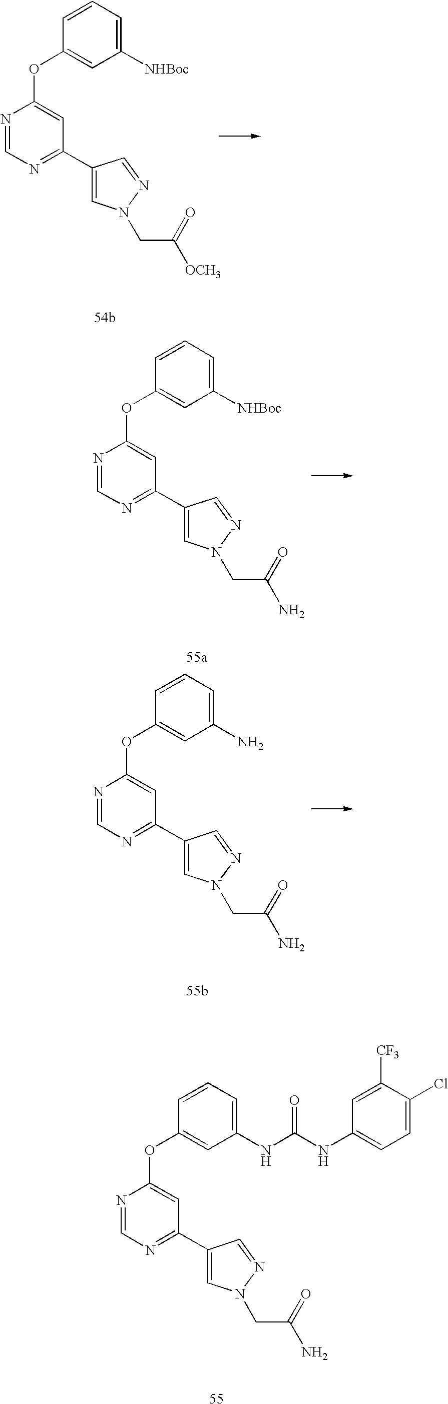 Step 1

Preparation of compound 55a: tert-Butyl 3-(6-(1-(2-amino-2-oxoethyl)-1H-pyrazol-4-yl)pyrimidin-4-yloxy)phenylcarbamate was prepared following the procedures described in preparation of Example 15. [M+H]⁺ 411.01.

Step 2

Preparation of compound 55b: 2-(4-(6-(3-Aminophenoxy)pyrimidin-4-yl)-1H-pyrazol-1-yl)acetamide was prepared following the procedures described in preparation of Example 1. [M+H]⁺ 310.91.

Step 3

Preparation of compound 55: 2-(4-(6-(3-(3-(4-Chloro-3-(trifluoromethyl)phenyl)ureido)phenoxy)pyrimidin-4-yl)-1H-pyrazol-1-yl)acetamide was prepared following the procedures described in preparation of Example 1. [M+H]⁺ 531.85; ¹H-NMR (400 MHz, DMSO) δ 9.21(s, 1H), 9.03 (s, 1H), 8.64 (s, 1H), 8.44 (s, 1H), 8.16 (s, 1H), 8.07 (d, 1H), 7.64-7.58 (m, 3H), 7.43-7.25 (m, 5H), 6.86-6.82 (m, 1H), 4.82 (s, 2H).

### EXAMPLE 56

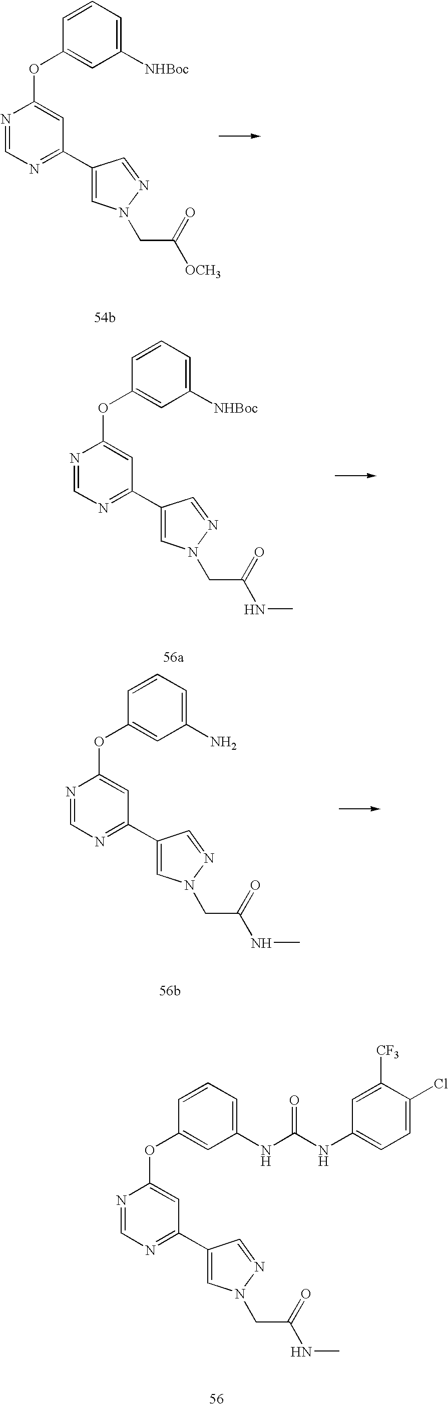 Step 1

Preparation of compound 56a: tert-Butyl 3-(6-(1-(2-(methylamino)-2-oxoethyl)-1H-pyrazol-4-yl)pyrimidin-4-yloxy)phenylcarbamate was prepared following the procedures described in preparation of Example 15. [M+H]⁺ 425.04.

Step 2

Preparation of compound 56b: 2-(4-(6-(3-Aminophenoxy)pyrimidin-4-yl)-1H-pyrazol-1-yl)-N-methylacetamide was prepared following the procedures described in preparation of Example 1. [M+H]⁺ 324.94.

Step 3

Preparation of compound 56: 2-(4-(6-(3-(3-(4-Chloro-3-(trifluoromethyl)phenyl)ureido)phenoxy)pyrimidin-4-yl)-1H-pyrazol-1-yl)-N-methylacetamide was prepared following the procedures described in preparation of Example 1. [M+H]⁺ 545.86; ¹H-NMR (400 MHz, DMSO) δ 9.20 (s, 1H), 9.03 (s, 1H), 8.64 (s, 1H), 8.46 (s, 1H), 8.17 (s, 1H), 8.07 (m, 2H), 7.64-7.58 (m, 2H), 7.43-7.25 (m, 4H), 6.85-6.82 (m, 1H), 4.83 (s, 2H), 2.61 (d, 3H).

### EXAMPLE 57

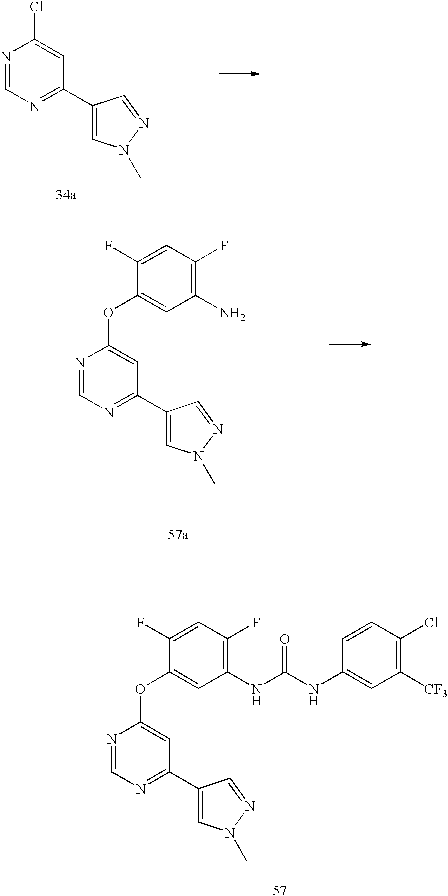 Step 1

Preparation of compound 57a: 2,4-Difluoro-5-[6-(1-methyl-1H-pyrazol-4-yl)-pyrimidin-4-yloxy]-phenylamine was prepared following the procedures described in preparation of Example 1. [M+H]⁺ 304.

Step 2

Preparation of compound 57: 1-(4-Chloro-3-(trifluoromethyl)phenyl)-3-(2,4-difluoro-5-(6-(1-methyl-1H-pyrazol-4-yl)pyrimidin-4-yloxy)phenyl)urea was prepared following the procedures described in preparation of Example 1. [M+H]⁺ 525; ¹H-NMR (400 MHz, DMSO) δ 9.51 (br s, 1H), 8.81 (br s, 1H), 8.64 (d, 1H), 8.49 (s, 1H), 8.19 (s, 1H), 8.10 (d, 1H), 8.06 (t, 1H), 7.61 (m, 3H), 7.52 (d, 1H), 3.91 (s, 3H).

### EXAMPLE 58

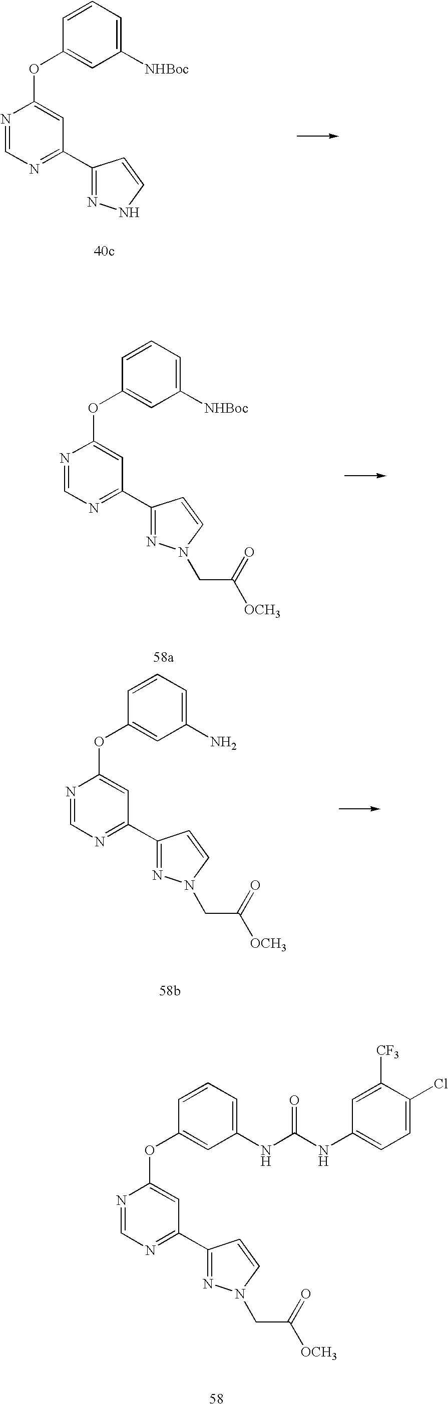 Step 1

Preparation of compound 58a: {3-[6-(3-tert-Butoxycarbonylamino-phenoxy)-pyrimidin-4-yl]-pyrazol-1-yl}-acetic acid methyl ester was prepared following the procedures described in preparation of Example 54. [M+H]⁺ 425.47.

Step 2

Preparation of compound 58b: {3-[6-(3-Amino-phenoxy)-pyrimidin-4-yl]-pyrazol-1-yl}-acetic acid methyl ester was prepared following the procedures described in preparation of Example 1. [M+H]⁺ 325.95.

Step 3

Preparation of compound 58: Methyl 2-(3-(6-(3-(3-(4-chloro-3-(trifluoromethyl)phenyl)ureido)phenoxy)pyrimidin-4-yl)-1H-pyrazol-1-yl)acetate was prepared following the procedures described in preparation of Example 1. [M+H]⁺ 546.88; ¹H-NMR (400 MHz, DMSO) δ 9.21 (s, 1H), 9.05 (s, 1H), 8.78 (s, 1H), 8.07 (d, 1H), 7.90 (d, 1H), 7.62-7.59 (m, 2H), 7.45 (t, 1H), 7.38 (t, 1H), 7.32-7.27 (m, 2H), 6.96 (d, 1H), 6.89-6.85 (m, 1H), 5.20 (s, 2H), 3.68 (s, 3H).

### EXAMPLE 59

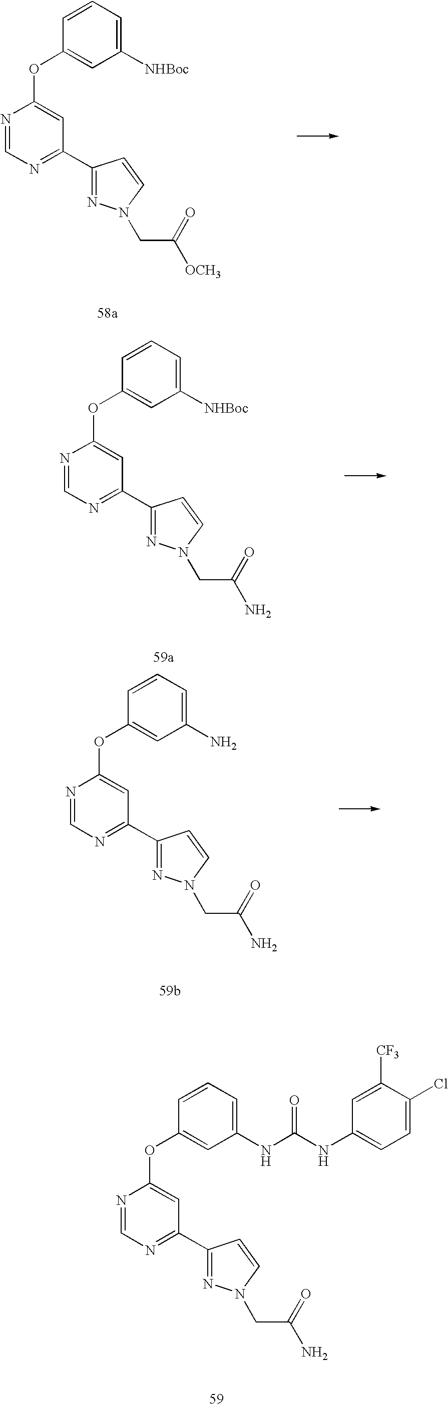 Step 1

Preparation of compound 59a: {3-[6-(1-Carbamoylmethyl-1H-pyrazol-3-yl)-pyrimidin-4-yloxy]-phenyl}-carbamic acid tert-butyl ester was prepared following the procedures described in preparation of Example 15. [M+H]⁺ 411.74.

Step 2

Preparation of compound 59b: 2-{3-[6-(3-Amino-phenoxy)-pyrimidin-4-yl]-pyrazol-1-yl}-acetamide was prepared following the procedures described in preparation of Example 1. [M+H]⁺ 310.75.

Step 3

Preparation of compound 59: 2-(3-(6-(3-(3-(4-Chloro-3-(trifluoromethyl)phenyl)ureido)phenoxy)pyrimidin-4-yl)-1H-pyrazol-1-yl)acetamide was prepared following the procedures described in preparation of Example 1. [M+H]⁺ 531.85; ¹H-NMR (400 MHz, DMSO) δ 9.22 (s, 1H), 9.05 (s, 1H), 8.77 (d, 1H), 8.07 (s, 1H), 7.84 (d, 1H), 7.62-7.54 (m, 3H), 7.44 (t, 1H), 7.38 (t, 1H), 7.32-7.27 (m, 3H), 6.91 (d, 1H), 6.88-6.85 (m, 1H), 4.86 (s, 2H).

### EXAMPLE 60

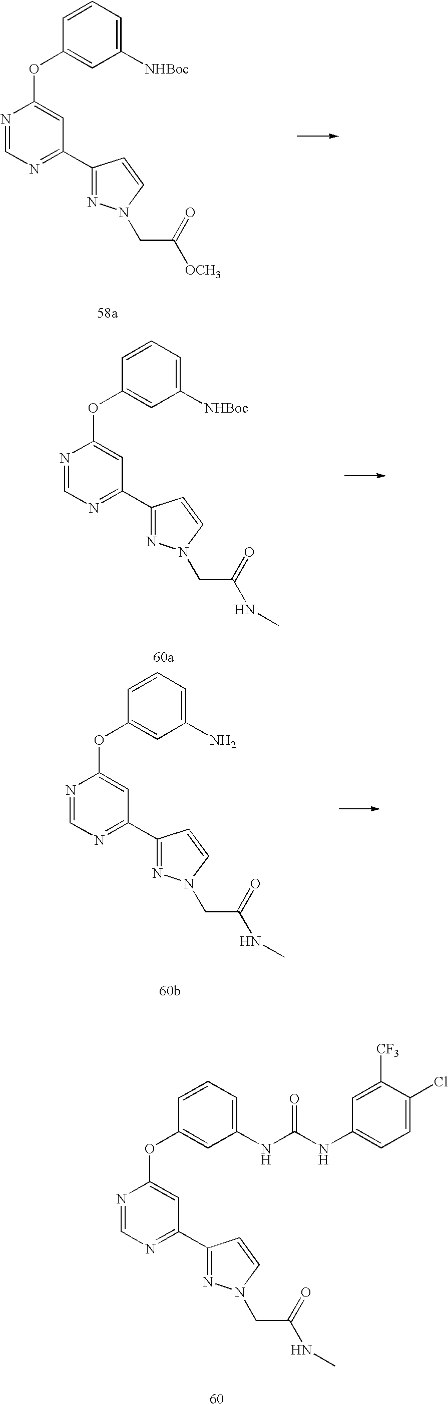 Step 1

Preparation of compound 60a: {3-[6-(1-Methylcarbamoylmethyl-1H-pyrazol-3-yl)-pyrimidin-4-yloxy]-phenyl}-carbamic acid tert-butyl ester was prepared following the procedures described in preparation of Example 15. [M+H]⁺ 424.71.

Step 2

Preparation of compound 60b: 2-{3-[6-(3-Amino-phenoxy)-pyrimidin-4-yl]-pyrazol-1-yl}-N-methyl-acetamide was prepared following the procedures described in preparation of Example 1. [M+H]⁺ 324.99.

Step 3

Preparation of compound 60: 2-(3-(6-(3-(3-(4-Chloro-3-(trifluoromethyl)phenyl)ureido)phenoxy)pyrimidin-4-yl)-1H-pyrazol-1-yl)-N-methylacetamide was prepared following the procedures described in preparation of Example 1. [M+H]⁺ 545.86; ¹H-NMR (400 MHz, DMSO) δ 9.21 (s, 1H), 9.05 (s, 1H), 8.77 (d, 1H), 8.07 (s, 1H), 7.85 (d, 1H), 7.64-7.58 (m, 2H), 7.44 (t, 1H), 7.38 (t, 1H), 7.32-7.27 (m, 3H), 6.92 (d, 1H), 6.88-6.85 (m, 1H), 4.86 (s, 2H), 2.60 (d, 3H).

### EXAMPLE 61

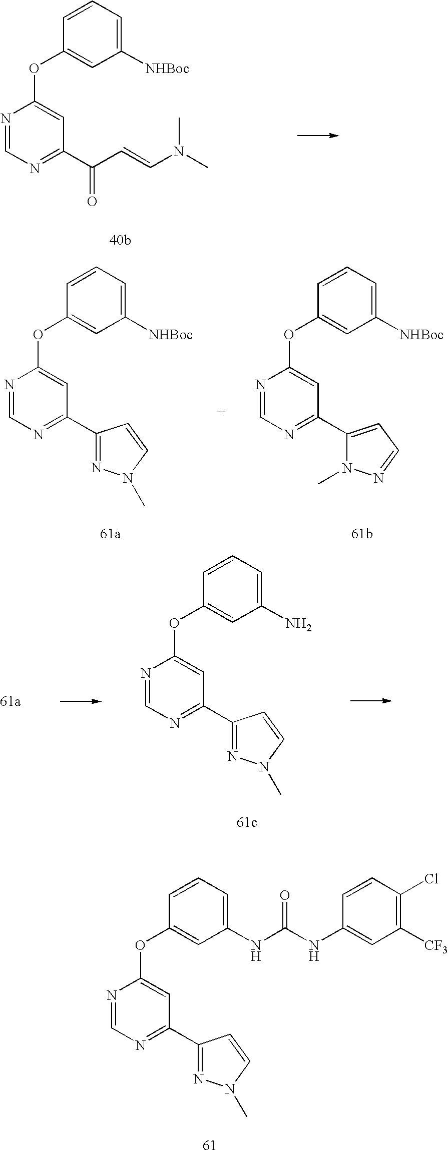 Step 1

Preparation of compounds 61a and 61b: {3-[6-(1-Methyl-1H-pyrazol-3-yl)-pyrimidin-4-yloxy]-phenyl}-carbamic acid tert-butyl ester and {3-[6-(2-Methyl-2H-pyrazol-3-yl)-pyrimidin-4-yloxy]-phenyl}-carbamic acid tert-butyl ester were prepared following the procedures described in preparation of Example 40. Data for 61a: [M+H]⁺ 368.20. Data for 61b: [M+H]⁺ 368.97.

Step 2

Preparation of compound 61b: 3-[6-(1-Methyl-1H-pyrazol-3-yl)-pyrimidin-4-yloxy]-phenylamine was prepared following the procedures described in preparation of Example 1. [M+H]⁺ 267.97.

Step 3

Preparation of compound 61: 1-(4-Chloro-3-(trifluoromethyl)phenyl)-3-(3-(6-(1-methyl-1H-pyrazol-3-yl)pyrimidin-4-yloxy)phenyl)urea was prepared following the procedures described in preparation of Example 1. [M+H]⁺ 488.81; ¹H-NMR (400 MHz, DMSO) δ 9.20 (s, 1H), 9.04 (s, 1H), 8.83 (d, 1H), 8.08 (d, 1H), 7.63-7.59 (m, 2H), 7.57 (d, 1H), 7.55 (d, 1H), 7.49 (t, 1H), 7.36 (t, 1H), 7.27-7.24 (d, 1H), 7.07 (d, 1H), 6.89-6.84 (m, 1H), 4.20 (s, 3H).

### EXAMPLE 62

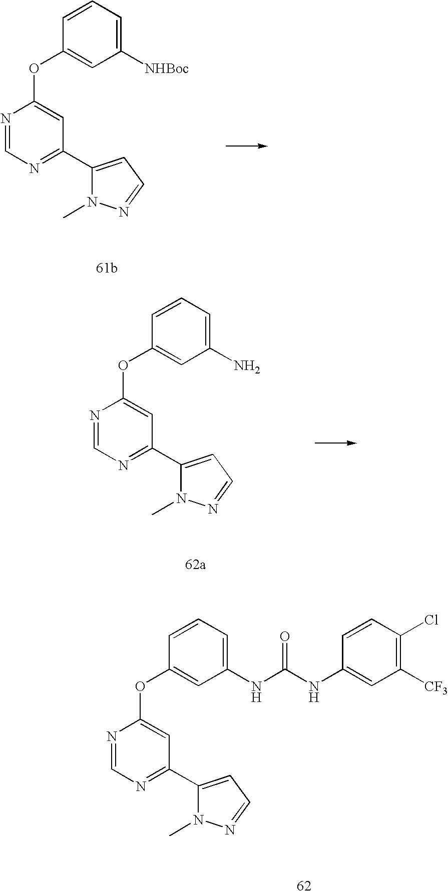 Step 1

Preparation of compound 62a: 3-[6-(2-Methyl-2H-pyrazol-3-yl)-pyrimidin-4-yloxy]-phenylamine was prepared following the procedures described in preparation of Example 1. [M+H]⁺ 267.69.

Step 2

Preparation of compound 62: 1-(4-Chloro-3-(trifluoromethyl)phenyl)-3-(3-(6-(1-methyl-1H-pyrazol-5-yl)pyrimidin-4-yloxy)phenyl)urea was prepared following the procedures described in preparation of Example 1. [M+H]⁺ 488.81; ¹H-NMR (400 MHz, DMSO) δ 9.22 (s, 1H), 9.06 (s, 1H), 8.76 (d, 1H), 8.07 (d, 1H), 7.83 (d, 1H), 7.64-7.56 (m, 2H), 7.39 (t, 1H), 7.31 (t, 1H), 7.32-7.26 (m, 2H), 6.90-6.84 (m, 2H), 3.91 (s, 3H).

### EXAMPLE 63

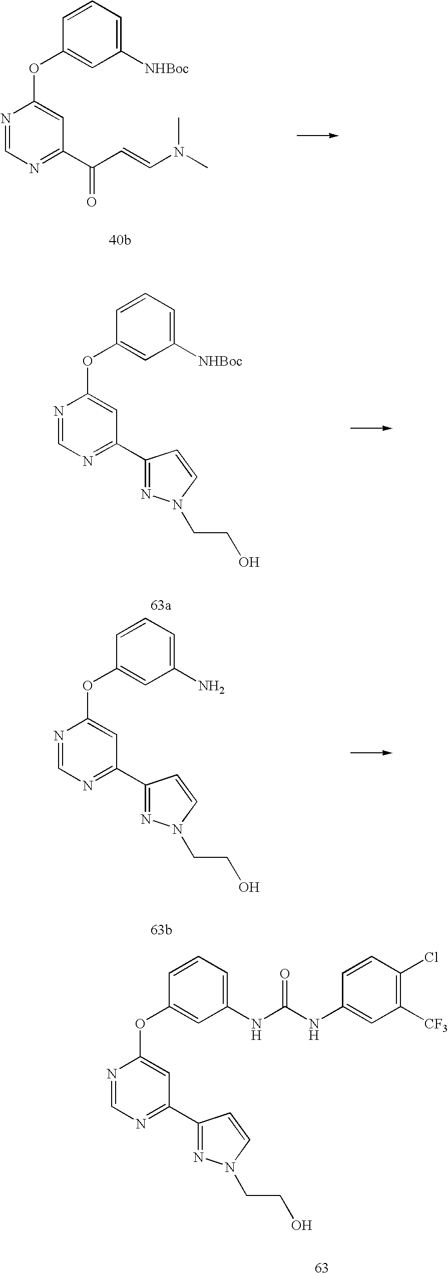 Step 1

Preparation of compound 63a: (3-{6-[1-(2-Hydroxy-ethyl)-1H-pyrazol-3-yl]-pyrimidin-4-yloxy}-phenyl)-carbamic acid tert-butyl ester was prepared following the procedures described in preparation of Example 40. [M+H]⁺ 397.98.

Step 2

Preparation of compound 63b: 2-{3-[6-(3-Amino-phenoxy)-pyrimidin-4-yl]-pyrazol-1-yl}-ethanol was prepared following the procedures described in preparation of Example 1. [M+H]⁺ 297.94.

Step 3

Preparation of compound 63: 1-(4-Chloro-3-(trifluoromethyl)phenyl)-3-(3-(6-(1-(2-hydroxyethyl)-1H-pyrazol-3-yl)pyrimidin-4-yloxy)phenyl)urea was prepared following the procedures described in preparation of Example 1. [M+H]⁺ 518.86; ¹H-NMR (400 MHz, DMSO) δ 9.23 (s, 1H), 9.06 (s, 1H), 8.82 (s, 1H), 8.08 (s, 1H), 7.64-7.56 (m, 4H), 7.48 (m, 1H), 7.38 (t, 1H), 7.26 (d, 1H), 7.04 (d, 1H), 6.88-6.85 (m, 1H), 4.81 (t, 1H), 4.71 (t, 2H), 3.71 (m, 2H).

### EXAMPLE 64

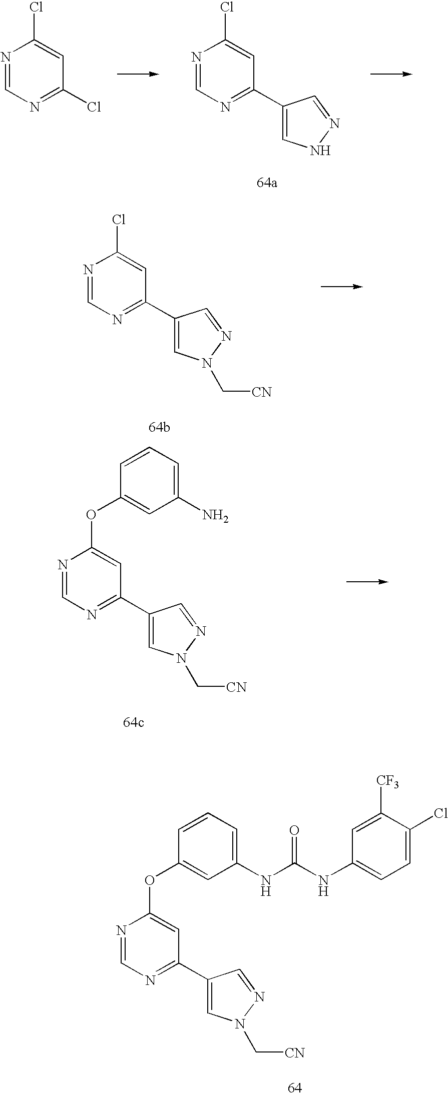 Step 1

Preparation of compound 64a: 4-Chloro-6-(1H-pyrazol-4-yl)-pyrimidine was prepared following the procedures described in preparation of Example 27. [M+H]⁺ 180.88.

Step 2

Preparation of compound 64b: [4-(6-Chloro-pyrimidin-4-yl)-pyrazol-1-yl]-acetonitrile was prepared following the procedures described in preparation of Example 54. [M+H]⁺ 219.88.

Step 3

Preparation of compound 64c: {4-[6-(3-Amino-phenoxy)-pyrimidin-4-yl]-pyrazol-1-yl}-acetonitrile was prepared following the procedures described in preparation of Example 1. [M+H]⁺ 292.85.

Step 4

Preparation of compound 64: 1-(4-Chloro-3-(trifluoromethyl)phenyl)-3-(3-(6-(1-(cyanomethyl)-1H-pyrazol-4-yl)pyrimidin-4-yloxy)phenyl)urea was prepared following the procedures described in preparation of Example 1. [M+H]⁺ 513.81; ¹H-NMR (400 MHz, DMSO) δ 9.20 (s, 1H), 9.03 (s, 1H), 8.67 (s, 1H), 8.59 (s, 1H), 8.33 (s, 1H), 8.07 (s, 1H), 7.60 (m, 2H), 7.44 (m, 2H), 7.38 (t, 1H), 7.26 (m, 1H), 6.85-6.83 (m, 1H), 5.66 (s, 2H).

### EXAMPLE 65

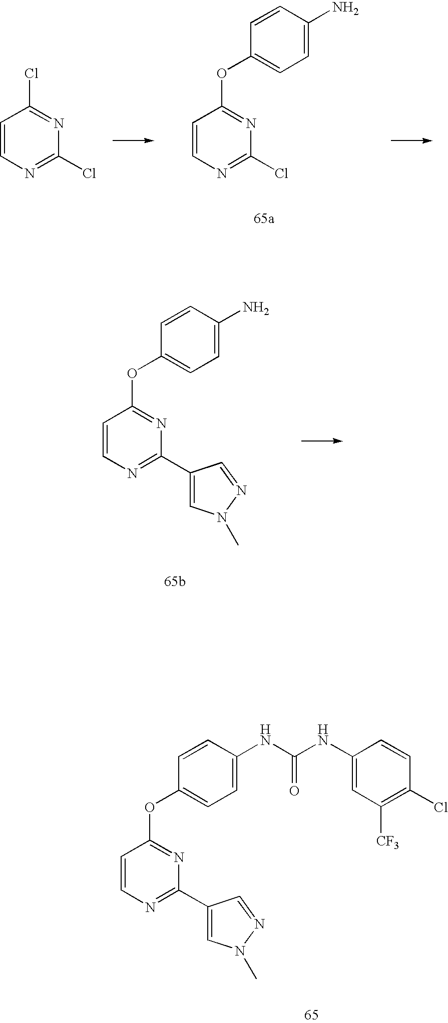 Step 1

Preparation of compound 65a: 4-(2-Chloro-pyrimidin-4-yloxy)-phenylamine was prepared following the procedures described in preparation of Example 1. [M+H]⁺ 221.88, 223.82; ¹H-NMR (400 MHz, DMSO) δ 8.53 (d, 1H), 8.63 (d, 1H,), 6.86 (d, 2H), 6.61 (d, 2H), 5.27 (s, 2H).

Step 2

Preparation of compound 65b: 4-[2-(1-Methyl-1H-pyrazol-4-yl)-pyrimidin-4-yloxy]-phenylamine was prepared following the procedures described in preparation of Example 27. [M+H]⁺ 268.96.

Step 3

Preparation of compound 65: 1-(4-Chloro-3-(trifluoromethyl)phenyl)-3-(4-(2-(1-methyl-1H-pyrazol-4-yl)pyrimidin-4-yloxy)phenyl)urea was prepared following the procedures described in preparation of Example 1. [M+H]⁺ 490.35; ¹H-NMR (400 MHz, DMSO) δ 9.19 (s, 1H), 8.95 (s, 1H), 8.57 (d, 1H), 8.15 (s, 1H), 8.10 (d, 1H), 7.84 (s, 1H), 7.56 (m, 4H), 7.20 (d, 2H), 6.68 (d, 1H), 3.88 (s, 3H).

### EXAMPLE 66

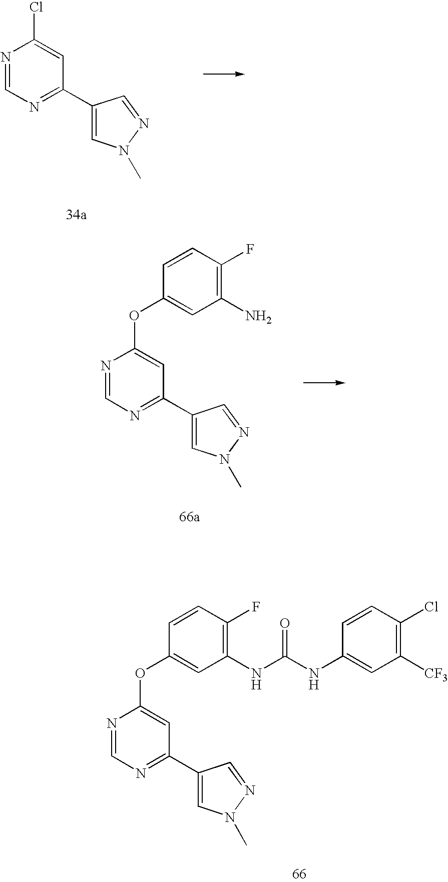 Step 1

Preparation of compound 66a: 2-Fluoro-5-[6-(1-methyl-1H-pyrazol-4-yl)-pyrimidin-4-yloxy]-phenylamine was prepared following the procedures described in preparation of Example 1. [M+H]⁺ 286.

Step 2

Preparation of compound 66: 1-(4-Chloro-3-(trifluoromethyl)phenyl)-3-(2-fluoro-5-(6-(1-methyl-1H-pyrazol-4-yl)pyrimidin-4-yloxy)phenyl)urea was prepared following the procedures described in preparation of Example 1. [M+H]⁺ 507; ¹H-NMR (400 MHz, DMSO) δ 9.56 (bs, 1H), 8.85 (bs, 1H), 8.64 (d, 1H), 8.47 (s, 1H), 8.16 (s, 1H), 8.09 (d, 1H), 7.98 (dd, 1H), 7.61 (t, 1H), 7.57 (dd, 1H), 7.38 (d, 1H), 7.35 (dd, 1H), 6.90 (m, 1H), 3.90 (s, 3H).

### EXAMPLE 67

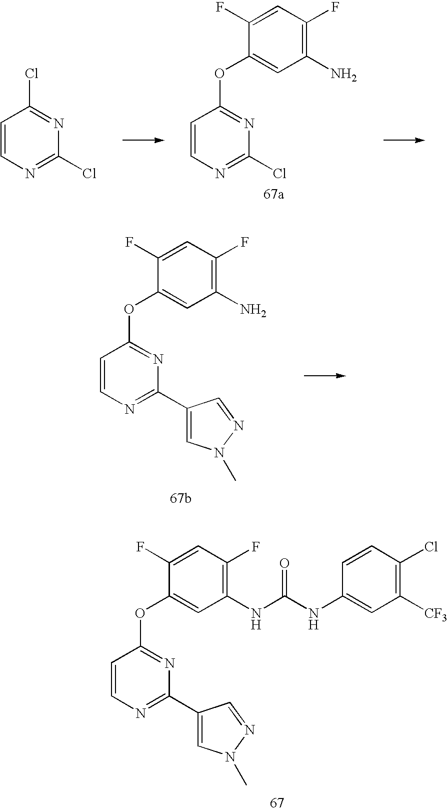 Step 1

Preparation of compound 67a: 5-(2-Chloro-pyrimidin-4-yloxy)-2,4-difluoro-phenylamine was prepared following the procedures described in preparation of Example 1. [M+H]⁺ 258.

Step 2

Preparation of compound 67b: 2,4-Difluoro-5-[2-(1-methyl-1H-pyrazol-4-yl)-pyrimidin-4-yloxy]-phenylamine was prepared following the procedures described in preparation of Example 27. [M+H]⁺ 304.

Step 3

Preparation of compound 67: 1-(4-Chloro-3-(trifluoromethyl)phenyl)-3-(2,4-difluoro-5-(2-(1-methyl-1H-pyrazol-4-yl)pyrimidin-4-yloxy)phenyl)urea was prepared following the procedures described in preparation of Example 1. [M+H]⁺ 525; ¹H-NMR (400 MHz, DMSO) δ 9.53 (br s, 1H), 8.83 (br s, 1H), 8.66 (d, 1H), 8.12 (m, 3H), 7.81 (s, 1H), 7.62 (m, 3H), 6.97 (d, 1H), 3.85 (s, 3H).

### EXAMPLE 68

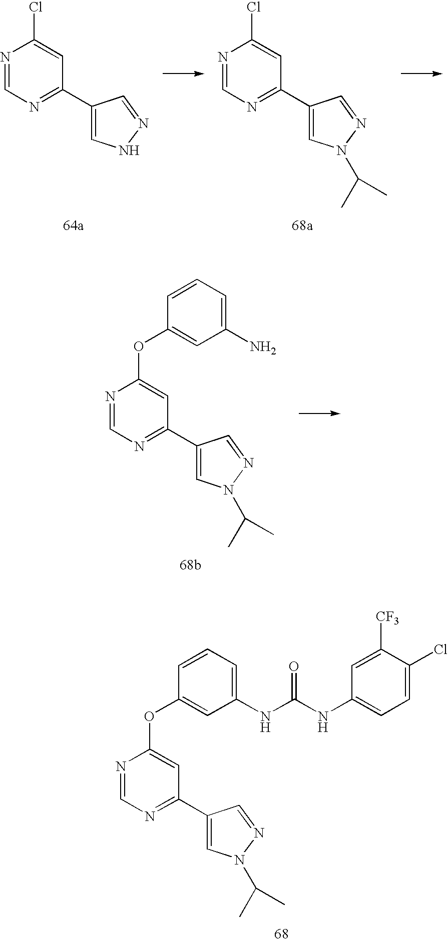 Step 1

Preparation of compound 68a: 4-Chloro-6-(1-isopropyl-1H-pyrazol-4-yl)-pyrimidine was prepared following the procedures described in preparation of Example 54. [M+H]⁺ 222.93.

Step 2

Preparation of compound 68b: 3-[6-(1-Isopropyl-1H-pyrazol-4-yl)-pyrimidin-4-yloxy]-phenylamine was prepared following the procedures described in preparation of Example 1. [M+H]⁺ 295.74.

Step 3

Preparation of compound 68: 1-(4-Chloro-3-(trifluoromethyl)phenyl)-3-(3-(6-(1-isopropyl-1H-pyrazol-4-yl)pyrimidin-4-yloxy)phenyl)urea was prepared following the procedures described in preparation of Example 1. [M+H]⁺ 516.22; ¹H-NMR (400 MHz, DMSO) δ 9.20 (s, 1H), 9.02 (s, 1H), 8.63 (s, 1H), 8.54 (s, 1H), 8.15 (s, 1H), 8.07 (d, 1H), 7.64-7.57 (m, 2H), 7.43 (s, 1H), 7.38 (s, 1H), 7.34 (t, 1H), 7.28-7.24 (m, 1H), 6.85-6.82 (m, 1H), 4.54 (sept, 1H), 1.44 (d, 6H).

### EXAMPLE 69

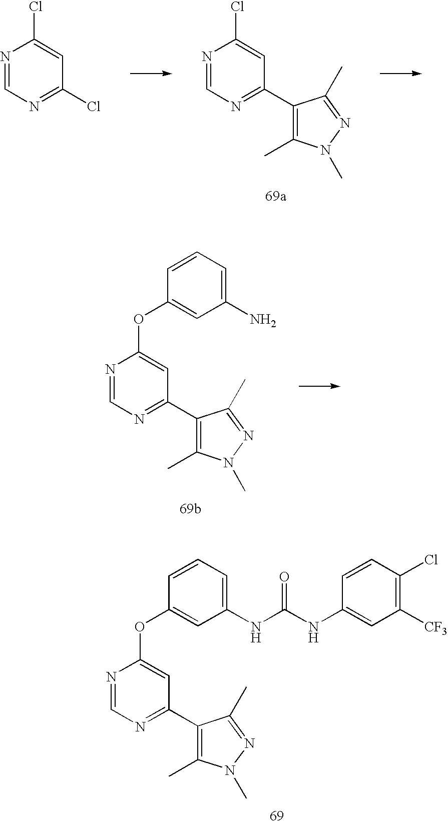 Step 1

Preparation of compound 69a: 4-Chloro-6-(1,3,5-trimethyl-1H-pyrazol-4-yl)-pyrimidine was prepared following the procedures described in preparation of Example 27. [M+H]⁺ 224.44.

Step 2

Preparation of compound 69b: 3-[6-(1,3,5-Trimethyl-1H-pyrazol-4-yl)-pyrimidin-4-yloxy]-phenylamine was prepared following the procedures described in preparation of Example 1. [M+H]⁺ 295.74.

Step 3

Preparation of compound 69: 1-(4-Chloro-3-(trifluoromethyl)phenyl)-3-(3-(6-(1,3,5-trimethyl-1H-pyrazol-4-yl)pyrimidin-4-yloxy)phenyl)urea was prepared following the procedures described in preparation of Example 1. [M+H]⁺ 519.14; ¹H-NMR (400 MHz, DMSO) δ 9.20 (s, 1H), 9.03 (s, 1H), 8.71 (d, 1H), 8.07 (d, 1H), 7.63-7.57 (m, 2H), 7.48 (t, 1H), 7.36 (t, 1H), 7.26-7.23 (m, 1H), 6.98 (d, 1H), 6.87-6.84 (m, 1H), 3.70 (s, 3H), 2.46 (s, 3H), 2.30 (s, 3H).

### EXAMPLE 70

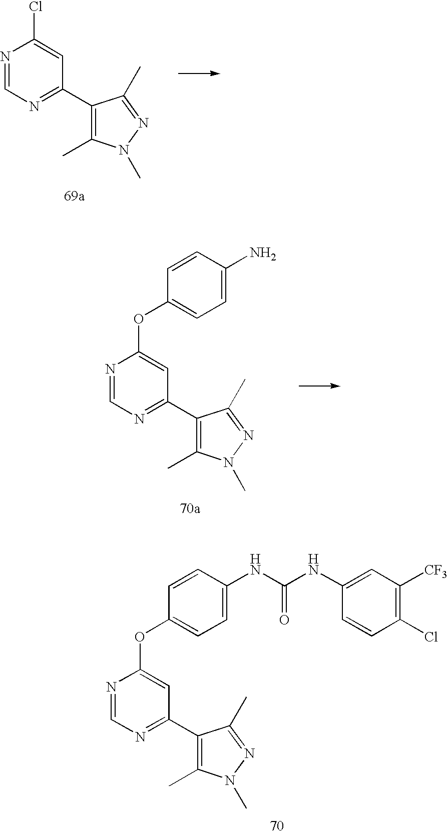 Step 1

Preparation of compound 70a: 4-[6-(1,3,5-Trimethyl-1H-pyrazol-4-yl)-pyrimidin-4-yloxy]-phenylamine was prepared following the procedures described in preparation of Example 1. [M+H]⁺ 295.79.

Step 2

Preparation of compound 70: 1-(4-Chloro-3-(trifluoromethyl)phenyl)-3-(4-(6-(1,3,5-trimethyl-1H-pyrazol-4-yl)pyrimidin-4-yloxy)phenyl)urea was prepared following the procedures described in preparation of Example 1. [M+H]⁺ 519.18; ¹H-NMR (400 MHz, DMSO) δ 9.18 (s, 1H), 8.93 (s, 1H), 8.70 (s, 1H), 8.10 (d, 1H), 7.66-7.58 (m, 2H), 7.52 (d, 2H), 7.17 (d, 2H), 6.95 (s, 1H), 3.70 (s, 3H), 2.45 (s, 3H), 2.29 (s, 3H).

### EXAMPLE 71

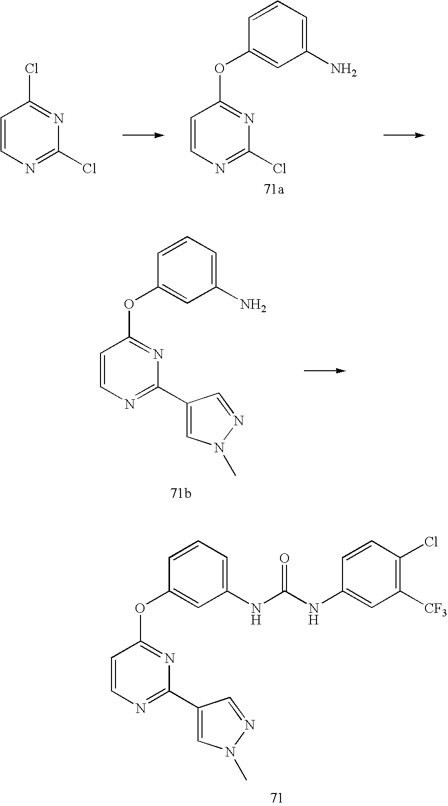 Step 1

Preparation of compound 71a: 3-(2-Chloro-pyrimidin-4-yloxy)-phenylamine was prepared following the procedures described in preparation of Example 1. [M+H]⁺ 221.78.

Step 2

Preparation of compound 71b: 3-[2-(1-Methyl-1H-pyrazol-4-yl)-pyrimidin-4-yloxy]-phenylamine was prepared following the procedures described in preparation of Example 27. [M+H]⁺ 267.88.

Step 3

Preparation of compound 71: 1-(4-Chloro-3-(trifluoromethyl)phenyl)-3-(3-(2-(1-methyl-1H-pyrazol-4-yl)pyrimidin-4-yloxy)phenyl)urea was prepared following the procedures described in preparation of Example 1. [M+H]⁺ 490.35; ¹H-NMR (400 MHz, DMSO) δ 9.21 (s, 1H), 9.04 (s, 1H), 8.59 (d, 1H), 8.20 (s, 1H), 8.07 (d, 1H), 7.86 (s, 1H), 7.60 (m, 2H), 7.51 (t, 1H), 7.39 (t, 1H), 7.30 (m, 1H), 6.90 (m, 1H), 6.72 (d, 1H), 3.85 (s, 3H).

### EXAMPLE 72

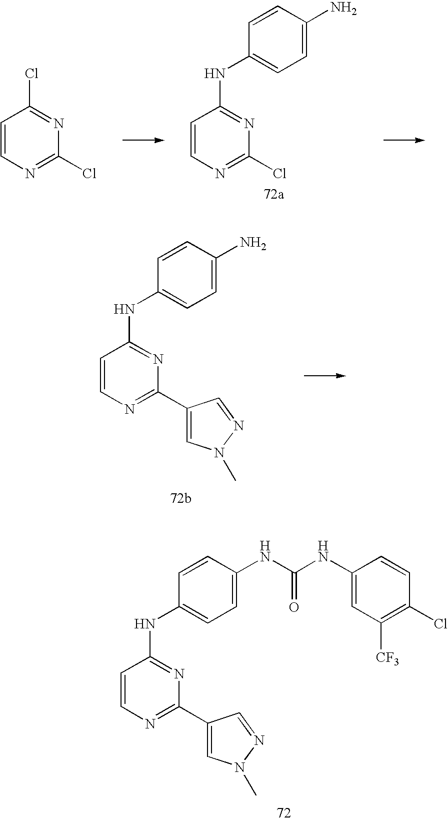 Step 1

Preparation of compound 72a: N-(2-Chloro-pyrimidin-4-yl)-benzene-1,4-diamine was prepared following the procedures described in preparation of Example 1. [M+H]⁺ 221.16.

Step 2

Preparation of compound 72b: N-[2-(1-Methyl-1H-pyrazol-4-yl)-pyrimidin-4-yl]-benzene-1,4-diamine was prepared following the procedures described in preparation of Example 1. [M+H]⁺ 267.11.

Step 3

Preparation of compound 72: 1-(4-Chloro-3-trifluoromethyl-phenyl)-3-{4-[2-(1-methyl-1H-pyrazol-4-yl)-pyrimidin-4-ylamino]-phenyl}-urea was prepared following the procedures described in preparation of Example 1. [M+H]⁺ 488.90.

### EXAMPLE 73

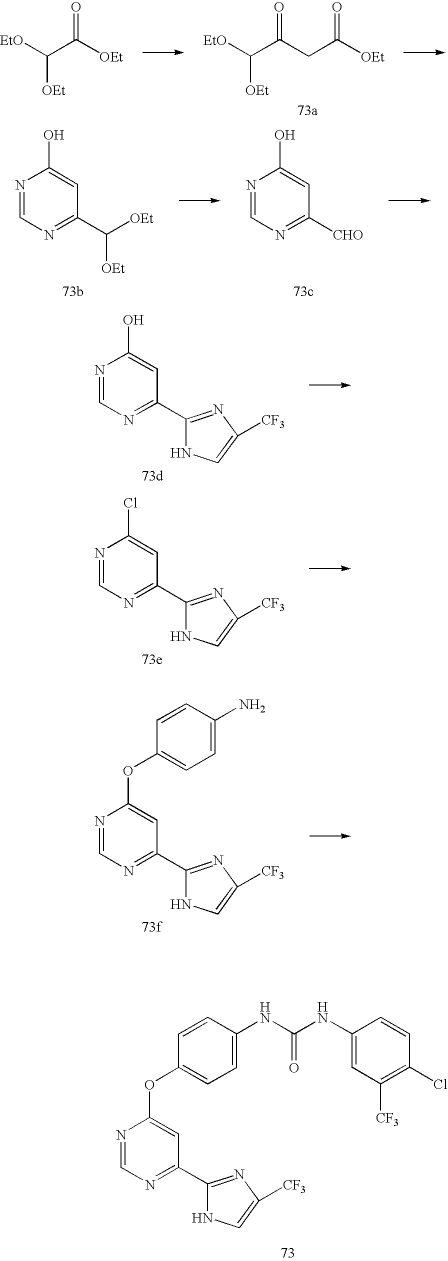 Step 1

### Preparation of compound 73a: Ethyl 4,4-diethoxy-3-oxobutanoate

Sodium (6.30 g, 274 mmol) was added in 500 mg portions over a 1 h period to a solution of ethyl 2,2-diethoxyacetate (30.0 g, 171 mmol) and ethyl acetate (50.0 g, 568 mmol) at room temperature under nitrogen. The reaction mixture was heated to 60° C. for 2 h then cooled to room temperature and stirred for 10 h. Methanol (5 mL) and water (50 mL) were carefully added prior to bringing the pH to 6 with 1 M HCl. The resulting solution was extracted with methylene chloride (3×100 mL) and the combined organic layers were washed with brine (2×100 mL), then concentrated under vacuum to give 40.7 g of ethyl 4,4-diethoxy-3-oxobutanoate as an orange liquid.

Step 2

### Preparation of compound 73b: 6-(Diethoxymethyl)pyrimidin-4-ol

A mixture of ethyl 4,4-diethoxy-3-oxobutanoate (40.7 g, 187 mmol), formamidine acetate (26.0 g, 250 mmol), KOH (19.0 g, 339 mmol) and EtOH (150 mL) was heated to 80° C. under nitrogen for 8 h. The reaction mixture was cooled to room temperature and the resulting precipitate was removed by vacuum filtration. The filtrate was concentrated under vacuum and the product was purified using column chromatography (hexanes to 1:1 hexanes/ethyl acetate) to give 17.0 g of 6-(diethoxymethyl)pyrimidin-4-ol as a yellow solid. [M+H]⁺ 199.00; ¹H-NMR (300 MHz, CD₃Cl) δ 8.22 (s, 1H), 6.76 (s, 1H), 5.26 (s, 1H), 3.67 (m, 4H), 1.27 (t, 6H).

Step 3.

### Preparation of compound 73c: 6-Hydroxypyrimidine-4-carbaldehyde

A solution of 6-(diethoxymethyl)pyrimidin-4-ol (5.00 g, 25.3 mmol) in 1M HCl (30 mL) was heated to 50° C. for 2 hours. The reaction mixture was cooled to room temperature, then concentrated under vacuum. Methanol (50 mL) and solid NaHCO₃ (10.0 g) were sequentially added. The inorganic salts were removed using vacuum filtration and the filtrated was concentrated under vacuum to give 3.04 g of 6-hydroxypyrimidine-4-carbaldehyde as a clear oil. [M+H]⁺ 125.00.

Step 4

### Preparation of compound 73d: 6-(5-(Trifluoromethyl)-1H-imidazol-2-yl)pyrimidin-4-ol

A mixture of 3,3-dibromo-1,1,1-trifluoropropan-2-one (6.00 g, 22.2 mmol), sodium acetate (3.80 g, 46.3 mmol) and water (30 mL) was heated to 100° C. for 40 minutes, then cooled to room temperature. A solution of 6-hydroxypyrimidine-4-carbaldehyde (3.20 g, 25.8 mmol) in MeOH (100 mL) and ammonium hydroxide (25 mL) were added sequentially prior to stirring at room temperature for 12 h. The resulting solution was concentrated under vacuum, then ethyl acetate (100 mL) and brine (100 mL) were added. The phases were separated and the aqueous layer was back extracted with ethyl acetate (2×100 mL). The combined organic layers were concentrated under vacuum and the product was purified using column chromatography (DCM to 4:1 DCM/methanol) to give 2.30 g of 6-(5-(trifluoromethyl)-1H-imidazol-2-yl)pyrimidin-4-ol as a yellow solid. [M+H]⁺ 231.0; ¹H-NMR (300 MHz, DMSO) δ 8.31 (br s, 1H), 7.92 (s, 1H), 6.83 (s, 1H).

Step 5

### Preparation of compound 73e: 4-Chloro-6-(4-(trifluoromethyl)-1H-imidazol-2-yl)pyrimidine

A mixture of 6-(4-(trifluoromethyl)-1H-imidazol-2-yl)pyrimidin-4-ol (3.20 g, 13.9 mmol) and POCl₃ (50 mL) was heated to 105° C. for 2 hours under nitrogen. The reaction mixture was cooled to room temperature, then concentrated under vacuum. Ethyl acetate (100 mL) and ammonium hydroxide (100 mL of a 10% v/v aqueous solution) were added. The phases were separated and the aqueous layer was back extracted with ethyl acetate (100 mL). The combined organic layers were washed with brine (2×100 mL), then concentrated under vacuum. The product was purified using column chromatography (hexanes to ethyl acetate) to give 2.00 g of 4-chloro-6-(4-(trifluoromethyl)-1H-imidazol-2-yl)pyrimidine as a yellow solid. [M+H]⁺ 249.0; ¹H-NMR (300 MHz, CD₃Cl) δ 10.74 (br s, 1H), 8.97 (s, 1H), 8.22 (s, 1H), 7.59 (s, 1H).

Step 6

Preparation of compound 73f: 4-(6-(5-(Trifluoromethyl)-1H-imidazol-2-yl)pyrimidin-4-yloxy)benzenamine was prepared following the procedures described in preparation of Example 4. [M+H]⁺ 322.00; ¹H-NMR (300 MHz, CD₃Cl) δ 10.66 (br s, 1H), 8.76 (s, 1H), 7.62 (s, 1H), 7.54 (s, 1H), 6.97 (d, 1H), 6.74 (d, 1H), 3.73 (s, 2H).

Step 7

Preparation of compound 73: 1-(4-Chloro-3-(trifluoromethyl)phenyl)-3-(4-(6-(4-(trifluoromethyl)-1H-imidazol-2-yl)pyrimidin-4-yloxy)phenyl)urea was prepared following the procedures described in preparation of Example 1. [M+H]⁺ 543.04, 544.29; ¹H-NMR (400 MHz, CD₃OD) δ 8.76 (d, 1H), 8.00 (d, 1H), 7.50 (d, 1H), 7.63 (m, 1H), 7.56 (m, 3H), 7.50 (d, 1H), 7.16 (m, 2H).

### EXAMPLE 74

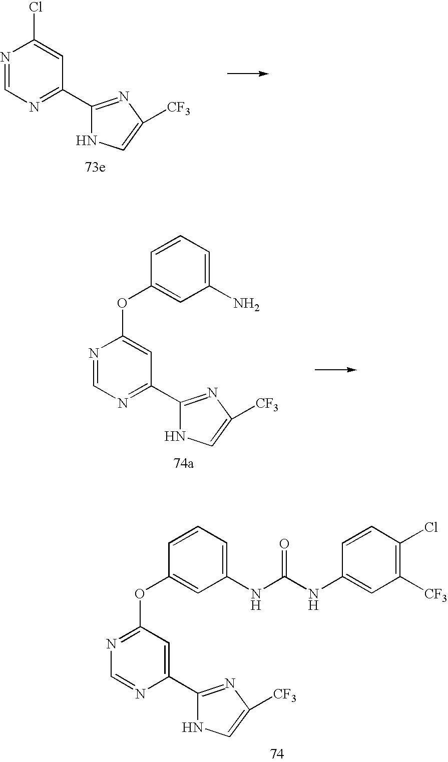 Step 1

Preparation of compound 74a: 3-[6-(4-Trifluoromethyl-1H-imidazol-2-yl)-pyrimidin-4-yloxy]-phenylamine was prepared following the procedures described in preparation of Example 4. [M+H]⁺ 321.73.

Step 2

Preparation of compound 74: 1-(4-Chloro-3-(trifluoromethyl)phenyl)-3-(3-(6-(4-(trifluoromethyl)-1H-imidazol-2-yl)pyrimidin-4-yloxy)phenyl)urea was prepared following the procedures described in preparation of Example 1. [M+H]⁺ 542.97, 544.28; ¹H-NMR (400 MHz, CD₃OD) δ 8.76 (d, 1H), 7.94 (d, 1H), 7.75 (s, 1H), 7.60 (m, 2H), 7.49 (m, 2H), 7.41 (d, 1H), 7.30 (d, 1H), 6.88 (dd, 1H).

### EXAMPLE 75

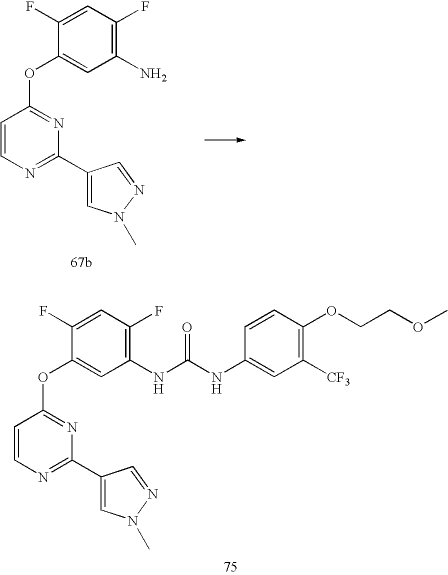

Preparation of compound 75: 1-(2,4-Difluoro-5-(2-(1-methyl-1H-pyrazol-4-yl)pyrimidin-4-yloxy)phenyl)-3-(4-(2-methoxyethoxy)-3-(trifluoromethyl)phenyl)urea was prepared following the procedures described in preparation of Example 24. [M+H]⁺ 565; ¹H-NMR (400 MHz, DMSO) δ 9.18 (s, 1H), 8.72 (bd, 1H), 8.65 (d, 1H), 8.14 (s, 1H), 7.82 (m, 2H), 7.62 (t, 1H), 7.49 (dt, 1H), 7.22 (m, 2H), 6.96 (d, 1H), 4.18 (m, 2H), 3.85 (s, 3H), 3.65 (m, 2H), 3.30 (s, 3H).

### EXAMPLE 76

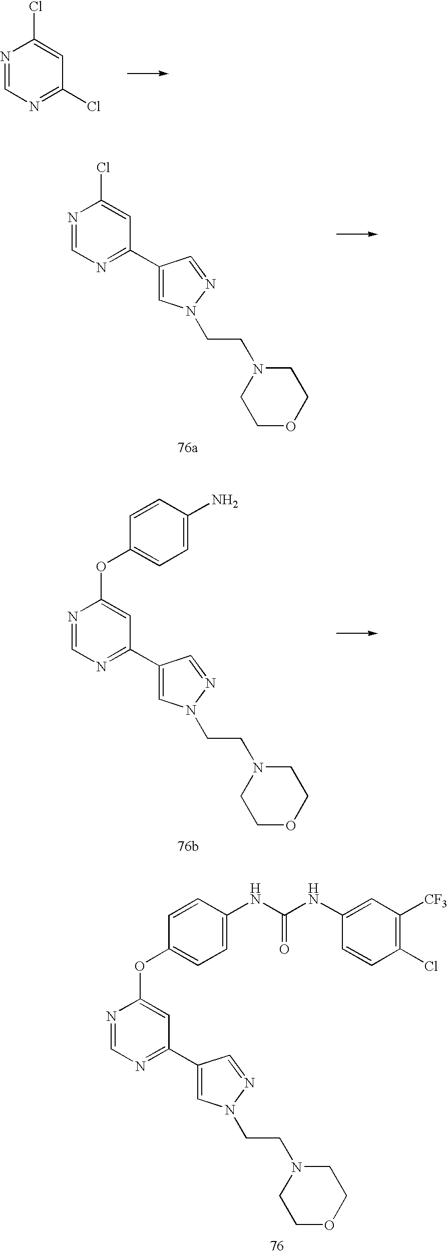 Step 1

Preparation of compound 76a: 4-{2-[4-(6-Chloro-pyrimidin-4-yl)-pyrazol-1-yl]-ethyl}-morpholine was prepared following the procedures described in preparation of Example 27. [M+H]⁺ 294.34.

Step 2

Preparation of compound 76b: 1-(4-Chloro-3-(trifluoromethyl)phenyl)-3-(4-(6-(1-(2-morpholinoethyl)-1H-pyrazol-4-yl)pyrimidin-4-yloxy)phenyl)urea was prepared following the procedures described in preparation of Example 1. [M+H]⁺ 367.69.

Step 3

Preparation of compound 76: 1-(4-Chloro-3-(trifluoromethyl)phenyl)-3-(4-(6-(1-(2-morpholinoethyl)-1H-pyrazol-4-yl)pyrimidin-4-yloxy)phenyl)urea was prepared following the procedures described in preparation of Example 1. [M+H]⁺ 589.20; ¹H-NMR (400 MHz, DMSO) δ 9.18 (s, 1H), 8.93 (s, 1H), 8.61 (d, 1H), 8.48 (s, 1H), 8.15 (s, 1H), 8.10 (d, 1H), 7.66-7.58 (m, 2H), 7.52-7.49 (d, 2H), 7.32 (s, 1H), 7.16-7.12 (d, 2H), 4.27 (t, 2H), 3.60-3.50 (m, 4H), 2.72 (t, 2H), 2.45-2.38 (m, 4H).

### EXAMPLE 77

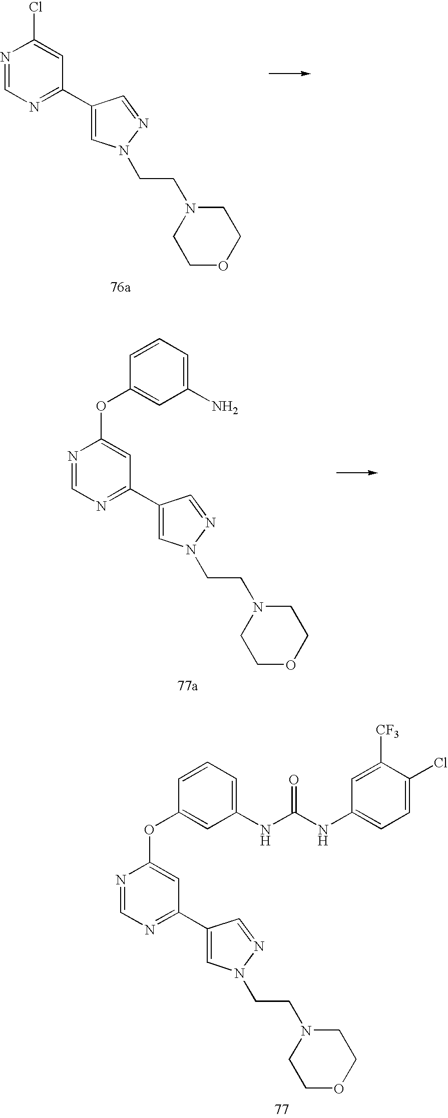 Step 1

Preparation of compound 77a: 3-{6-[1-(2-Morpholin-4-yl-ethyl)-1H-pyrazol-4-yl]-pyrimidin-4-yloxy}-phenylamine was prepared following the procedures described in preparation of Example 1. [M+H]⁺ 367.46.

Step 2

Preparation of compound 77: 1-(4-Chloro-3-(trifluoromethyl)phenyl)-3-(3-(6-(1-(2-morpholinoethyl)-1H-pyrazol-4-yl)pyrimidin-4-yloxy)phenyl)urea was prepared following the procedures described in preparation of Example 1. [M+H]⁺ 590.35; ¹H-NMR (400 MHz, DMSO) δ 9.20 (s, 1H), 9.03 (s, 1H), 8.63 (s, 1H), 8.49 (s, 1H), 8.15 (s, 1H), 8.07 (d, 1H), 7.64-7.57 (m, 2H), 7.44 (t, 1H), 7.38-7.32 (m, 2H), 7.28-7.25 (m, 1H), 6.85-6.82 (m, 1H), 4.27 (t, 2H), 3.52 (m, 4H), 2.72 (t, 2H), 2.41 (m, 4H).

### EXAMPLE 78

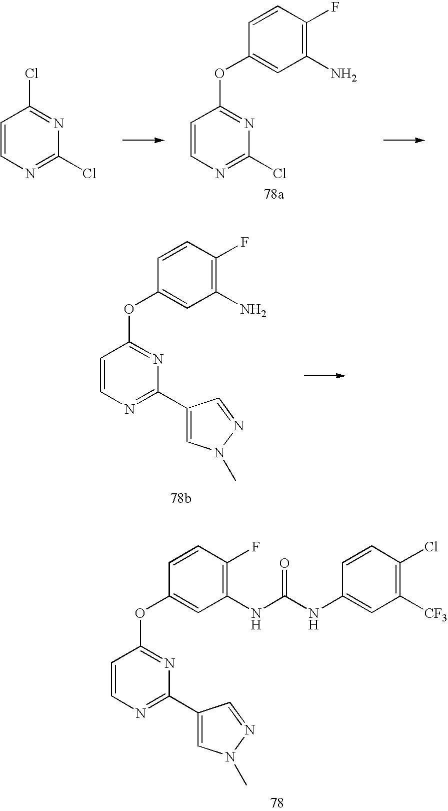 Step 1

Preparation of compound 78a: 5-(2-Chloro-pyrimidin-4-yloxy)-2-fluoro-phenylamine was prepared following the procedures described in preparation of Example 1. [M+H]⁺ 240.

Step 2

Preparation of compound 78b: 2-Fluoro-5-[2-(1-methyl-1H-pyrazol-4-yl)-pyrimidin-4-yloxy]-phenylamine was prepared following the procedures described in preparation of Example 27. [M+H]⁺ 286.

Step 3

Preparation of compound 78: 1-(4-Chloro-3-(trifluoromethyl)phenyl)-3-(2-fluoro-5-(2-(1-methyl-1H-pyrazol-4-yl)pyrimidin-4-yloxy)phenyl)urea was prepared following the procedures described in preparation of Example 1. [M+H]⁺ 507; ¹H-NMR (400 MHz, DMSO) δ 9.61 (s, 1H), 8.90 (br d, 1H), 8.61 (d, 1H), 8.21 (s, 1H), 8.08 (m, 2H), 7.87 (s, 1H), 7.62 (m, 2H), 7.38 (dd, 1H), 6.97 (m, 1H), 6.78 (d, 1H), 3.85 (s, 3H).

### EXAMPLE 79

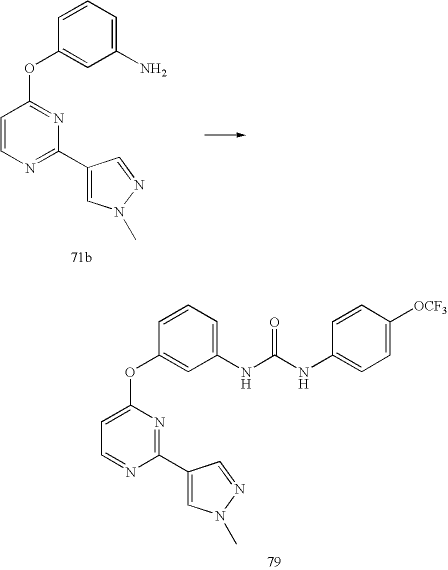

Preparation of compound 79: 1-(3-(2-(1-Methyl-1H-pyrazol-4-yl)pyrimidin-4-yloxy)phenyl)-3-(4-(trifluoromethoxy)phenyl)urea was prepared following the procedures described in preparation of Example 1. [M+H]⁺ 471.87; ¹H-NMR (400 MHz, DMSO) δ 8.93 (s, 1H), 8.93 (s, 1H), 8.00 (dd, 1H), 8.20 (s, 1H), 7.86 (s, 1H), 7.53 (m, 3H), 7.37 (t, 1H), 7.27 (m, 3H), 6.88 (m, 1H), 6.72 (dd, 1H), 3.86 (s, 3H).

### EXAMPLE 80

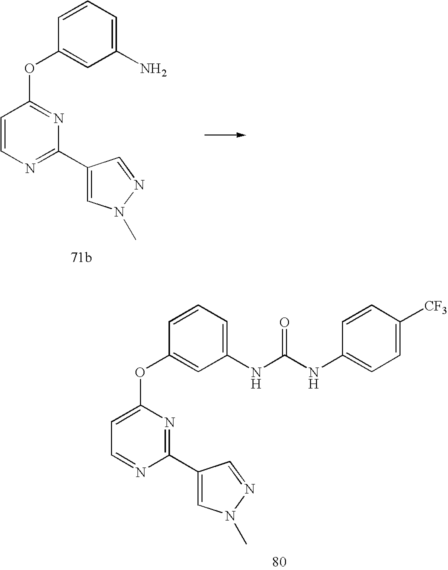

Preparation of compound 80: 1-(3-(2-(1-Methyl-1H-pyrazol-4-yl)pyrimidin-4-yloxy)phenyl)-3-(4-(trifluoromethyl)phenyl)urea was prepared following the procedures described in preparation of Example 1. [M+H]⁺ 455.94; ¹H-NMR (400 MHz, CD₃OD) δ 8.53 (d, 1H), 8.13 (s, 1H), 7.97 (s, 1H), 7.61 (m, 2H), 7.56 (m, 3H), 7.39 (t, 1H), 7.28 (m, 1H), 6.89 (m, 1H), 6.75 (d, 1H), 3.89 (s, 3H).

### EXAMPLE 81

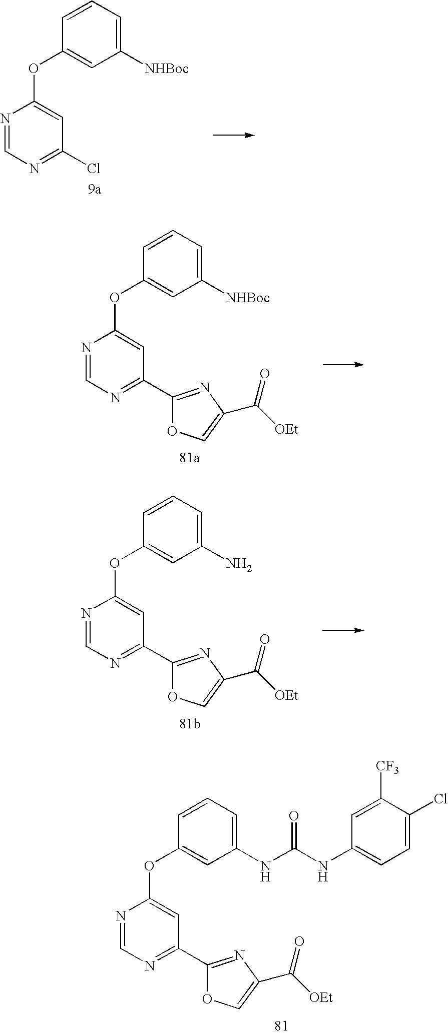 Step 1

### Preparation of compound 81a: 2-[6-(3-tert-Butoxycarbonylamino-phenoxy)-pyrimidin-4-yl]-oxazole-4-carboxylic acid ethyl ester

A mixture of 2a (5.00 g, 15.5 mmol), ethyl 2-chlorooxazole-4-carboxylate (5.00 g, 28.5 mmol), tetrabutylammonium fluoride (25.0 g, 95.6 mmol) and toluene (100 mL) was stirred for 5 minutes at room temperature under nitrogen prior to the addition of hexabutyldistannane (10.0 g, 17.2 mmol). The reaction mixture was purged with nitrogen gas, then tetrakis(triphenylphosphine)palladium(0) (1.50 g, 1.30 mmol) was added. The solution was heated to 110° C. for 3.5 h prior to cooling to room temperature. Ethyl acetate (100 mL) was added and the mixture filtered through a 5 cm plug of silica gel. The filtrate was concentrated under vacuum and the product was purified using column chromatography (hexanes to 3:1 hexanes/ethyl acetate) to give 250 mg of 2-[6-(3-tert-butoxycarbonylamino-phenoxy)-pyrimidin-4-yl]-oxazole-4-carboxylic acid ethyl ester as a yellow solid. [M+H]⁺ 427.04.

Step 2

Preparation of compound 81b: 2-[6-(3-Amino-phenoxy)-pyrimidin-4-yl]-oxazole-4-carboxylic acid ethyl ester was prepared following the procedures described in preparation of Example 1. [M+H]⁺ 326.84.

Step 3

Preparation of compound 81: Ethyl 2-(6-(3-(3-(4-chloro-3-(trifluoromethyl)phenyl)ureido)phenoxy)pyrimidin-4-yl)oxazole-4-carboxylate was prepared following the procedures described in preparation of Example 1. [M+H]⁺ 547.75; ¹H-NMR (400 MHz, DMSO) δ 9.23 (s, 1H), 9.12 (s, 1H), 9.09 (s, 1H), 8.94 (d, 1H), 8.07 (s, 1H), 7.61-7.58 (m, 3H), 7.52 (t, 1H), 7.40 (t, 1H), 7.31-7.27 (m, 1H), 6.92-6.89 (m, 1H), 4.32 (q, 2H), 1.29 (t, 3H).

### EXAMPLE 82

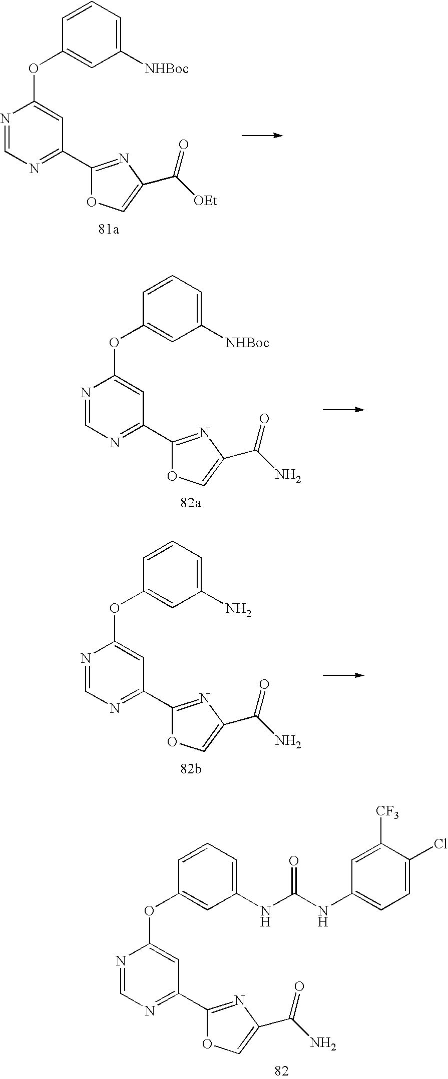 Step 1

Preparation of compound 82a: {3-[6-(4-Carbamoyl-oxazol-2-yl)-pyrimidin-4-yloxy]-phenyl}-carbamic acid tert-butyl ester was prepared following the procedures described in preparation of Example 15. [M+H]⁺ 397.79.

Step 2

Preparation of compound 82b: 2-[6-(3-Amino-phenoxy)-pyrimidin-4-yl]-oxazole-4-carboxylic acid amide was prepared following the procedures described in preparation of Example 1. [M+H]⁺ 297.85.

Step 3

Preparation of compound 82: 2-(6-(3-(3-(4-Chloro-3-(trifluoromethyl)phenyl)ureido)phenoxy)pyrimidin-4-yl)oxazole-4-carboxamide was prepared following the procedures described in preparation of Example 1. [M+H]⁺ 518.71; ¹H-NMR (400 MHz, DMSO) δ 9.23 (s, 1H), 9.09 (s, 1H), 8.93 (d, 1H), 8.84 (s, 1H), 8.08 (s, 1H), 7.85 (m, 1H), 7.64-7.58 (m, 4H), 7.52 (t, 1H), 7.42 (t, 1H), 7.31-7.28 (m, 1H), 6.92-6.89 (m, 1H).

### EXAMPLE 83

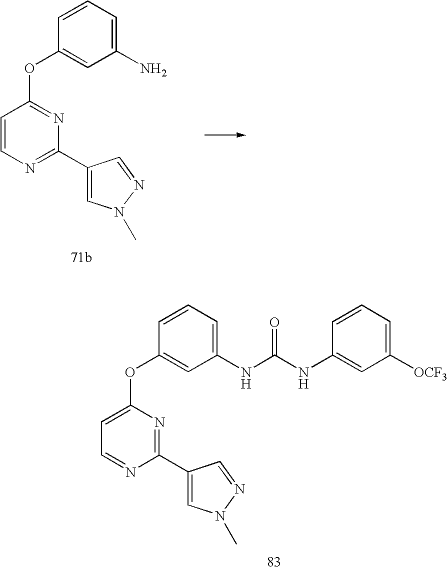

Preparation of compound 83: 1-(3-(2-(1-Methyl-1H-pyrazol-4-yl)pyrimidin-4-yloxy)phenyl)-3-(3-(trifluoromethoxy)phenyl)urea was prepared following the procedures described in preparation of Example 1. [M+H]⁺ 471.90.

### EXAMPLE 84

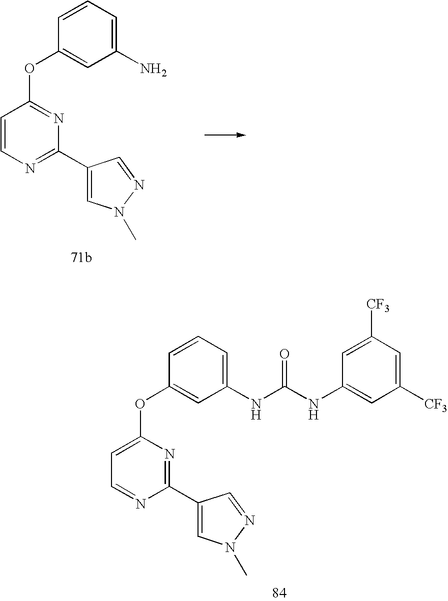

Preparation of compound 84: 1-(3,5-Bis(trifluoromethyl)phenyl)-3-(3-(2-(1-methyl-1H-pyrazol-4-yl)pyrimidin-4-yloxy)phenyl)urea was prepared following the procedures described in preparation of Example 1. [M+H]⁺ 523.73.

### EXAMPLE 85

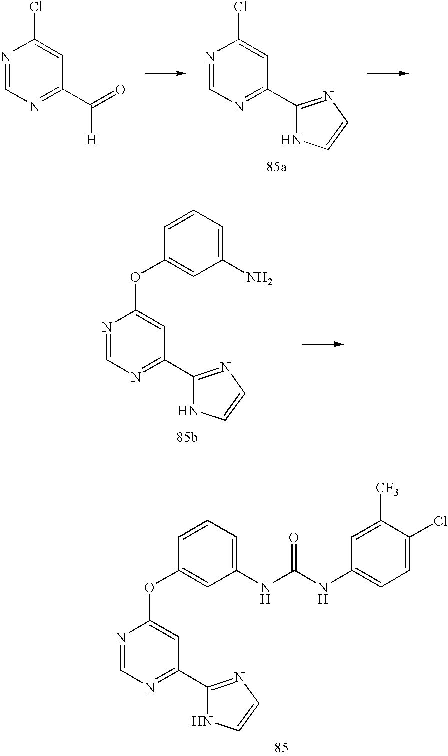 Step 1

Preparation of compound 85a: 4-Chloro-6-(1H-imidazol-2-yl)pyrimidine was prepared following the procedures described in preparation of Example 73. [M+H]⁺ 181.07.

Step 2

Preparation of compound 85b: 3-(6-(1H-Imidazol-2-yl)pyrimidin-4-yloxy)aniline was prepared following the procedures described in preparation of Example 73. [M+H]⁺ 254.21.

Step 3

Preparation of compound 85: 1-(3-(6-(1H-Imidazol-2-yl)pyrimidin-4-yloxy)phenyl)-3-(4-chloro-3-(trifluoromethyl)phenyl)urea was prepared following the procedures described in preparation of Example 1. [M+H]⁺ 477.21; ¹H-NMR (400 MHz, DMSO) δ 13.18 (br s, 1H), 9.27 (s, 1H), 9.11 (s, 1H), 8.80 (s, 1H), 8.07 (s, 1H), 7.63-7.55 (m, 3H), 7.50 (s, 1H), 7.41-7.27 (m, 3H), 7.15 (s, 1H), 6.90-6.87 (d, 1H).

### EXAMPLE 86

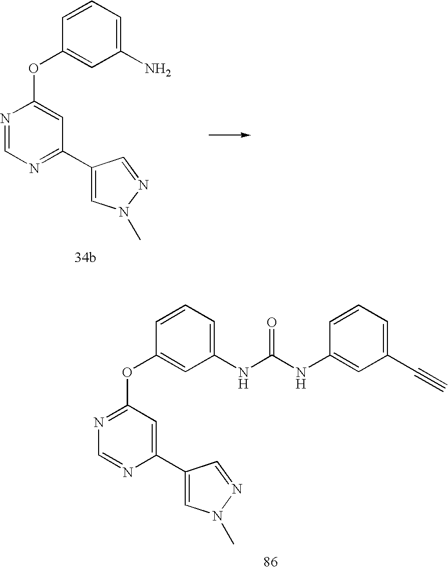

Preparation of compound 86: 1-(3-Ethynylphenyl)-3-(3-(6-(1-methyl-1H-pyrazol-4-yl)pyrimidin-4-yloxy)phenyl)urea was prepared following the procedures described in preparation of Example 24. [M+H]⁺ 411.65.

### EXAMPLE 87

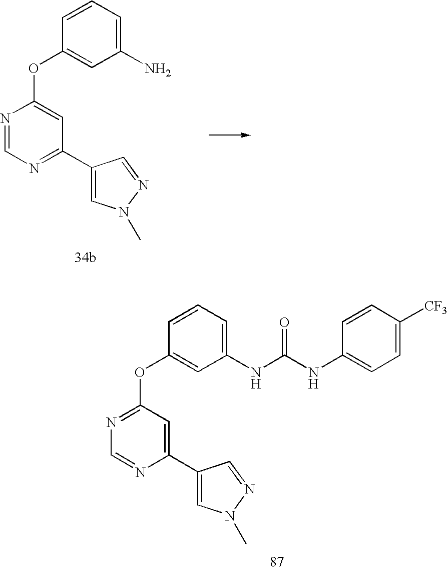

Preparation of compound 87: 1-(3-(6-(1-Methyl-1H-pyrazol-4-yl)pyrimidin-4-yloxy)phenyl)-3-(4-(trifluoromethyl)phenyl)urea was prepared following the procedures described in preparation of Example 1. [M+H]⁺ 455.80; ¹H-NMR (400 MHz, DMSO) δ 9.14 (s, 1H), 8.97 (s, 1H), 8.93 (s, 1H), 8.45 (s, 1H), 8.15 (s, 1H), 7.62 (m, 4H), 7.45 (m, 1H), 7.36 (m, 2H), 7.27 (m, 1H), 6.82 (m, 1H), 3.89 (s, 3H).

### EXAMPLE 88

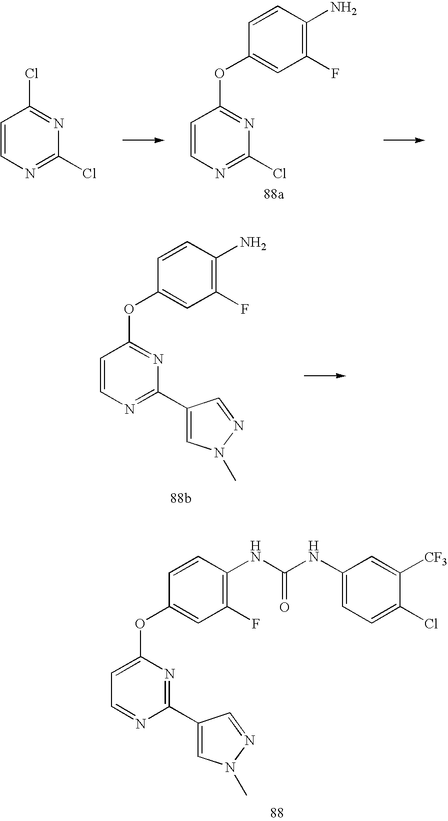 Step 1

Preparation of compound 88a: 4-(2-Chloro-pyrimidin-4-yloxy)-2-fluoro-phenylamine was prepared following the procedures described in preparation of Example 1. [M+H]⁺ 240.15.

Step 2

Preparation of compound 88b: 2-Fluoro-4-[2-(1-methyl-1H-pyrazol-4-yl)-pyrimidin-4-yloxy]-phenylamine was prepared following the procedures described in preparation of Example 27. [M+H]⁺ 286.09.

Step 3

Preparation of compound 88: 1-(4-Chloro-3-(trifluoromethyl)phenyl)-3-(2-fluoro-4-(2-(1-methyl-1H-pyrazol-4-yl)pyrimidin-4-yloxy)phenyl)urea was prepared following the procedures described in preparation of Example 1. [M+H]⁺ 506.83, 508.09; ¹H-NMR (400 MHz, DMSO) δ 9.49 (s, 1H), 8.70 (d, 1H), 8.59 (d, 1H), 8.11 (m, 3H), 7.83 (s, 1H), 7.61 (m, 2H), 7.33 (dd, 1H), 7.10 (m, 1H), 6.78 (d, 1H), 3.85 (s, 3H).

### EXAMPLE 89

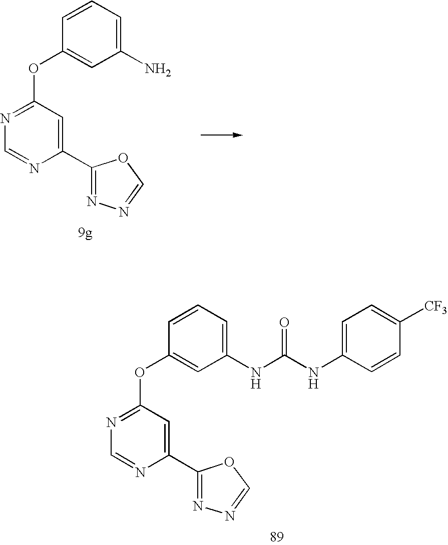

Preparation of compound 89: 1-(3-(6-(1,3,4-Oxadiazol-2-yl)pyrimidin-4-yloxy)phenyl)-3-(4-(trifluoromethyl)phenyl)urea was prepared following the procedures described in preparation of Example 1. [M+H]⁺ 442.78.

### EXAMPLE 90

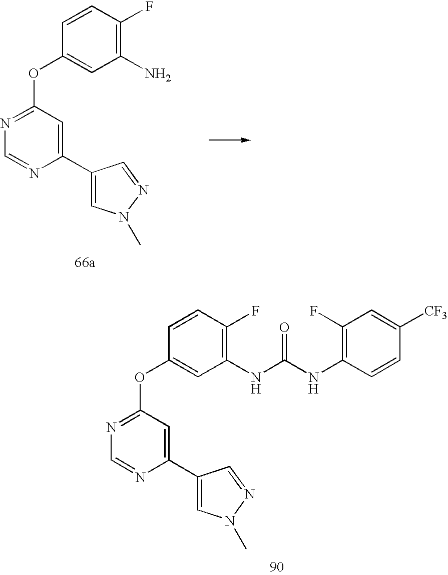

Preparation of compound 90: 1-(2-Fluoro-4-(trifluoromethyl)phenyl)-3-(2-fluoro-5-(6-(1-methyl-1H-pyrazol-4-yl)pyrimidin-4-yloxy)phenyl)urea was prepared following the procedures described in preparation of Example 24. [M+H]⁺ 491.62; ¹H-NMR (400 MHz, DMSO) δ 9.45 (d, 1H), 9.36 (d, 1H), 8.62 (s, 1H), 8.45 (s, 1H), 8.39 (t, 1H), 8.14 (s, 1H), 8.03 (dd, 1H), 7.70 (dd, 1H), 7.51 (d, 1H), 7.34 (m, 2H), 6.89 (m, 1H), 3.88 (s, 3H).

### EXAMPLE 91

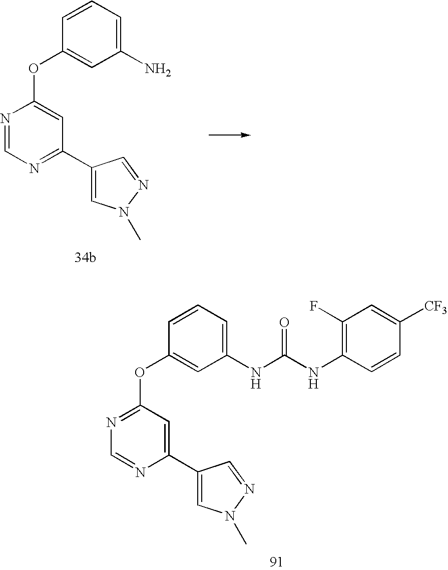

Preparation of compound 91: 1-(2-Fluoro-4-(trifluoromethyl)phenyl)-3-(3-(6-(1-methyl-1H-pyrazol-4-yl)pyrimidin-4-yloxy)phenyl)urea was prepared following the procedures described in preparation of Example 24. [M+H]⁺ 473.53; ¹H-NMR (400 MHz, DMSO) δ 9.35 (s, 1H), 8.94 (d, 1H), 8.63 (s, 1H), 8.45 (s, 1H), 8.39 (t, 1H), 8.14 (s, 1H), 7.68 (dd, 1H), 7.50 (d, 1H), 7.45 (m, 1H), 7.36 (m, 2H), 7.23 (dd, 1H), 6.86 (dd, 1H), 3.89 (s, 3H).

### EXAMPLE 92

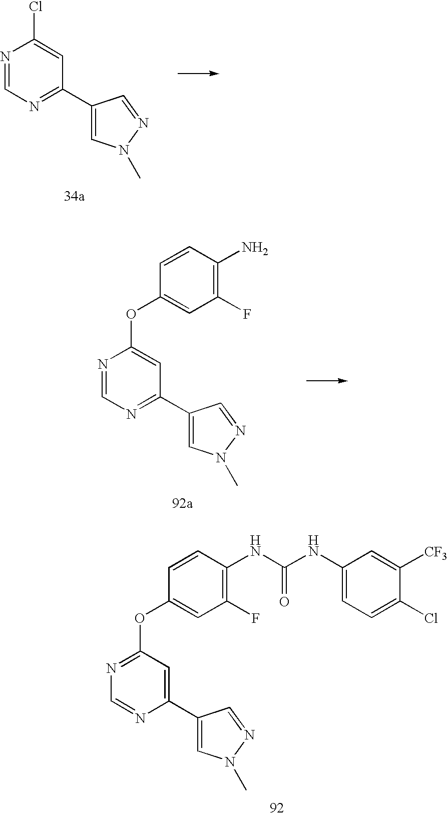 Step 1

Preparation of compound 92a: 2-Fluoro-4-[6-(1-methyl-1H-pyrazol-4-yl)-pyrimidin-4-yloxy]-phenylamine was prepared following the procedures described in preparation of Example 1. [M+H]⁺ 285.79.

Step 2

Preparation of compound 92: 1-(4-Chloro-3-(trifluoromethyl)phenyl)-3-(2-fluoro-4-(6-(1-methyl-1H-pyrazol-4-yl)pyrimidin-4-yloxy)phenyl)urea was prepared following the procedures described in preparation of Example 1. [M+H]⁺ 506.90, 508.29; ¹H-NMR (400 MHz, DMSO) δ 9.48 (s, 1H), 8.67 (s, 1H), 8.63 (s, 1H), 8.45 (s, 1H), 8.14 (s, 1H), 8.10 (s, 1H), 8.06 (m, 1H), 7.61 (m, 2H), 7.37 (s, 1H), 7.30 (dd, 1H), 7.05 (d, 1H), 3.89 (s, 3H).

### EXAMPLE 93

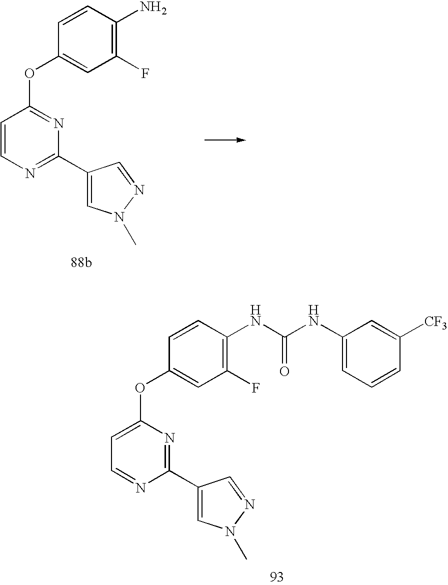

Preparation of compound 93: 1-(2-Fluoro-4-(2-(1-methyl-1H-pyrazol-4-yl)pyrimidin-4-yloxy)phenyl)-3-(3-(trifluoromethyl)phenyl)urea was prepared following the procedures described in preparation of Example 1. [M+H]⁺ 472.9, 473.8.

### EXAMPLE 94

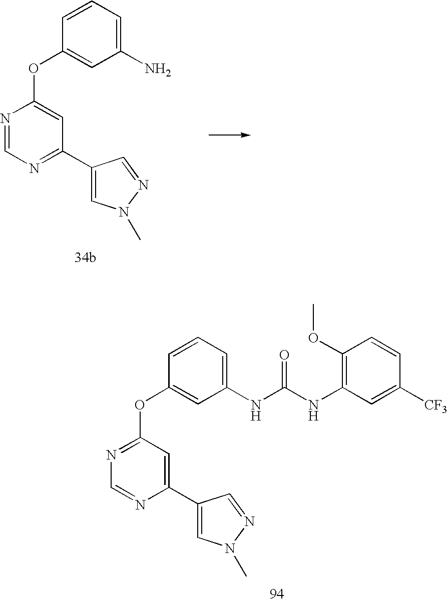

Preparation of compound 94: 1-(2-Methoxy-5-(trifluoromethyl)phenyl)-3-(3-(6-(1-methyl-1H-pyrazol-4-yl)pyrimidin-4-yloxy)phenyl)urea was prepared following the procedures described in preparation of Example 24. [M+H]⁺ 485.60.

### EXAMPLE 95

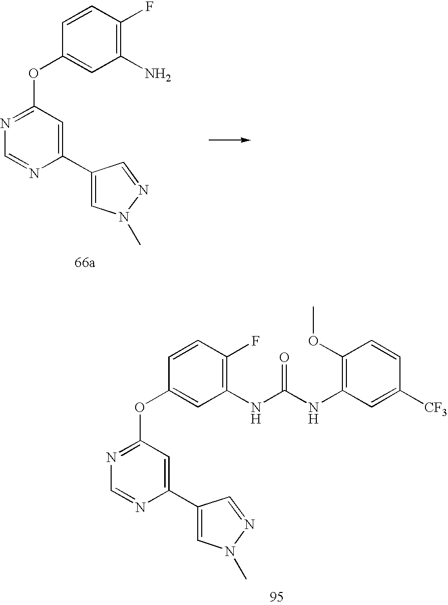

Preparation of compound 95: 1-(2-Fluoro-5-(6-(1-methyl-1H-pyrazol-4-yl)pyrimidin-4-yloxy)phenyl)-3-(2-methoxy-5-(trifluoromethyl)phenyl)urea was prepared following the procedures described in preparation of Example 24. [M+H]⁺ 503.48.

### EXAMPLE 96

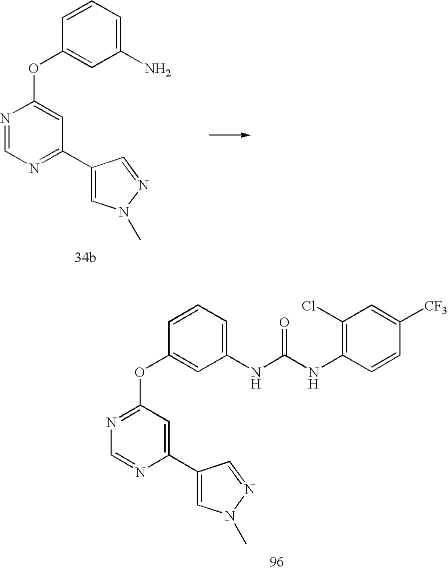

Preparation of compound 96: 1-(2-Chloro-4-(trifluoromethyl)phenyl)-3-(3-(6-(1-methyl-1H-pyrazol-4-yl)pyrimidin-4-yloxy)phenyl)urea was prepared following the procedures described in preparation of Example 24. [M+H]⁺ 489.06, 490.33; ¹H-NMR (400 MHz, DMSO) δ 9.76 (s, 1H), 8.64 (s, 2H), 8.43 (m, 2H), 8.14 (s, 1H), 7.85 (s, 1H), 7.64 (d, 1H), 7.47 (s, 1H), 7.37 (m, 2H), 7.23 (d, 1H), 6.85 (d, 1H), 3.89 (s, 3H).

### EXAMPLE 97

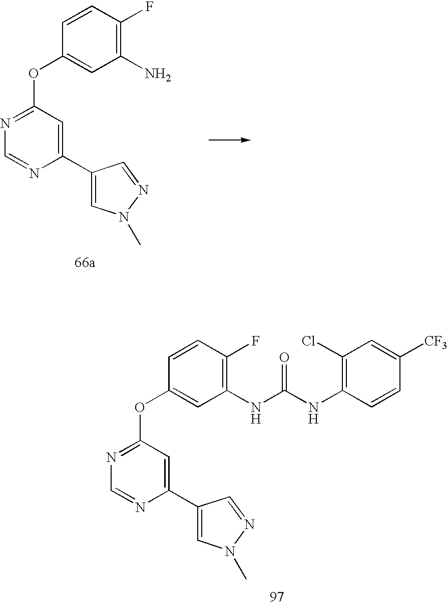

Preparation of compound 97: 1-(2-Chloro-4-(trifluoromethyl)phenyl)-3-(2-fluoro-5-(6-(1-methyl-1H-pyrazol-4-yl)pyrimidin-4-yloxy)phenyl)urea was prepared following the procedures described in preparation of Example 24. [M+H]⁺ 506.92, 508.26; ¹H-NMR (400 MHz, DMSO) δ 9.73 (d, 1H), 9.16 (s, 1H), 8.63 (d, 1H), 8.45 (s, 1H), 8.38 (d, 1H), 8.14 (s, 1H), 8.02 (dd, 1H), 7.86 (d, 1H), 7.64 (dd, 1H), 7.34 (m, 2H), 6.91 (m, 1H), 3.89 (s, 3H).

### EXAMPLE 98

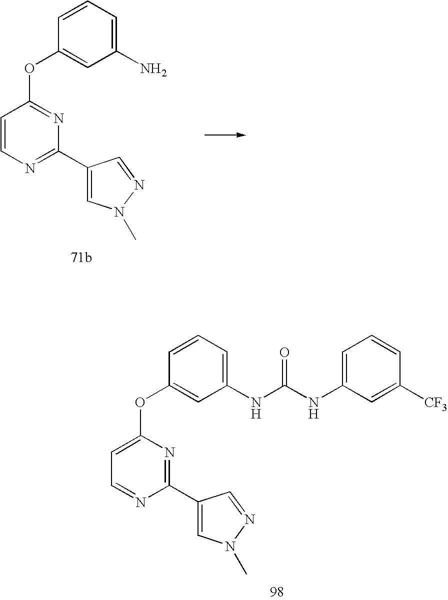

Preparation of compound 98: 1-(3-(2-(1-Methyl-1H-pyrazol-4-yl)pyrimidin-4-yloxy)phenyl)-3-(3-(trifluoromethyl)phenyl)urea was prepared following the procedures described in preparation of Example 1. [M+H]⁺ 455.13.

### EXAMPLE 99

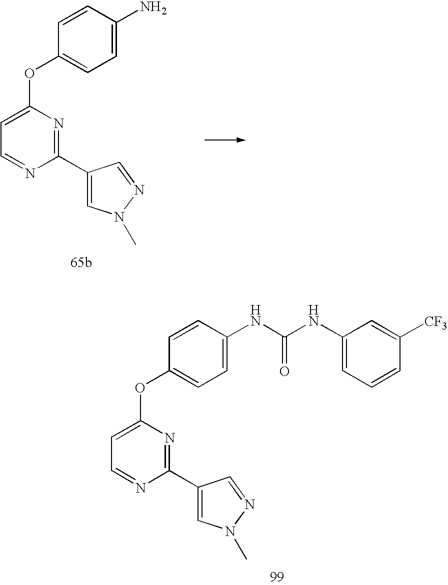

Preparation of compound 99: 1-(4-(2-(1-Methyl-1H-pyrazol-4-yl)pyrimidin-4-yloxy)phenyl)-3-(3-(trifluoromethyl)phenyl)urea was prepared following the procedures described in preparation of Example 1. [M+H]⁺ 455.13; ¹H-NMR (400 MHz, DMSO) δ 9.07 (s, 1H), 8.90 (s, 1H), 8.56 (d, 1H), 8.16 (s, 1H), 8.02 (s, 1H), 7.84 (s, 1H), 7.54 (m, 2H), 7.50 (t, 2H), 7.29 (d, 1H), 7.18 (d, 1H), 6.69 (d, 1H), 3.85 (s, 3H).

### EXAMPLE 100

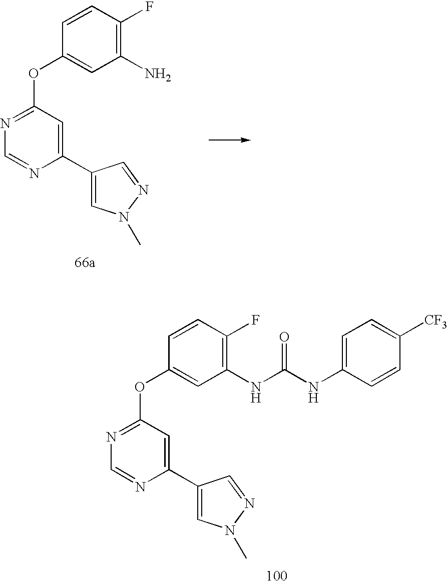

Preparation of compound 100: 1-(2-Fluoro-5-(6-(1-methyl-1H-pyrazol-4-yl)pyrimidin-4-yloxy)phenyl)-3-(4-(trifluoromethyl)phenyl)urea was prepared following the procedures described in preparation of Example 1. [M+H]⁺ 473.73; ¹H-NMR (400 MHz, DMSO) δ 9.49 (s, 1H), 8.83 (d, 1H), 8.62 (d, 1H), 8.46 (s, 1H), 8.15 (s, 1H), 8.00 (dd, 1H), 7.62 (m, 4H), 7.34 (m, 2H), 6.89 (m, 1H), 3.89 (s, 3H).

### EXAMPLE 101

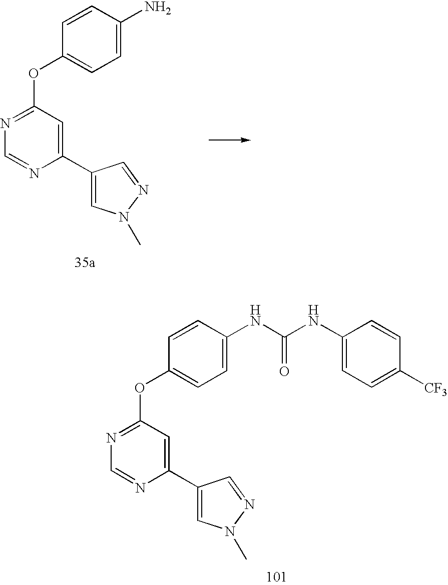

Preparation of compound 101: 1-(4-(6-(1-Methyl-1H-pyrazol-4-yl)pyrimidin-4-yloxy)phenyl)-3-(4-(trifluoromethyl)phenyl)urea was prepared following the procedures described in preparation of Example 1. [M+H]⁺ 455.69.

### EXAMPLE 102

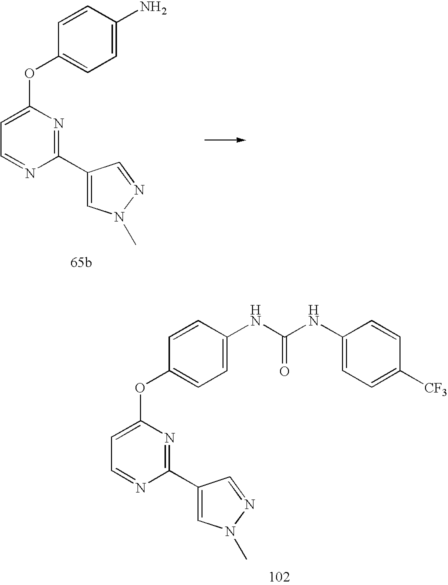

Preparation of compound 102: 1-(4-(2-(1-Methyl-1H-pyrazol-4-yl)pyrimidin-4-yloxy)phenyl)-3-(4-(trifluoromethyl)phenyl)urea was prepared following the procedures described in preparation of Example 1. [M+H]⁺ 455.93.

### EXAMPLE 103

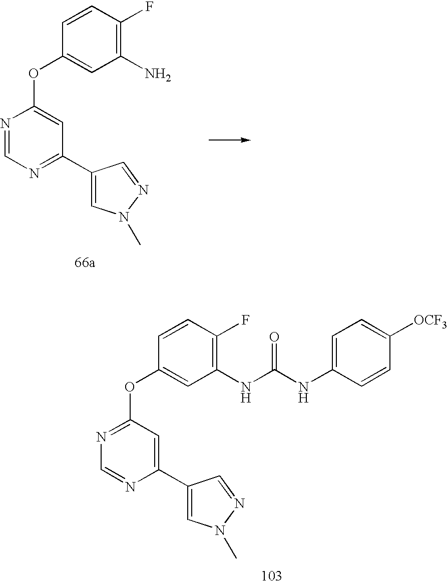

Preparation of compound 103: 1-(2-Fluoro-5-(6-(1-methyl-1H-pyrazol-4-yl)pyrimidin-4-yloxy)phenyl)-3-(4-(trifluoromethoxy)phenyl)urea was prepared following the procedures described in preparation of Example 1. [M+H]⁺ 488.91.

### EXAMPLE 104

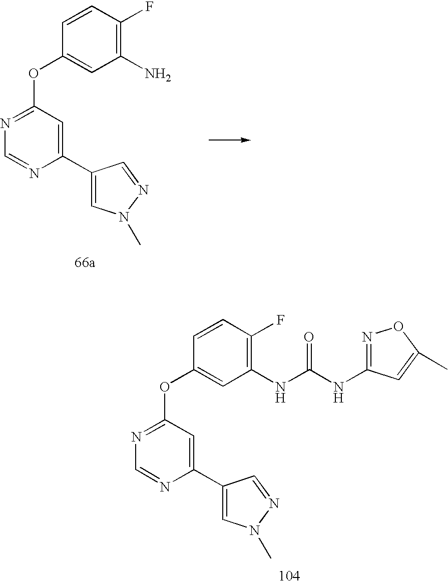

Preparation of compound 104: 1-(2-Fluoro-5-(6-(1-methyl-1H-pyrazol-4-yl)pyrimidin-4-yloxy)phenyl)-3-(5-methylisoxazol-3-yl)urea was prepared following the procedures described in preparation of Example 24. [M+H]⁺ 409.68.

### EXAMPLE 105

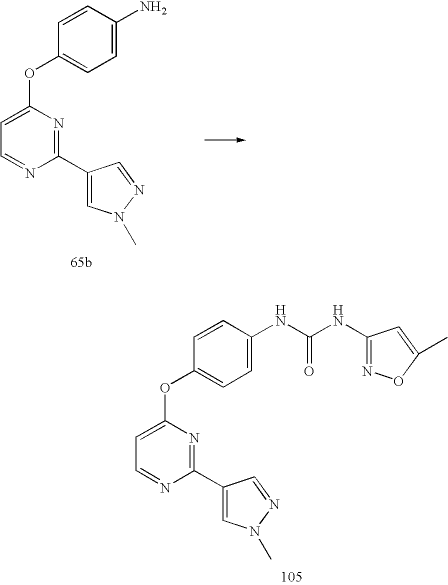

Preparation of compound 105: 1-(4-(2-(1-Methyl-1H-pyrazol-4-yl)pyrimidin-4-yloxy)phenyl)-3-(5-methylisoxazol-3-yl)urea was prepared following the procedures described in preparation of Example 24. [M+H]⁺ 391.90.

### EXAMPLE 106

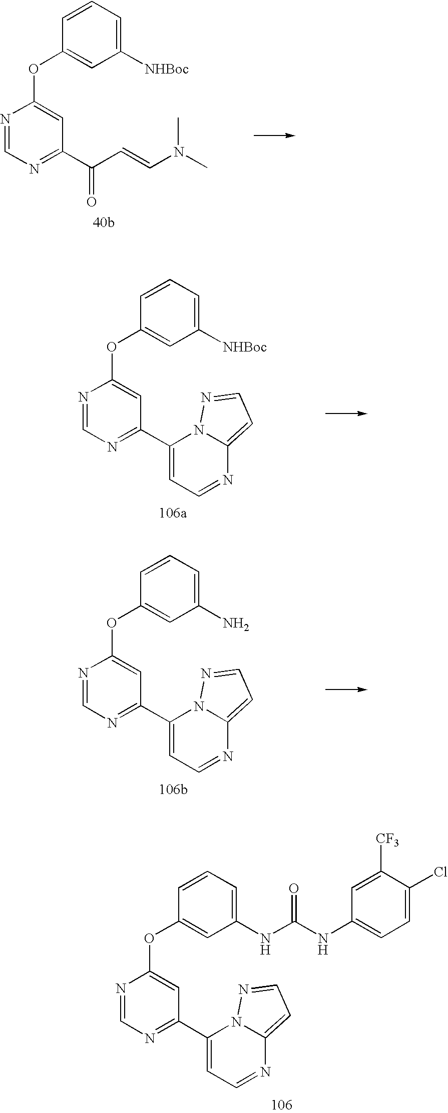 Step 1

Preparation of compound 106a: [3-(6-Pyrazolo[1,5-a]pyrimidin-7-yl-pyrimidin-4-yloxy)-phenyl]-carbamic acid tert-butyl ester was prepared following the procedures described in preparation of Example 40. [M+H]⁺ 404.97.

Step 2

Preparation of compound 106b: 3-(6-Pyrazolo[1,5-a]pyrimidin-7-yl-pyrimidin-4-yloxy)-phenylamine was prepared following the procedures described in preparation of Example 1. [M+H]⁺ 304.82.

Step 3

Preparation of compound 106: 1-(4-Chloro-3-(trifluoromethyl)phenyl)-3-(3-(6-(pyrazolo[1,5-a]pyrimidin-7-yl)pyrimidin-4-yloxy)phenyl)urea was prepared following the procedures described in preparation of Example 1. [M+H]⁺ 525.83; ¹H-NMR (400 MHz, DMSO) δ 9.21 (s, 1H), 9.06 (s, 1H), 9.00 (t, 1H), 8.85 (s, 1H), 8.77 (dd, 1H), 8.41 (dd, 1H), 8.08 (s, 1H), 7.95 (dd, 1H), 7.64-7.53 (m, 3H), 7.39 (t, 1H), 7.32-7.29 (m, 1H), 6.97 (dd, 1H), 6.95-6.90 (m, 1H).

### EXAMPLE 107

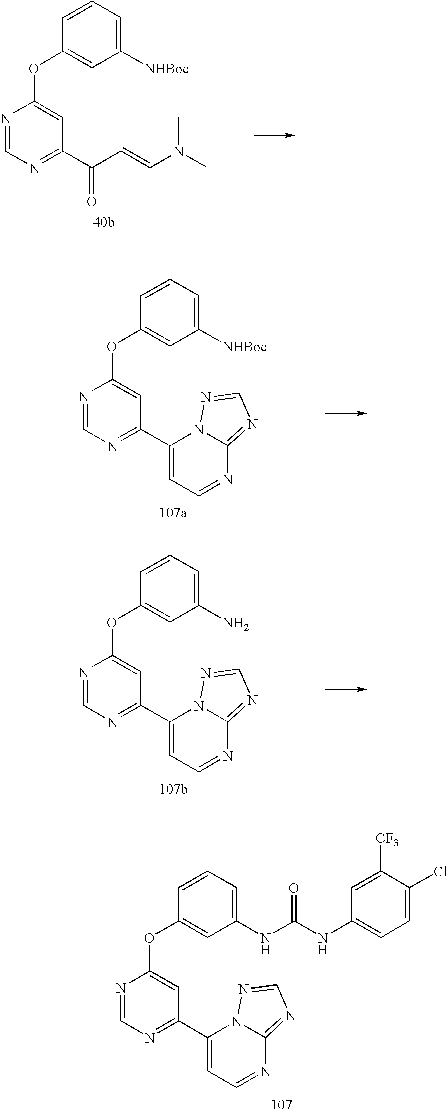 Step 1

Preparation of compound 107a: [3-(6-[1,2,4]Triazolo[1,5-a]pyrimidin-7-yl-pyrimidin-4-yloxy)-phenyl]-carbamic acid tert-butyl ester was prepared following the procedures described in preparation of Example 40. [M+H]⁺ 406.02.

Step 2

Preparation of compound 107b: 3-(6-[1,2,4]Triazolo[1,5-a]pyrimidin-7-yl-pyrimidin-4-yloxy)-phenylamine was prepared following the procedures described in preparation of Example 1. [M+H]⁺ 305.99.

Step 3

Preparation of compound 107: 1-(3-(6-([1,2,4]Triazolo[1,5-a]pyrimidin-7-yl)pyrimidin-4-yloxy)phenyl)-3-(4-chloro-3-(trifluoromethyl)phenyl)urea was prepared following the procedures described in preparation of Example 1. [M+H]⁺ 526.90; ¹H-NMR (400 MHz, DMSO) δ 9.21 (s, 1H), 9.10 (d, 1H), 9.06 (s, 1H), 9.04 (s, 1H), 8.88 (s, 1H), 8.73 (dd, 1H), 8.21 (d, 1H), 8.08 (d, 1H), 7.64-7.60 (m, 2H), 7.55 (m, 1H), 7.40 (t, 1H), 7.32-7.29 (m, 1H), 6.95-6.92 (m, 1H).

### EXAMPLE 108

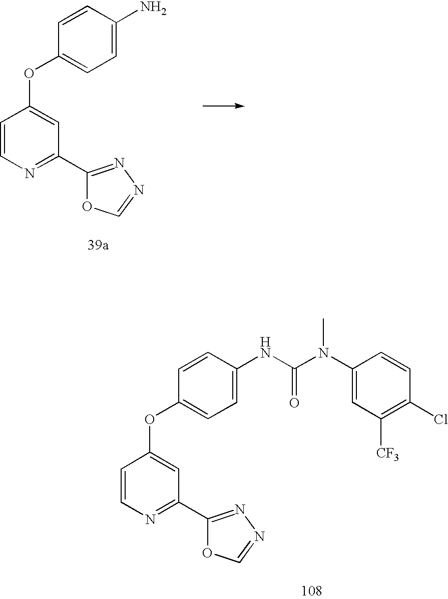

Preparation of compound 108: 3-(4-(2-(1,3,4-Oxadiazol-2-yl)pyridin-4-yloxy)phenyl)-1-(4-chloro-3-(trifluoromethyl)phenyl)-1-methylurea was prepared following the procedures described in preparation of Example 24. [M+H]⁺ 489.76; ¹H-NMR (400 MHz, CD₃OD) δ 9.40 (s, 1H), 8.70 (s, 1H), 8.63 (d, 1H), 7.83 (d, 1H), 7.72 (d, 1H), 7.65 (dd, 1H), 7.58 (d, 2H), 7.51 (d, 1H), 7.16 (m, 3H), 3.32 (d, 3H).

### EXAMPLE 109

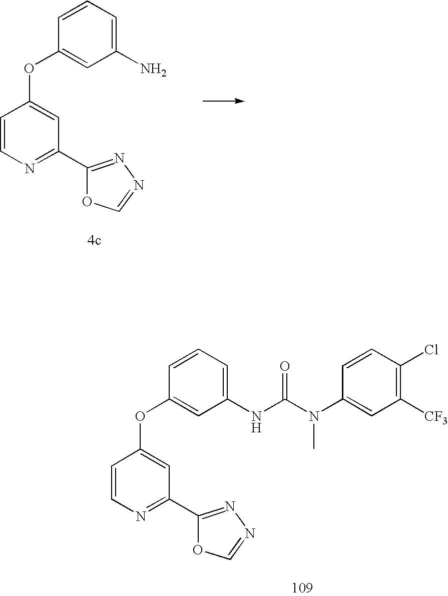

Preparation of compound 109: 3-(3-(2-(1,3,4-Oxadiazol-2-yl)pyridin-4-yloxy)phenyl)-1-(4-chloro-3-(trifluoromethyl)phenyl)-1-methylurea was prepared following the procedures described in preparation of Example 24. [M+H]⁺ 489.65; ¹H-NMR (400 MHz, CD₃OD) δ 9.40 (s, 1H), 8.73 (br s, 1H), 8.65 (d, 1H), 7.80 (d, 1H), 7.70 (d, 1H), 7.63 (dd, 1H), 7.56 (d, 1H), 7.46-7.32 (m, 3H), 7.19 (dd, 1H), 6.87 (m, 1H), 3.10 (d, 3H).

### EXAMPLE 110

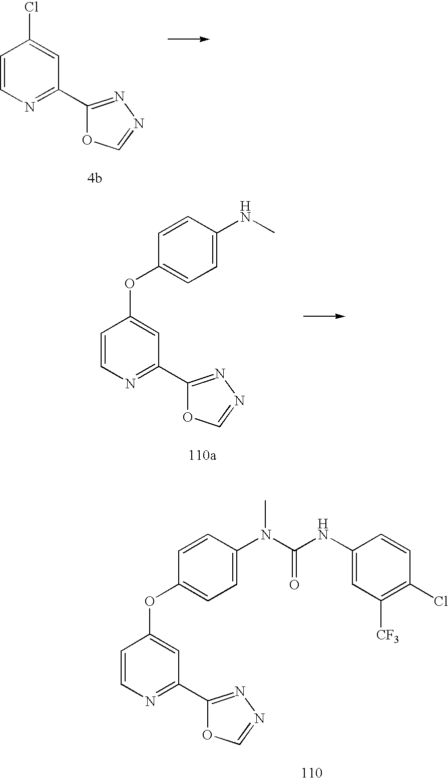 Step 1

Preparation of compound 110c: 4-(2-(1,3,4-Oxadiazol-2-yl)pyridin-4-yloxy)-N-methylaniline was prepared following the procedures described in preparation of Example 1. [M+H]⁺ 269.14.

Step 2

Preparation of compound 110: 1-(4-(2-(1,3,4-Oxadiazol-2-yl)pyridin-4-yloxy)phenyl)-3-(4-chloro-3-(trifluoromethyl)phenyl)-1-methylurea was prepared following the procedures described in preparation of Example 1. [M+H]⁺ 489.99; ¹H-NMR (400 MHz, CD₃OD) δ 9.11 (s, 1H), 8.59 (d, 1H), 7.92 (d, 1H), 7.78 (d, 1H), 7.62 (dd, 1H), 7.54-7.48 (m, 3H), 7.43 (d, 1H), 7.29 (d, 2H), 7.21 (dd, 1H), 3.36 (s, 3H).

### EXAMPLE 111

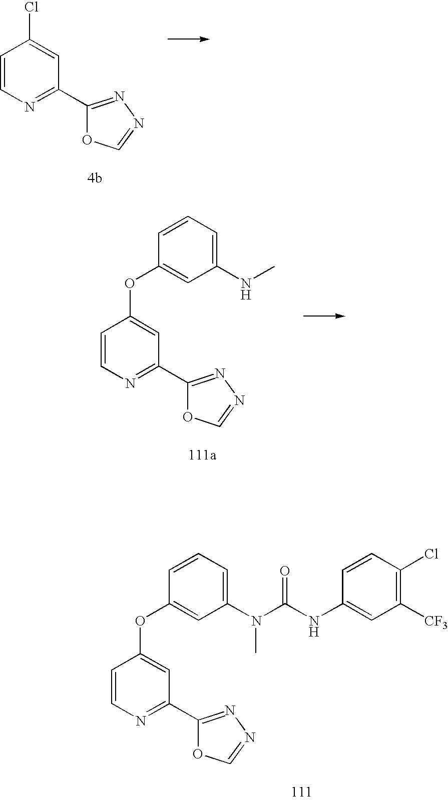 Step 1

Preparation of compound 111a: 3-(2-(1,3,4-Oxadiazol-2-yl)pyridin-4-yloxy)-N-methylaniline was prepared following the procedures described in preparation of Example 1. [M+H]⁺ 269.08.

Step 2

Preparation of compound 111: 1-(3-(2-(1,3,4-Oxadiazol-2-yl)pyridin-4-yloxy)phenyl)-3-(4-chloro-3-(trifluoromethyl)phenyl)urea was prepared following the procedures described in preparation of Example 1. [M+H]⁺ 490.10; ¹H-NMR (400 MHz, CD₃OD) δ 9.10 (s, 1H), 8.57 (d, 1H), 7.89 (d, 1H), 7.80 (d, 1H), 7.64-7.56 (m, 3H), 7.43 (d, 1H), 7.36 (m, 1H), 7.27 (t, 1H), 7.22 (dd, 1H), 7.16 (dd, 1H), 3.35 (d, 3H).

### EXAMPLE 112

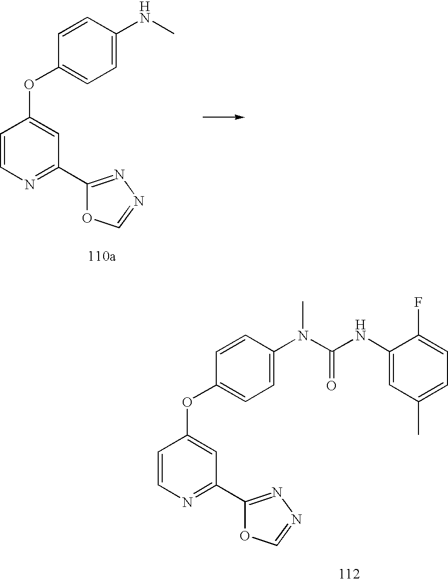

Preparation of compound 112: 3-(2-Fluoro-5-methyl-phenyl)-1-methyl-1-[4-(2-[1,3,4]oxadiazol-2-yl-pyridin-4-yloxy)-phenyl]-urea was prepared following the procedures described in preparation of Example 24. [M+H]⁺ 420.32; ¹H-NMR (400 MHz, CD₃OD) δ 9.10 (s, 1H), 8.59 (d, 1H), 7.78 (d, 1H), 7.54 (d, 2H), 7.45 (d, 1H), 7.32 (d, 2H), 7.20 (dd, 1H), 6.97-6.89 (m, 3H), 3.31 (s, 3H), 2.28 (s, 3H).

### EXAMPLE 113

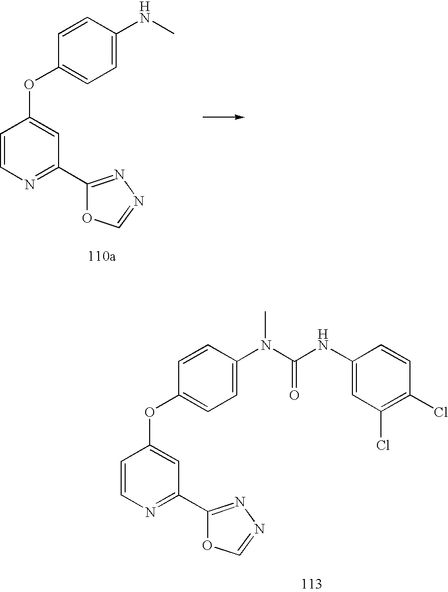

Preparation of compound 113: 3-(3,4-Dichloro-phenyl)-1-methyl-1-[4-(2-[1,3,4]oxadiazol-2-yl-pyridin-4-yloxy)-phenyl]-urea was prepared following the procedures described in preparation of Example 1. [M+H]⁺ 456.99; ¹H-NMR (400 MHz, CD₃OD) δ 9.11 (s, 1H), 8.60 (d, 1H), 7.83 (d, 1H), 7.55 (d, 1H), 7.50 (d, 2H), 7.41 (d, 1H), 7.32 (m, 2H), 7.25 (d, 1H), 7.22 (t, 1H), 7.20 (d, 1H), 3.31 (s, 3H).

### EXAMPLE 114

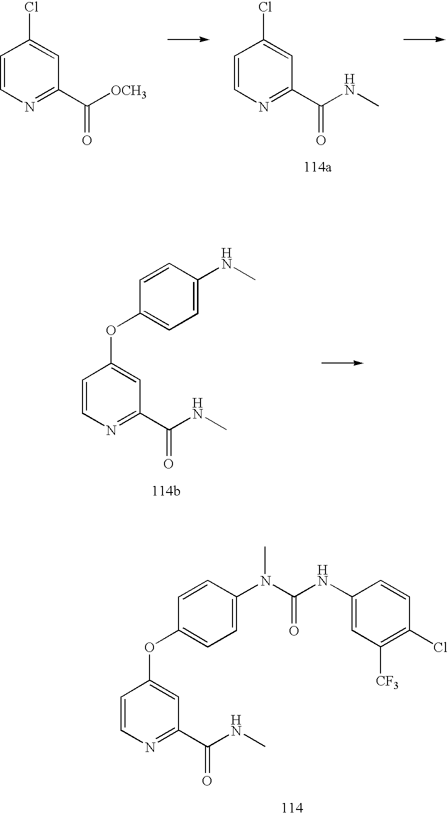 Step 1

Preparation of compound 114a: 4-Chloro-pyridine-2-carboxylic acid methylamide was prepared following the procedures described in preparation of Example 15. [M+H]⁺ 171.08.

Step 2

Preparation of compound 114b: 4-(4-Methylamino-phenoxy)-pyridine-2-carboxylic acid methylamide was prepared following the procedures described in preparation of Example 1. [M+H]⁺ 258.11.

Step 3

Preparation of compound 114: 4-{4-[3-(4-Chloro-3-trifluoromethyl-phenyl)-1-methyl-ureido]-phenoxy}-pyridine-2-carboxylic acid methylamide was prepared following the procedures described in preparation of Example 1. [M+H]⁺ 478.92; ¹H-NMR (400 MHz, CD₃OD) δ 8.48 (d, 1H), 7.93 (d, 1H), 7.64 (d, 1H), 7.60 (d, 1H), 7.50-7.42 (m, 4H), 7.23 (d, 2H), 7.14 (dd, 1H), 7.21 (dd, 1H), 3.35 (s, 3H), 2.93 (s, 3H).

### EXAMPLE 115

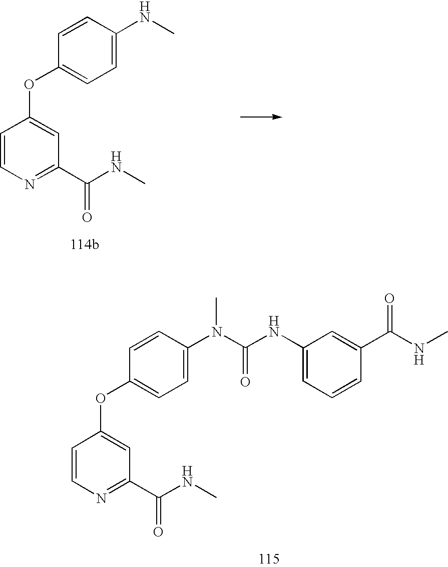

Preparation of compound 115: 4-{4-[1-Methyl-3-(3-methylcarbamoyl-phenyl)-ureido]-phenoxy}-pyridine-2-carboxylic acid methylamide was prepared following the procedures described in preparation of Example 24. [M+H]⁺ 434.16.

### EXAMPLE 116

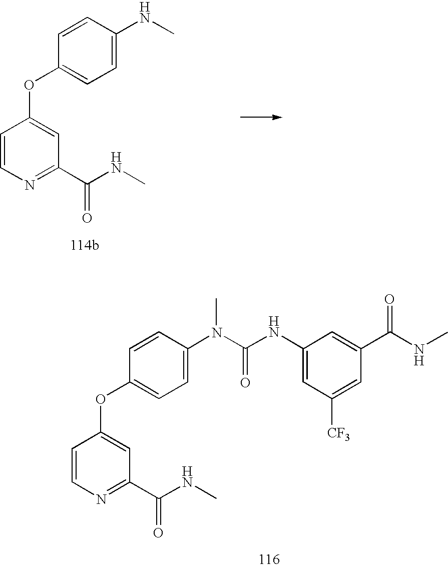

Preparation of compound 116: 4-{4-[1-Methyl-3-(3-methylcarbamoyl-5-trifluoromethyl-phenyl)-ureido]-phenoxy}-pyridine-2-carboxylic acid methylamide was prepared following the procedures described in preparation of Example 24. [M+H]⁺ 502.99; ¹H-NMR (400 MHz, DMSO) δ 8.82-8.78 (m, 2H), 8.60 (dd, 1H), 8.52 (d, 1H), 8.20 (s, 1H), 8.09 (s, 1H), 7.72 (s, 1H), 7.50 (d, 1H), 7.46 (d, 2H), 7.25 (d, 2H), 7.20 (dd, 1H), 3.33 (s, 3H), 3.30 (s, 3H), 2.77 (d, 3H).

### EXAMPLE 117

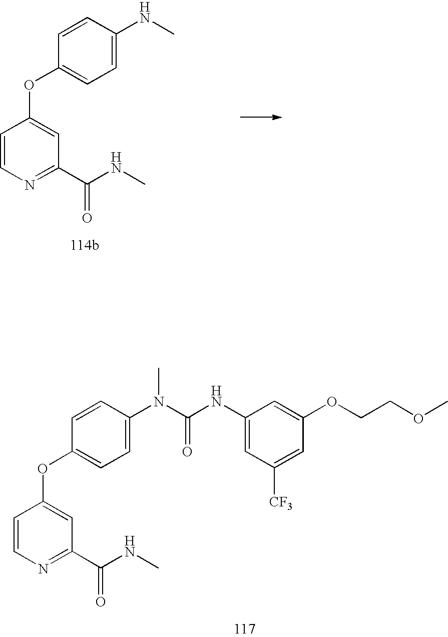

Preparation of compound 117: 4-(4-{3-[3-(2-Methoxy-ethoxy)-5-trifluoromethyl-phenyl]-1-methyl-ureido}-phenoxy)-pyridine-2-carboxylic acid methylamide was prepared following the procedures described in preparation of Example 24. [M+H]⁺ 518.78; ¹H-NMR (400 MHz, CD₃OD) δ 8.48 (d, 2H), 7.64 (d, 1H), 7.62 (d, 1H), 7.54-7.44 (m, 4H), 7.24 (d, 2H), 7.15 (dd, 1H), 7.11 (d, 1H), 4.17 (t, 2H), 3.75 (t, 2H), 3.42 (s, 3H), 3.30 (s, 3H), 2.93 (s, 3H).

### EXAMPLE 118

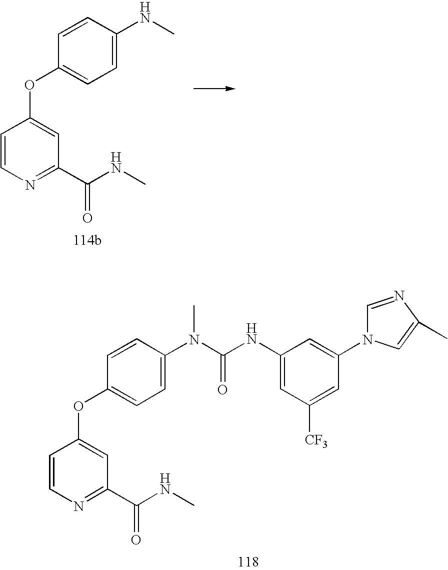

Preparation of compound 118: 4-(4-{1-Methyl-3-[3-(4-methyl-imidazol-1-yl)-5-trifluoromethyl-phenyl]-ureido}-phenoxy)-pyridine-2-carboxylic acid methylamide was prepared following the procedures described in preparation of Example 24. [M+H]⁺ 526.20; ¹H-NMR (400 MHz, CD₃OD) δ 8.50 (d, 2H), 8.08 (d, 1H), 7.90 (br s, 1H), 7.83 (br s, 1H), 7.61 (d, 1H), 7.54-7.46 (m, 4H), 7.32 (s, 1H), 7.26 (d, 2H), 7.17 (dd, 1H), 3.30 (s, 3H), 2.94 (s, 3H), 2.25 (s, 3H).

### EXAMPLE 119

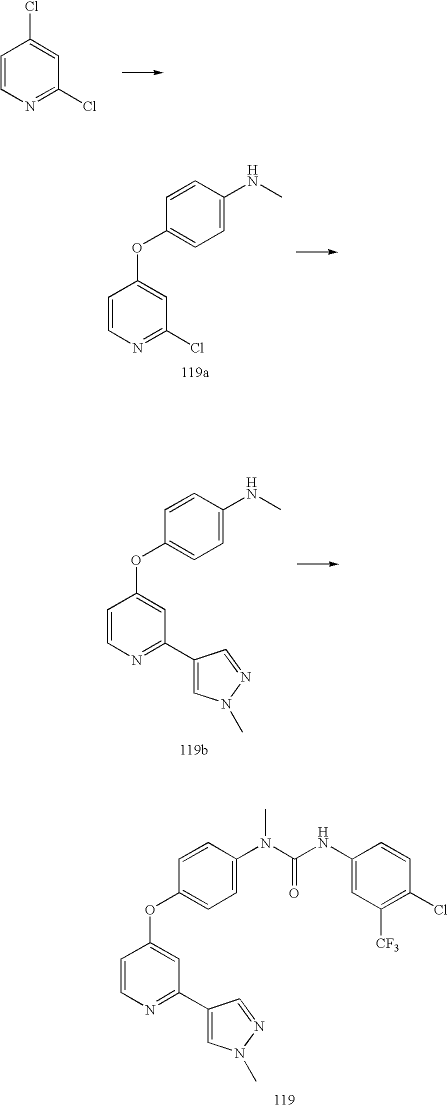 Step 1

Preparation of compound 119a: [4-(2-Chloro-pyridin-4-yloxy)-phenyl]-methyl-amine was prepared following the procedures described in preparation of Example 1. [M+H]⁺ 234.99.

Step 2

Preparation of compound 119b: Methyl-{4-[2-(1-methyl-1H-pyrazol-4-yl)-pyridin-4-yloxy]-phenyl}-amine was prepared following the procedures described in preparation of Example 27. [M−H]⁺ 279.55.

Step 3

Preparation of compound 119: 3-(4-Chloro-3-trifluoromethyl-phenyl)-1-methyl-1-{4-[2-(1-methyl-1H-pyrazol-4-yl)-pyridin-4-yloxy]-phenyl}-urea was prepared following the procedures described in preparation of Example 1. [M+H]⁺ 501.97; ¹H-NMR (400 MHz, CD₃OD) δ 8.34 (d, 1H), 8.11 (s, 1H), 7.96 (s, 1H), 7.92 (d, 1H), 7.62 (dd, 1H), 7.48-7.42 (m, 4H), 7.27 (d, 1H), 7.25 (d, 2H), 6.81 (dd, 1H), 3.93 (s, 3H), 3.37 (s, 3H).

### EXAMPLE 120

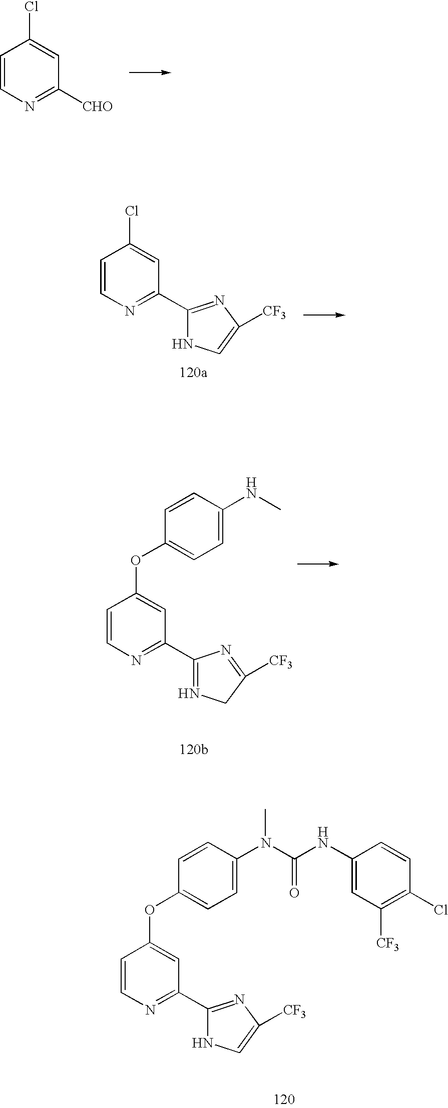 Step 1

Preparation of compound 120a: 4-Chloro-2-(4-trifluoromethyl-1H-imidazol-2-yl)-pyridine was prepared following the procedures described in preparation of Example 73. [M+H]⁺ 248.11.

Step 2

Preparation of compound 120b: Methyl-{4-[2-(4-trifluoromethyl-1H-imidazol-2-yl)-pyridin-4-yloxy]-phenyl}-amine was prepared following the procedures described in preparation of Example 1. [M−H]⁺ 335.05.

Step 3

Preparation of compound 120: 3-(4-Chloro-3-trifluoromethyl-phenyl)-1-methyl-1-{4-[2-(4-trifluoromethyl-1H-imidazol-2-yl)-pyridin-4-yloxy]-phenyl}-urea was prepared following the procedures described in preparation of Example 1. [M+H]⁺ 555.78; ¹H-NMR (400 MHz, DMSO) δ 13.55 (s, 1H), 8.72 (s, 1H), 8.57 (d, 1H), 8.04 (d, 1H), 7.86 (d, 1H), 7.79 (dd, 1H), 7.56-7.52 (m, 2H), 7.47 (d, 2H), 7.28 (d, 2H), 7.10 (dd, 1H), 3.30 (d, 3H).

The following compounds can generally be made using the methods described above. It is expected that these compounds when made will have activity similar to those that have been made in the examples above. The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All IUPAC names and SMILES strings herein were generated using CambridgeSoft's ChemDrew 10.0.
CN(N═C1)C═C1C2=NC═NC(OC3=CC(NC(NC4=CC(C(F)(F)F)═CC═C4)=O)═CC(F)═C3)=C2
CN(N═C1)C═C1C2=NC═NC(OC3=CC(NC(NC4=CC(C(F)(F)F)═C(C1)C═C4)=O)═CC(F)═C3)=C2
CN(N═C1)C═C1C2=NN═CC(OC3=CC(NC(NC4=CC(C(F)(F)F)═CC═C4)=O)═C(F)C═C3)=C2
CN(N═C1)C═C1C2=CN═CC(OC3=CC(NC(NC4=CC(C(F)(F)F)═C(C1)C═C4)=O)═C(F)C═C3)=N2
CN(N═C1)C═C1C2=NC═CC(OC3=CC(NC(NC4=CC(C(F)(F)F)═CC═C4)=O)═CC(F)═C3)=N2
CN(N═C1)C═C1C2=CN═NC(OC3=CC(NC(NC4=CC(C(F)(F)F)═C(C1)C═C4)=O)═C(F)C═C3)=C2
CN(N═C1)C═C1C2=NC═NC(OC3=CC(NC(NC4=CC(C(F)(F)F)═CC═C4)=O)═C(F)C═C3)=C2F
CN(N═C1)C═C1C2=CC═NC(OC3=CC(NC(NC4=CC(C(F)(F)F)═C(C1)C═C4)=O)═C(F)C═C3)=N2
CN(N═C1)C═C1C2=NN═CC(OC3=CC(NC(NC4=CC(C(F)(F)F)═CC═C4)=O)═C(F)C═C3)=C2F
CN(C═N1)C═C1C2=NC═CC(OC3=CC(NC(NC4=CC(C(F)(F)F)═C(C1)C═C4)=O)═C(F)C═C3)=N2
CN(N═C1)C═C1C2=NC═CC(OC3=CC(NC(NC4=CC(C(F)(F)F)═C(C1)C═C4)=O)═CC(F)═C3)=N2
CN(N═C1)C═C1C2=NC═NC(OC3=CC(NC(NC4=CC(C(F)(F)F)═C(C)C═C4)=O)═C(F)C═C3)=C2F
CN(C═N1)C═C1C2=NC═NC(OC3=CC(NC(NC4=CC(C(F)(F)F)═CC═C4)=O)═C(F)C═C3)=C2
CN(N═C1)C═C1C2=NC═C(F)C(OC3=CC(NC(NC4=CC(C(F)(F)F)═C(C1)C═C4)=O)═CC═C3)=N2
CN(N═C1)C═C1C2=NC═NC(OC3=CC(F)═C(NC(NC4=CC(C(F)(F)F)═CC═C4)=O)C(F)═C3)=C2
CN(N═C1)C═C1C2=NN═CC(OC3=CC═C(NC(NC4=CC(C(F)(F)F)═C(C1)C═C4)=O)C(F)═C3)=C2
CN(N═C1)C═C1C2=NN═CC(OC3=CC═C(NC(NC4=CC(C(F)(F)F)═CC═C4)=O)C═C3)=C2
CN(N═C1)C═C1C2=NC═CC(OC3=CC(F)═C(NC(NC4=CC(C(F)(F)F)═C(C1)C═C4)=O)C(F)═C3)=N2
CN(N═C1)C═C1C2=CN═NC(OC3=CC═C(NC(NC4=CC═C(C(F)(F)F)C═C4)=O)C(F)═C3)=C2
CN(N═C1)C═C1C2=CN═NC(OC3=CC═C(NC(NC4=CC(C(F)(F)F)═C(C1)C═C4)=O)C(F)═C3)=N2
CN(C═N1)C═C1C2=NC═CC(OC3=CC═C(NC(NC4=CC(C(F)(F)F)═CC═C4)=O)C(F)═C3)=N2
CN(N═C1)C═C1C2=NC═NC(OC3=CC═C(NC(NC4=CC(C(F)(F)F)═C(C1)C═C4)=O)C(F)═C3)=N2
CN(N═C1)C═C1C2=CN═CC(OC3=CC═C(NC(NC4=CC(C(F)(F)F)═C(C1)C═C4)=O)C(F)═C3)=N2
CN(N═C1)C═C1C2=NC═C(F)C(OC3=CC═C(NC(NC4=CC(C(F)(F)F)═CC═C4)=O)C(F)═C3)=N2
CN(C═N1)C═C1C2=NC═NC(OC3=CC═C(NC(NC4=CC(C(F)(F)F)═C(C1)C═C4)=O)C(F)═C3)=C2
CN(N═C1)C═C1C2=CC═NC(OC3=CC═C(NC(NC4=CC(C(F)(F)F)═C(C1)C═C4)=O)C(F)═C3)=N2
FC1=C(N(CC)C(NC2=CC(C(F)(F)F)═CC═C2)=O)C═CC(OC3=CC(C4=NN═C4)=NC═C3)=C1
FC1=C(N(C)C(C2=CC(C(F)(F)F)═C(C1)C═C2)=O)C═CC(OC3=CC(C(NC)═O)═NC═C3)=C1
FC1=C(C(N(C)C2=CC(C(F)(F)F)═C(C1)C═C2)=O)C═CC(OC3=CC(C4=NN═C(C)O4)=NC═C3)=C1
FC1=C(NC(N(C)C2=CC(C(F)(F)F)═CC(C(F)(F)F)═C2)=O)C═CC(OC3=CC(C(NC)═O)═NC═C3F)═C
FC1=C(N(C)C(NC2=CC═C(C(F)(F)F)C═C2)=O)C═CC(OC3=CC(C4=CN(C)N═C4)=NC═C3)=C1
O═C(NCC)C1=NC═CC(OC2=CC═C(NC(N(C)C3=CC(C(F)(F)F)═C(C1)C═C3)=O)C═C2)=C1
O═C(NC1=CC(C(F)(F)F)═C(C1)C═C1)N(C)C(C═C2)=CC═C2C3=CC(C4=NN═CO4)=NC═C3F
O═C(NC)C1=NC═CC(OC2=CC═C(NC(N(C)C3=CC(C(F)(F)F)═C(C1)C═C3)=O)C═C2)=C1F
FC1=C(NC(NC2=CC(OC(F)(F)F)═CC═C2)=O)C═CC(OC3=C(F)C(C4=NN═CO4)=NC═C3)=C1
O═C(NC)C1=NC═CC(OC2=CC═C(NC(N(C(C)C)C3=CC(C(F)(F)F)═C(C1)C═C3)=O)C═C2)=C1
O═C(N(CC)C1=CC═C(C1)C═C1)NC2=C(F)C═CC(OC3=CC(C(NC)═O)═NC═C3)=C2
O═C(N(C)C1=CC(C(F)(F)F)═C(C1)C═C1)NC2=C(F)C═CC(OC3=CC(C4=CN(C)N═C4)=NC═C3)=C2F
O═C(NC1=CC(C(F)(F)F)═C(C1)C═C1)N(C)C2=C(F)C═CC(OC3=CC(C(NCC)═O)═NC═C3)=C2
O═C(NC1=CC(C(F)(F)F)═C(C1)C═C1)N(C)C2=C(F)C═CC(OC3=CC(C4=NC═CN4)=NC═C3)=C2
O═C(NC1=CC(C(F)(F)F)═CC═C1)N(C)C2=C(F)C═CC(OC3=CC(C(NC)═O)═NC═C3)=C2
O═C(NC1=CC(C(F)(F)F)═C(C1)C═C1)N(C)C2=CC(F)═CC(OC3=CC(C4=NC(C(F)(F)F)═CN4)=NC═C3)=C2
O═C(NC1=CC(C(F)(F)F)═C(C1)C═C1)N(C)C2=CC═CC(OC3=CC(C(NC)═O)═NC═C3F)═C2
O═C(N(C)C1=CC(C(F)(F)F)═C(C1)C═C1)NC2=CC═CC(OC3=CC(C4=NC═CN4)=NC═C3F)═C2
O═C(NC1=CC(C(F)(F)F)═C(C1)C═C1)N(C)C2=C(F)C═CC(OC3=CC(C4=CN(C)N═C4)=NC═C3)=C2
O═C(NC1=CC(C(F)(F)F)═C(C1)C═C1)N(C)C2=C(F)C═CC(OC3=CC(C4=NN═C(C)O4)=NC═C3)=C2
O═C(NC1=CC(C(F)(F)F)═C(C1)C═C1)NC2=C(F)C═CC(OC3=CC(C4=CN(CC)N═C4)=NC═N3)=C2
O═C(NC1=CC(C(F)(F)F)═C(C1)C═C1)NC2=CC═CC(OC3=CC(C4=CSN═N4)=NC═N3)=C2
O═C(NC1=CC(C(F)(F)F)═CC═C1)NC2=C(F)C═CC(OC3=CC(C4=CSC═N4)=NN═C3)=C2
O═C(NC1=CC(C(F)(F)F)═C(C1)C═C1)NC2=C(F)C═CC(OC3=NC(C4=CSN═C4)=CN═C3)=C2
O═C(NC1=CC(C(F)(F)F)═C(C1)C═C1)NC2=CC(F)═CC(OC3=NC(C4=CON═C4)=NC═C3)=C2
O═C(NC1=CC(C(F)(F)F)═C(C1)C═C1)NC2=C(F)C═CC(OC3=CC(C4=NC═NN4C)═NC═N3)=C2
O═C(NC1=CC(C(F)(F)F)═CC═C1)NC2=C(F)C═CC(OC3=C(F)C(C4=COC(C)=N4)=NC═N3)=C2
O═C(NC1=CC(C(F)(F)F)═C(C1)C═C1)NC2=C(F)C═CC(OC3=NC(C4=C(C)NN═C4)=CC═N3)=C2
O═C(NC1=CC(C(F)(F)F)═C(C1)C═C1)NC2=C(F)C═CC(OC3=C(F)C(C4=NN═CO4)=NN═C3)=C2
O═C(NC1=CC(C(F)(F)F)═C(C1)C═C1)NC2=CC(F)═CC(OC3=NC(C4=CNC═N4)=NC═C3)=C2
CN(C═N1)N═C1C2=NC═NC(OC3=CC(NC(NC4=CC(C(F)(F)F)═C(C1)C═C4)=O)═C(F)C═C3)=C2F
O═C(NC1=CC(C(F)(F)F)═C(C1)C═C1)NC2=CC═CC(OC3=NC(C4=CNC═C4)=NC═C3F)═C2
O═C(N(C)C1=CC═C(C1)C═C1)NC2=C(F)C═CC(OC3=CC(C(NC)═O)═NC═N3)=C2
O═C(NC1=CC(C(F)(F)F)═C(C1)C═C1)N(C)C2=C(F)C═CC(OC3=NC(C4=NN═CO4)=NC═C3)=C2
O═C(NC1=CC(C(F)(F)F)═C(C1)C═C1)N(C)C2=C(F)C═CC(OC3=NC(C(NC)═O)═CC═C3)=C2
O═C(NC1=CC(C(F)(F)F)═C(C1)C═C1)N(C)C2=C(F)C═CC(OC3=NC(C4=CN(C)C═N4)=NC═C3)=C2
O═C(NC1=CC(C(F)(F)F)═CC═C1)N(C)C2=C(F)C═CC(OC3=NC(C(NC)═O)═CC═N3)=C2
O═C(NC1=CC(C(F)(F)F)═C(C1)C═C1)N(C)C2=CC(F)═CC(OC3=CC(C4=NC(C(F)(F)F)═CN4)=NC═N3)=C2
O═C(NC1=CC(C(F)(F)F)═C(C1)C═C1)N(C)C2=CC═CC(OC3=CC(C(NC)═O)═CC═N3)=C2
O═C(N(C)C1=CC(C(F)(F)F)═C(C1)C═C1)NC2=C(F)C═CC(OC3=CC(C4=NC(C)═C(C)N4C)═NC═N3)=C2
O═C(NC1=CC(C(F)(F)F)═C(C1)C═C1)N(C)C2=C(F)C═CC(OC3=NC(C4=CN(C)N═C4)=CC═N3)=C2
O═C(NC1=CC(C(F)(F)F)═C(C1)C═C1)N(C)C2=C(F)C═CC(OC3=CC(C4=CN═C(C)O4)=NC═N3)=C2
O═C(N(C)C1=CC═C(C1)C═C1)NC2=C(F)C═CC(OC3=CC(C(NC)═O)═CC═N3)=C2
O═C(NC1=CC(C(F)(F)F)═C(C1)C═C1)N(C)C2=CC(F)═CC(OC3=CC(C4=NN═CO4)=CC═N3)=C2
O═C(NC1=CC(C(F)(F)F)═C(C1)C═C1)N(C)C2=C(F)C═CC(OC3=NC(C(NC(C)C)═O)═CC═C3)=C2
O═C(NC1=CC(C(F)(F)F)═CC═C1)N(C)C2=C(F)C═CC(OC3=NC(C4=CN(C)C═N4)=CC═C3)=C2
O═C(NC1=CC(C(F)(F)F)═C(C1)C═C1)N(C)C2=C(F)C═CC(NC3=CC(C(NC)═O)═CC═N3)=C2
O═C(NC1=CC(C(F)(F)F)═C(C1)C═C1)N(C)C2=CC(F)═CC(OC3=NC(C4=NC(C)═CN4)=CC═C3)=C2
O═C(NC1=CC(C(F)(F)F)═C(C1)C═C1)N(C)C2=C(F)C═CC(OC3=CC(C(NC)═O)═CC═N3)=C2
O═C(N(C)C1=CC(C(F)(F)F)═C(C1)C═C1)NC2=C(F)C═CC(OC3=CC(C4=CN(C)C═N4)=CC═N3)=C2
O═C(N(C)C1=CC(C(F)(F)F)═C(C1)C═C1)NC2=C(F)C═CC(OC3=CC(C4=CN(C)N═C4)=C(F)C═N3)=C2
O═C(NC1=CC(C(F)(F)F)═C(C1)C═C1)N(C)C2=C(F)C═CC(OC3=CC(C4=CN═C(C)S4)=CC═N3)=C2
O═C(NC1=CC(OCCN2CCCC2)=C(C1)C═C1)NC3=C(F)C═CC(OC4=CC(C(NC)═O)═NC═C4)=C3
O═C(NC1=CC(N2C═CC═C2)=CC═C1)NC3=C(F)C═CC(OC4=NC(C5=NN═CO5)=CC═N4)=C3
O═C(NC1=CC(C(F)(F)F)═C(OCC(F)(F)F)C═C1)NC2=CC═CC(OC3=NC(C(NC)═O)═CC═C3)=C2
O═C(NC1=CC(OCCOC)═C(C(F)(F)F)C═C1)NC2=C(F)C═CC(OC3=NC(C4=CN(C)C═N4)=NC═C3)=C2
O═C(NC1=C(OCCN(C)C)C(C(F)(F)F)═CC═C₁)NC2=CC(F)═CC(OC3=CC(C(N(C)C)═O)═NC═C3)=C2
O═C(NC1=CC(N2C═CC═C2)=CC═C₁)NC3=CC═CC(OC4=CC(C5=NC(C(F)(F)F)═CN5)=NC═N4)=C3
O═C(NC1=C(OC)C(C(F)(F)F)═C(C1)C═C₁)NC2=CC═CC(OC3=CC(C(NC)═O)═CC═N3)=C2
O═C(NC1=CC(N2CCCC2)=CC═C1)NC3=C(F)C═CC(OC4=CC(C5=CN(C)C═N5)=NC═N4)=C3
O═C(NC1=CC(OCCNC)═C(C1)C═C₁)NC2=C(F)C═CC(OC3=NC(C4=CN(C)N═C4)=CC═N3)=C2
O═C(NC1=CC(OC(C)C)═CC═C₁)NC2=C(F)C═CC(OC3=NC(C4=NN═C(C)O4)=CC═C3)=C2
FC1=C(NC(NC2=CC(C(F)(F)F)═CC(OC(C)C)═C2)=O)C═CC(OC3=CC(C4=NN═CO4)=NC═C3)=C1
FC1=C(NC(NC2=CC(OCCN3CCN(C)CC3)=CC═C2)=O)C═CC(OC4=CC(C(NC)═O)═NC═C4)=C1
FC1=C(NC(NC2=CC(N3C═CC═C3)=CC═C2)=O)C═CC(OC4=NC(C5=NN═C(C)O5)=NC═C4)=C1
FC1=C(NC(NC2=CC(OCCN(C)C)═CC(C(F)(F)F)═C2)=O)C═CC(OC3=CC(C(NC)═O)═NC═C3F)═C1
FC1=C(NC(NC2=CC(OCCOC)═C(C(F)(F)F)C═C2)=O)C═CC(OC3=CC(C4=CN(C)N═C4)=NC═C3)=C1
O═C(NCC)C1=NC═CC(OC2=CC═C(NC(NC3=CC(OCC(C)C)═C(C1)C(F)═C3)=O)C═C2)=C1
O═C(NC1=CC(OCC(F)(F)F)═C(C1)C═C1)NC(C═C2)=CC═C2OC3=CC(C4=NN═CO4)=NC═C3F
O═C(NC)C1=NC═CC(OC2=CC═C(NC(NC3=CC(C(F)(F)F)═C(OC)C═C3)=O)C═C2)=C1F
FC1=C(NC(N(C)C2=CC(OC(F)(F)F)═CC(OC(F)(F)F)═C2)=O)C═CC(OC3=C(F)C(C4=NN═CO4)=NC═C3)=C1
O═C(NC)C1=NC═CC(OC2=CC═C(NC(N(C)C3=CC(N(C)C)═CC═C3)=O)C═C2)=C1

The activity of the compounds in Examples 1-120 as protein kinase inhibitors is illustrated in the following assays. The other compounds listed above, which have not yet been made and/or tested, are predicted to have activity in these assays as well.

### Biological Activity Assay

In Vitro B-Raf/Mek1 Composite Kinase Assay

2.5 μl of B-Raf kinase buffer (20 mM MOPS [pH 7.2], 25 mM sodium glycerophosphate, 2 mM EGTA [pH 8.0], 1 mM sodium orthovanadate, 1 mM dithiothreitol, 10 mM MgCl₂, 0.03% Brij-35, 0.3 mg/ml bovine serum albumin) containing 1 ng of recombinant, N-terminal GST-tagged human B-Raf protein kinase (Δ1-415, Upstate Inc., cat. #14-530) is dispensed to wells of a 1536 multi-well white solid plate. 60 nl of 100× concentration of test compound in DMSO is dispensed to the well by passive pin transfer and incubated for 15 minutes at room temperature (approx. 22° C.). 2.5 μl of B-Raf kinase buffer containing 12.5 ng of recombinant N-terminal GST-tagged, C-terminal His6-tagged human Mek1 (inactive, Upstate Inc., cat. #14-420) and 2 μM ATP is then dispensed and the kinase reaction allowed to incubate at 30° C. for 2 hours. The assay plates are sealed and maintained in a humidified environment. After 2 hours, 2.5 μl of PKLight protein kinase assay reagent (Cambrex, cat. #LT07-501) is dispensed. After an additional 5 minute incubation at room temperature, luminescence activity is measured on a Molecular Devices Analyst multi-mode plate reader or other suitable plate reader. Kinase inhibition results in less ATP depletion, and therefore increased luminescence signal. Negative control activity is measured with DMSO lacking any test compound. The positive control is [N-(3-trifluoromethyl-4-chlorophenyl)-N′-(4-(2-methylcarbamoyl-pyridin-4-yl)oxyphenyl)urea], aka Bay 43-9006. Efficacy is measured as a percentage of positive control activity.

In vitro vegfr2 and PDGFRβ Kinase Assays

2.5 μl of ADP Quest assay buffer (DiscoverX Inc., cat. #90-0071) containing 20 ng VEGFR2 kinase (Invitrogen Inc, cat. #PV3660) or 25 ng PDGFRβ kinase (Invitrogen Inc., cat. #P3082) is dispensed to wells of a 1536 multi-well, black solid plate. 60 nl of 100× concentration of test compound in DMSO is dispensed to the well by passive pin transfer and incubated for 10 minutes at room temperature (approx. 22° C.). 2.5 μl of ADP Quest assay buffer containing 0.25 μg of poly Glu:Tyr (4:1) substrate peptide (Upstate Inc., cat. #12-440) and 60 μM ATP is then dispensed and the kinase reaction allowed to incubate at 30° C. for 2 hours. The assay plates are sealed and maintained in a humidified environment. After the 2 hour incubation, 2 μl of ADP Quest assay reagent A, followed by 2 μl of assay reagent B, is added. After an additional 30 minute incubation at room temperature, fluorescence intensity is measured on a Molecular Devices Aquest multi-mode plate reader or other suitable plate reader (fluorescence excitation filter: 530/25 [Peak (nm)/FWHM passband (nm)]; dichroic beamsplitter: 561 nm longpass; fluorescence emission filter: 580/10 [Peak (nm)/FWHM passband (nm)]. The assay measures the conversion of a non-fluoresecent molecule to fluorescent resorutin, which correlates with kinase activity. Negative control activity is measured with DMSO lacking any test compound. The positive control is [N-(3-trifluoromethyl-4-chlorophenyl)-N′-(4-(2-methylcarbamoyl-pyridin-4-yl)oxyphenyl)urea], aka Bay 43-9006. Efficacy is measured as a percentage of positive control activity.

IC₅₀ data were obtained for the compounds provided herein. Data for selected compounds is shown in Table 1 below. Compounds not test were designated NT as shown in Table 1.

TABLE 1
Biological Activity

| | B-Raf Kinase | VEGFR2 Assay | PDGFRβ Assay |
|---|---|---|---|
| Exam- | Assay IC₅₀ μM | IC₅₀ μM | IC₅₀ μM |
| ple | − indicates ≦10 μM | − indicates ≦10 μM | − indicates ≦10 μM |
| # | + indicates >10 μM | + indicates >10 μM | + indicates >10 μM |
| 1 | − | − | − |
| 2 | + | + | + |
| 3 | + | − | − |
| 4 | NT | NT | NT |
| 5 | − | − | + |
| 6 | − | + | + |
| 7 | + | NT | NT |
| 8 | − | − | − |
| 9 | − | − | − |
| 10 | − | − | − |
| 11 | − | − | − |
| 12 | − | − | − |
| 13 | − | − | − |
| 14 | − | − | − |
| 15 | − | − | − |
| 16 | − | − | − |
| 17 | − | − | − |
| 18 | + | − | − |
| 19 | + | − | − |
| 20 | − | − | − |
| 21 | − | − | − |
| 22 | − | − | − |
| 23 | − | − | − |
| 24 | − | − | − |
| 25 | − | − | − |
| 26 | − | − | − |
| 27 | − | − | − |
| 28 | − | − | − |
| 29 | − | − | − |
| 30 | + | − | − |
| 31 | − | − | − |
| 32 | − | − | − |
| 33 | − | − | − |
| 34 | − | − | − |
| 35 | − | − | − |
| 36 | − | + | − |
| 37 | − | − | − |
| 38 | − | − | − |
| 39 | − | − | − |
| 40 | − | − | − |
| 41 | − | − | − |
| 42 | − | − | − |
| 43 | − | − | − |
| 44 | − | − | − |
| 45 | − | − | − |
| 46 | − | − | − |
| 47 | − | − | − |
| 48 | − | − | − |
| 49 | − | + | + |
| 50 | − | − | − |
| 51 | − | − | − |
| 52 | − | − | − |
| 53 | + | − | − |
| 54 | − | − | − |
| 55 | − | − | NT |
| 56 | − | − | − |
| 57 | − | − | − |
| 58 | − | − | − |
| 59 | − | − | − |
| 60 | − | − | − |
| 61 | − | + | + |
| 62 | − | − | − |
| 63 | − | + | + |
| 64 | − | − | − |
| 65 | − | − | − |
| 66 | − | − | − |
| 67 | − | − | − |
| 68 | − | + | − |
| 69 | − | + | + |
| 70 | + | + | − |
| 71 | − | − | − |
| 72 | − | − | − |
| 73 | − | + | − |
| 74 | − | + | − |
| 75 | − | − | − |
| 76 | − | − | − |
| 77 | − | − | − |
| 78 | − | − | − |
| 79 | − | − | − |
| 80 | − | − | − |
| 81 | − | − | − |
| 82 | − | − | − |
| 83 | − | − | − |
| 84 | − | − | − |
| 85 | − | − | − |
| 86 | − | − | − |
| 87 | − | − | − |
| 88 | − | − | − |
| 89 | − | − | − |
| 90 | − | − | − |
| 91 | − | − | − |
| 92 | − | − | − |
| 93 | − | − | − |
| 94 | − | − | − |
| 95 | − | − | − |
| 96 | − | − | − |
| 97 | − | − | − |
| 98 | − | − | − |
| 99 | − | − | − |
| 100 | − | − | − |
| 101 | + | − | − |
| 102 | − | − | − |
| 103 | NT | NT | NT |
| 104 | − | − | − |
| 105 | + | − | − |
| 106 | − | − | − |
| 107 | − | − | − |
| 108 | + | + | + |
| 109 | − | + | + |
| 110 | − | − | − |
| 111 | + | + | − |
| 112 | − | + | − |
| 113 | − | + | − |
| 114 | − | − | − |
| 115 | − | + | + |
| 116 | + | − | − |
| 117 | + | − | − |
| 118 | − | − | − |
| 119 | − | − | − |
| 120 | − | − | − |

### In Vivo Assay

In Vivo Anti-Tumor Activity

Acclimated female Balb/C nu/nu mice are injected subcutaneously into the flank with cell pellets of 5×10⁶ HT-29 human colon carcinoma tumor cells (ATCC #HTB-38). The mice are fed sterile rodent chow and water ad libitum, and kept in pathogen-free, sterile-filtered cages with half-day light/dark cycles. Tumors are allowed to grow until they reach an average size of 100-150 mm³, determined by caliper measurement. One day before beginning treatment, mice are randomized by tumor size into evenly distributed cohorts. Mice are dosed once daily by oral gavage (10 ml/kg) with compounds formulated as solutions in Cremaphor EL:Ethanol:Water, at a 1:1:6 ratio. Control mice are dosed with the formulation vehicle only. Tumor size and mouse weight are monitored every three to four days. At the end of the study, treatment efficacy is determined by the T/C value, calculated by dividing the average tumor size of a compound-treated cohort by the average tumor size of the control-treated cohort, expressed as a percentage. Therefore, anti-tumor efficacy is inversely proportional to the T/C.

TABLE 2
In Vivo Activity

| Example # | Dose | T/C (%) | Survival |
|---|---|---|---|
| 34 | 60 mg/kg | 20 | 10/10 |
| 65 | 60 mg/kg | 25 | 10/10 |
| 66 | 60 mg/kg | 11 | 10/10 |
| 87 | 60 mg/kg | 15 | 10/10 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I 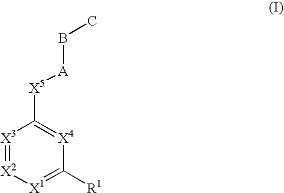
or a salt, ester, or prodrug thereof, wherein:
X¹-X⁴ are each independently selected from the group consisting of C(R²) and N, wherein at least one of X¹-X⁴ are N;
X⁵ is selected from the group consisting of C(R³)(R⁴), N(R³), O and S(O)ₘ;
m is 0, 1 or 2;
A and C are each independently selected from the group consisting of aryl and heteroaryl, either of which may be optionally substituted;
B is selected from the group consisting of —N(H)C(O)N(H)— and N(H)C(O)N(H)CH₂;
R¹ is selected from the group consisting of heteroaryl and heterocycloalkyl, either of which may be optionally substituted;
R² is selected from the group consisting of alkenyl, alkoxy, alkoxyalkyl, alkyl, alkynyl, amido, amino, aminoalkyl, cyano, cyanoalkenyl, ester, ether, halo, haloalkyl, haloalkoxy, hydrogen, hydroxy, hydroxyalkyl and nitro, any of which may be optionally substituted; and
R³ and R⁴ are each independently selected from the group consisting of lower alkyl and hydrogen;
and with the proviso that:
when X¹ is N, X²-X⁴ are each C(R²), X⁵ is O or S and B is —NHC(O)NH—, then A cannot be phenyl unless C is phenyl substituted with —Oⱼ(CH₂)ₖX⁶, wherein X⁶ is heterocycloalkyl;
j is 0 or 1;
k is 1, 2 or 3;
and with the further proviso that when B is —NHC(O)NH— and X⁵ is O, then A cannot be naphthalene; and
and with the further proviso that when B is —NHC(O)NH— and X⁵ is O or N, then C cannot be pyrazole.

2. A compound of any one of structural Formulas II, III or IV: 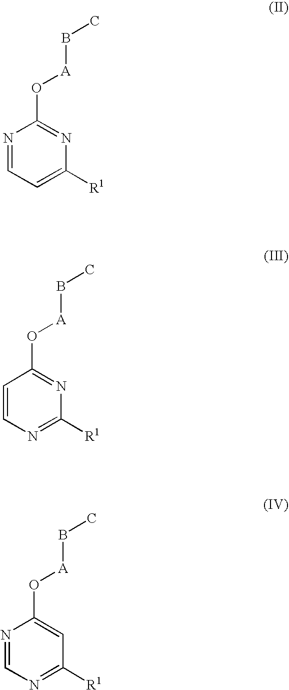
or a salt, ester, or prodrug thereof, wherein:
A is selected from the group consisting of aryl and heteroaryl, either of which may be optionally substituted;
C is selected from the group consisting of aryl and 6-membered heteroaryl, either of which may be optionally substituted;
B is selected from the group consisting of —N(H)C(O)N(H)— and —N(H)C(O)N(H)CH₂—; and
R¹ is selected from the group consisting of heteroaryl and heterocycloalkyl, either of which may be optionally substituted.

3. The compound as recited in claim 2, or a salt, ester, or prodrug thereof, wherein:
A is selected from the group consisting of aryl and heteroaryl, which may be optionally substituted;
C is selected from the group consisting of aryl and 6-membered heteroaryl, which may be optionally substituted;
B is selected from the group consisting of —N(H)C(O)N(H)— and —N(H)C(O)N(H)CH₂—;
R¹ is 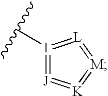
I, J, K, L and M are each independently selected from the group consisting of C(R⁵)(R⁶), S(O)ₙ, O and N(R⁷);
n is 0, 1 or 2;
R⁵ and R⁶ are each independently selected from the group consisting of alkenyl, alkoxy, alkoxyalkyl, alkyl, alkynyl, amido, amidoalkyl, amino, aminoalkyl, aminoalkylamino, cyanoalkyl, cyanoalkenyl, cycloalkyl, ester, esteralkyl, halo, haloalkyl, haloalkoxy, heteroarylalkyl, heterocycloalkenyl, heterocycloalkyl, heterocycloalkylalkyl, heterocycloalkylalkoxy, heterocycloalkylalkylthio, hydrogen, hydroxy, hydroxyalkyl, nitro and null, any of which may be optionally substituted; and
R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted.

4. The compound as recited in claim 3, or a salt, ester, or prodrug thereof, wherein:
R¹ is selected from the group consisting of 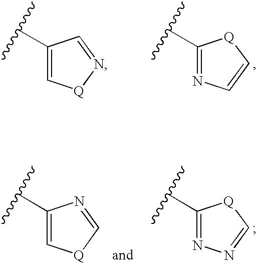
Q is selected from the group consisting of S(O)ₙ, O and N(R⁷); and
n is 0, 1 or 2.

5. The compound as recited in claim 4, or a salt, ester, or prodrug thereof, wherein:
A and C are optionally substituted phenyl;
B is —N(H)C(O)N(H)—;
R¹ is selected from the group consisting of 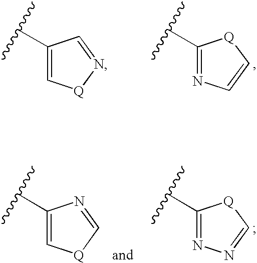
Q is selected from the group consisting of S(O)ₙ, O and N(R⁷);
n is 0, 1 or 2; and
R⁷ is selected from the group consisting of alkyl, alkylamino, amidoalkyl, cyanoalkyl, ester, esteralkyl, haloalkyl, heterocycloalkylalkyl, hydrogen, hydroxyalkyl and null, which may be optionally substituted.

6. The compound as recited in claim 5, or a salt, ester, or prodrug thereof, wherein:
A and C are phenyl optionally substituted with halo or haloalkyl;
B is —N(H)C(O)N(H)—;
R¹ is 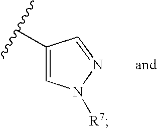
R⁷ is selected from the group consisting of alkyl, alkylamino, amidoalkyl, cyanoalkyl, ester, esteralkyl, haloalkyl, heterocycloalkylalkyl, hydrogen, hydroxyalkyl and null, which may be optionally substituted.

7. A compound of structural Formula V: 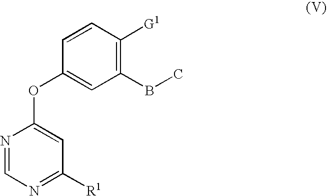
or a salt, ester, or prodrug thereof, wherein:
C is phenyl optionally substituted with halo or haloalkyl;
B is —N(H)C(O)N(H)—;
R¹ is 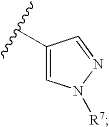
R⁷ is alkyl; and
G¹ is halo.

8. A compound of structural Formula VI 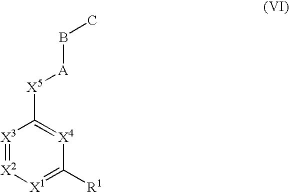
or a salt, ester, or prodrug thereof, wherein:
X¹-X⁴ are each independently selected from the group consisting of C(R²) and N;
X⁵ is selected from the group consisting of C(R³)(R⁴), N(R³), O and S(O)ₘ;
m is 0, 1 or 2;
A and C are each independently selected from the group consisting of aryl and heteroaryl, either of which may be optionally substituted;
B is selected from the group consisting of —N(R⁸)C(O)N(R⁹)— and
—N(R¹⁰)C(O)N(R¹⁰)CH₂—;
R¹ is selected from the group consisting of heteroaryl and heterocycloalkyl, either of which may be optionally substituted;
R² is selected from the group consisting of alkenyl, alkoxy, alkoxyalkyl, alkyl, alkynyl, amido, amino, aminoalkyl, cyano, cyanoalkenyl, ester, ether, halo, haloalkyl, haloalkoxy, hydrogen, hydroxy, hydroxyalkyl, nitro and null, any of which may be optionally substituted;
R³ and R⁴ are each independently selected from the group consisting of lower alkyl and hydrogen;
R⁸ is selected from the group consisting of lower alkyl, cycloalkyl and heterocycloalkyl, any of which may be optionally substituted;
R⁹ is selected from the group consisting of lower alkyl, cycloalkyl, heterocycloalkyl and hydrogen, any of which may be optionally substituted; and
R¹⁰ is selected from the group consisting of lower alkyl, cycloalkyl, heterocycloalkyl and hydrogen, any of which may be optionally substituted.

9. A compound of any one of structural Formulas II, III, IV or VII: 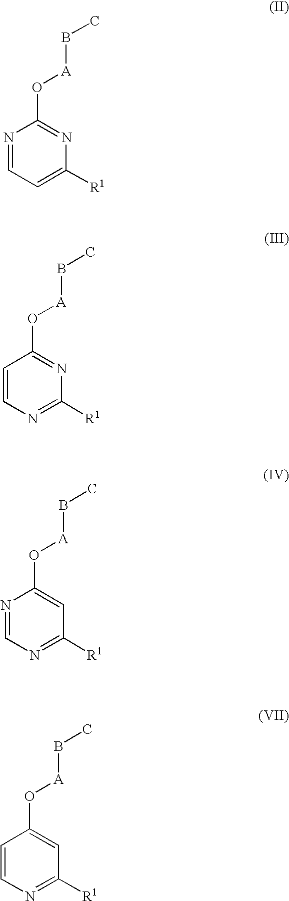
or a salt, ester, or prodrug thereof, wherein:
A and C are each independently selected from the group consisting of aryl and heteroaryl, any of which may be optionally substituted;
B is selected from the group consisting of —N(R⁸)C(O)N(R⁹)— and
—N(R¹⁰)C(O)N(R¹⁰)CH₂—; and
R¹ is selected from the group consisting of heteroaryl and heterocycloalkyl, either of which may be optionally substituted.

10. The compound as recited in claim 9, or a salt, ester, or prodrug thereof, wherein:
R¹ is 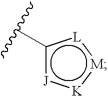
I, J, K, L and M are each independently selected from the group consisting of C(R⁵)(R⁶), S(O)ₙ, O and N(R⁷);
n is 0, 1 or 2;
R⁵ and R⁶ are each independently selected from the group consisting of alkenyl, alkoxy, alkoxyalkyl, alkyl, alkynyl, amido, amidoalkyl, amino, aminoalkyl, aminoalkylamino, cyanoalkyl, cyanoalkenyl, cycloalkyl, ester, esteralkyl, halo, haloalkyl, haloalkoxy, heteroarylalkyl, heterocycloalkenyl, heterocycloalkyl, heterocycloalkylalkyl, heterocycloalkylalkoxy, heterocycloalkylalkylthio, hydrogen, hydroxy, hydroxyalkyl, nitro and null, any of which may be optionally substituted; and
R⁷ is selected from the group consisting of alkenyl, alkoxyalkyl, alkoxycarbonyl, alkyl, alkylamino, alkylene, alkynyl, amidoalkyl, cyanoalkenyl, cyanoalkyl, cycloalkyl, ester, esteralkyl, haloalkyl, haloalkoxy, haloalkylcarbonyl, heteroarylalkyl, heterocycloalkenyl, heterocycloalkyl, heterocycloalkylalkyl, heterocycloalkylalkoxy, heterocycloalkylalkylthio, hydrogen, hydroxyalkyl and null, any of which may be optionally substituted.

11. The compound as recited in claim 10, or a salt, ester, or prodrug thereof, wherein:
R¹ is selected from the group consisting of 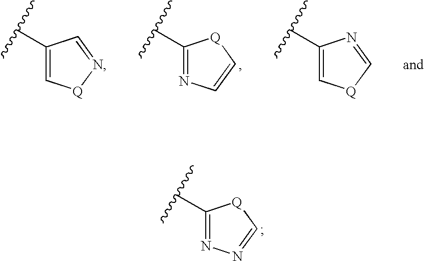
and
Q is selected from the group consisting of S(O)ₙ, O and N(R⁷); and
n is 0, 1 or 2.

12. A compound selected from the group consisting of Examples 1 to 120.

13. A compound as recited in claim 1, or a salt, ester, or prodrug thereof, for use as a medicament.

14. A compound as recited in claim 8, or a salt, ester, or prodrug thereof, for use as a medicament.

15. A compound as recited in claim 1, or a salt, ester, or prodrug thereof, for use in the manufacture of a medicament for the prevention or treatment of a disease or condition ameliorated by the inhibition of protein kinase.

16. A compound as recited in claim 8, or a salt, ester, or prodrug thereof, for use in the manufacture of a medicament for the prevention or treatment of a disease or condition ameliorated by the inhibition of protein kinase.

17. A pharmaceutical composition comprising a compound as recited in claim 1, or a salt, ester, or prodrug thereof, together with a pharmaceutically acceptable carrier.

18. A pharmaceutical composition comprising a compound as recited in claim 8, or a salt, ester, or prodrug thereof, together with a pharmaceutically acceptable carrier.

19. The pharmaceutical composition as recited in claim 17, useful for the treatment or prevention of a protein kinase-mediated disease.

20. The pharmaceutical composition as recited in claim 18, useful for the treatment or prevention of a protein kinase-mediated disease.

21. A method of inhibition of protein kinase comprising contacting a protein kinase with a compound as recited in claim 1.

22. A method of inhibition of protein kinase comprising contacting a protein kinase with a compound as recited in claim 8.

23. A method of treatment of a protein kinase-mediated disease comprising the administration of a therapeutically effective amount of a compound as recited in claim 1, or a salt, ester, or prodrug thereof, to a patient in need thereof.

24. A method of treatment of a protein kinase-mediated disease comprising the administration of a therapeutically effective amount of a compound as recited in claim 8, or a salt, ester, or prodrug thereof, to a patient in need thereof.

25. The method as recited in claim 23 wherein said disease is selected from the group consisting of cancers, hematological and non-hematologic malignancies, autoimmune diseases, hematopoiesis, malignancies of the skin, psoriasis, dry eye and glaucoma.

26. The method as recited in claim 24 wherein said disease is selected from the group consisting of cancers, hematological and non-hematologic malignancies, autoimmune diseases, hematopoiesis, malignancies of the skin, psoriasis, dry eye and glaucoma.

27. A method of treatment of a protein kinase-mediated disease comprising the administration of:
a. a therapeutically effective amount of a compound as recited in claim 1, or a salt, ester, or prodrug thereof; and
b. another therapeutic agent.

28. A method of treatment of a protein kinase-mediated disease comprising the administration of:
a. a therapeutically effective amount of a compound as recited in claim 8, or a salt, ester, or prodrug thereof; and
b. another therapeutic agent.

29. The method as recited in claim 27 wherein said disease is selected from the group consisting of cancers, hematological and non-hematologic malignancies, autoimmune diseases, hematopoiesis, malignancies of the skin, psoriasis, dry eye and glaucoma.

30. The method as recited in claim 28 wherein said disease is selected from the group consisting of cancers, hematological and non-hematologic malignancies, autoimmune diseases, hematopoiesis, malignancies of the skin, psoriasis, dry eye and glaucoma.
